(19) United States
(12) Patent Application Publication
(10) Pub. No.: US 2025/0311624 A1
(43) Pub. Date: Oct. 2, 2025

(54) CONDENSED CYCLIC COMPOUND, LIGHT-EMITTING DEVICE INCLUDING THE SAME, AND ELECTRONIC APPARATUS INCLUDING THE LIGHT-EMITTING DEVICE

(71) Applicants: Samsung Electronics Co., Ltd., Suwon-si (KR); Research & Business Foundation SUNGKYUNKWAN UNIVERSITY, Suwon-si (KR)

(72) Inventors: Sangmo KIM, Suwon-si (KR); Junyeob LEE, Suwon-si (KR); Jihoon KANG, Suwon-si (KR); Joonghyuk KIM, Suwon-si (KR); Junyoung MOON, Suwon-si (KR); Yongsik JUNG, Suwon-si (KR)

(21) Appl. No.: 19/027,913

(22) Filed: Jan. 17, 2025

(30) Foreign Application Priority Data
Mar. 26, 2024 (KR) 10-2024-0041291
Jan. 13, 2025 (KR) 10-2025-0004971

(51) Int. Cl. H10K 85/60; C07D 487/06; C09K 11/06; H10K 50/12; H10K 85/30

(52) U.S. Cl. CPC H10K 85/657; C07D 487/06; C09K 11/06; H10K 85/346; H10K 85/6572; C09K 2211/1007; C09K 2211/1018; H10K 50/121

(57) ABSTRACT

A condensed cyclic compound represented by Formula 1: Formula 1

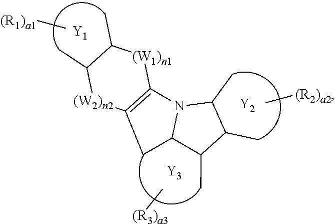

wherein in Formula 1, ring Y₁ to ring Y₃ are each independently a C₅-C₆₀ carbocyclic group or a C₃-C₆₀ heterocyclic group, W₁ is O, S, N(T₁₁), C(T₁₂)(T₁₃), or Si(T₁₂)(T₁₃), W₂ is O, S, N(T₂₁), C(T₂₂)(T₂₃), or Si(T₂₂)(T₂₃), n1 and n2 are each independently 0 or 1, and the sum of n1 and n2 is 1. Formula 1 is the same as described in the detailed description. A light-emitting device including the condensed cyclic compound, and an electronic apparatus including the light-emitting device are also disclosed.

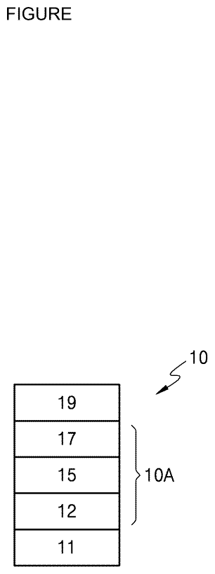

### CROSS-REFERENCE TO RELATED APPLICATION

This application claims priority to Korean Patent Application No. 10-2024-0041291, filed on Mar. 26, 2024, and No. 10-2025-0004917, filed on Jan. 13, 2025, in the Korean Intellectual Property Office, all benefits accruing therefrom under 35 U.S.C. § 119, the entire contents of which are herein incorporated by reference.

### BACKGROUND

### 1. Field

The disclosure relates to a condensed cyclic compound, a light-emitting device including the same, and an electronic apparatus including the light-emitting device.

### 2. Description of the Related Art

Organic light-emitting devices are self-emissive devices that, in comparison to conventional devices, have wide viewing angles, high contrast ratios, short response times, and excellent characteristics in terms of luminance, driving voltage, and response speed, and produce full-color images.

A need remains for luminescent materials for organic light-emitting devices that emit blue light and have a lower driving voltage and improved external quantum efficiency.

### SUMMARY

Provided are a condensed cyclic compound, a light-emitting device employing the same, and an electronic apparatus including the light-emitting device.

Additional aspects will be set forth in part in the description which follows and, in part, will be apparent from the description, or may be learned by practice of the presented embodiments of the disclosure.

According to an aspect of the disclosure, provided is a condensed cyclic compound represented by Formula 1:

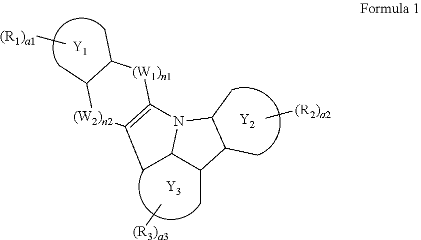

wherein, in Formula 1,
ring Y₁ to ring Y₃ are each independently a C₅-C₆₀ carbocyclic group or a C₃-C₆₀ heterocyclic group,
W₁ is O, S, N(T₁₁), C(T₁₂)(T₁₃), or Si(T₁₂)(T₁₃),
W₂ is O, S, N(T₂₁), C(T₂₂)(T₂₃), or Si(T₂₂)(T₂₃),
n1 and n2 are each independently 0 or 1,
when n1 is 0, *—(W₁)ₙ₁—*′ is a single bond,
when n2 is 0, *—(W₂)ₙ₂—*′ is a single bond,
the sum of n1 and n2 is 1.

R₁ to R₃, T₁₁ to T₁₃, and T₂₁ to T₂₃ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), wherein R₁ in the number of a1, R₂ in the number of a2, and R₃ in the number of a3 may each be identical to or different from each other,
a1 to a3 are each independently an integer from 0 to 30.

Two or more of R₁ to R₃, T₁₁ to T₁₃, and T₂₁ to T₂₃ may optionally be linked to each other to form a substituted or unsubstituted C₅-C₃₀ carbocyclic group or a substituted or unsubstituted C₁-C₃₀ heterocyclic group.

At least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is
deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group,
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof,
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof,
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉), or
any combination thereof, and
Q₁ to Q₉, Q₁₁ to Q₁₉, Q₂₁ to Q₂₉, and Q₃₁ to Q₃₉ are each independently hydrogen, deuterium, —F, or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with deuterium, —F, a cyano group, a C₁-C₆₀ alkyl group, a C₆-C₆₀ aryl group, or any combination thereof.

According to another aspect of the disclosure, a light-emitting device includes a first electrode, a second electrode, and an interlayer arranged between the first electrode and the second electrode. The interlayer includes an emission layer and the interlayer includes at least one condensed cyclic compound described above.

According to another aspect of the disclosure, an electronic apparatus includes the light-emitting device.

### BRIEF DESCRIPTION OF THE DRAWINGS

The above and other aspects, features, and advantages of certain embodiments of the disclosure will be more apparent from the following description taken in conjunction with the accompanying drawings, in which:

FIGURE is a schematic cross-sectional view of an embodiment of a light-emitting device.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the FIGURES, to explain aspects. As used herein, the term “and/or” includes any and all combinations of one or more of the associated listed items. Expressions such as “at least one of,” when preceding a list of elements, modify the entire list of elements and do not modify the individual elements of the list.

It will be understood that when an element is referred to as being “on” another element, it can be directly on the other element or intervening elements may be present therebetween. In contrast, when an element is referred to as being “directly on” another element, there are no intervening elements present.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, “a”, “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the FIGURES. It will be understood that relative terms are intended to encompass different orientations of the device in addition to the orientation depicted in the FIGURES. For example, if the device in one of the FIGURES is turned over, elements described as being on the “lower” side of other elements would then be oriented on “upper” sides of the other elements. The term “lower,” can therefore, encompasses both an orientation of “lower” and “upper,” depending on the particular orientation of the FIGURE. Similarly, if the device in one of the FIGURES is turned over, elements described as “below” or “beneath” other elements would then be oriented “above” the other elements. The terms “below” or “beneath” can, therefore, encompass both an orientation of above and below.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10% or 5% of the stated value.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

Disclosed is a condensed cyclic compound, a light-emitting device including the same, and an electronic apparatus including the light-emitting device. The light-emitting device including the condensed cyclic compound emits blue light and has a lower driving voltage and improved external quantum efficiency than a conventional organic light-emitting device.

In an example, an organic light-emitting device may include an anode, a cathode, and an interlayer that is arranged between the anode and the cathode and includes an emission layer. A hole transport region may be located between the anode and the emission layer, and an electron transport region may be located between the emission layer and the cathode. Holes provided from the anode may move toward the emission layer through the hole transport region, and electrons provided from the cathode may move toward the emission layer through the electron transport region. The holes and the electrons recombine in the emission layer to produce excitons. The excitons may transition from an excited state to a ground state, thus generating light. The disclosed condensed cyclic compound may be included in the emission layer of an organic light-emitting device.

### Description of Condensed Cyclic Compound

A condensed cyclic compound according to the disclosure may be represented by Formula 1:

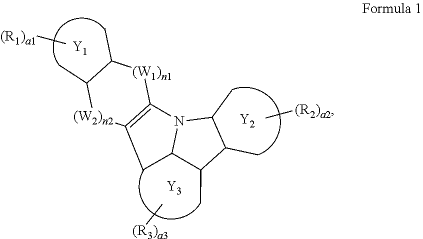

wherein, in Formula 1, ring Y₁ to ring Y₃ may each independently be a C₅-C₆₀ carbocyclic group or a C₃-C₆₀ heterocyclic group.

In an embodiment, ring Y₁ to ring Y₃ may each independently be a benzene group, a naphthalene group, a phenanthrene group, a pyridine group, a pyrimidine group, a pyridazine group, a pyrazine group, a quinoline group, an isoquinoline group, a dibenzofuran group, a dibenzothiophene group, a carbazole group, a fluorene group, a dibenzosilole group, an azadibenzofuran group, an azadibenzothiophene group, an azacarbazole group, an azafluorene group, an azadibenzosilole group, or an indolocarbazole group.

In one or more embodiments, ring Y₁ and ring Y₃ may each independently be a benzene group, a naphthalene group, a phenanthrene group, a dibenzofuran group, a dibenzothiophene group, a carbazole group, a fluorene group, or an indolocarbazole group.

In one or more embodiments, ring Y₂ may be a group represented by Formulae 2-1 to 2-6:

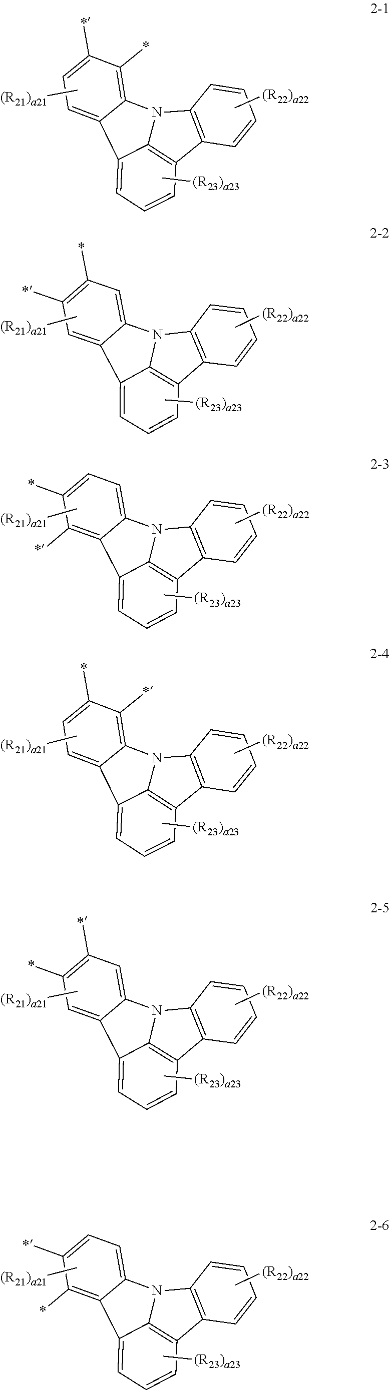

wherein, in Formulae 2-1 to 2-6,
R₂₁ to R₂₃ are each the same as described in connection with R₂,
a21 may be an integer from 0 to 2,
a22 may be an integer from 0 to 4,
a23 may be an integer from 0 to 3,
* indicates a binding site to N in Formula 1, and
*′ indicates a binding site to a neighboring atom in ring Y₃ in Formula 1.

In Formula 1, W₁ may be a single bond, O, S, N(T₁₁), C(T₁₂)(T₁₃), or Si(T₁₂)(T₁₃), and W₂ may be a single bond, O, S, N(T₂₁), C(T₂₂)(T₂₃), or Si(T₂₂)(T₂₃). T₁₁ to T₁₃ and T₂₁ to T₂₃ are the same as described above.

In Formula 1, n1 and n2 may each independently be 0 or 1, and when n1 is 0, *—(W₁)ₙ₁—*′ may not be present, when n2 is 0, *—(W₂)ₙ₂—*′ may not be present, and the sum of n1 and n2 may be 1.

For example, in Formula 1,
i) n1 may be 1, and n2 may be 0, or
ii) n1 may be 0, and n2 may be 1.

In an embodiment, the condensed cyclic compound may satisfy Condition 1A or

### Condition 1B:

Condition 1A

### n1 is 1, n2 is 0, and W₁ is O or S; or

Condition 1B

### n1 is 0, n2 is 1, and W₂ is O or S.

In an embodiment, the condensed cyclic compound may satisfy at least one of Conditions 1C to 1E:

### Condition 1C

at least one of O and S is included;

### Condition 1D

at least one of O and S is included, and simultaneously, a tert-butyl group is included; and

### Condition 1E

at least one of O and S is included, and simultaneously, a carbazolyl group is included.

In Formula 1, R₁ to R₃, T₁₁ to T₁₃, and T₂₁ to T₂₃ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), wherein R₁ in the number of a1, R₂ in the number of a2, and R₃ in the number of a3 may each be identical to or different from each other.

In one or more embodiments, R₁ to R₃, T₁₁ to T₁₃, and T₂₁ to T₂₃ may each independently be:
hydrogen, deuterium, —F, or a cyano group;
a C₁-C₂₀ alkyl group, a phenyl group, a biphenyl group, a dibenzofuranyl group, a dibenzothiophenyl group, a carbazolyl group, a fluorenyl group, or a dibenzosilolyl group, each unsubstituted or substituted with deuterium, —F, a cyano group, a C₁-C₂₀ alkyl group, a phenyl group, a biphenyl group, a dibenzofuranyl group, a dibenzothiophenyl group, a carbazolyl group, a fluorenyl group, a dibenzosilolyl group, —N(Q₁)(Q₂), or any combination thereof; or
—N(Q₁)(Q₂), and
Q₁ and Q₂ may each independently be a C₁-C₂₀ alkyl group, a phenyl group, a biphenyl group, a dibenzofuranyl group, a dibenzothiophenyl group, a carbazolyl group, a fluorenyl group, or a dibenzosilolyl group, each unsubstituted or substituted with deuterium, —F, a cyano group, a C₁-C₂₀ alkyl group, a phenyl group, a dibenzofuranyl group, a dibenzothiophenyl group, a carbazolyl group, a fluorenyl group, a dibenzosilolyl group, or any combination thereof.

In one or more embodiments, R₁ to R₃, T₁₁ to T₁₃, and T₂₁ to T₂₃ may each independently be:
hydrogen, deuterium, —F, or a cyano group;
a C₁-C₂₀ alkyl group unsubstituted or substituted with deuterium, —F, a cyano group, or any combination thereof;
a phenyl group unsubstituted or substituted with deuterium, —F, a cyano group, a C₁-C₂₀ alkyl group, a phenyl group, —N(Q₁)(Q₂), or any combination thereof; or
a carbazolyl group unsubstituted or substituted with deuterium, —F, a cyano group, a C₁-C₂₀ alkyl group, a phenyl group, a biphenyl group, or any combination thereof, and
Q₁ and Q₂ may each independently be a C₁-C₂₀ alkyl group, a phenyl group, a biphenyl group, a dibenzofuranyl group, a dibenzothiophenyl group, a carbazolyl group, a fluorenyl group, or a dibenzosilolyl group, each unsubstituted or substituted with deuterium, —F, a cyano group, a C₁-C₂₀ alkyl group, a phenyl group, a dibenzofuranyl group, a dibenzothiophenyl group, a carbazolyl group, a fluorenyl group, a dibenzosilolyl group, or any combination thereof.

In one or more embodiments, R₁ to R₃, T₁₁ to T₁₃, and T₂₁ to T₂₃ may each independently be:
hydrogen, deuterium, —F, or a cyano group;
a methyl group, an ethyl group, an n-butyl group, a sec-butyl group, or a tert-butyl group, each unsubstituted or substituted with deuterium, —F, a cyano group, or any combination thereof;
a phenyl group unsubstituted or substituted with deuterium, —F, a cyano group, a methyl group, an ethyl group, an n-butyl group, a sec-butyl group, a tert-butyl group, a phenyl group, —N(Q₁)(Q₂), or any combination thereof; or
a carbazolyl group unsubstituted or substituted with deuterium, —F, a cyano group, a methyl group, an ethyl group, an n-butyl group, a sec-butyl group, a tert-butyl group, a phenyl group, a biphenyl group, or any combination thereof, and
Q₁ and Q₂ may each independently be a C₁-C₂₀ alkyl group, a phenyl group, a biphenyl group, a dibenzofuranyl group, a dibenzothiophenyl group, a carbazolyl group, a fluorenyl group, or a dibenzosilolyl group, each unsubstituted or substituted with deuterium, —F, a cyano group, a C₁-C₂₀ alkyl group, a phenyl group, a dibenzofuranyl group, a dibenzothiophenyl group, a carbazolyl group, a fluorenyl group, a dibenzosilolyl group, or any combination thereof.

In Formula 1, a1 to a3 may each independently be an integer from 0 to 30.

In Formula 1, a1 to a3 indicate the numbers of R₁ to R₃, respectively and when a1 is 2 or more, two or more of R₁ may be identical to or different from each other, when a2 is 2 or more, two or more of R₂ may be identical to or different from each other, and when a3 is 2 or more, two or more of R₃ may be identical to or different from each other.

In Formula 1, two or more of R₁ to R₃, T₁₁ to T₁₃, and T₂₁ to T₂₃ may optionally be linked to each other to form a substituted or unsubstituted C₅-C₃₀ carbocyclic group or a substituted or unsubstituted C₁-C₃₀ heterocyclic group.

In the description of Formula 1, at least one substituent of each of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
any combination thereof, and
Q₁ to Q₉, Q₁₁ to Q₁₉, Q₂₁ to Q₂₉, and Q₃₁ to Q₃₉ may each independently be hydrogen, deuterium, —F, or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with deuterium, —F, a cyano group, a C₁-C₆₀ alkyl group, a C₆-C₆₀ aryl group, or any combination thereof.

In an embodiment, the condensed cyclic compound may be represented by, for example, any one of Formulae 3A and 3B:

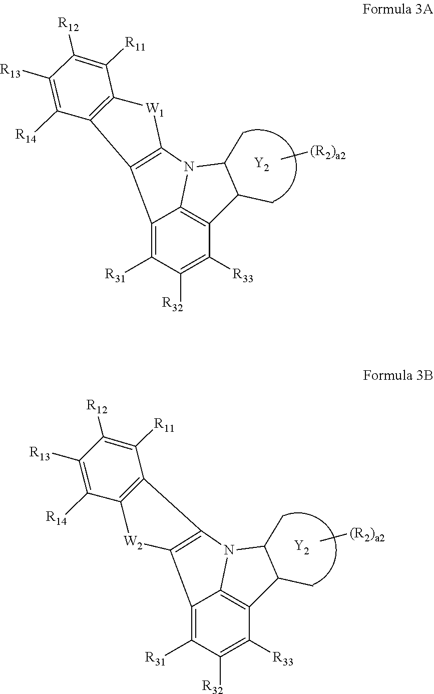

wherein, in Formulae 3A and 3B,
W₁, W₂, Y₂, R₂, and a2 are each the same as described above,
R₁₁ to R₁₄ are each the same as described in connection with R₁, and
R₃₁ to R₃₃ are each the same as described in connection with R₃.

In an embodiment, the condensed cyclic compound may be represented by any one of Formulae 3-1 to 3-12:

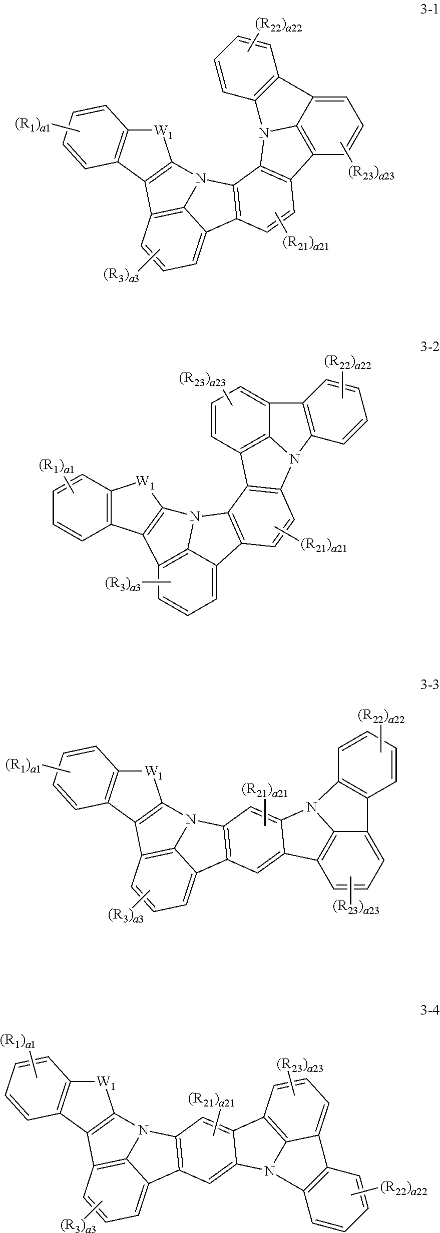

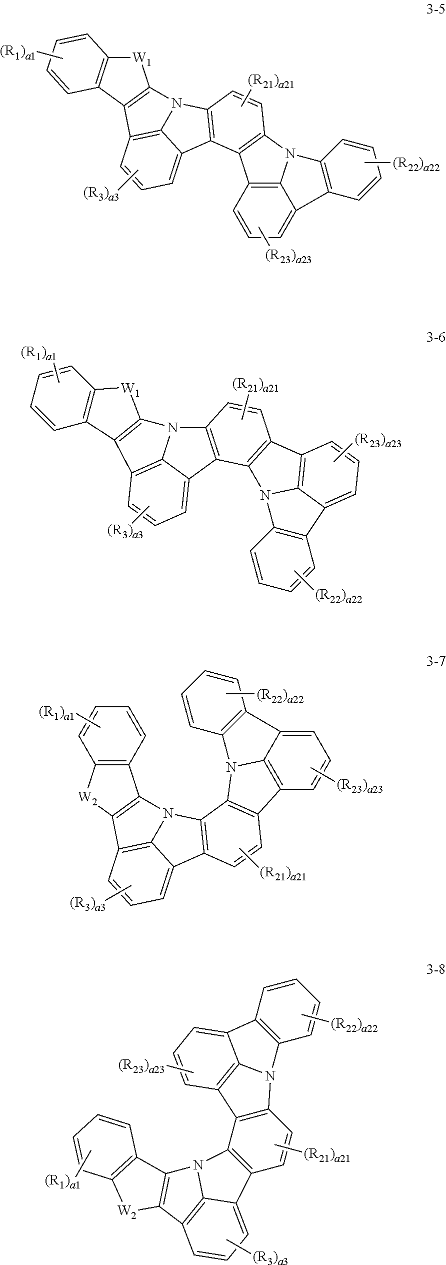

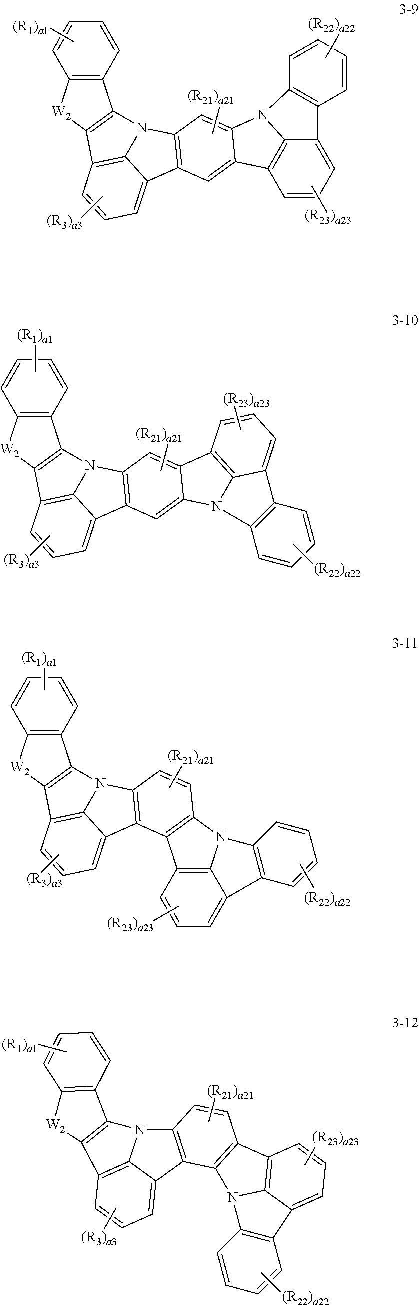

wherein, in Formulae 3-1 to 3-12,
W₁, W₂, R₁, R₃, a1, and a3 are each the same as described above,
R₂₁ to R₂₃ are each the same as described in connection with R₂,
a21 may be an integer from 0 to 2,
a22 may be an integer from 0 to 4, and
a23 may be an integer from 0 to 3.

In an embodiment, the condensed cyclic compound represented by Formula 1 may include:
i) at least one deuterium;
ii) at least one tert-butyl group;
iii) at least one deuterium and at least one tert-butyl group; or
iv) at least one carbazolyl group.

In one or more embodiments, the condensed cyclic compound represented by Formula 1 may have a symmetrical structure.

In one or more embodiments, the condensed cyclic compound represented by Formula 1 may have an asymmetrical structure.

In one or more embodiments, the condensed cyclic compound may be a multiple resonance thermally activated delayed fluorescence material.

In one or more embodiments, the condensed cyclic compound may be represented by one of Compounds 1 to 172:

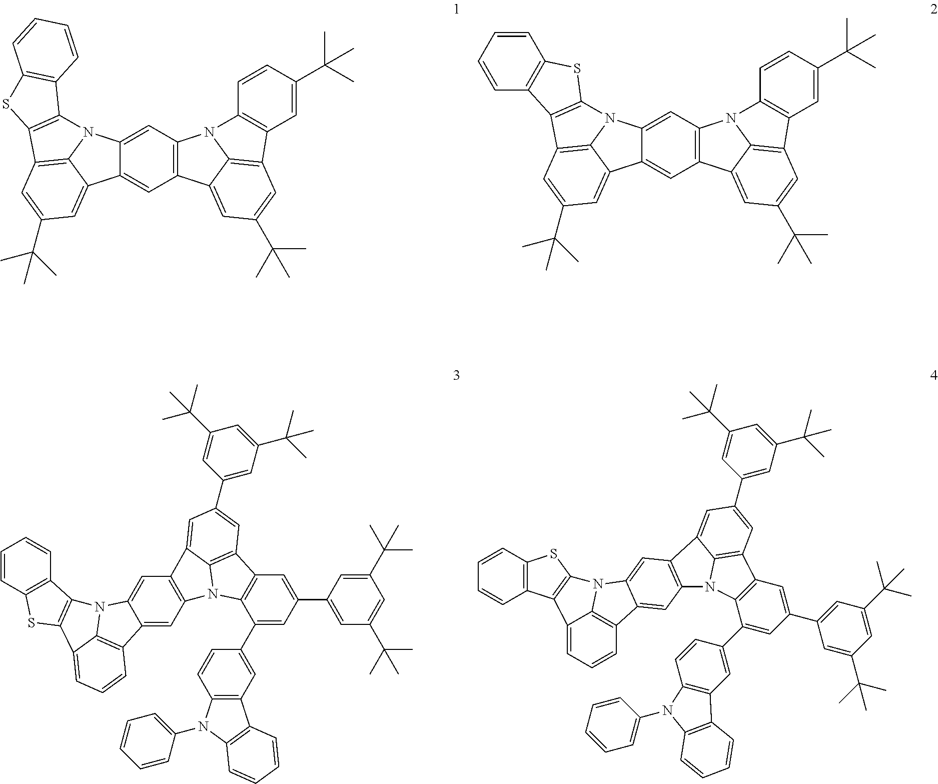 [SMILES: CC(C)(C)c1ccc2c(c1)c1cc(C(C)(C)C)cc3c4cc5c6cc(C(C)(C)C)cc7c8sc9ccccc9c8n(c5cc4n2c13)c67 ; CC(C)(C)c1ccc2c(c1)c1cc(C(C)(C)C)cc3c4cc5c6cc(C(C)(C)C)cc7c8c9ccccc9sc8n(c5cc4n2c13)c67 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4sc5ccccc5c4n6c23)cc(C(C)(C)C)c1 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4c5ccccc5sc4n6c23)cc(C(C)(C)C)c1]

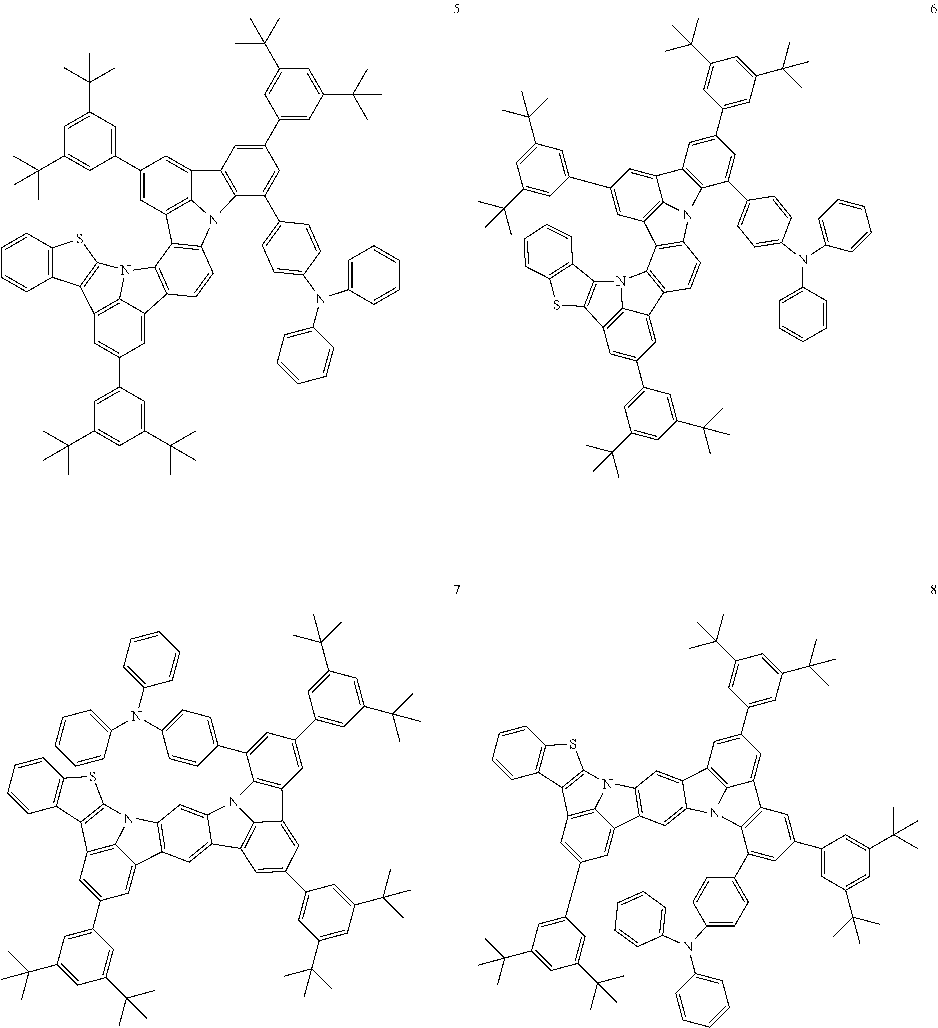

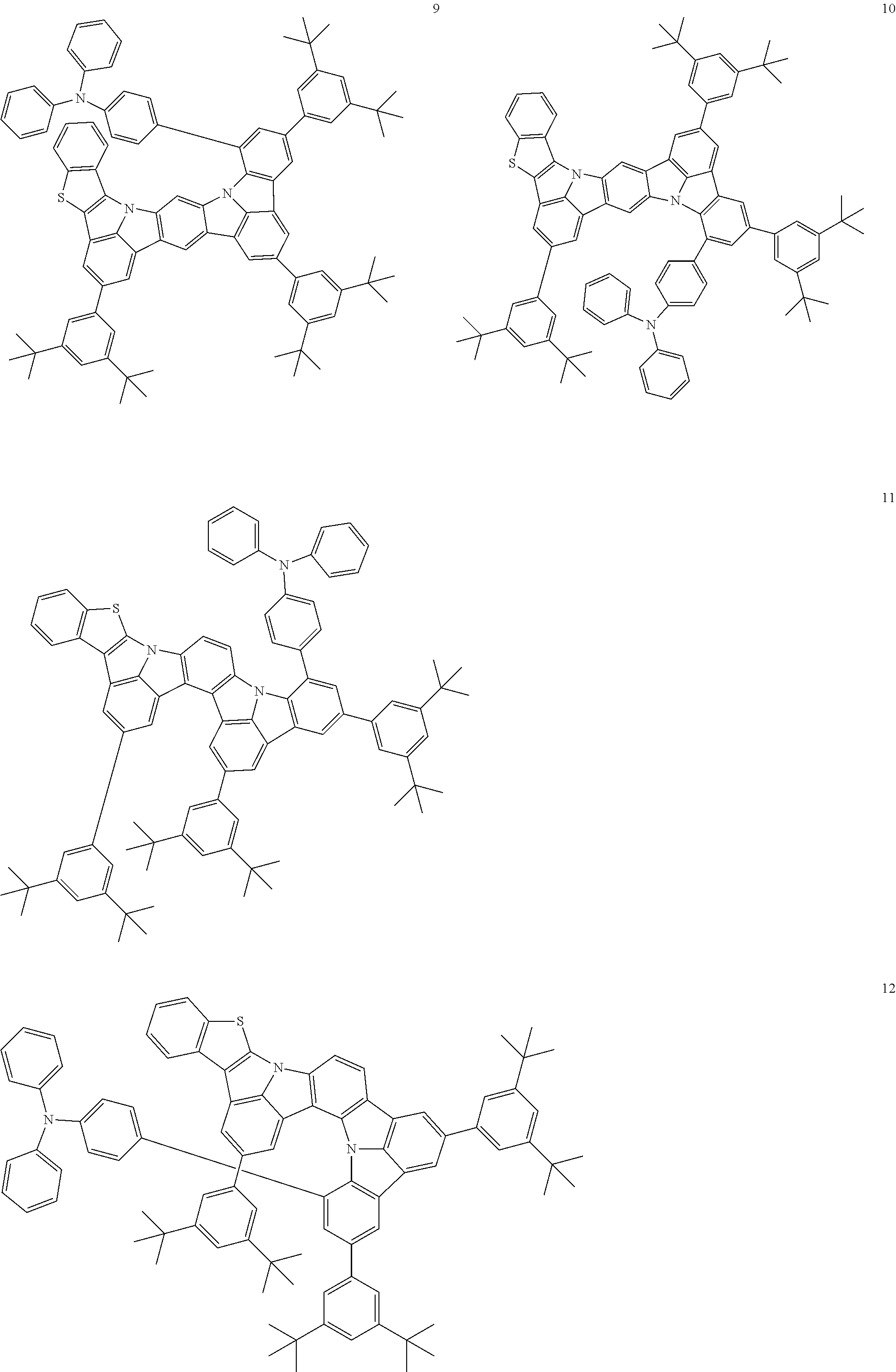

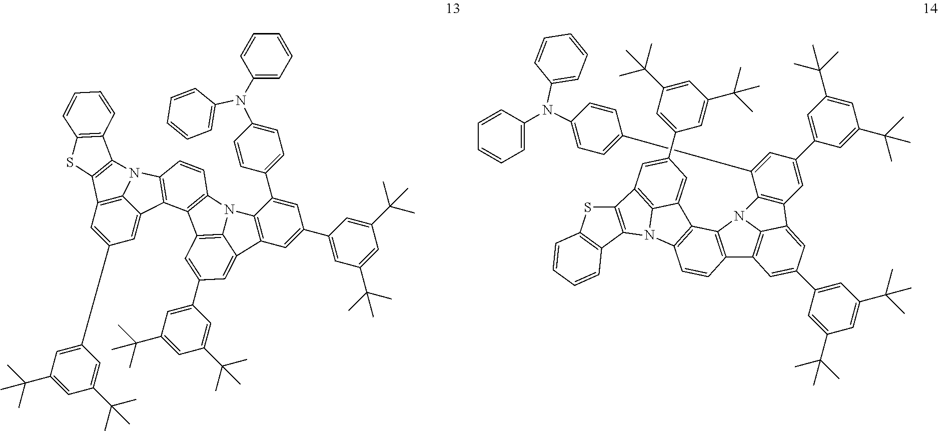 [SMILES: CC(C)(C)c1cc(-c2cc(-c3ccc(N(c4ccccc4)c4ccccc4)cc3)c3c(c2)c2cc(-c4cc(C(C)(C)C)cc(C(C)(C)C)c4)cc4c5c6c7cc(-c8cc(C(C)(C)C)cc(C(C)(C)C)c8)cc8c9sc%10ccccc%10c9n(c6ccc5n3c24)c87)cc(C(C)(C)C)c1 ; CC(C)(C)c1cc(-c2cc(-c3ccc(N(c4ccccc4)c4ccccc4)cc3)c3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9sc%10ccccc%10c9n6c87)c5n3c24)cc(C(C)(C)C)c1]

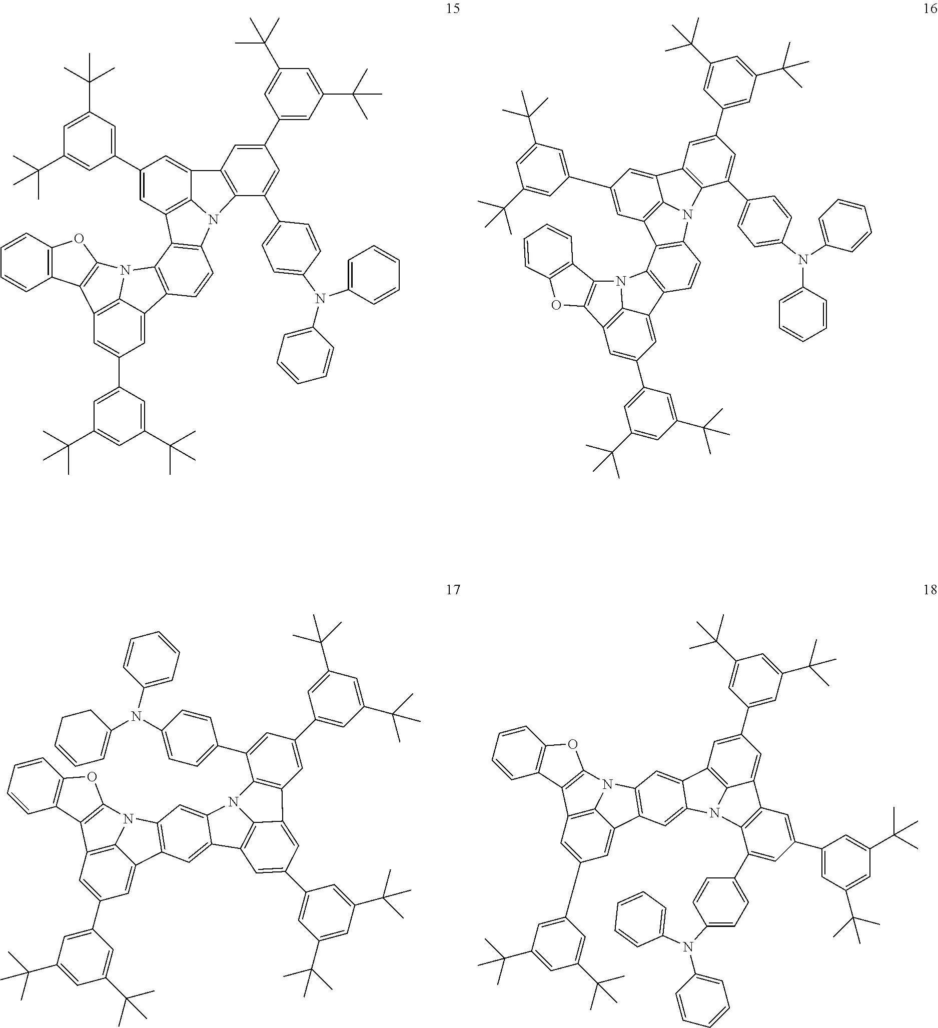

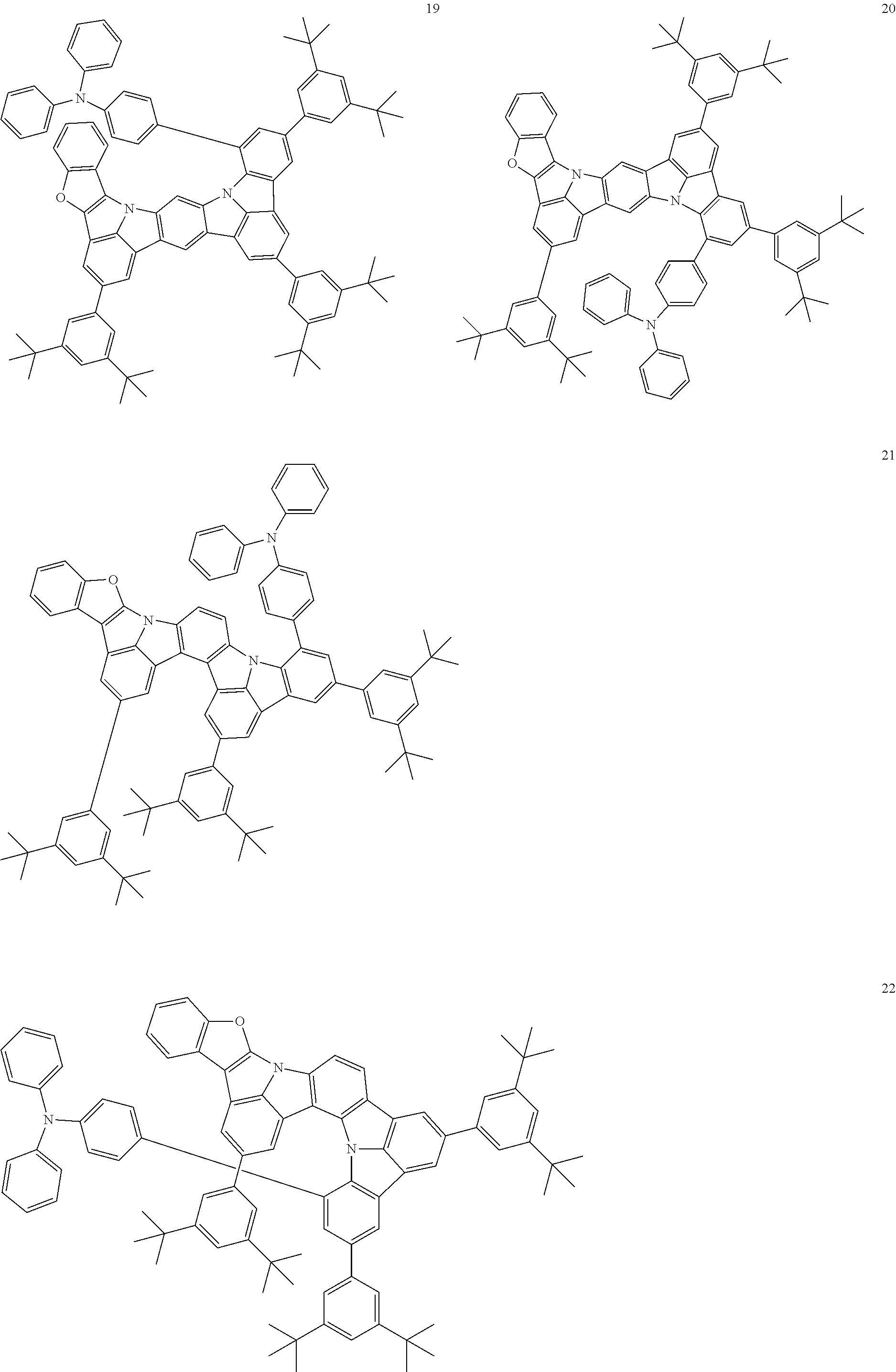

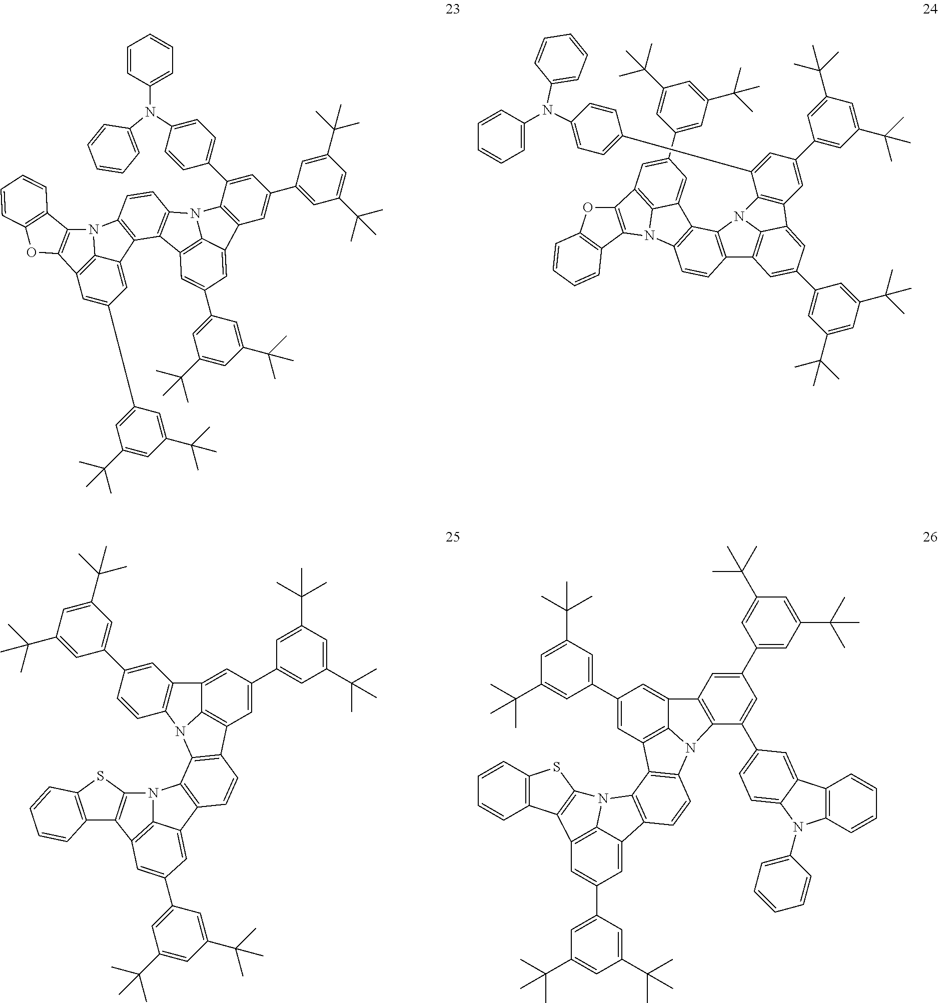

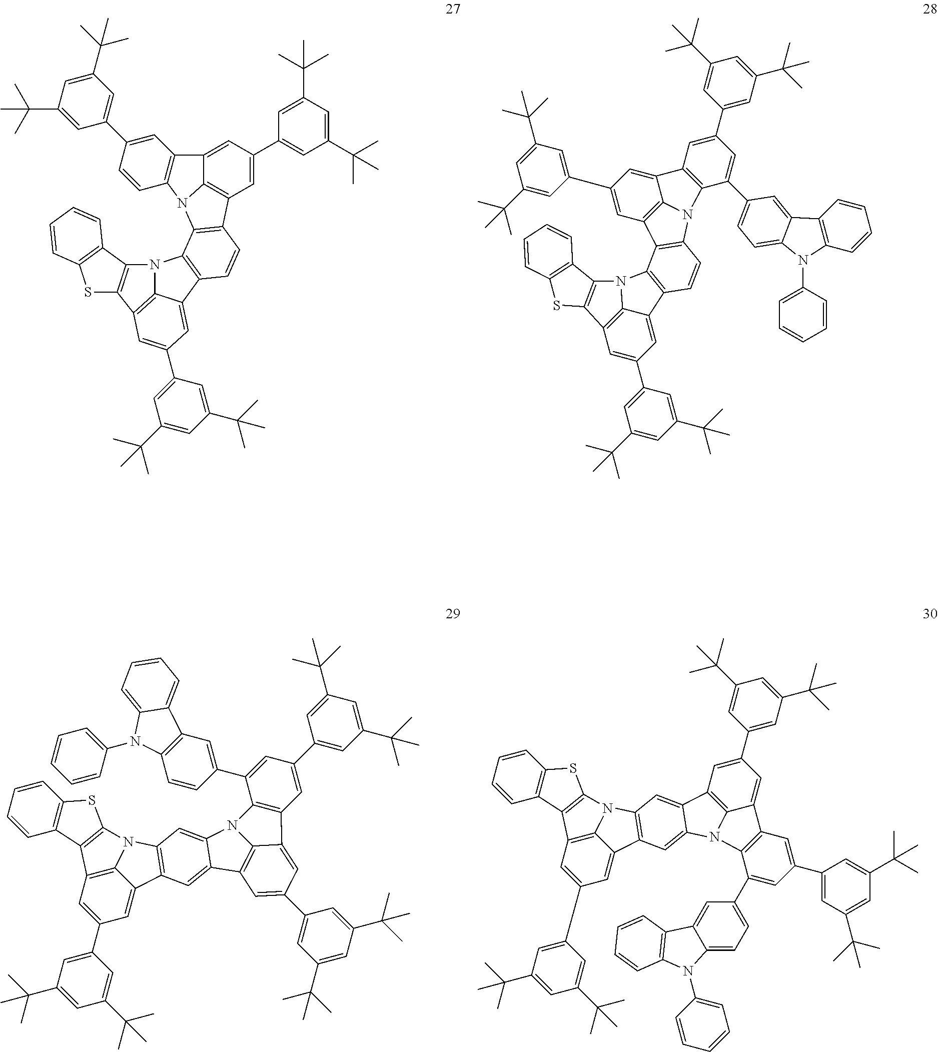

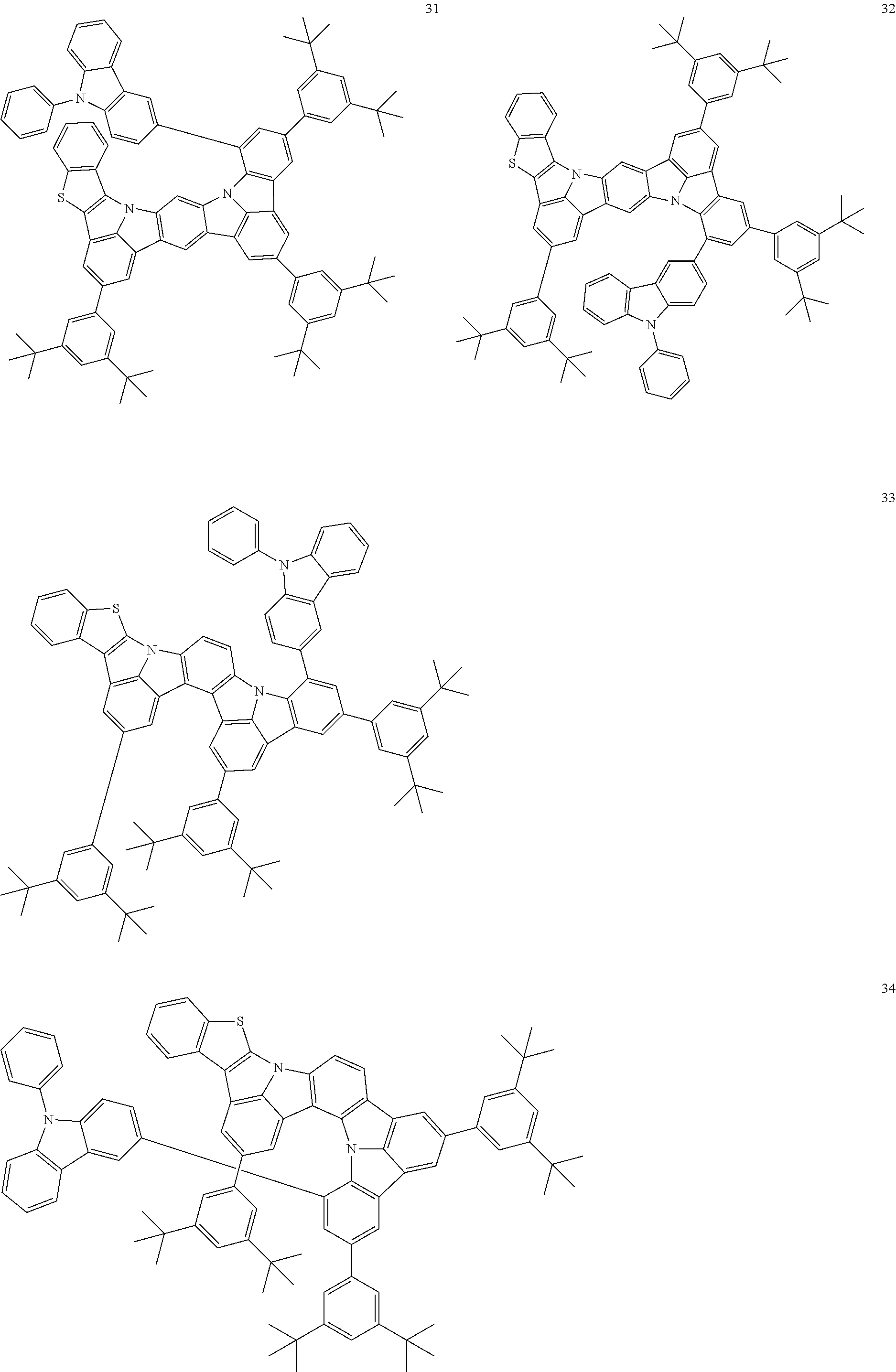

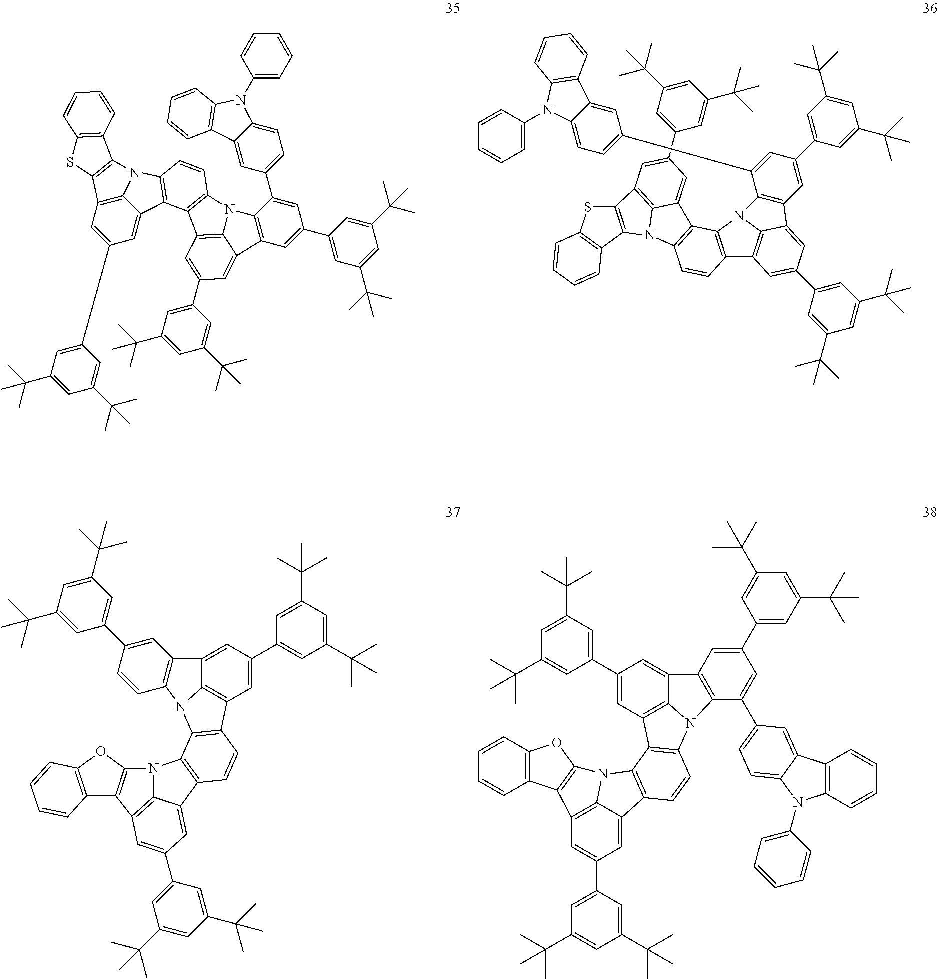

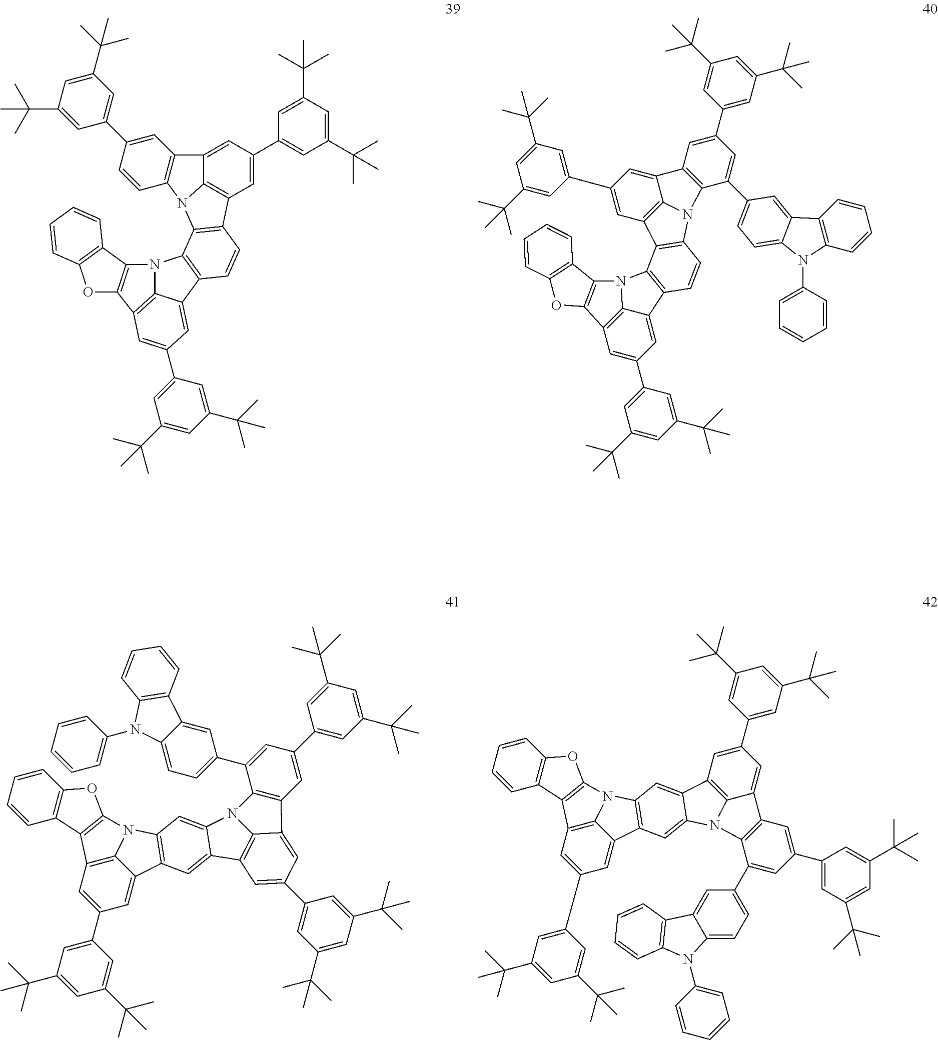

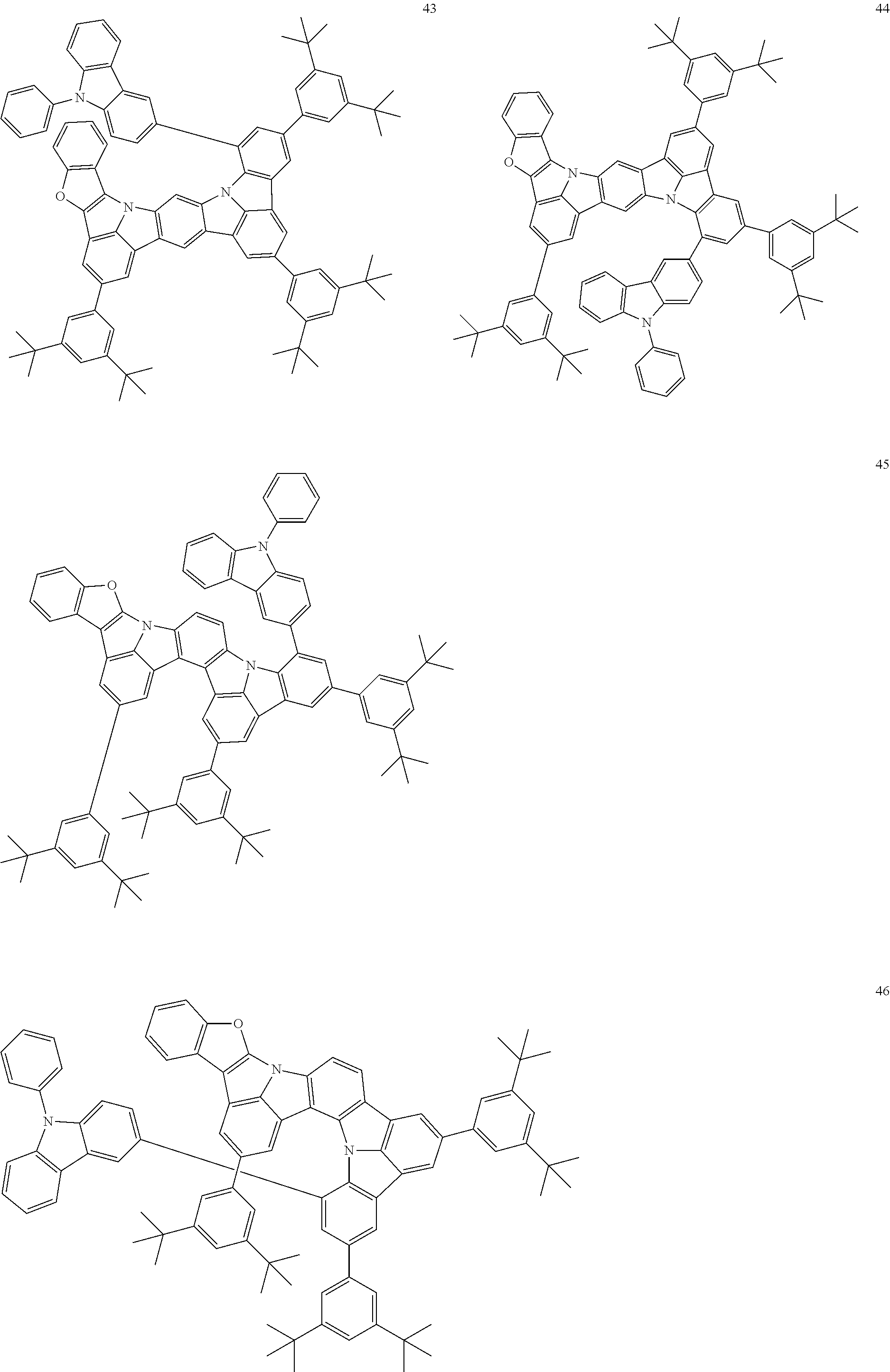

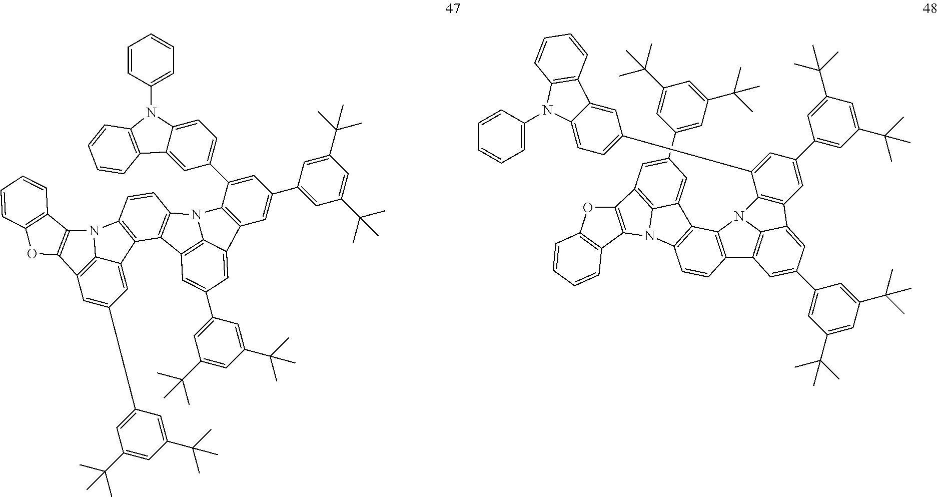 [SMILES: CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5c6c7cc(-c8cc(C(C)(C)C)cc(C(C)(C)C)c8)cc8c9oc%10ccccc%10c9n(c6ccc5n3c24)c87)cc(C(C)(C)C)c1 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9oc%10ccccc%10c9n6c87)c5n3c24)cc(C(C)(C)C)c1]

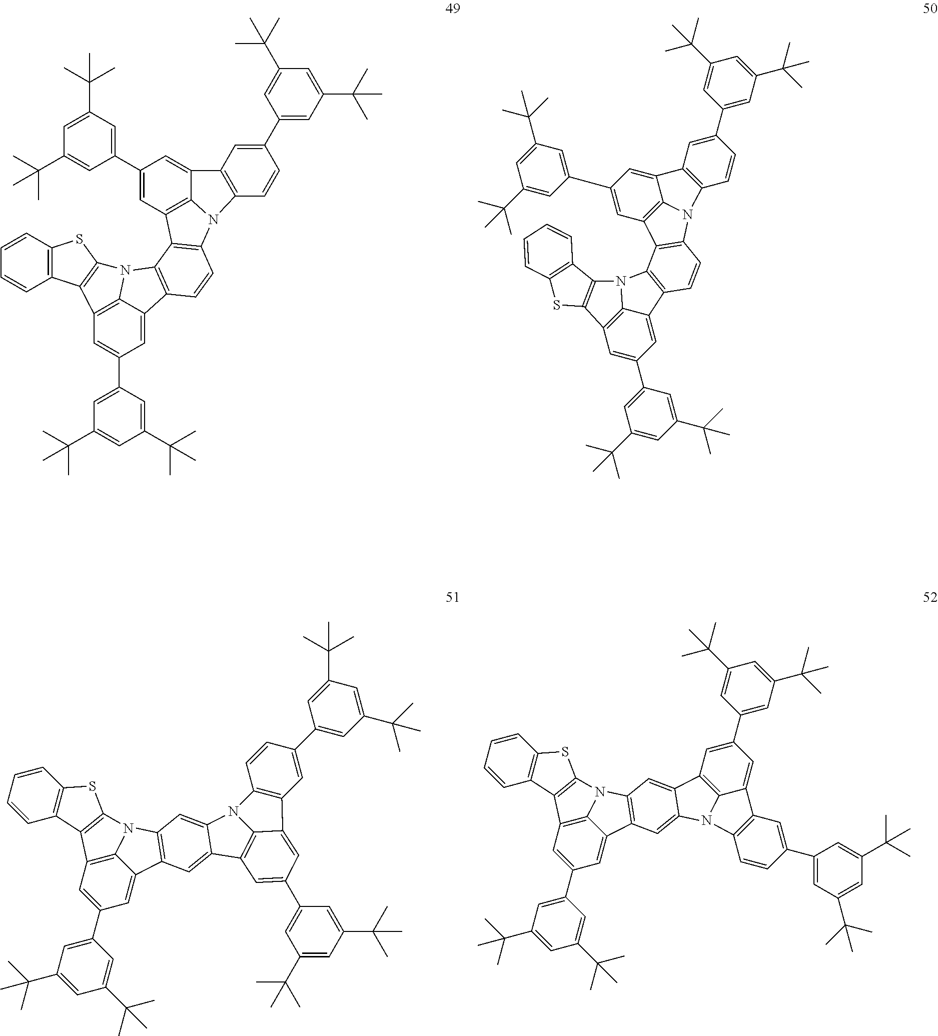

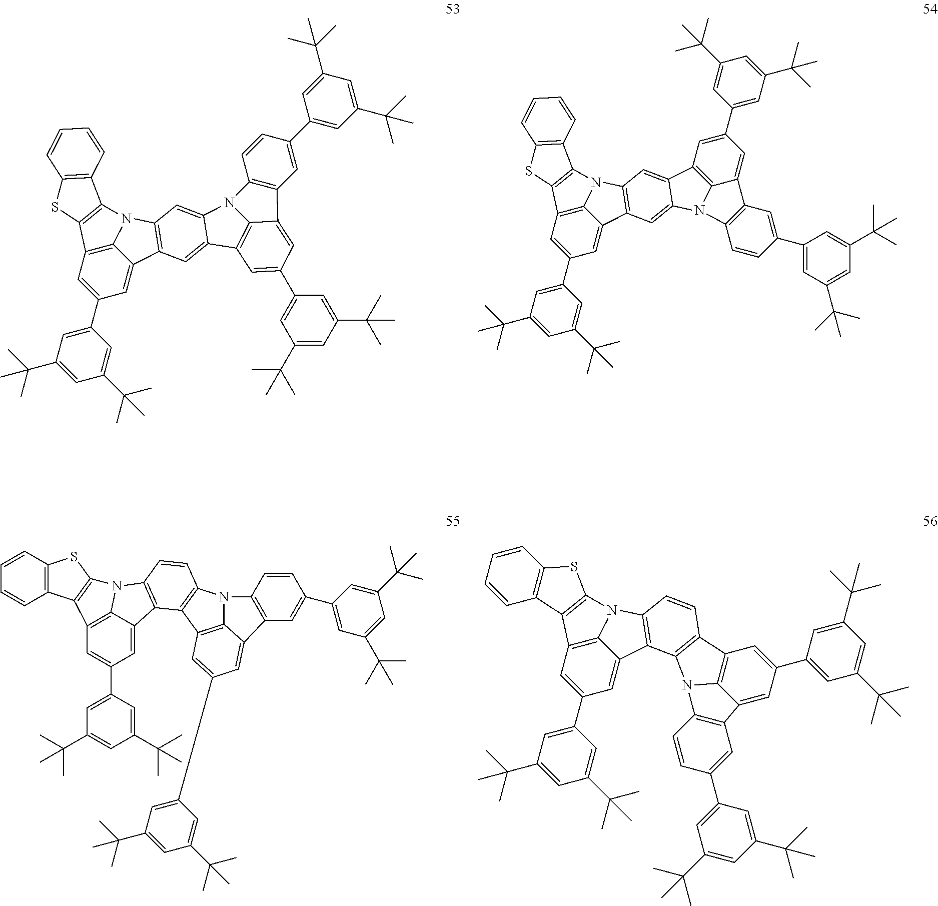

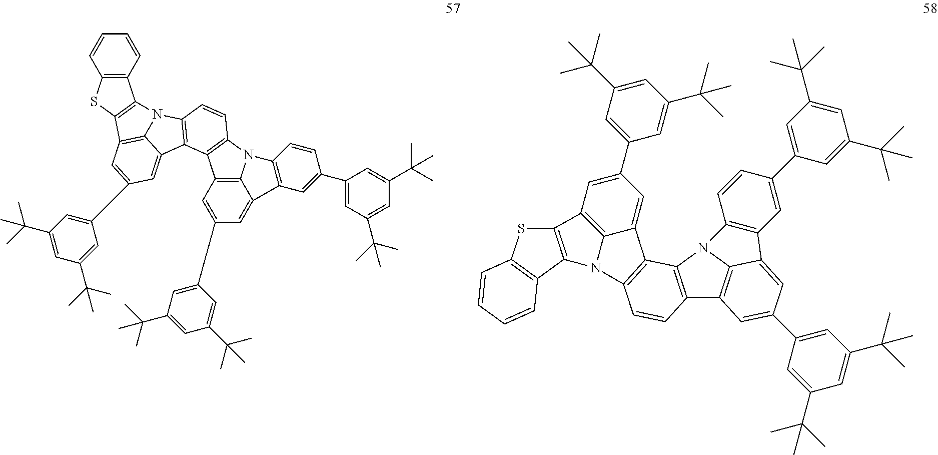 [SMILES: CC(C)(C)c1cc(-c2ccc3c(c2)c2cc(-c4cc(C(C)(C)C)cc(C(C)(C)C)c4)cc4c5c6c7cc(-c8cc(C(C)(C)C)cc(C(C)(C)C)c8)cc8c9sc%10ccccc%10c9n(c6ccc5n3c24)c87)cc(C(C)(C)C)c1 ; CC(C)(C)c1cc(-c2ccc3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9sc%10ccccc%10c9n6c87)c5n3c24)cc(C(C)(C)C)c1]

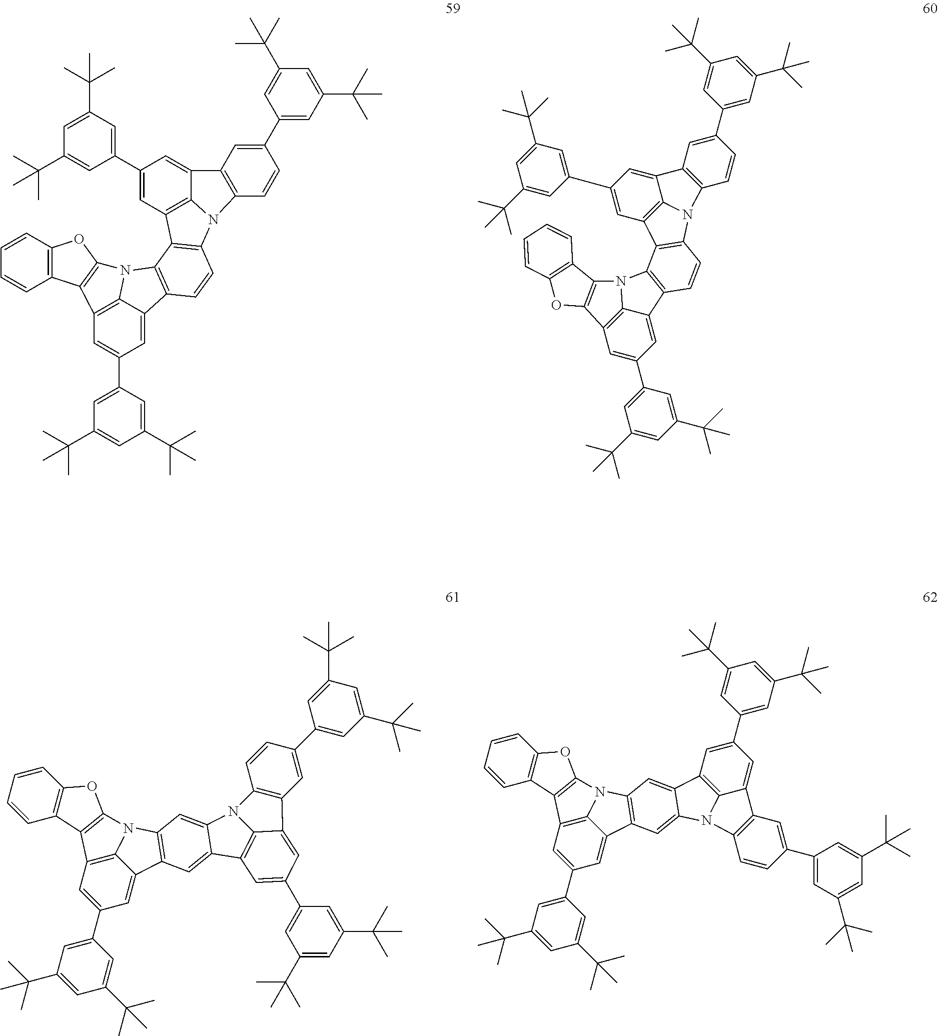

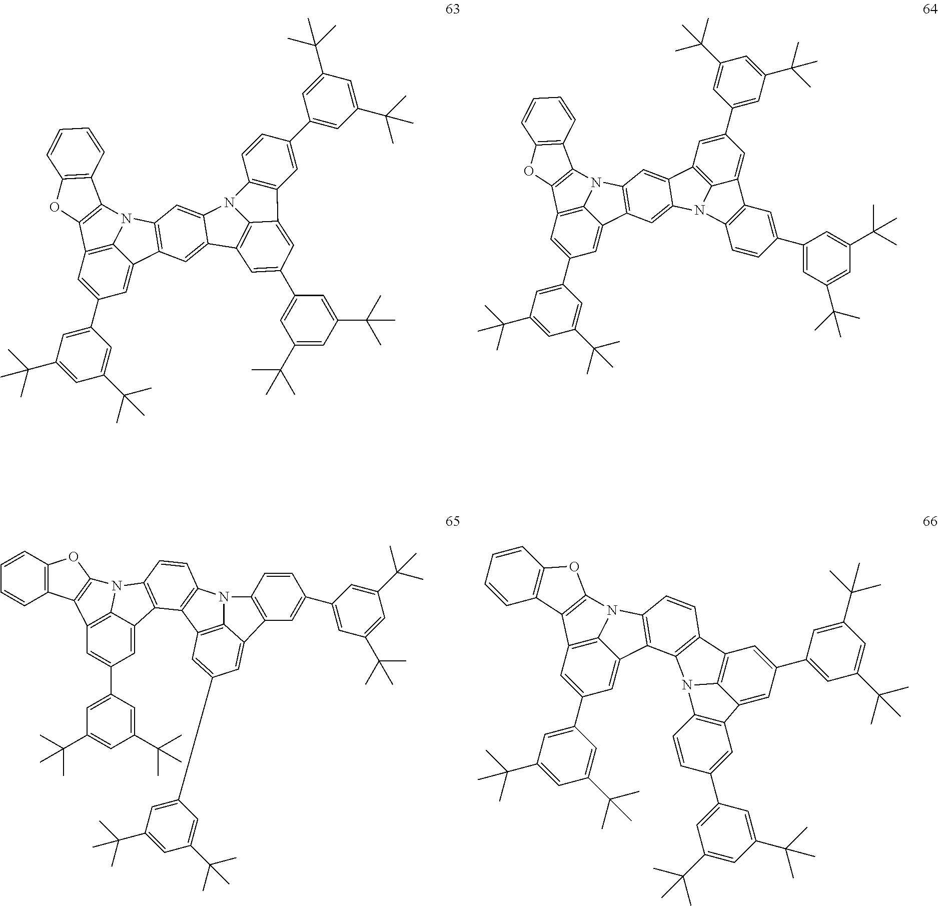

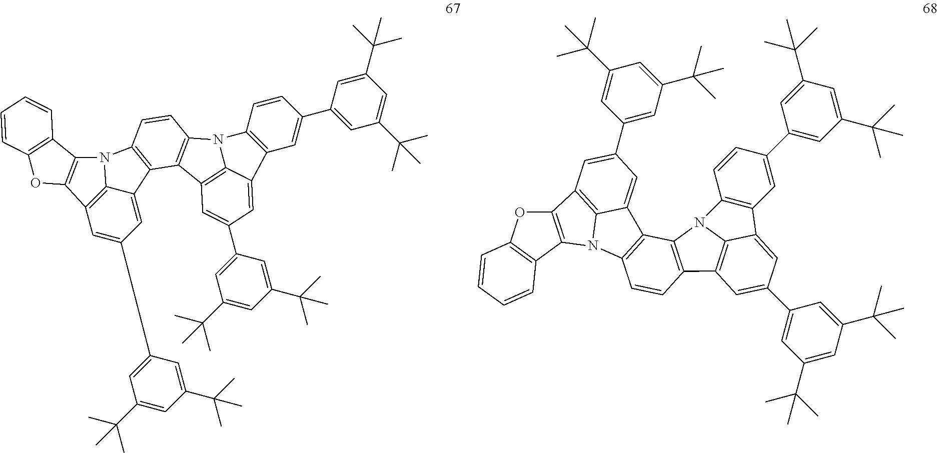 [SMILES: CC(C)(C)c1cc(-c2ccc3c(c2)c2cc(-c4cc(C(C)(C)C)cc(C(C)(C)C)c4)cc4c5c6c7cc(-c8cc(C(C)(C)C)cc(C(C)(C)C)c8)cc8c9oc%10ccccc%10c9n(c6ccc5n3c24)c87)cc(C(C)(C)C)c1 ; CC(C)(C)c1cc(-c2ccc3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9oc%10ccccc%10c9n6c87)c5n3c24)cc(C(C)(C)C)c1]

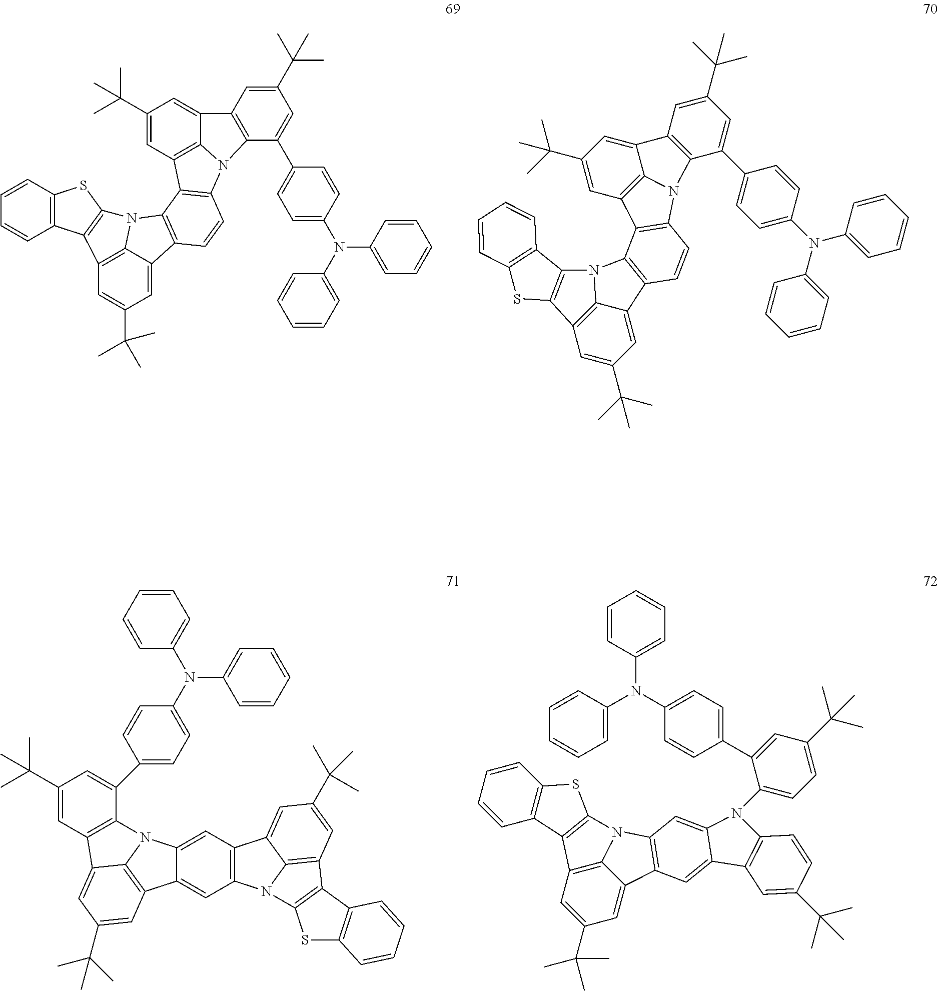 [SMILES: CC(C)(C)c1cc(-c2ccc(N(c3ccccc3)c3ccccc3)cc2)c2c(c1)c1cc(C(C)(C)C)cc3c4c(ccc5c6cc(C(C)(C)C)cc7c8c9ccccc9sc8n(c67)c54)n2c13 ; CC(C)(C)c1cc(-c2ccc(N(c3ccccc3)c3ccccc3)cc2)c2c(c1)c1cc(C(C)(C)C)cc3c4c(ccc5c6cc(C(C)(C)C)cc7c8sc9ccccc9c8n(c67)c54)n2c13 ; CC(C)(C)c1cc(-c2ccc(N(c3ccccc3)c3ccccc3)cc2)c2c(c1)c1cc(C(C)(C)C)cc3c4cc5c(cc4n2c31)c1cc(C(C)(C)C)cc2c3c4ccccc4sc3n5c12 ; CC(C)(C)c1ccc(-n2c3ccc(C(C)(C)C)cc3c3cc4c5cc(C(C)(C)C)cc6c7c8ccccc8sc7n(c4cc32)c56)c(-c2ccc(N(c3ccccc3)c3ccccc3)cc2)c1]

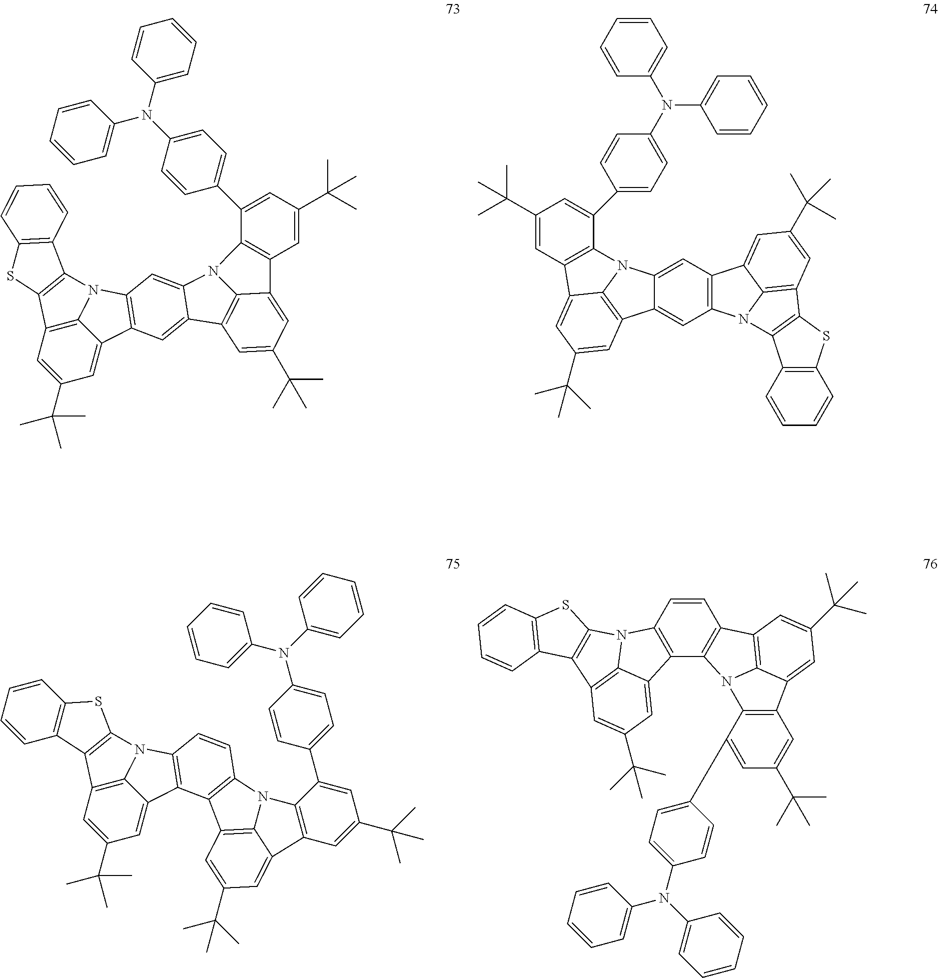 [SMILES: CC(C)(C)c1cc(-c2ccc(N(c3ccccc3)c3ccccc3)cc2)c2c(c1)c1cc(C(C)(C)C)cc3c4cc5c6cc(C(C)(C)C)cc7c8sc9ccccc9c8n(c5cc4n2c31)c67 ; CC(C)(C)c1cc(-c2ccc(N(c3ccccc3)c3ccccc3)cc2)c2c(c1)c1cc(C(C)(C)C)cc3c4cc5c(cc4n2c31)c1cc(C(C)(C)C)cc2c3sc4ccccc4c3n5c12 ; CC(C)(C)c1cc(-c2ccc(N(c3ccccc3)c3ccccc3)cc2)c2c(c1)c1cc(C(C)(C)C)cc3c4c5c6cc(C(C)(C)C)cc7c8c9ccccc9sc8n(c5ccc4n2c13)c67 ; CC(C)(C)c1cc(-c2ccc(N(c3ccccc3)c3ccccc3)cc2)c2c(c1)c1cc(C(C)(C)C)cc3c4ccc5c(c6cc(C(C)(C)C)cc7c8c9ccccc9sc8n5c76)c4n2c13]

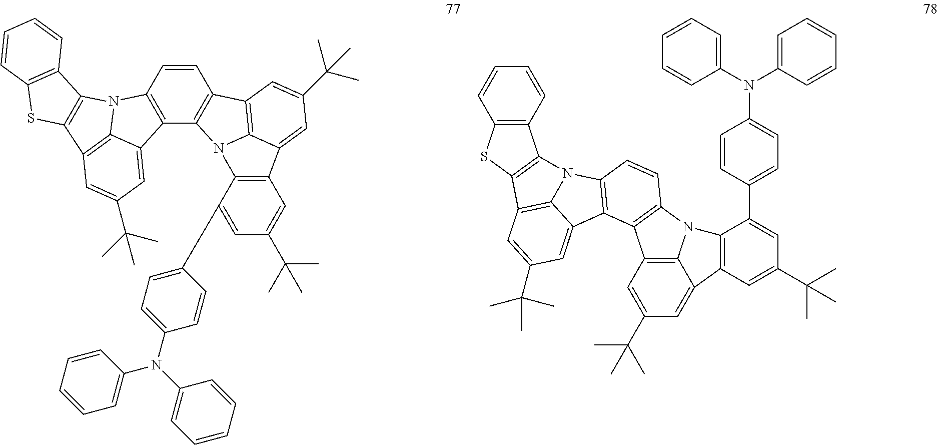 [SMILES: CC(C)(C)c1cc(-c2ccc(N(c3ccccc3)c3ccccc3)cc2)c2c(c1)c1cc(C(C)(C)C)cc3c4ccc5c(c6cc(C(C)(C)C)cc7c8sc9ccccc9c8n5c76)c4n2c13 ; CC(C)(C)c1cc(-c2ccc(N(c3ccccc3)c3ccccc3)cc2)c2c(c1)c1cc(C(C)(C)C)cc3c4c5c6cc(C(C)(C)C)cc7c8sc9ccccc9c8n(c5ccc4n2c13)c76]

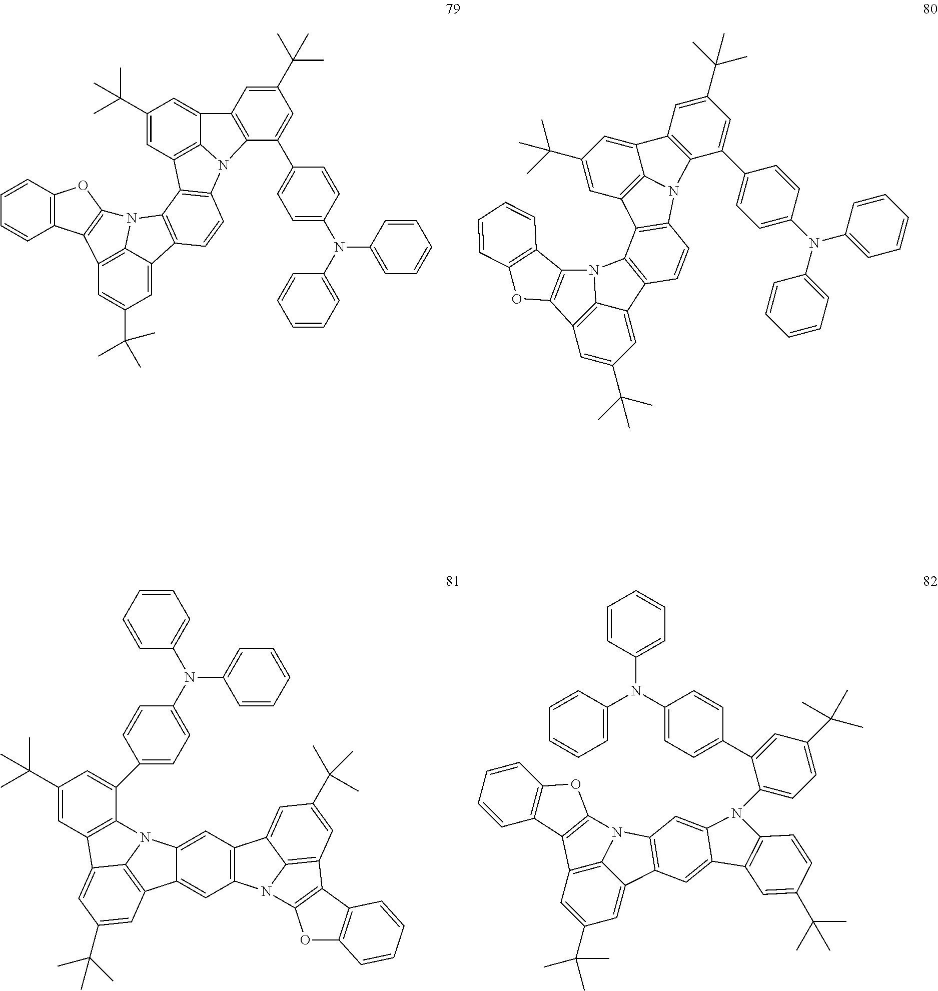 [SMILES: CC(C)(C)c1cc(-c2ccc(N(c3ccccc3)c3ccccc3)cc2)c2c(c1)c1cc(C(C)(C)C)cc3c4c(ccc5c6cc(C(C)(C)C)cc7c8c9ccccc9oc8n(c67)c54)n2c13 ; CC(C)(C)c1cc(-c2ccc(N(c3ccccc3)c3ccccc3)cc2)c2c(c1)c1cc(C(C)(C)C)cc3c4c(ccc5c6cc(C(C)(C)C)cc7c8oc9ccccc9c8n(c67)c54)n2c13 ; CC(C)(C)c1cc(-c2ccc(N(c3ccccc3)c3ccccc3)cc2)c2c(c1)c1cc(C(C)(C)C)cc3c4cc5c(cc4n2c31)c1cc(C(C)(C)C)cc2c3c4ccccc4oc3n5c12 ; CC(C)(C)c1ccc(-n2c3ccc(C(C)(C)C)cc3c3cc4c5cc(C(C)(C)C)cc6c7c8ccccc8oc7n(c4cc32)c56)c(-c2ccc(N(c3ccccc3)c3ccccc3)cc2)c1]

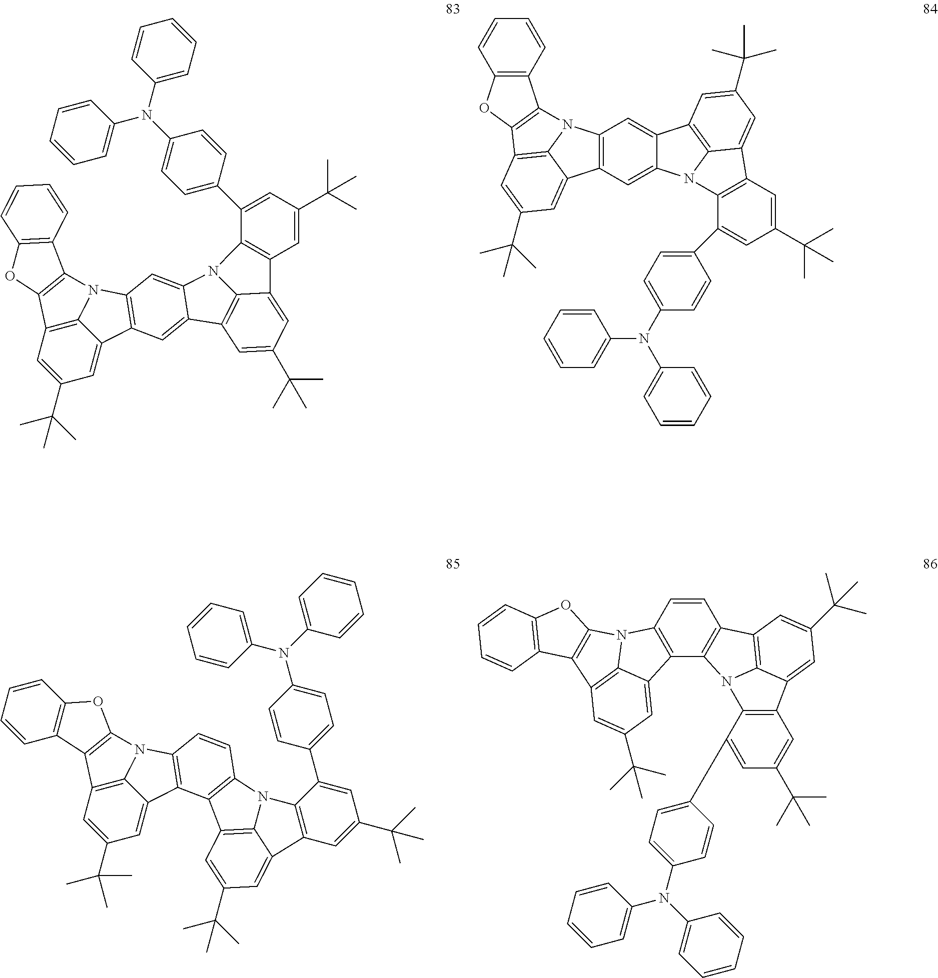 [SMILES: CC(C)(C)c1cc(-c2ccc(N(c3ccccc3)c3ccccc3)cc2)c2c(c1)c1cc(C(C)(C)C)cc3c4cc5c6cc(C(C)(C)C)cc7c8oc9ccccc9c8n(c5cc4n2c31)c67 ; CC(C)(C)c1cc(-c2ccc(N(c3ccccc3)c3ccccc3)cc2)c2c(c1)c1cc(C(C)(C)C)cc3c4cc5c(cc4n2c31)c1cc(C(C)(C)C)cc2c3oc4ccccc4c3n5c12 ; CC(C)(C)c1cc(-c2ccc(N(c3ccccc3)c3ccccc3)cc2)c2c(c1)c1cc(C(C)(C)C)cc3c4c5c6cc(C(C)(C)C)cc7c8c9ccccc9oc8n(c5ccc4n2c13)c67 ; CC(C)(C)c1cc(-c2ccc(N(c3ccccc3)c3ccccc3)cc2)c2c(c1)c1cc(C(C)(C)C)cc3c4ccc5c(c6cc(C(C)(C)C)cc7c8c9ccccc9oc8n5c76)c4n2c13]

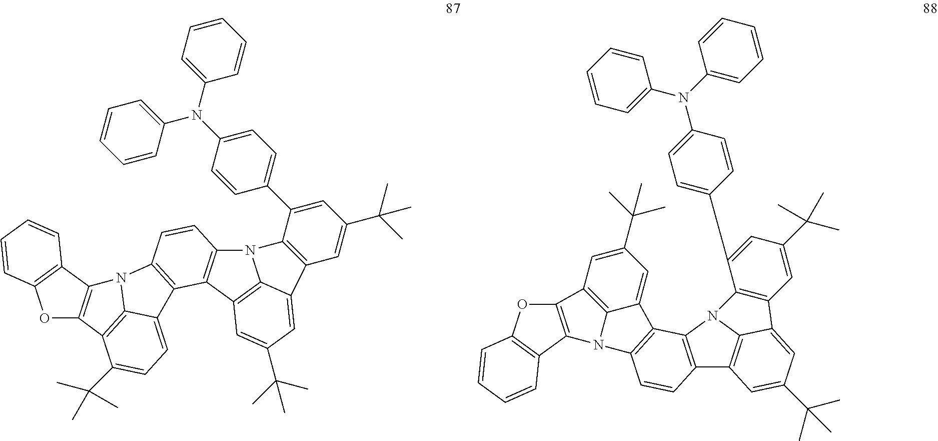 [SMILES: CC(C)(C)c1cc(-c2ccc(N(c3ccccc3)c3ccccc3)cc2)c2c(c1)c1cc(C(C)(C)C)cc3c4c5c6ccc(C(C)(C)C)c7c8oc9ccccc9c8n(c5ccc4n2c13)c67 ; CC(C)(C)c1cc(-c2ccc(N(c3ccccc3)c3ccccc3)cc2)c2c(c1)c1cc(C(C)(C)C)cc3c4ccc5c(c6cc(C(C)(C)C)cc7c8oc9ccccc9c8n5c76)c4n2c13]

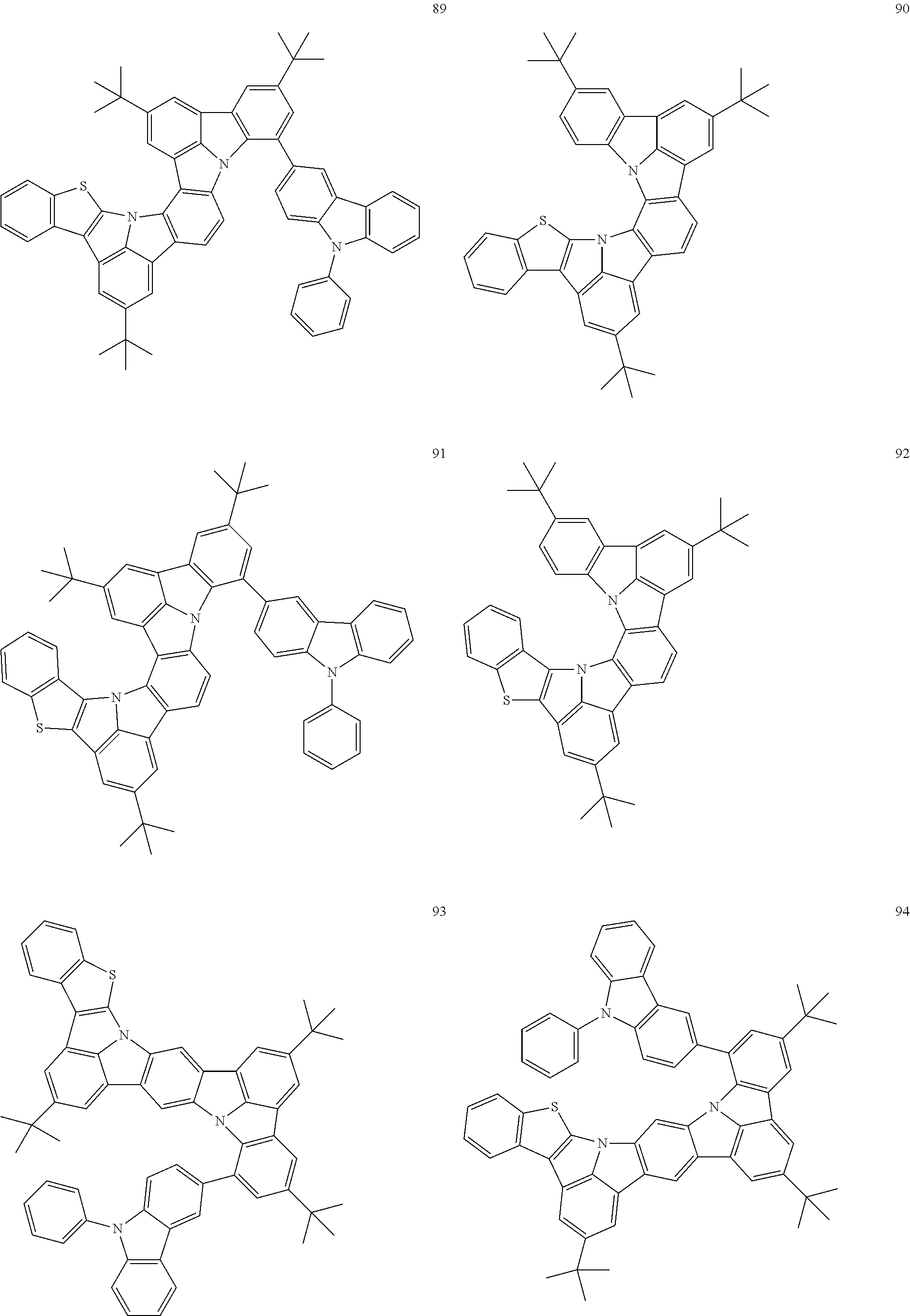

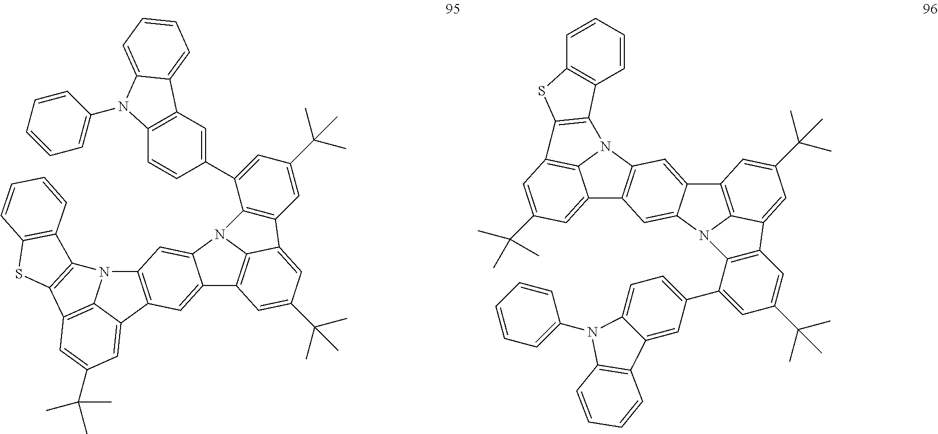 [SMILES: CC(C)(C)c1cc(-c2ccc3c(c2)c2ccccc2n3-c2ccccc2)c2c(c1)c1cc(C(C)(C)C)cc3c4cc5c6cc(C(C)(C)C)cc7c8sc9ccccc9c8n(c5cc4n2c31)c67 ; CC(C)(C)c1cc(-c2ccc3c(c2)c2ccccc2n3-c2ccccc2)c2c(c1)c1cc(C(C)(C)C)cc3c4cc5c(cc4n2c31)c1cc(C(C)(C)C)cc2c3sc4ccccc4c3n5c12]

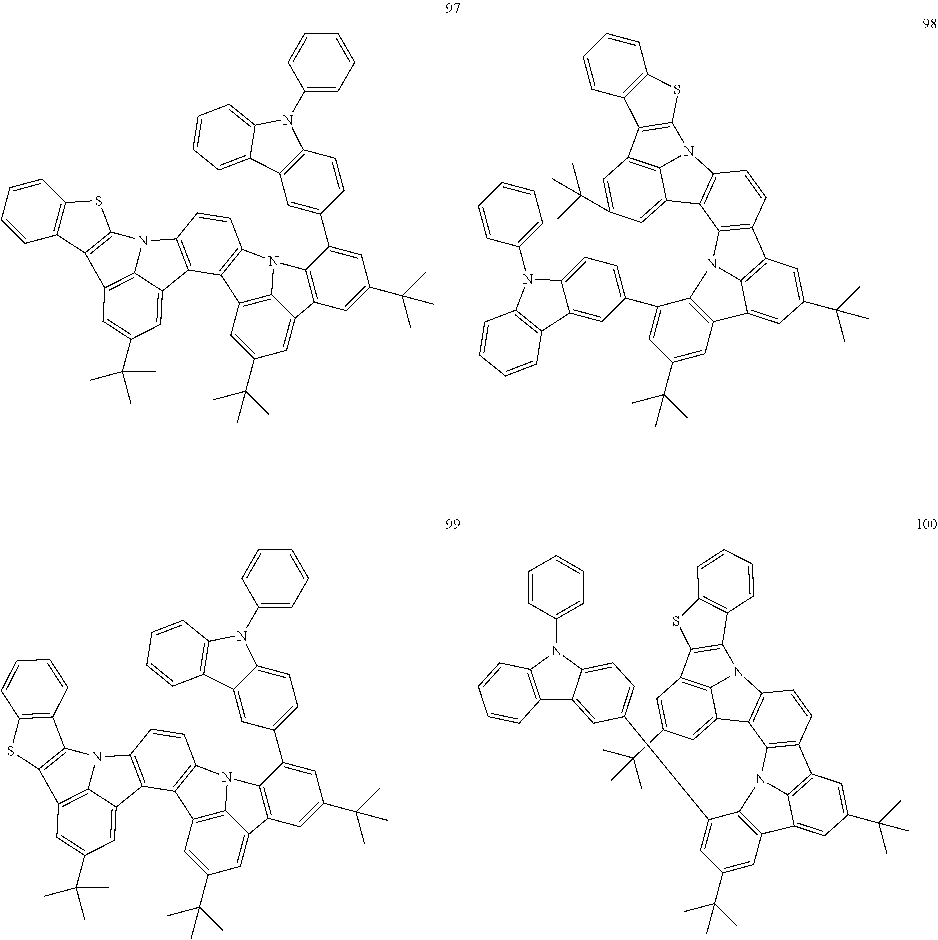 [SMILES: CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8c9ccccc9sc8n(c5ccc4n2c13)c67 ; CC(C)(C)c1cc(-c2ccc3c(c2)c2ccccc2n3-c2ccccc2)c2c(c1)c1cc(C(C)(C)C)cc3c4ccc5c(c6cc(C(C)(C)C)cc7c8c9ccccc9sc8n5c76)c4n2c13 ; CC(C)(C)c1cc(-c2ccc3c(c2)c2ccccc2n3-c2ccccc2)c2c(c1)c1cc(C(C)(C)C)cc3c4c5c6cc(C(C)(C)C)cc7c8sc9ccccc9c8n(c5ccc4n2c13)c76 ; CC(C)(C)c1cc(-c2ccc3c(c2)c2ccccc2n3-c2ccccc2)c2c(c1)c1cc(C(C)(C)C)cc3c4ccc5c(c6cc(C(C)(C)C)cc7c8sc9ccccc9c8n5c76)c4n2c13]

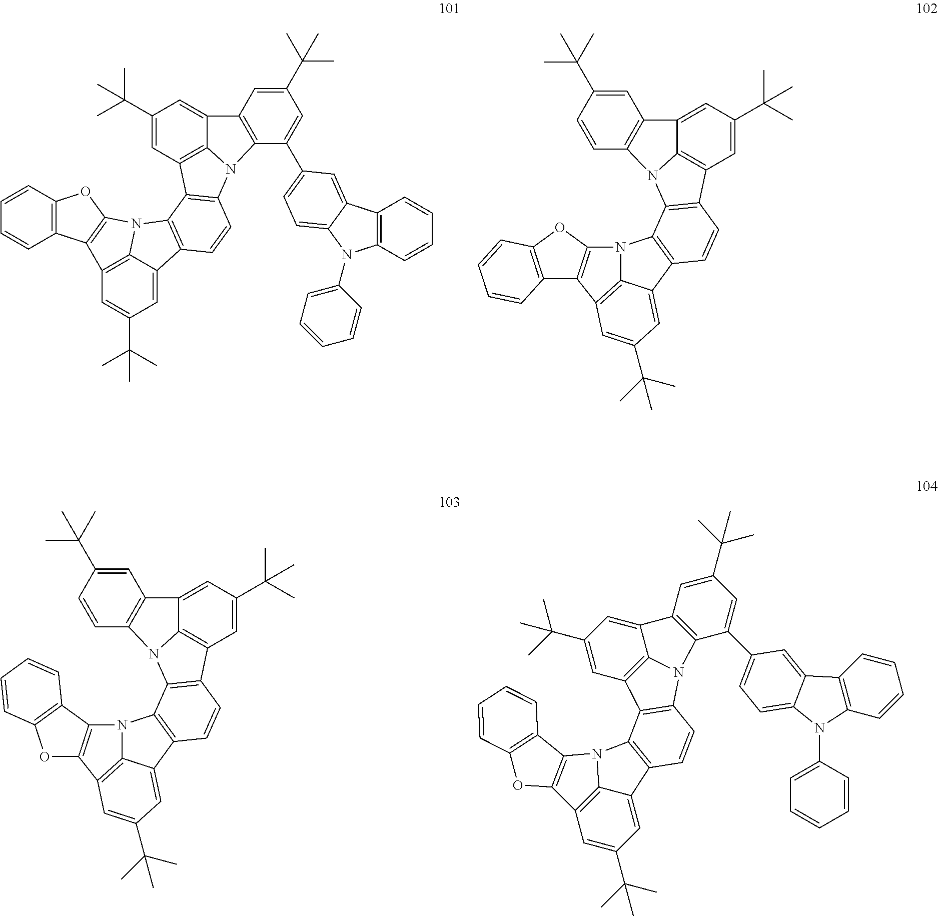 [SMILES: CC(C)(C)c1cc(-c2ccc3c(c2)c2ccccc2n3-c2ccccc2)c2c(c1)c1cc(C(C)(C)C)cc3c4c(ccc5c6cc(C(C)(C)C)cc7c8c9ccccc9oc8n(c67)c54)n2c13 ; CC(C)(C)c1ccc2c(c1)c1cc(C(C)(C)C)cc3c4ccc5c6cc(C(C)(C)C)cc7c8c9ccccc9oc8n(c67)c5c4n2c13 ; CC(C)(C)c1ccc2c(c1)c1cc(C(C)(C)C)cc3c4ccc5c6cc(C(C)(C)C)cc7c8oc9ccccc9c8n(c67)c5c4n2c13 ; CC(C)(C)c1cc(-c2ccc3c(c2)c2ccccc2n3-c2ccccc2)c2c(c1)c1cc(C(C)(C)C)cc3c4c(ccc5c6cc(C(C)(C)C)cc7c8oc9ccccc9c8n(c67)c54)n2c13]

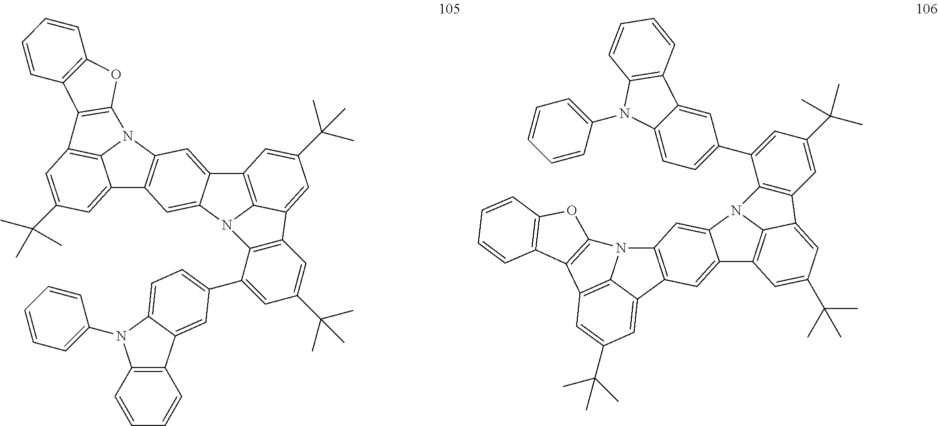 [SMILES: CC(C)(C)c1cc(-c2ccc3c(c2)c2ccccc2n3-c2ccccc2)c2c(c1)c1cc(C(C)(C)C)cc3c4cc5c(cc4n2c31)c1cc(C(C)(C)C)cc2c3c4ccccc4oc3n5c12 ; CC(C)(C)c1cc(-c2ccc3c(c2)c2ccccc2n3-c2ccccc2)c2c(c1)c1cc(C(C)(C)C)cc3c4cc5c6cc(C(C)(C)C)cc7c8c9ccccc9oc8n(c5cc4n2c31)c67]

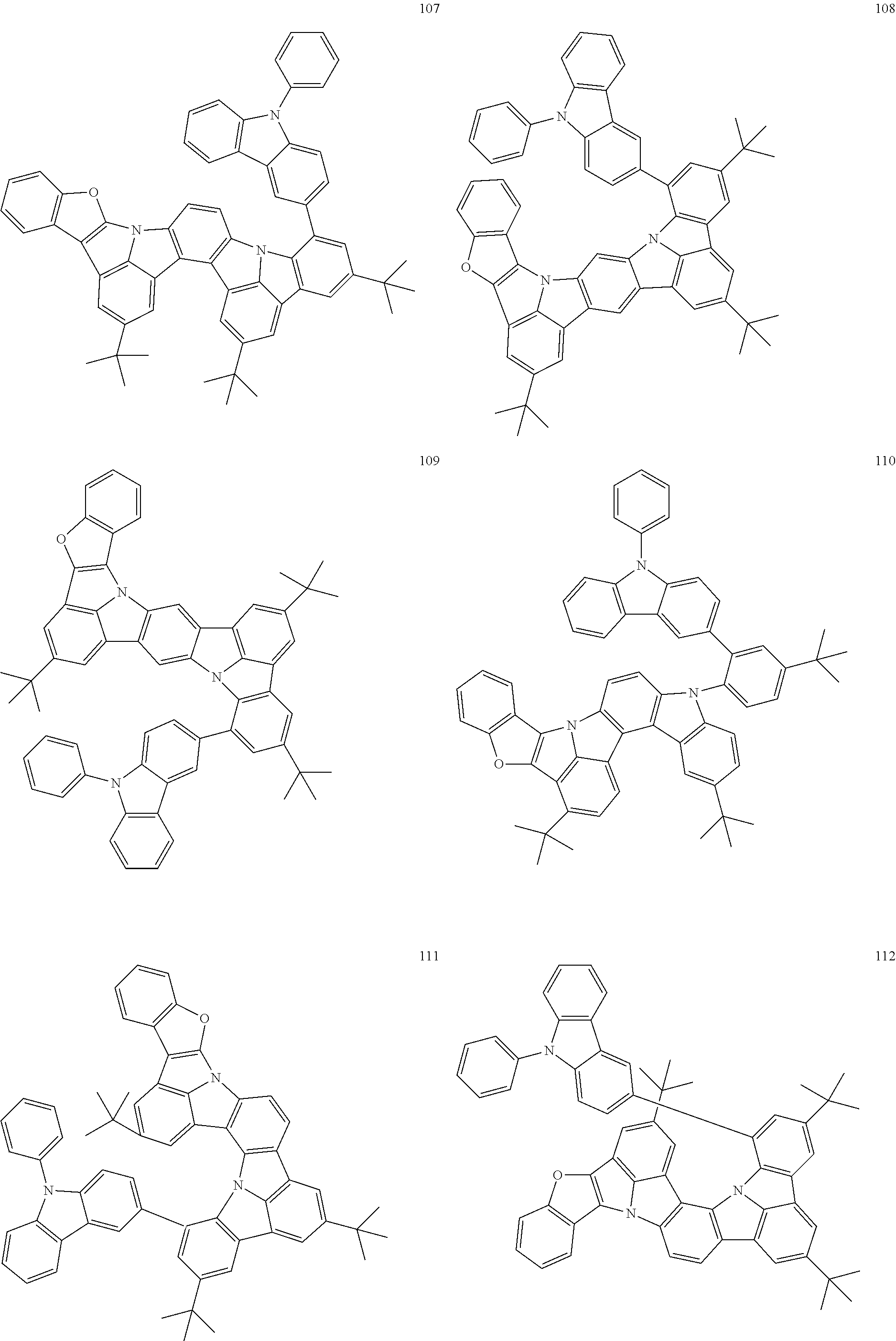

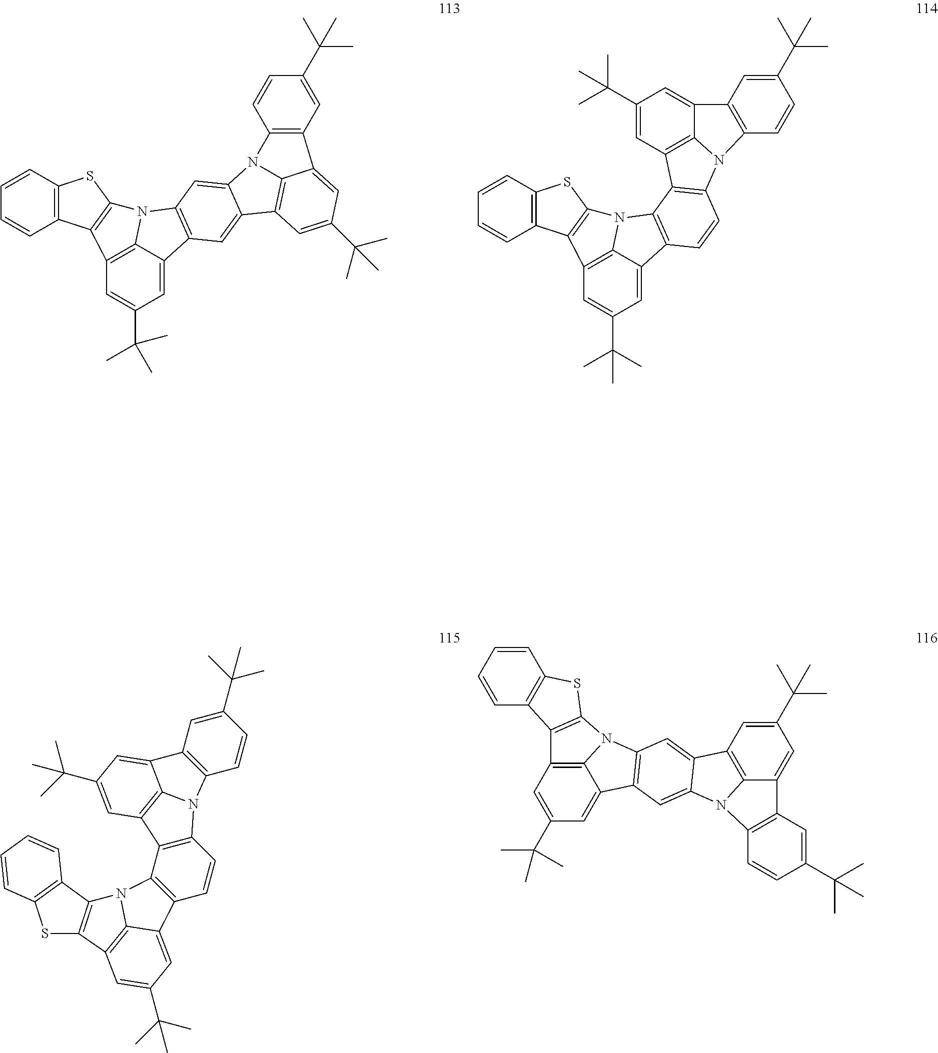 [SMILES: CC(C)(C)c1ccc2c(c1)c1cc(C(C)(C)C)cc3c4cc5c6cc(C(C)(C)C)cc7c8c9ccccc9sc8n(c5cc4n2c13)c67 ; CC(C)(C)c1ccc2c(c1)c1cc(C(C)(C)C)cc3c4c(ccc5c6cc(C(C)(C)C)cc7c8c9ccccc9sc8n(c67)c54)n2c13 ; CC(C)(C)c1ccc2c(c1)c1cc(C(C)(C)C)cc3c4c(ccc5c6cc(C(C)(C)C)cc7c8sc9ccccc9c8n(c67)c54)n2c13 ; CC(C)(C)c1ccc2c(c1)c1cc(C(C)(C)C)cc3c4cc5c(cc4n2c13)c1cc(C(C)(C)C)cc2c3c4ccccc4sc3n5c12]

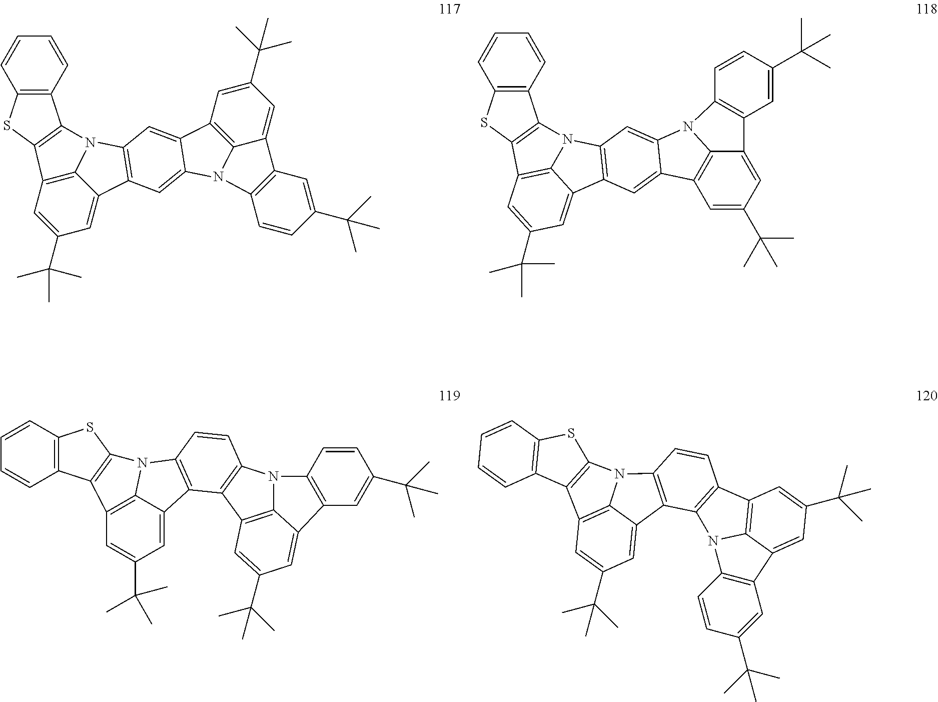 [SMILES: CC(C)(C)c1ccc2c(c1)c1cc(C(C)(C)C)cc3c4cc5c(cc4n2c13)c1cc(C(C)(C)C)cc2c3sc4ccccc4c3n5c12 ; CC(C)(C)c1ccc2c(c1)c1cc(C(C)(C)C)cc3c4cc5c6cc(C(C)(C)C)cc7c8sc9ccccc9c8n(c5cc4n2c13)c67 ; CC(C)(C)c1ccc2c(c1)c1cc(C(C)(C)C)cc3c4c5c6cc(C(C)(C)C)cc7c8c9ccccc9sc8n(c5ccc4n2c13)c67 ; CC(C)(C)c1ccc2c(c1)c1cc(C(C)(C)C)cc3c4ccc5c(c6cc(C(C)(C)C)cc7c8c9ccccc9sc8n5c76)c4n2c13]

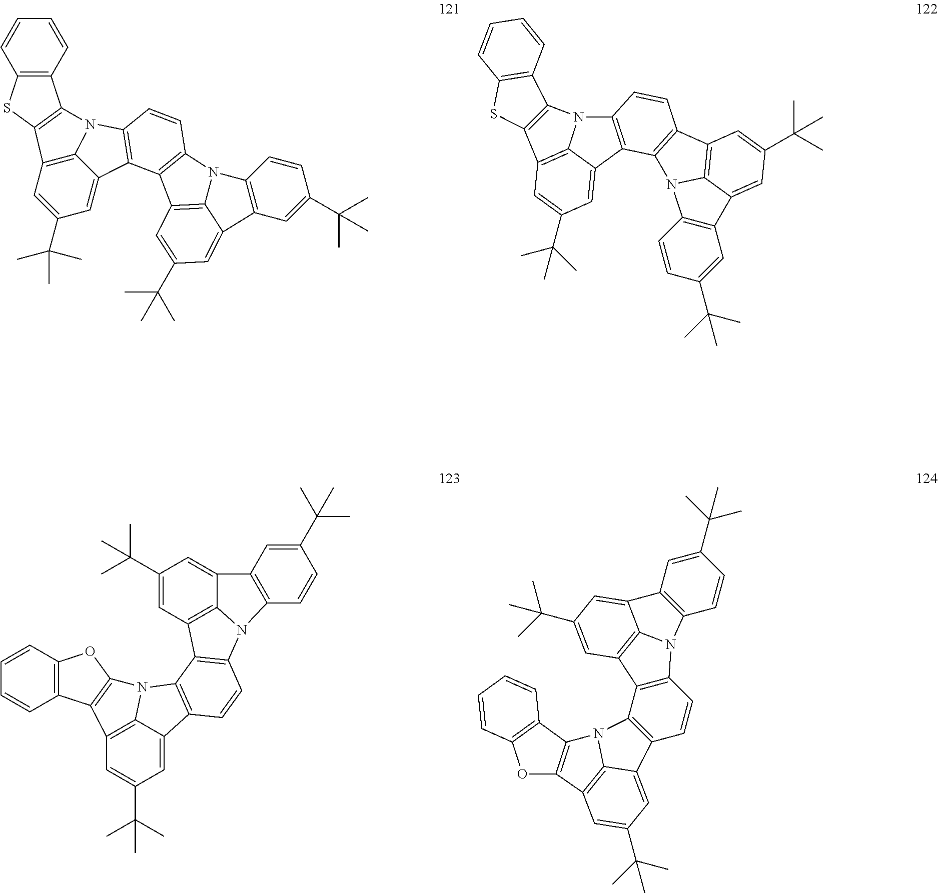 [SMILES: CC(C)(C)c1ccc2c(c1)c1cc(C(C)(C)C)cc3c4c5c6cc(C(C)(C)C)cc7c8sc9ccccc9c8n(c5ccc4n2c13)c76 ; CC(C)(C)c1ccc2c(c1)c1cc(C(C)(C)C)cc3c4ccc5c(c6cc(C(C)(C)C)cc7c8sc9ccccc9c8n5c76)c4n2c13 ; CC(C)(C)c1ccc2c(c1)c1cc(C(C)(C)C)cc3c4c(ccc5c6cc(C(C)(C)C)cc7c8c9ccccc9oc8n(c67)c54)n2c13 ; CC(C)(C)c1ccc2c(c1)c1cc(C(C)(C)C)cc3c4c(ccc5c6cc(C(C)(C)C)cc7c8oc9ccccc9c8n(c67)c54)n2c13]

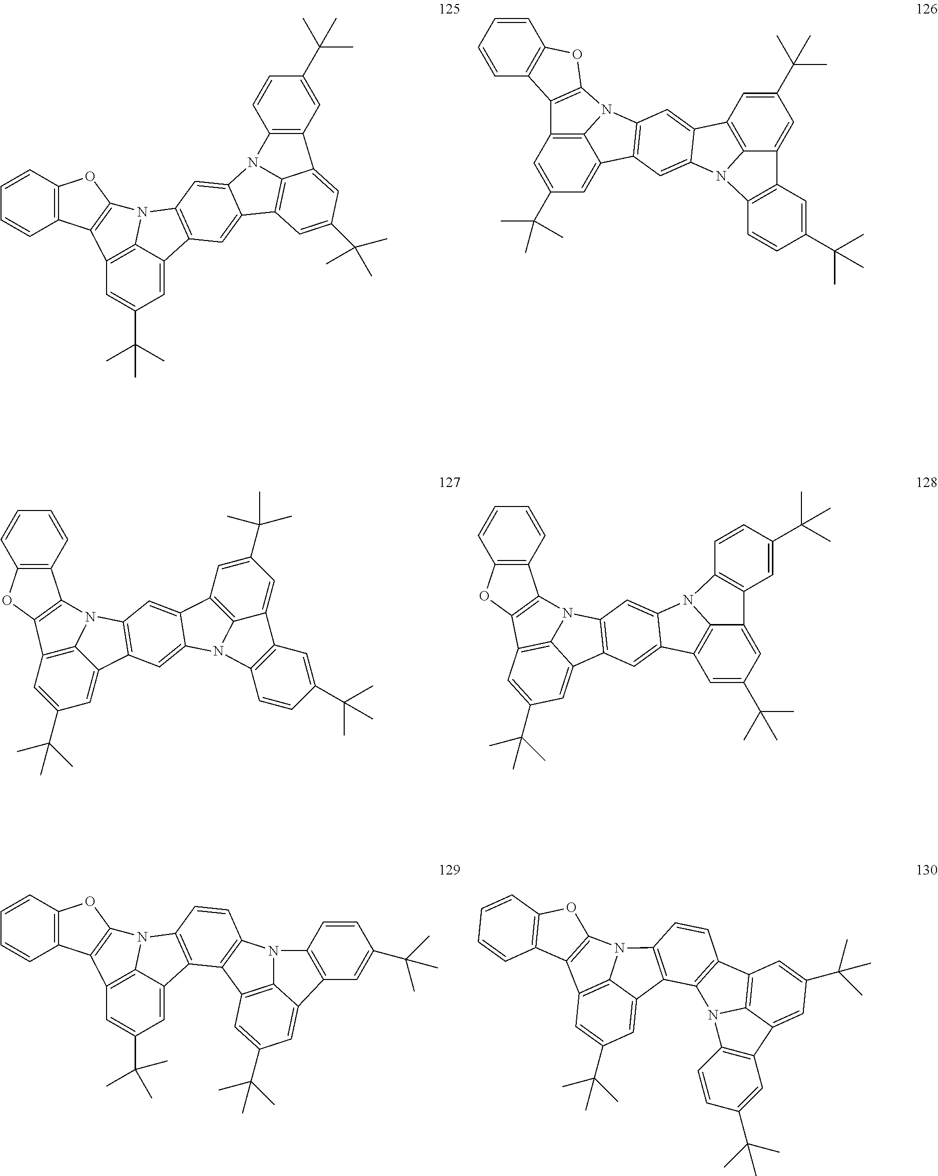

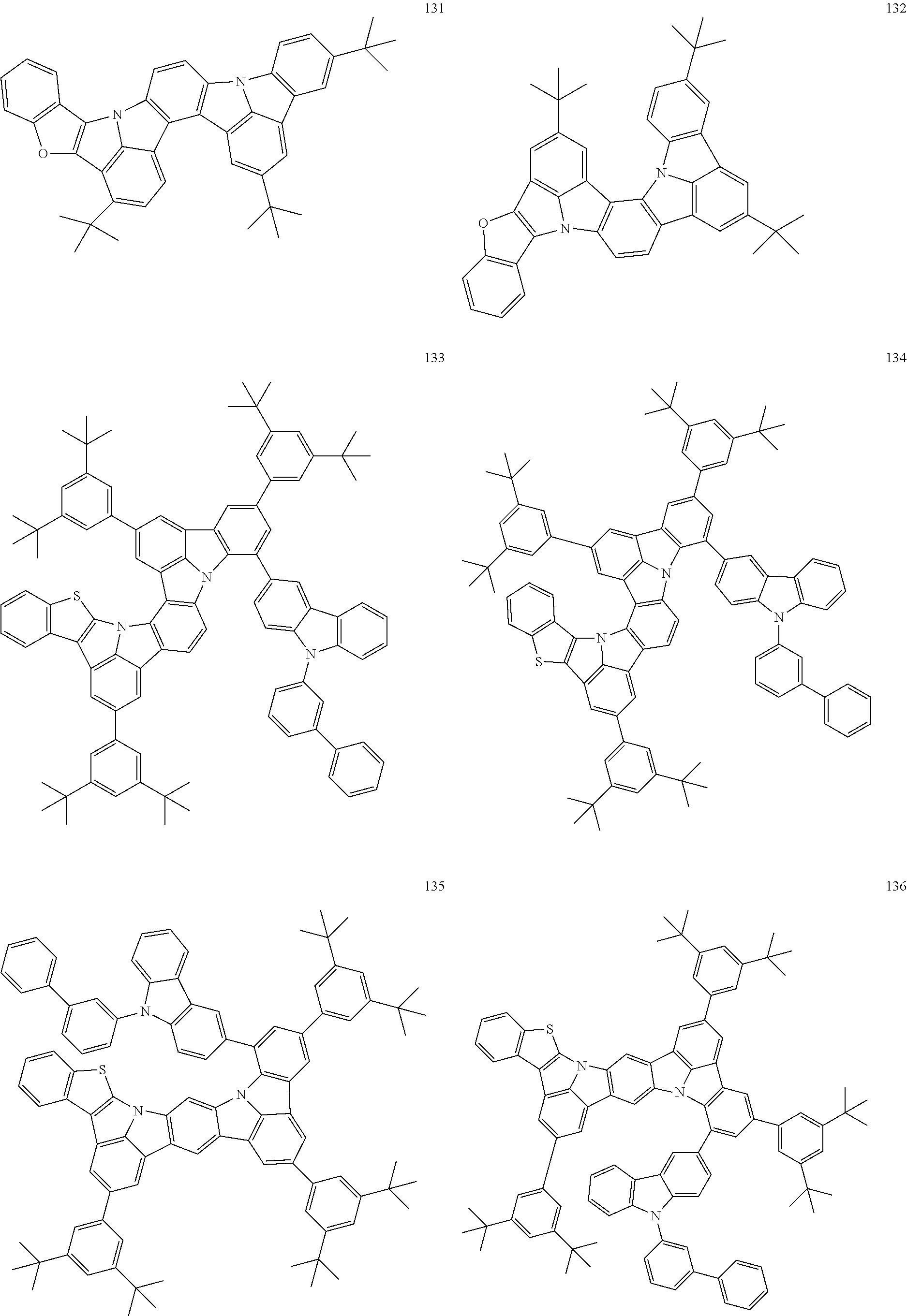

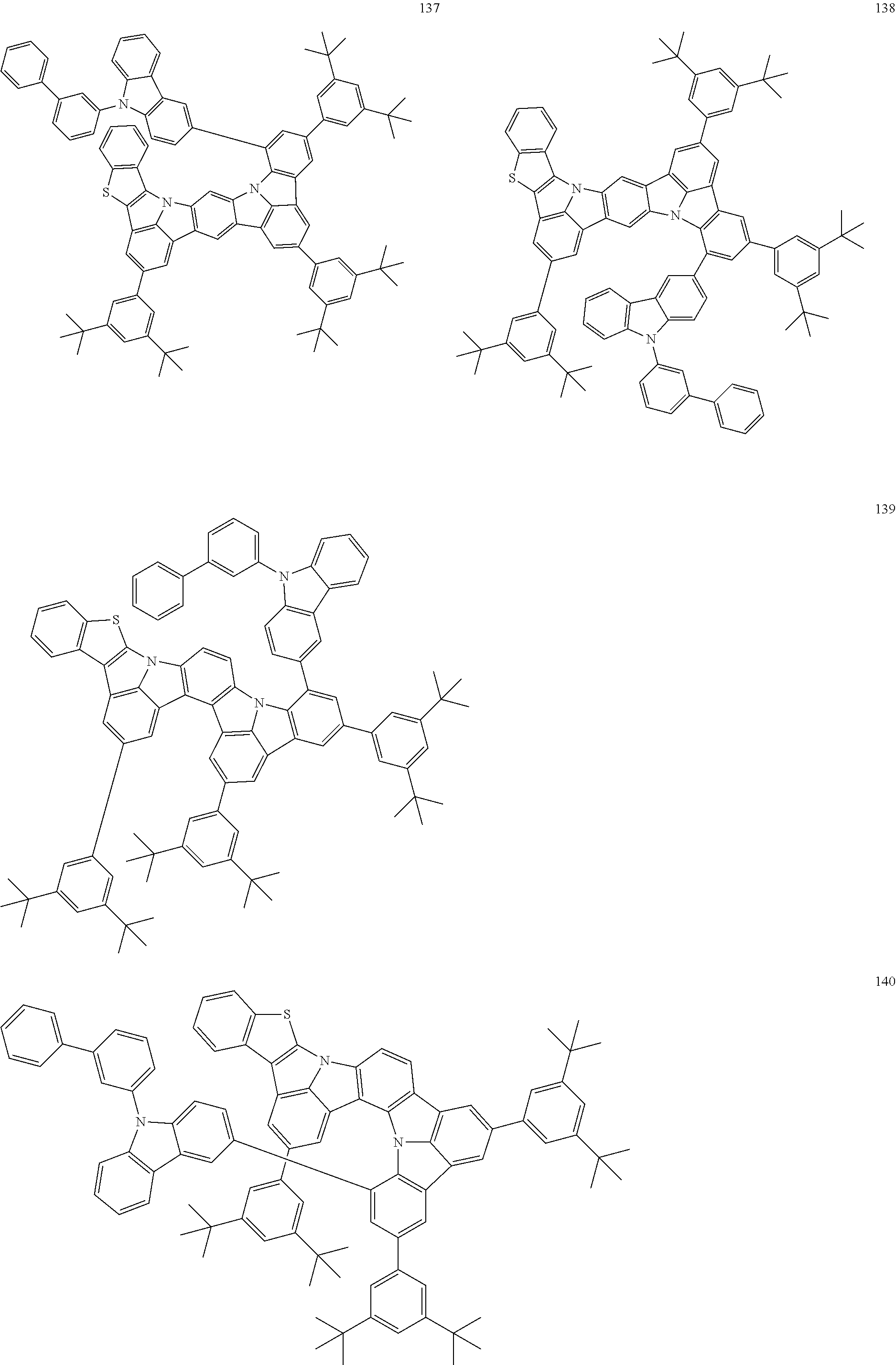

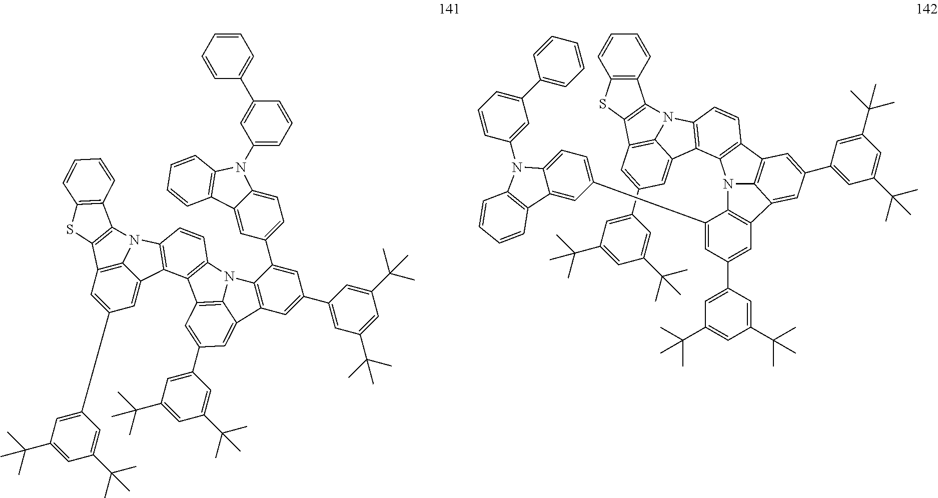 [SMILES: CC(C)(C)c1cc(-c2cc(-c3ccc4c(c3)c3ccccc3n4-c3cccc(-c4ccccc4)c3)c3c(c2)c2cc(-c4cc(C(C)(C)C)cc(C(C)(C)C)c4)cc4c5c6c7cc(-c8cc(C(C)(C)C)cc(C(C)(C)C)c8)cc8c9sc%10ccccc%10c9n(c6ccc5n3c24)c87)cc(C(C)(C)C)c1 ; CC(C)(C)c1cc(-c2cc(-c3ccc4c(c3)c3ccccc3n4-c3cccc(-c4ccccc4)c3)c3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9sc%10ccccc%10c9n6c87)c5n3c24)cc(C(C)(C)C)c1]

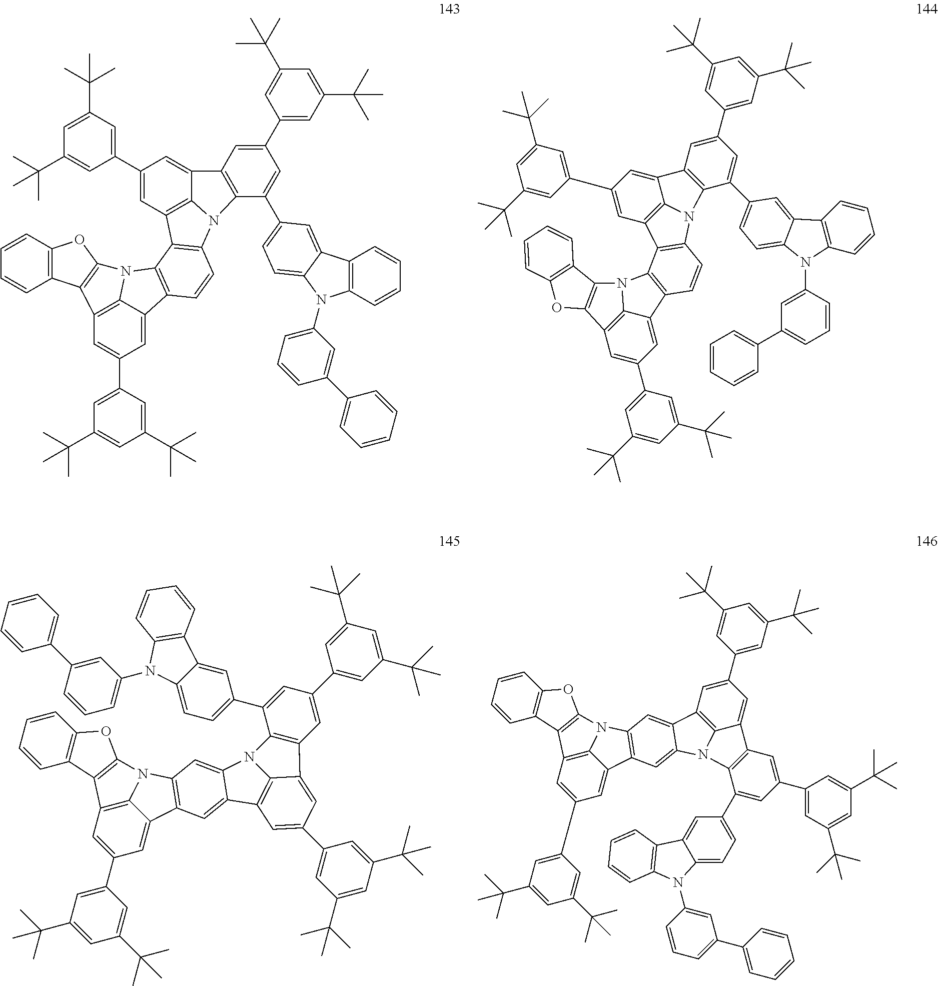

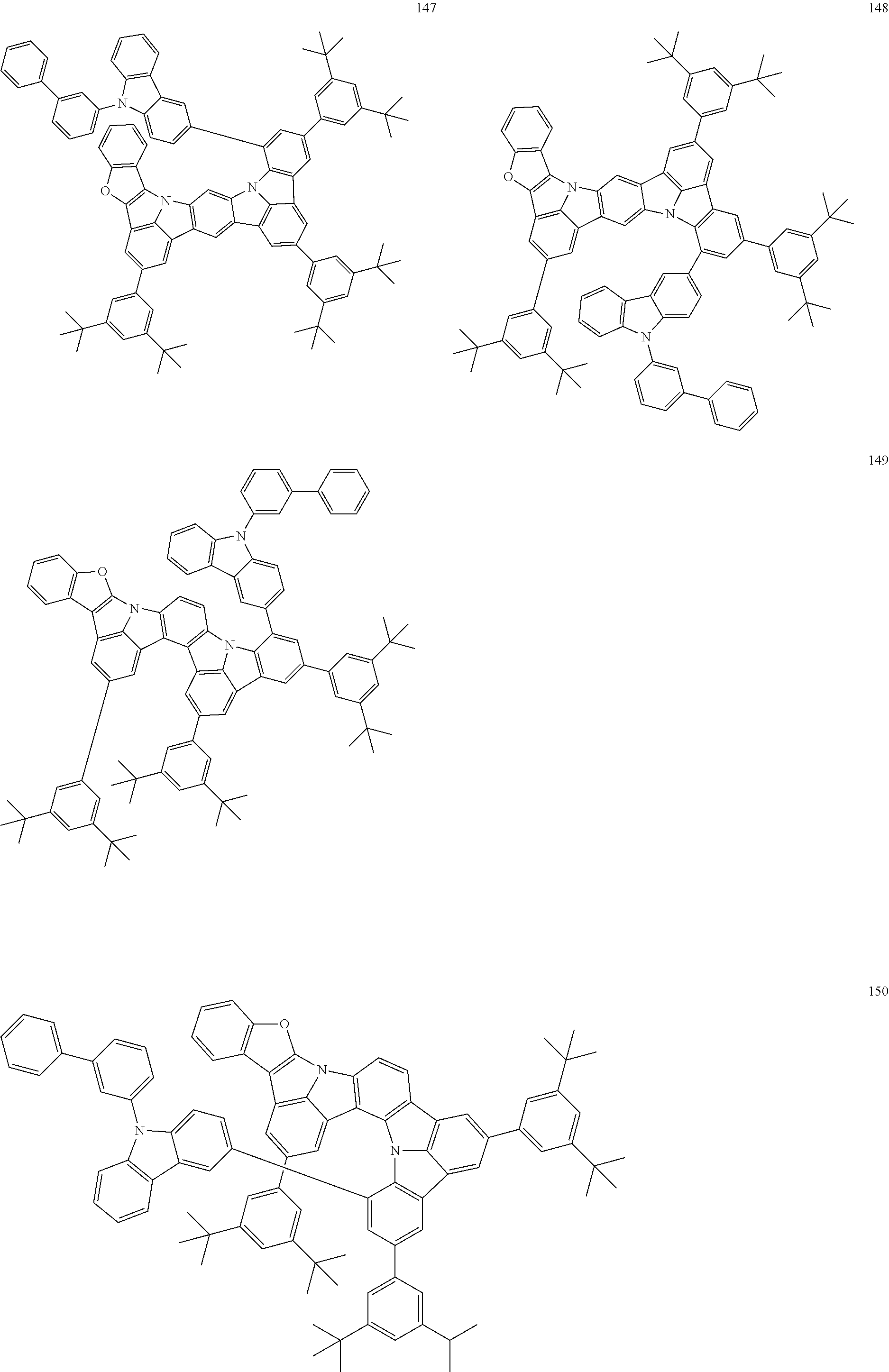

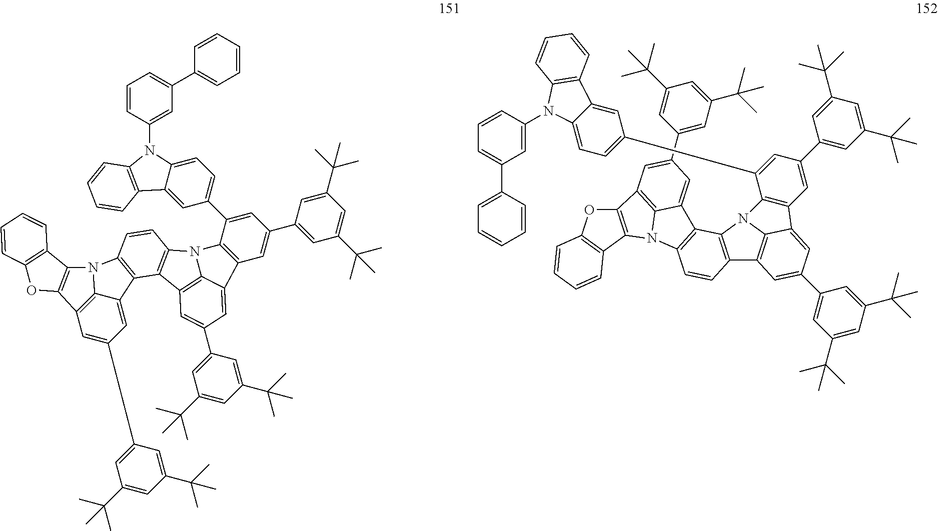 [SMILES: CC(C)(C)c1cc(-c2cc(-c3ccc4c(c3)c3ccccc3n4-c3cccc(-c4ccccc4)c3)c3c(c2)c2cc(-c4cc(C(C)(C)C)cc(C(C)(C)C)c4)cc4c5c6c7cc(-c8cc(C(C)(C)C)cc(C(C)(C)C)c8)cc8c9oc%10ccccc%10c9n(c6ccc5n3c24)c87)cc(C(C)(C)C)c1 ; CC(C)(C)c1cc(-c2cc(-c3ccc4c(c3)c3ccccc3n4-c3cccc(-c4ccccc4)c3)c3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9oc%10ccccc%10c9n6c87)c5n3c24)cc(C(C)(C)C)c1]

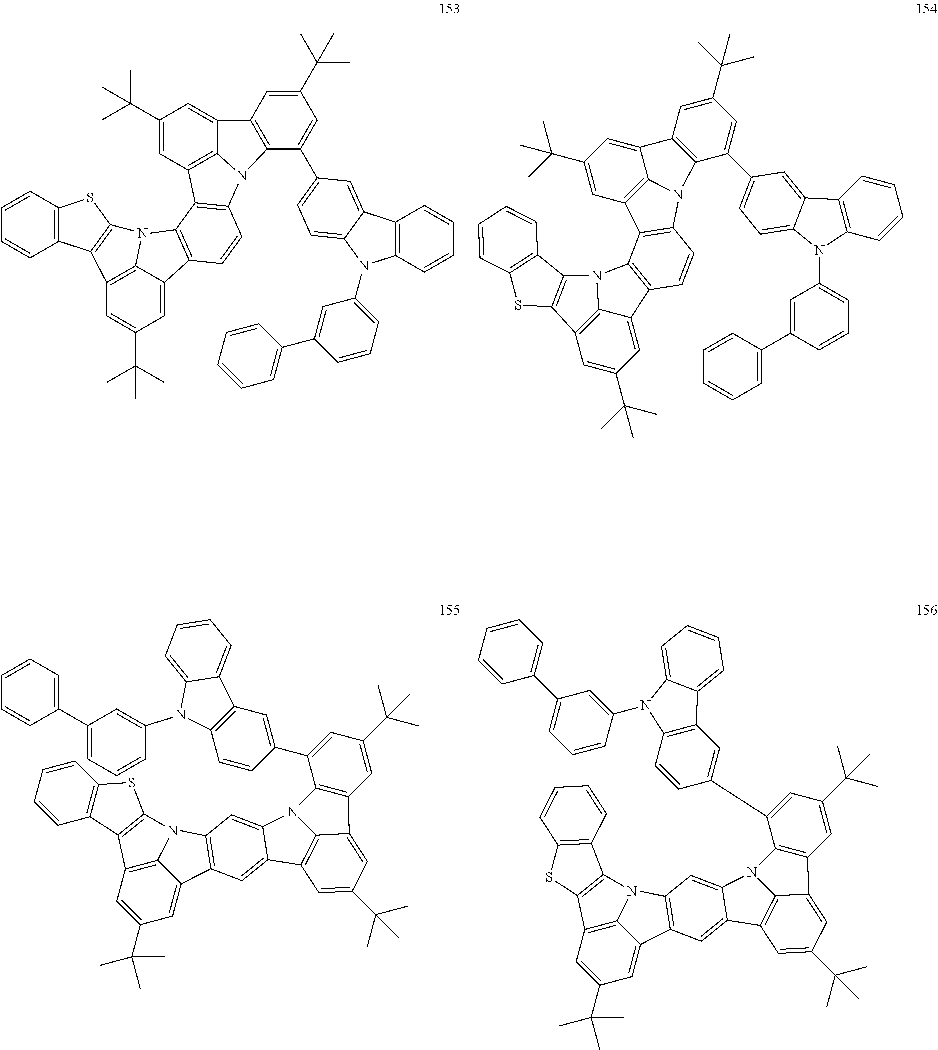

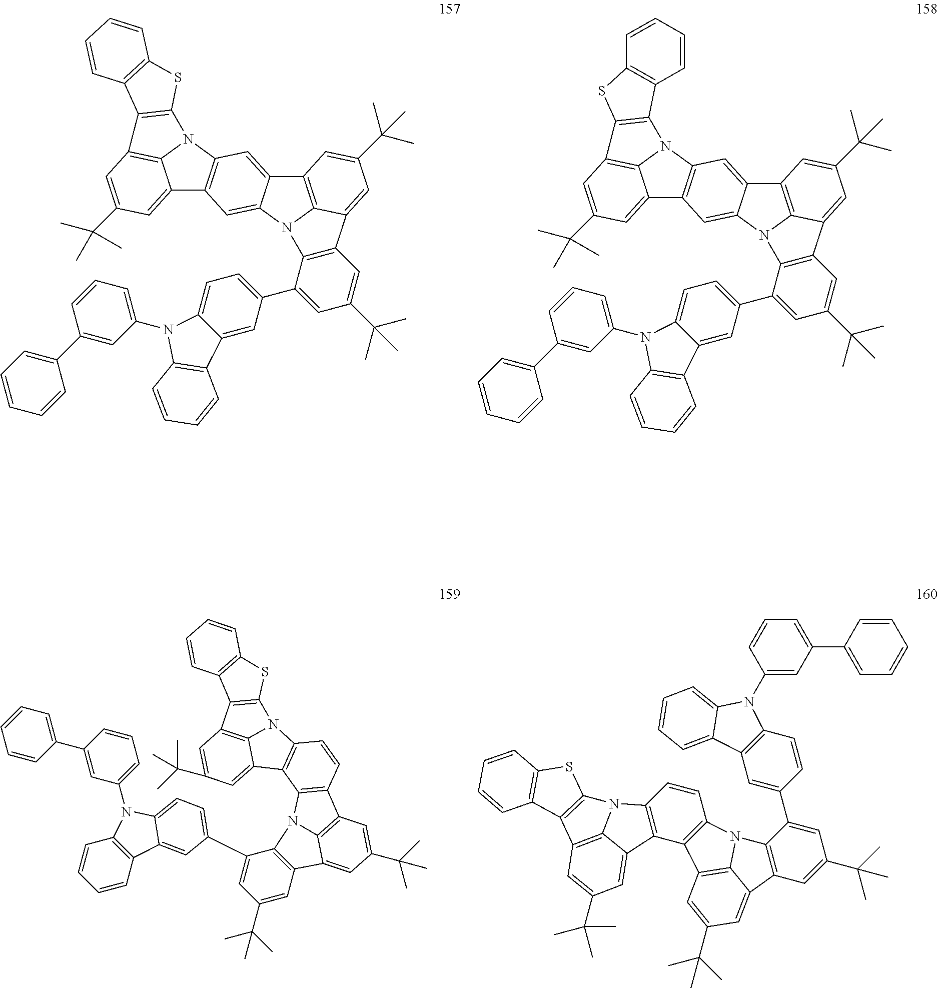

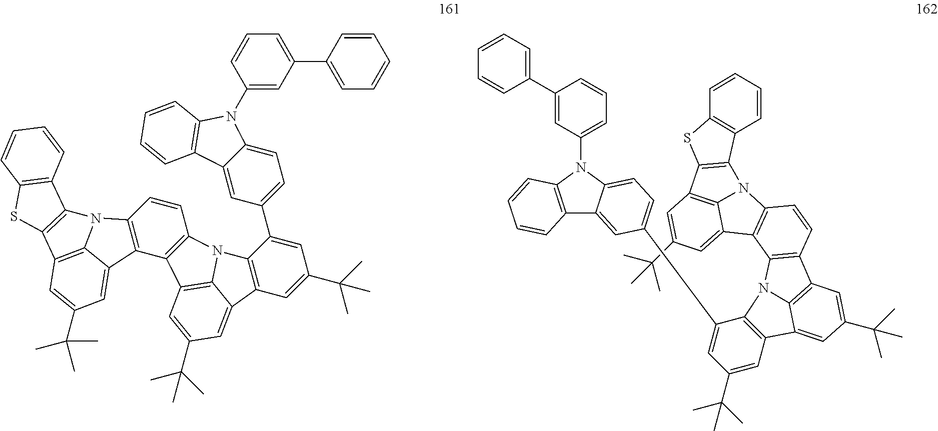 [SMILES: CC(C)(C)c1cc(-c2ccc3c(c2)c2ccccc2n3-c2cccc(-c3ccccc3)c2)c2c(c1)c1cc(C(C)(C)C)cc3c4c5c6cc(C(C)(C)C)cc7c8sc9ccccc9c8n(c5ccc4n2c13)c76 ; CC(C)(C)c1cc(-c2ccc3c(c2)c2ccccc2n3-c2cccc(-c3ccccc3)c2)c2c(c1)c1cc(C(C)(C)C)cc3c4ccc5c(c6cc(C(C)(C)C)cc7c8sc9ccccc9c8n5c76)c4n2c13]

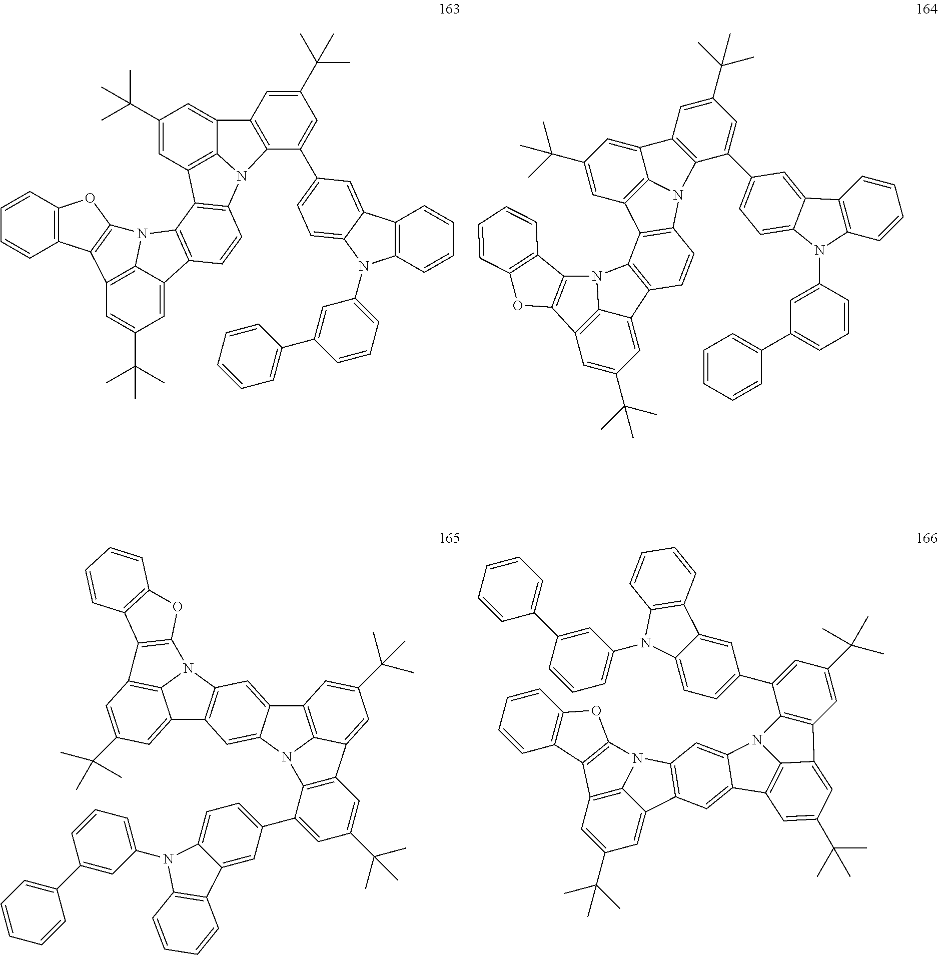

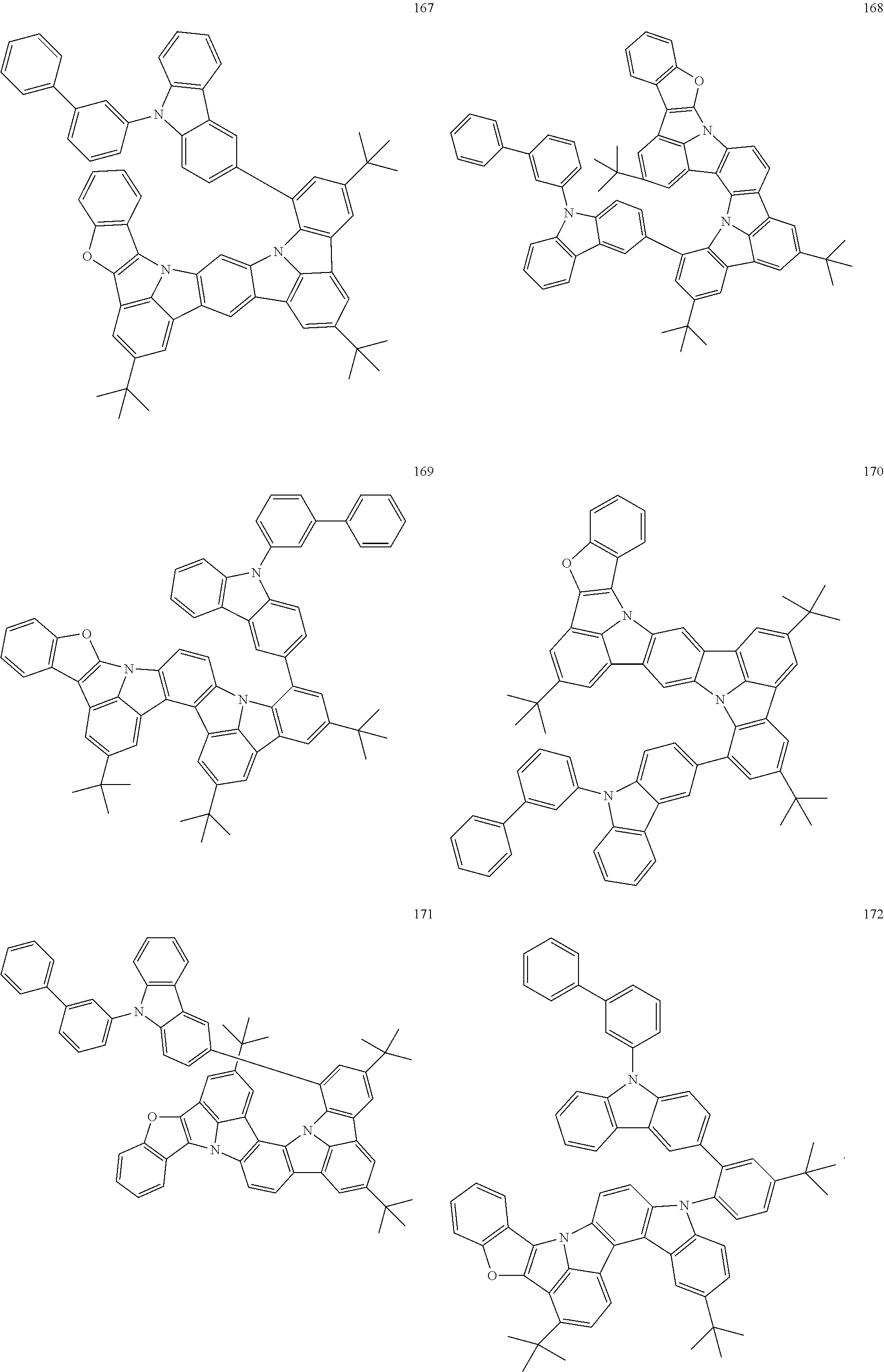

In Formula 1, at least one of W₁ and W₂ may include O or S, and the sum of n1 and n2 may be 1. As such, in the condensed cyclic compound represented by Formula 1, may lower the triplet energy level to improve the fluorescence emission mechanism, and accordingly, an electronic device employing the condensed cyclic compound, for example, a light-emitting device may have an improved driving voltage and increased external quantum efficiency.

In an embodiment, a full width at half maximum (FWHM) in the emission spectrum of the condensed cyclic compound may be about 5 nanometers (nm) to about 40 nm, for example, about 10 nm to about 35 nm.

In one or more embodiments, an emission peak wavelength in the emission spectrum of the condensed cyclic compound may be about 390 nm to about 500 nm, for example, about 390 nm to about 480 nm.

In one or more embodiments, a singlet (S₁) energy level of the condensed cyclic compound may be about 2.6 electronvolts (eV) to about 3.2 eV.

In one or more embodiments, a triplet (T₁) energy level of the condensed cyclic compound may be about 1.6 eV to about 3.2 eV. For example, the triplet (T₁) energy level of the condensed cyclic compound may be about 1.6 eV to about 3.2 eV, about 1.6 eV to about 2.8 eV, or about 1.6 eV to about 2.6 eV.

In one or more embodiments, the absolute value of the difference between the singlet (S₁) energy level and the triplet (T₁) energy level of the condensed cyclic compound may be about 0 eV to about 1 eV.

In one or more embodiments, a highest occupied molecular orbital (HOMO) energy level of the condensed cyclic compound may be about −4.0 eV to about −6.5 eV.

Synthesis methods of the condensed cyclic compound may be recognized by one of ordinary skill in the art by referring to the Synthesis Examples provided below.

### Light-Emitting Device

The condensed cyclic compound may be used as a material of an interlayer of a light-emitting device, for example, an emission layer of the interlayer, and according to another aspect of the disclosure, provided is a light-emitting device including: a first electrode; a second electrode; and an interlayer arranged between the first electrode and the second electrode. The interlayer may include an emission layer and the interlayer may include at least one of the condensed cyclic compound described above.

When the light-emitting device includes an interlayer including at least one of the condensed cyclic compound described above, the triplet energy thereof may be lowered while maintaining a small FWHM. In this manner, the delayed fluorescence may be suppressed, and the fluorescence emission mechanism may be improved. Accordingly, the light-emitting device may emit blue light shifted to a shorter wavelength and have improved driving voltage, external quantum efficiency, luminescence efficiency, and/or lifespan characteristics.

The condensed cyclic compound may be used between a pair of electrodes of the light-emitting device. For example, the condensed cyclic compound may be included in the emission layer. In this case, the emission layer may further include a host. The amount (weight) of the host may be greater than the amount (weight) of the condensed cyclic compound. The emission layer may emit red light, green light, or blue light. For example, the emission layer may emit blue light.

In an embodiment, a Commission International de l'Eclairage (CIE) y value (CIEy value) of light emitted from the emission layer may be about 0.010 to about 0.170, about 0.040 to about 0.170, about 0.050 to about 0.170, about 0.060 to about 0.170, about 0.010 to about 0.165, about 0.040 to about 0.165, about 0.050 to about 0.165, about 0.060 to about 0.165, about 0.070 to about 0.165, or about 0.08 to about 0.165.

In one or more embodiments, the emission peak wavelength of light emitted from the emission layer may be about 440 nm to about 470 nm, about 445 nm to about 470 nm, about 450 nm to about 470 nm, about 455 nm to about 470 nm, about 460 nm to about 470 nm, about 440 nm to about 465 nm, about 445 nm to about 465 nm, about 450 nm to about 465 nm, about 455 nm to about 465 nm, or about 460 nm to about 465 nm.

The emission layer may further include a host. The host may be understood by referring to the description of the host provided herein.

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

For example, in the organic light-emitting device, the first electrode may be an anode, the second electrode may be a cathode, and the interlayer may further include a hole transport region between the first electrode and the emission layer and an electron transport region between the emission layer and the second electrode, wherein the hole transport region may include a hole injection layer, a hole transport layer, an electron-blocking layer, an auxiliary layer, or any combination thereof, and the electron transport region may include a buffer layer, a hole-blocking layer, an electron transport layer, an electron injection layer, or any combination thereof.

The term “interlayer” as used herein refers to a single layer and/or a plurality of layers arranged between the first electrode and the second electrode of the organic light-emitting device. The “interlayer” may include not only organic compounds but also organometallic complexes including metals.

In some embodiments, the emission layer may include a first Embodiment or a second Embodiment:

### First Embodiment

The emission layer may include at least one condensed cyclic compound described above, and the condensed cyclic compound may function as an emitter, for example, a delayed fluorescence emitter or a prompt fluorescence emitter. That is, the condensed cyclic compound may be an emitter. For example, a ratio of a luminescent component emitted from the condensed cyclic compound with respect to all luminescent components of the emission layer may be 80% or more, 85% or more, 90% or more, or 95% or more. Light emitted from the condensed cyclic compound may be blue light. The emission layer may further include a sensitizer, and the sensitizer may be different from the condensed cyclic compound. The sensitizer may be an organometallic compound, a delayed fluorescence material, a prompt fluorescence material, or any combination thereof. The amount (weight) of the sensitizer may be about 0.01 parts by weight to about 20 parts by weight based on 100 parts by weight of the emission layer.

### Second Embodiment

The emission layer may include at least one condensed cyclic compound described above, and the condensed cyclic compound may function as a sensitizer or an auxiliary dopant. That is, the condensed cyclic compound may be a sensitizer or an auxiliary dopant. The sensitizer may facilitate effective transfer of excitons of a host to an emitter. The emission layer may further include an emitter, and the emitter may be different from the condensed cyclic compound. The emitter may be an organometallic compound, a delayed fluorescence material, a prompt fluorescence material, or any combination thereof.

In the specification, the emitter refers to a material capable of receiving excitons from a host, a sensitizer, and/or an auxiliary dopant and emitting light by the transition of the excitons to the ground state.

In the first and second embodiments, the amount (weight) of the condensed cyclic compound may be about 0.01 parts by weight to about 40 parts by weight, about 0.1 parts by weight to about 20 parts by weight, or about 1 part by weight to about 20 parts by weight based on 100 parts by weight of the emission layer.

In the first and second embodiments, the organometallic compound may include a transition metal and n ligands bonded to the transition metal, wherein n may be an integer from 1 to 4.

In an embodiment, the transition metal in the organometallic compound may be platinum (Pt) or palladium (Pd), n may be 1, and the ligand may be a tetradentate ligand. The tetradentate ligand may include, for example, a carbene moiety bonded to the transition metal.

In one or more embodiments, the organometallic compound may include a transition metal and a tetradentate ligand bonded to the transition metal, wherein the transition metal may be platinum or palladium, and the tetradentate ligand may include a carbene moiety bonded to the transition metal.

In one or more embodiments, the transition metal in the organometallic compound may be iridium (Ir) or osmium (Os), n may be 3, and at least one of the n ligands may be a bidentate ligand including —F, a cyano group, or any combination thereof or a bidentate ligand including a carbene moiety bonded to the transition metal. For example, the bidentate ligand may further include an imidazole group or a triazole group.

In one or more embodiments, the organometallic compound may be an organometallic compound represented by Formula 3 and/or an organometallic compound represented by Formula 5. Formulae 3 and 5 will be described in detail later.

In the first and second embodiments, the delayed fluorescence material may be, for example, a thermally activated delayed fluorescence material. In another embodiment, the delayed fluorescence material may be a multiple resonance thermally activated delayed fluorescence material.

The multiple resonance thermally activated delayed fluorescence material may be a polycyclic compound i) which does not include a transition metal, and ii) includes a core in which two or more C₃-C₆₀ cyclic groups are condensed with each other. In this regard, two C₃-C₆₀ cyclic groups of the core may be condensed with each other while sharing a boron (B) or a nitrogen (N) atom.

In an embodiment, the delayed fluorescence material may be a polycyclic compound represented by Formula 4. Formula 4 will be described in detail later.

The prompt fluorescence material in the first and second embodiments may be an amino group-containing compound, a styryl group-containing compound, etc. For example, the prompt fluorescence material may include a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group(tetracene group), a picene group, a perylene group, a pentaphene group, an indenoanthracene group, a group represented by one of Formulae 501-1 to 501-21, or any combination thereof:

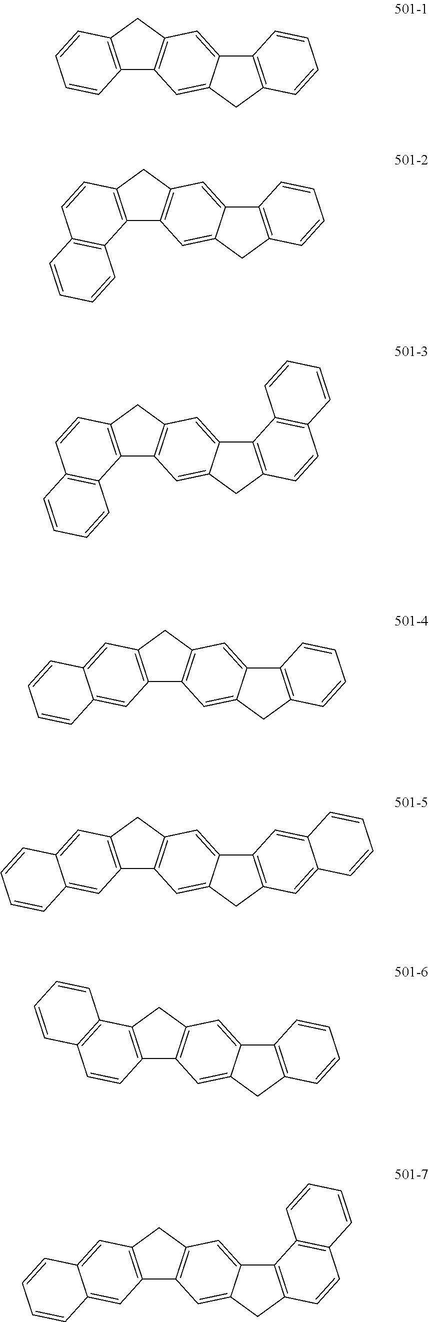

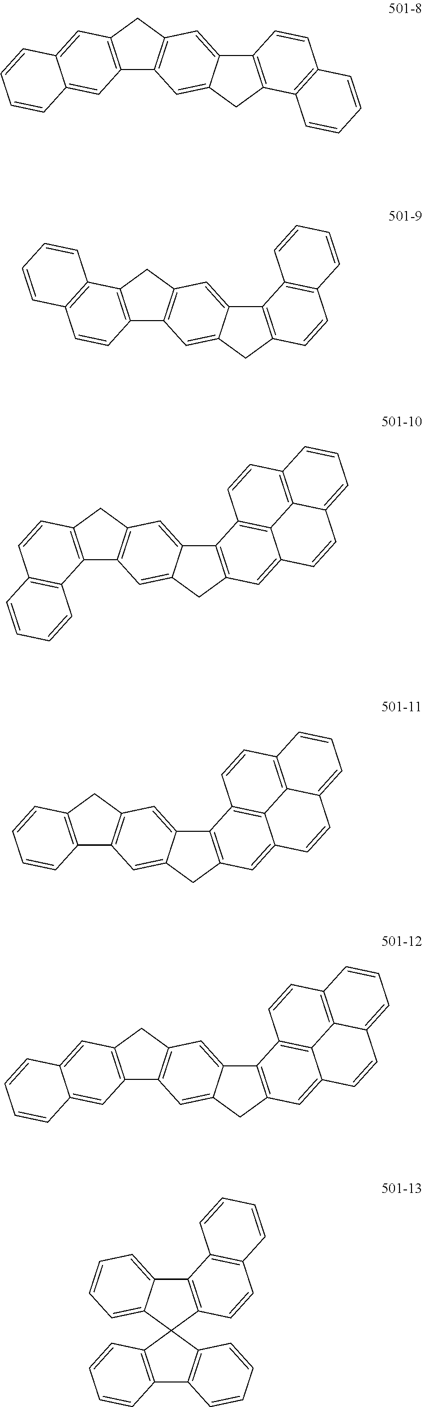

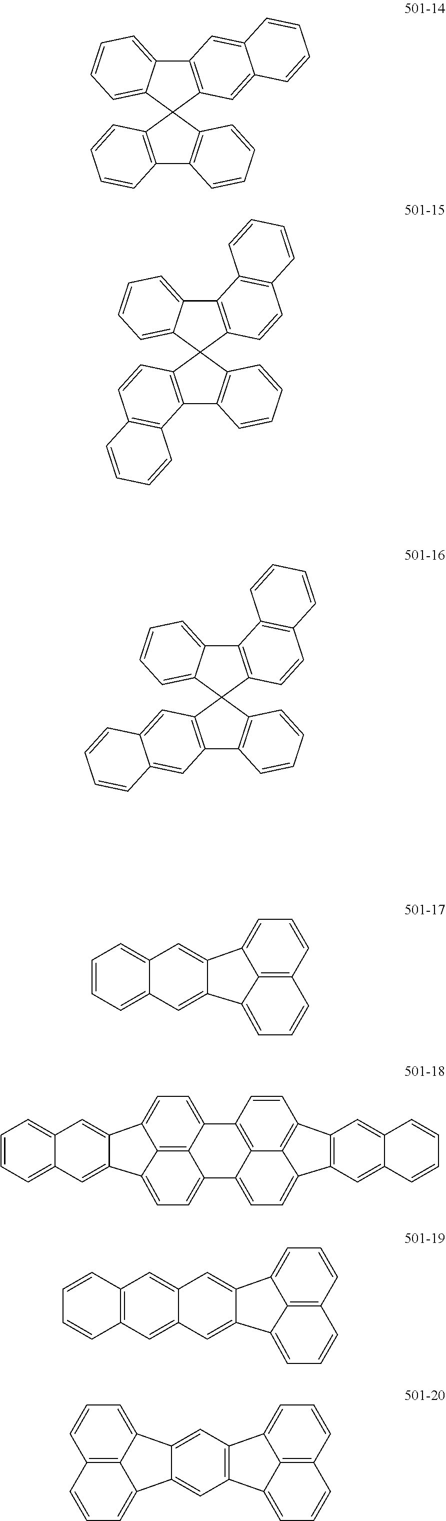

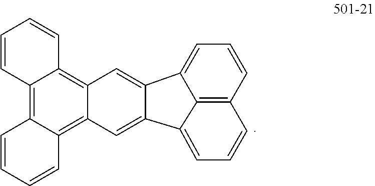 [SMILES: c1cc2c3c(cccc3c1)-c1cc3c4ccccc4c4ccccc4c3cc1-2]

In one or more embodiments, the prompt fluorescence material may include a compound represented by Formula 501A or 501B:

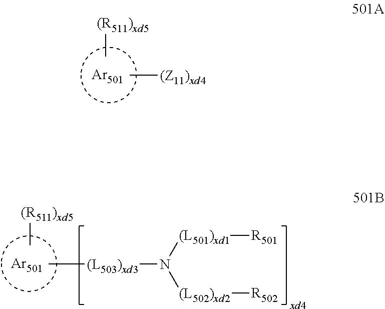

wherein, in Formulae 501A and 501B,
Ar₅₀₁ may be a naphthalene group, a fluorene group, a spiro-bifluorene group, a benzofluorene group, a dibenzofluorene group, a phenanthrene group, an anthracene group, a fluoranthene group, a triphenylene group, a pyrene group, a chrysene group, a naphthacene group, a picene group, a perylene group, a pentaphene group, an indenoanthracene group, a bisanthracene group, or a group represented by one of Formulae 501-1 to 501-21,
R₅₁₁ may be hydrogen, deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, or —Si(Q₅₀₁)(Q₅₀₂)(Q₅₀₃),
xd5 may be an integer from 0 to 10,
L₅₀₁ to L₅₀₃ may each independently be:
a single bond; and
a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, or a divalent non-aromatic condensed heteropolycyclic group, each unsubstituted or substituted with deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₅₀₁)(Q₅₀₂)(Q₅₀₃), or any combination thereof,
xd1 to xd3 may each independently be 1, 2, or 3,
R₅₀₁ and R₅₀₂ may each independently be a phenyl group, a naphthyl group, a fluorenyl group, a spiro-bifluorenyl group, a benzofluorenyl group, a dibenzofluorenyl group, a phenanthrenyl group, an anthracenyl group, a pyrenyl group, a chrysenyl group, a pyridinyl group, a pyrazinyl group, a pyrimidinyl group, a pyridazinyl group, a quinolinyl group, an isoquinolinyl group, a quinoxalinyl group, a quinazolinyl group, a carbazole group, a triazinyl group, a dibenzofuranyl group, a dibenzothiophenyl group, or a dibenzosilolyl group, each unsubstituted or substituted with deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₅₀₁)(Q₀₂)(Q₀₃), or any combination thereof,
Z₁₁ may be a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, a hydroxyl group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₅₀₁)(Q₀₂)(Q₀₃), or any combination thereof,
xd4 may be 1, 2, 3, 4, 5, or 6, and
Q₅₀₁ to Q₅₀₃ may each independently be hydrogen, a C₁-C₆₀ alkyl group, a C₁-C₆₀ alkoxy group, a C₆-C₆₀ aryl group, a C₁-C₆₀ heteroaryl group, a monovalent non-aromatic condensed polycyclic group, or a monovalent non-aromatic condensed heteropolycyclic group.

In one or more embodiments, the prompt fluorescence material may include a compound represented by Formula 501A or 501B, wherein in Formula 501A, xd4 may be 1, 2, 3, 4, 5, or 6, and in Formula 501B, xd4 may be 2, 3, or 4.

### Description of Host

The host in the emission layer may include a hole-transporting compound, an electron-transporting compound, a bipolar compound, or any combination thereof. The host may not include a transition metal.

For example, the host in the emission layer may include a hole-transporting compound and an electron-transporting compound, wherein the hole-transporting compound and the electron-transporting compound may be different from each other.

In an embodiment, the hole-transporting compound may include at least one π electron-rich C₃-C₆₀ cyclic group and may not include an electron-transporting group. Examples of the electron-transporting groups may include a cyano group, a fluoro group, a π electron-depleted nitrogen-containing cyclic group, a phosphine oxide group, a sulfoxide group, etc.

The “π electron-depleted nitrogen-containing cyclic group” as used herein may be a C₁-C₆₀ heterocyclic group including at least one *—N=*′ moiety as a ring-forming moiety. Examples of the π-electron deficient nitrogen-containing cyclic group may include a triazine group, an imidazole group, and the like.

The “π electron-rich C₃-C₆₀ cyclic group” as used herein may be a C₃-C₆₀ cyclic group which does not include a *—N=*′ moiety as a ring-forming moiety. Examples of the π electron-rich C₃-C₆₀ cyclic group may include a benzene group, a naphthalene group, a triphenylene group, a dibenzofuran group, a dibenzothiophene group, a carbazole group, an indolodibenzofuran group, an indolodibenzothiophene group, an indolocarbazole group, a naphthobenzofuran group, a naphthobenzothiophene group, a benzocarbazole group, a phenanthrenobenzofuran group, a phenanthrenobenzothiophene group, a naphthocarbazole group, a dinaphthofuran group, a dinaphthothiophene group, a dibenzocarbazole group, and the like.

For example, the hole-transporting compound may include two or more carbazole groups.

In an embodiment, the electron-transporting compound may be a compound including at least one electron-transporting group. The electron-transporting group may be a cyano group, a fluoro group, a π electron-depleted nitrogen-containing C₁-C₆₀ cyclic group, a phosphine oxide group, a sulfoxide group, or a combination thereof. In an embodiment, the electron-transporting compound may include a triazine group.

For example, the electron-transporting compound may include at least one electron-transporting group (for example, a triazine group) and at least one π electron-rich C₃-C₆₀ cyclic group (for example, a benzene group, a naphthalene group, a triphenylene group, a dibenzofuran group, a dibenzothiophene group, a carbazole group, an indolodibenzofuran group, an indolodibenzothiophene group, an indolocarbazole group, a naphthobenzofuran group, a naphthobenzothiophene group, a benzocarbazole group, a phenanthrenobenzofuran group, a phenanthrenobenzothiophene group, a naphthocarbazole group, a dinaphthofuran group, a dinaphthothiophene group, a dibenzocarbazole group, or any combination thereof).

In an embodiment, the hole-transporting compound may be a compound represented by Formula 6:

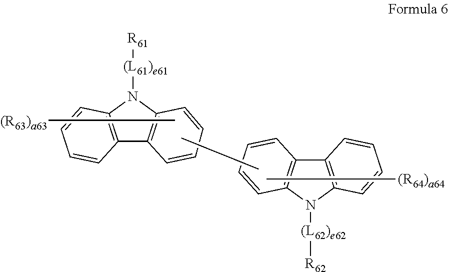

wherein, in Formula 6,
L₆₁ and L₆₂ may each independently be a π electron-rich C₃-C₆₀ cyclic group unsubstituted or substituted with deuterium, a C₁-C₂₀ alkyl group, a deuterated C₁-C₂₀ alkyl group, a phenyl group, a deuterated phenyl group, a (C₁-C₂₀ alkyl)phenyl group, a biphenyl group, a deuterated biphenyl group, a (C₁-C₂₀ alkyl)biphenyl group, —Si(Q₃₃)(Q₃₄)(Q₃₅), or any combination thereof,
e61 and e62 may each independently be an integer from 1 to 6,
R₆₁ to R₆₄ may each independently be:
hydrogen, deuterium, a C₁-C₂₀ alkyl group, or a deuterated C₁-C₂₀ alkyl group;
a π electron-rich C₃-C₆₀ cyclic group unsubstituted or substituted with deuterium, a C₁-C₂₀ alkyl group, a deuterated C₁-C₂₀ alkyl group, a phenyl group, a deuterated phenyl group, a (C₁-C₂₀ alkyl)phenyl group, a biphenyl group, a deuterated biphenyl group, a (C₁-C₂₀ alkyl)biphenyl group, —Si(Q₃₃)(Q₃₄)(Q₃₅), or any combination thereof; or
—Si(Q₃)(Q₄)(Q₅).

In Formula 6, a63 and a64 may each independently be an integer from 0 to 7.

Q₃ to Q₅ and Q₃₃ to Q₃₅ may each be the same as described herein.

In one or more embodiments, the hole-transporting compound may be a compound represented by Formula 6-1, 6-2, or 6-3:

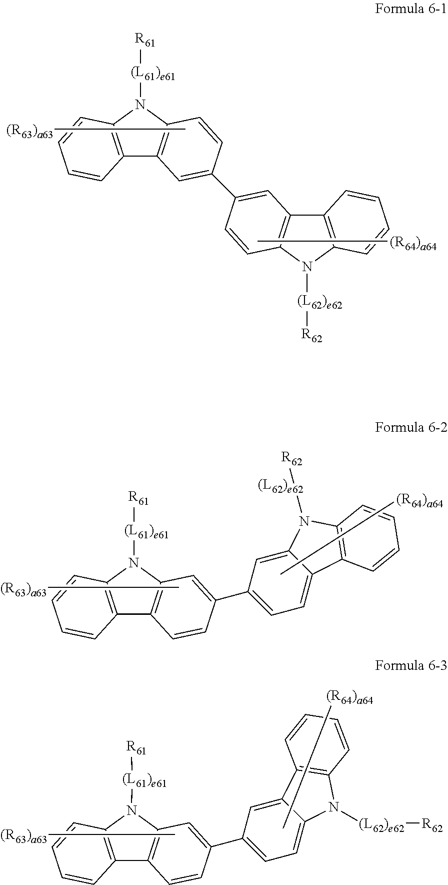

wherein, in Formulae 6-1 to 6-3, L₆₁, L₆₂, R₆₁ to R₆₄, e61, e62, a63, and a64 are respectively the same as those described herein.

In one or more embodiments, the hole-transporting compound may be represented by one of Compounds HTH1 to HTH6:

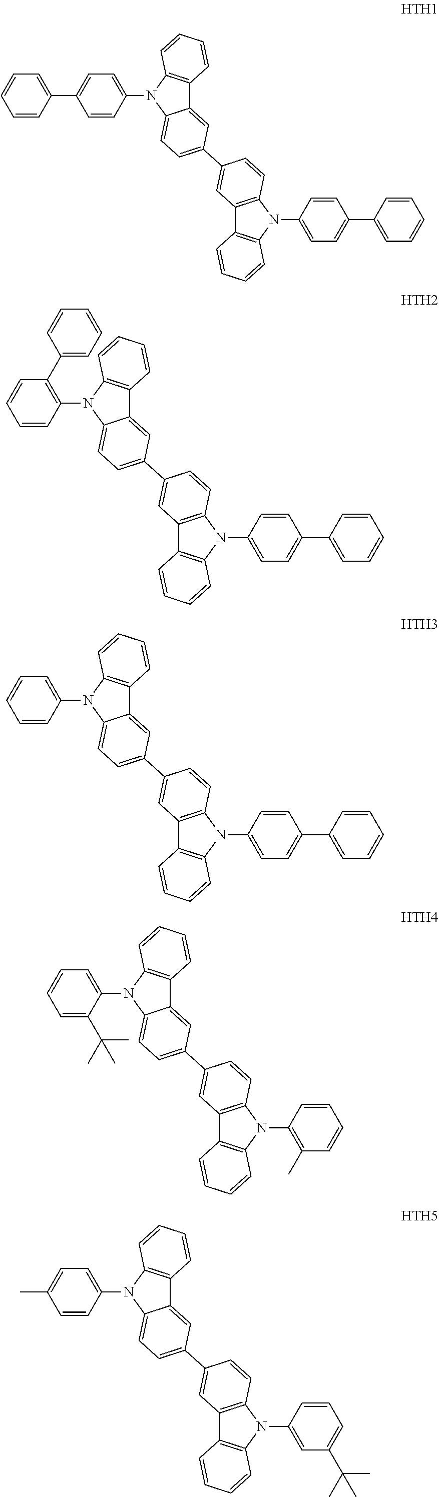

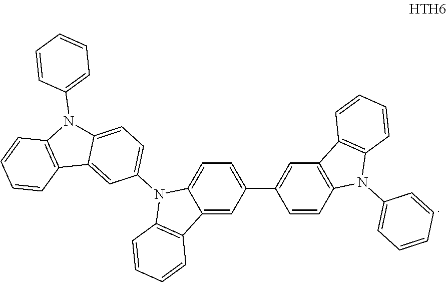 [SMILES: c1ccc(-n2c3ccccc3c3cc(-c4ccc5c(c4)c4ccccc4n5-c4ccc5c(c4)c4ccccc4n5-c4ccccc4)ccc32)cc1]

In one or more embodiments, the electron-transporting compound may be a compound represented by Formula 7:

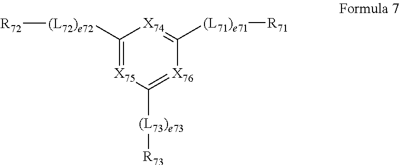

wherein, in Formula 7,
X₇₄ may be C(R₇₄) or N, X₇₅ may be C(R₇₅) or N, X₇₆ may be C(R₇₆) or N, and at least one of X₇₄ to X₇₆ may be N,
L₇₁ to L₇₃ may each independently be a C₅-C₃₀ carbocyclic group or a C₁-C₃₀ heterocyclic group, each unsubstituted or substituted with deuterium, —F, a cyano group, a C₁-C₂₀ alkyl group, a deuterated C₁-C₂₀ alkyl group, a fluorinated C₁-C₂₀ alkyl group, a phenyl group, a deuterated phenyl group, a fluorinated phenyl group, a (C₁-C₂₀ alkyl)phenyl group, a biphenyl group, a deuterated biphenyl group, a fluorinated biphenyl group, a (C₁-C₂₀ alkyl)biphenyl group, —Si(Q₃₃)(Q₃₄)(Q₃₅), or any combination thereof,
e71 to e73 may each independently be an integer from 1 to 10, and
R₇₁ to R₇₆ may each independently be:
hydrogen, deuterium, —F, or a cyano group;
a C₁-C₂₀ alkyl group that is unsubstituted or substituted with deuterium, —F, a cyano group, or any combination thereof;
a C₅-C₃₀ carbocyclic group or a C₁-C₃₀ heterocyclic group, each unsubstituted or substituted with deuterium, —F, a cyano group, a C₁-C₂₀ alkyl group, a deuterated C₁-C₂₀ alkyl group, a fluorinated C₁-C₂₀ alkyl group, a phenyl group, a deuterated phenyl group, a fluorinated phenyl group, a (C₁-C₂₀ alkyl)phenyl group, a biphenyl group, a deuterated biphenyl group, a fluorinated biphenyl group, a (C₁-C₂₀ alkyl)biphenyl group, —Si(Q₃₃)(Q₃₄)(Q₃₅), or any combination thereof; or
—Si(Q₃)(Q₄)(Q₅).
Q₃ to Q₅ and Q₃₃ to Q₃₅ may each be the same as described herein.

In one or more embodiments, X₇₄ to X₇₆ in Formula 7 may each be N.

In one or more embodiments, L₇₁ to L₇₃ in Formula 7 may each independently be a benzene group, a naphthalene group, a triphenylene group, a dibenzofuran group, a dibenzothiophene group, a carbazole group, an indolodibenzofuran group, an indolodibenzothiophene group, an indolocarbazole group, a naphthobenzofuran group, a naphthobenzothiophene group, a benzocarbazole group, a phenanthrenobenzofuran group, a phenanthrenobenzothiophene group, a naphthocarbazole group, a dinaphthofuran group, a dinaphthothiophene group, or a dibenzocarbazole group, each unsubstituted or substituted with deuterium, —F, a cyano group, a C₁-C₂₀ alkyl group, a deuterated C₁-C₂₀ alkyl group, a fluorinated C₁-C₂₀ alkyl group, a phenyl group, a deuterated phenyl group, a fluorinated phenyl group, a (C₁-C₂₀ alkyl)phenyl group, a biphenyl group, a deuterated biphenyl group, a fluorinated biphenyl group, a (C₁-C₂₀ alkyl)biphenyl group, —Si(Q₃₃)(Q₃₄)(Q₃₅), or any combination thereof.

In one or more embodiments, in Formula 7, at least one of L₇₁ in the number of e71, at least one of L₇₂ in the number of e72, at least one of L₇₃ in the number of e73, or any combination thereof may each independently be a dibenzofuran group, a dibenzothiophene group, a carbazole group, an indolodibenzofuran group, an indolodibenzothiophene group, an indolocarbazole group, a naphthobenzofuran group, a naphthobenzothiophene group, a benzocarbazole group, a phenanthrenobenzofuran group, a phenanthrenobenzothiophene group, a naphthocarbazole group, a dinaphthofuran group, a dinaphthothiophene group, or a dibenzocarbazole group, each unsubstituted or substituted with deuterium, —F, a cyano group, a C₁-C₂₀ alkyl group, a deuterated C₁-C₂₀ alkyl group, a fluorinated C₁-C₂₀ alkyl group, a phenyl group, a deuterated phenyl group, a fluorinated phenyl group, a (C₁-C₂₀ alkyl)phenyl group, a biphenyl group, a deuterated biphenyl group, a fluorinated biphenyl group, a (C₁-C₂₀ alkyl)biphenyl group, —Si(Q₃₃)(Q₃₄)(Q₃₅), or any combination thereof.

In one or more embodiments, in Formula 7, at least one of L₇₁ in the number of e71, at least one of L₇₂ in the number of e72, at least one of L₇₃ in the number of e73, or any combination thereof may include a carbazole group, an indolocarbazole group, a benzocarbazole group, a naphthocarbazole group, or a dibenzocarbazole group, wherein a nitrogen atom of a pyrrole group of, the carbazole group, the indolocarbazole group, the benzocarbazole group, the naphtho carbazole group, or the dibenzocarbazole group, may be linked to a carbon atom of a 6-membered ring including X₇₄ to X₇₆ in Formula 7 via a single bond or with neighboring L₇₁, L₇₂, and/or L₇₃ located therebetween.

In one or more embodiments, e71 to e73 in Formula 7 indicate the numbers of L₇₁ to L₇₃, respectively, and may each independently be 1, 2, 3, 4, or 5.

In one or more embodiments, R₇₁ to R₇₆ in Formula 7 may each independently be:
hydrogen, deuterium, —F, or a cyano group;
a C₁-C₂₀ alkyl group that is unsubstituted or substituted with deuterium, —F, a cyano group, or any combination thereof;
a benzene group, a naphthalene group, a triphenylene group, a dibenzofuran group, a dibenzothiophene group, a carbazole group, an indolodibenzofuran group, an indolodibenzothiophene group, an indolocarbazole group, a naphthobenzofuran group, a naphthobenzothiophene group, a benzocarbazole group, a phenanthrenobenzofuran group, a phenanthrenobenzothiophene group, a naphthocarbazole group, a dinaphthofuran group, a dinaphthothiophene group, or a dibenzocarbazole group, each unsubstituted or substituted with deuterium, —F, a cyano group, a C₁-C₂₀ alkyl group, a deuterated C₁-C₂₀ alkyl group, a fluorinated C₁-C₂₀ alkyl group, a phenyl group, a deuterated phenyl group, a fluorinated phenyl group, a (C₁-C₂₀ alkyl)phenyl group, a biphenyl group, a deuterated biphenyl group, a fluorinated biphenyl group, a (C₁-C₂₀ alkyl)biphenyl group, —Si(Q₃₃)(Q₃₄)(Q₃₅), or any combination thereof; or
—Si(Q₃)(Q₄)(Q₅).

In one or more embodiments, the electron-transporting compound may be represented by one of Compounds ETH1 to ETH7:

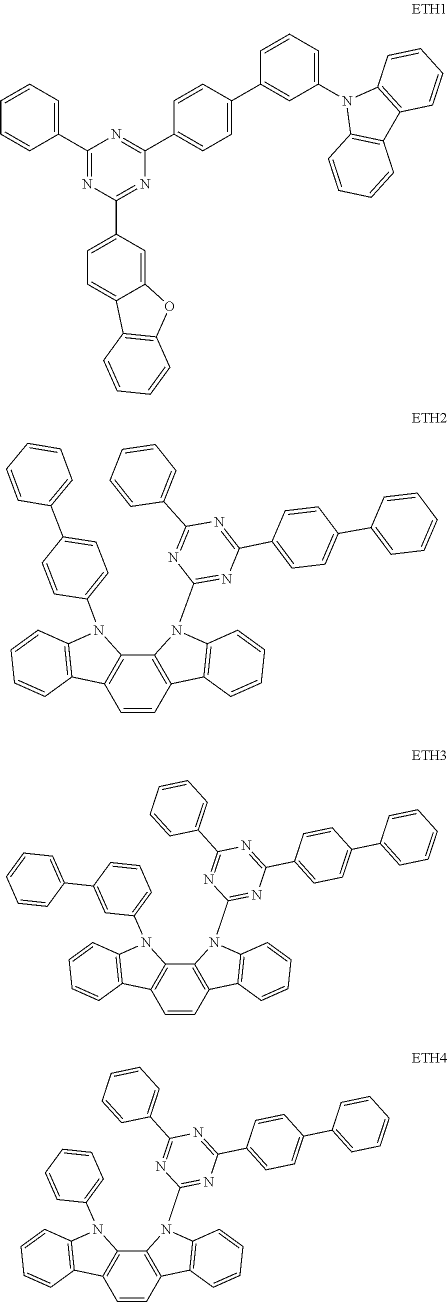 [SMILES: c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2ccc(-c3nc(-c4ccccc4)nc(-n4c5ccccc5c5ccc6c7ccccc7n(-c7ccc(-c8ccccc8)cc7)c6c54)n3)cc2)cc1 ; c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1 ; c1ccc(-c2ccc(-c3nc(-c4ccccc4)nc(-n4c5ccccc5c5ccc6c7ccccc7n(-c7ccccc7)c6c54)n3)cc2)cc1]

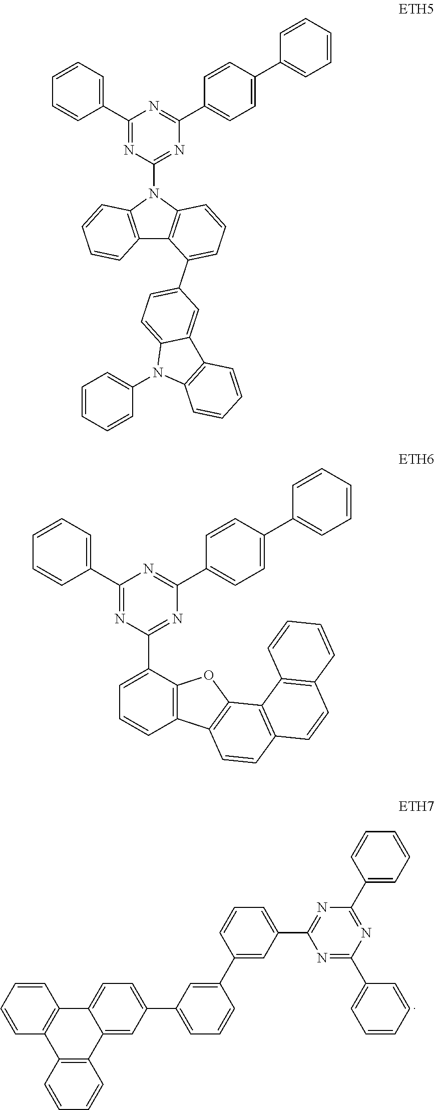 [SMILES: c1ccc(-c2ccc(-c3nc(-c4ccccc4)nc(-n4c5ccccc5c5c(-c6ccc7c(c6)c6ccccc6n7-c6ccccc6)cccc54)n3)cc2)cc1 ; c1ccc(-c2ccc(-c3nc(-c4ccccc4)nc(-c4cccc5c4oc4c5ccc5ccc6ccccc6c54)n3)cc2)cc1 ; c1ccc(-c2nc(-c3ccccc3)nc(-c3cccc(-c4cccc(-c5ccc6c7ccccc7c7ccccc7c6c5)c4)c3)n2)cc1]

### Description of Formula 3

In the first and second embodiments, the organometallic compound may be an organometallic compound represented by Formula 3:

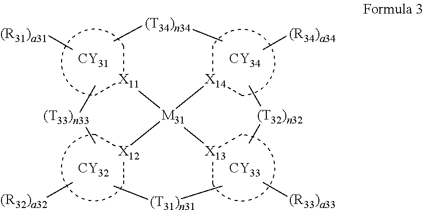

wherein, in Formula 3,
M₃₁ may be a transition metal,
X₁₁ to X₁₄ may each independently be C or N,
two bonds of a bond between X₁₁ and M₃₁, a bond between X₁₂ and M₃₁, a bond between X₁₃ and M₃₁, and a bond between X₁₄ and M₃₁ may each be a coordinate bond, and the other two bonds may each be a covalent bond,
ring CY₃₁ to ring CY₃₄ may each independently be a C₅-C₃₀ carbocyclic group or a C₁-C₃₀ heterocyclic group,
T₃₁ may be a single bond, a double bond, *—N(R₃₅ₐ)—*′, *—B(R₃₅ₐ)—*′, *—P(R₃₅ₐ)—*′, *—C(R₃₅ₐ)(R_{35b})—*′, *—Si(R₃₅ₐ)(R_{35b})—*′, *—Ge(R₃₅ₐ)(R_{35b})—*′, *—S—*′, *—Se—*′, *—O—*′, *—C(═O)—*′, *—S(═O)—*′, *—S(═O)₂—*′, *—C(R₃₅ₐ)=*′, *=C(R₃₅ₐ)—*′, *—C(R₃₅ₐ)═C(R_{35b})—*′, *—C(═S)—*′, *—C≡C—*′, a C₅-C₃₀ carbocyclic group unsubstituted or substituted with at least one R₁₀ₐ, or a C₁-C₃₀ heterocyclic group unsubstituted or substituted with at least one R₁₀ₐ,
T₃₂ may be a single bond, a double bond, *—N(R₃₆ₐ)—*′, *—B(R₃₆ₐ)—*′, *—P(R₃₆ₐ)—*′, *—C(R₃₆ₐ)(R_{36b})—*′, *—Si(R₃₆ₐ)(R_{36b})—*′, *—Ge(R₃₆ₐ)(R_{36b})—*′, *—S—*′, *—Se—*′, *—O—*′, *—C(═O)—*′, *—S(═O)—*′, *—S(═O)₂—*′, *—C(R₃₆ₐ)=*′, *=C(R₃₆ₐ)—*′, *—C(R₃₆ₐ)═C(R_{36b})—*′, *—C(═S)—*′, *—C≡C—*′, a C₅-C₃₀ carbocyclic group unsubstituted or substituted with at least one R₁₀ₐ, or a C₁-C₃₀ heterocyclic group unsubstituted or substituted with at least one R₁₀ₐ,
T₃₃ may be a single bond, a double bond, *—N(R₃₇ₐ)—*′, *—B(R₃₇ₐ)—*′, *—P(R₃₇ₐ)—*′, *—C(R₃₇ₐ)(R_{37b})—*′, *—Si(R₃₇ₐ)(R_{37b})—*′, *—Ge(R₃₇ₐ)(R_{37b})—*′, *—S—*′, *—Se—*′, *—O—*′, *—C(═O)—*′, *—S(═O)—*′, *—S(═O)₂—*′, *—C(R₃₇ₐ)=*′, *=C(R₃₇ₐ)—*′, *—C(R₃₇ₐ)═C(R_{37b})—*′, *—C(═S)—*′, *—C≡C—*′ a C₅-C₃₀ carbocyclic group unsubstituted or substituted with at least one R₁₀ₐ, or a C₁-C₃₀ heterocyclic group unsubstituted or substituted with at least one R₁₀ₐ,
T₃₄ may be a single bond, a double bond, *—N(R₃₈ₐ)—*′, *—B(R₃₈ₐ)—*′, *—P(R₃₈ₐ)—*′, *—C(R₃₈a)(R_{38b})—*′, *—Si(R₃₈ₐ)(R_{38b})—*′, *—Ge(R₃₈ₐ)(R_{38b})—*′, *—S—*′, *—Se—*′, *—O—*′, *—C(═O)—*′, *—S(═O)—*′, *—S(═O)₂—*′, *—C(R₃₈ₐ)=*′, *=C(R₃₈ₐ)—*′, *—C(R₃₈ₐ)═C(R_{38b})—*′, *—C(═S)—*′, *—C≡C—*′, a C₅-C₃₀ carbocyclic group unsubstituted or substituted with at least one R₁₀ₐ, or a C₁-C₃₀ heterocyclic group unsubstituted or substituted with at least one R₁₀ₐ,
n31 to n34 may each independently be an integer from 0 to 5, and three or more of n31 to n34 may each independently be an integer from 1 to 5,
when n31 is 0, T₃₁ may not be present, when n32 is 0, T₃₂ may not be present, when n33 is 0, T₃₃ may not be present, and when n34 is 0, T₃₄ may not be present,
when n31 is 2 or more, two or more of T₃₁ may be identical to or different from each other, when n32 is 2 or more, two or more of T₃₂ may be identical to or different from each other, when n33 is 2 or more, two or more of T₃₃ may be identical to or different from each other, and when n34 is 2 or more, two or more of T₃₄ may be identical to or different from each other,
R₃₁ to R₃₄, R₃₅ₐ, R_{35b}, R₃₆ₐ, R_{36b}, R₃₇ₐ, R_{37b}, R₃₈ₐ, and R_{38b} may each independently be hydrogen, deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
a31 to a34 may each independently be an integer from 0 to 20,
two or more of R₃₁ in the number of a31 may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
two or more of R₃₂ in the number of a32 may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
two or more of R₃₃ in the number of a33 may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
two or more of R₃₄ in the number of a34 may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
two or more of R₃₁ to R₃₄, R₃₅ₐ, R_{35b}, R₃₆ₐ, R_{36b}, R₃₇ₐ, R_{37b}, R₃₈ₐ, and R_{38b} may optionally be bonded to each other to form a C₅-C₃₀ carbocyclic group unsubstituted or substituted with at least one R₁₀ₐ or a C₁-C₃₀ heterocyclic group unsubstituted or substituted with at least one R₁₀ₐ,
R₁₀ₐ is the same as described in connection with R₃₁,
* and *′ each indicate a binding site to a neighboring atom,
a substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
any combination thereof, and
Q₁ to Q₉, Q₁₁ to Q₁₉, Q₂₁ to Q₂₉, and Q₃₁ to Q₃₉ may each independently be: hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; a C₁-C₆₀ alkyl group unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₁-C₁₀ heterocycloalkyl group; a C₃-C₁₀ cycloalkenyl group; a C₁-C₁₀ heterocycloalkenyl group; a C₆-C₆₀ aryl group unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₁-C₆₀ heteroaryl group; a monovalent non-aromatic condensed polycyclic group; or a monovalent non-aromatic condensed heteropolycyclic group.

In an embodiment, M₃₁ in Formula 3 may be Pt, Pd, or Au.

In one or more embodiments, M₃₁ in Formula 3 may be Pt or Pd.

In one or more embodiments, in Formula 3, a bond between X₁₁ and M₃₁ may be a coordinate bond.

In one or more embodiments, in Formula 3, X₁₁ may be C, and a bond between X₁₁ and M₃₁ may be a coordinate bond. That is, X₁₁ in Formula 3 may be C in a carbene moiety.

In one or more embodiments, ring CY₃₁ to ring CY₃₄ in Formula 3 may each independently be i) a first ring, ii) a second ring, iii) a condensed ring in which two or more first rings are condensed with each other, iv) a condensed ring in which two or more second rings are condensed with each other, or v) a condensed ring in which at least one first ring is condensed with at least one second ring,
the first ring may be a cyclopentane group, a cyclopentadiene group, a furan group, a thiophene group, a pyrrole group, a silole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, or a triazasilole group, and
the second ring may be an adamantane group, a norbornane group, a norbornene group, a cyclohexane group, a cyclohexene group, a benzene group, a pyridine group, a pyrimidine group, a pyrazine group, a pyridazine group, a triazine group, an oxazine group, a thiazine group, a dihydropyrazine group, a dihydropyridine group, or a dihydroazasilane group.

In an embodiment, R₃₁ to R₃₄, R₃₅ₐ, R_{35b}, R₃₆ₐ, R_{36b}, R₃₇ₐ, R_{37b}, R₃₈ₐ, and R_{38b} may each independently be:
hydrogen, deuterium, —F, —Cl, —Br, —I, —CD₃, —CD₂H, —CDH₂, —CF₃, —CF₂H, —CFH₂, a cyano group (CN), a nitro group, an amino group, a C₁-C₂₀ alkyl group, or a C₁-C₂₀ alkoxy group;
a C₁-C₂₀ alkyl group or a C₁-C₂₀ alkoxy group, each substituted with at least one of deuterium, —F, —Cl, —Br, —I, —CD₃, —CD₂H, —CDH₂, —CF₃, —CF₂H, —CFH₂, a cyano group (CN), a nitro group, an amino group, and a phenyl group; or
a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, or an anthracenyl group, each unsubstituted or substituted with at least one of deuterium, —F, —Cl, —Br, —I, —CD₃, —CD₂H, —CDH₂, —CF₃, —CF₂H, —CFH₂, a cyano group (CN), a nitro group, an amino group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, and an anthracenyl group.

In an embodiment, the organometallic compound represented by Formula 3 may be an organometallic compound represented by Formula 3-1 or 3-2:

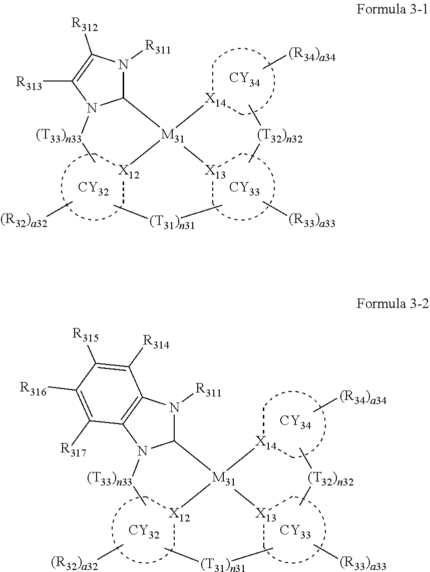

In Formula 3-1, a bond between carbon of an imidazole group and M₃₁ may be a coordinate bond. That is, the imidazole group in Formula 3-1 may include a carbene moiety bonded to M₃₁.

In Formula 3-2, a bond between carbon of a benzimidazole group and M₃₁ may be a coordinate bond. That is, the benzimidazole group in Formula 3-2 may include a carbene moiety bonded to M₃₁.

In Formulae 3-1 and 3-2,
M₃₁, CY₃₂, CY₃₃, CY₃₄, X₁₂, X₁₃, X₁₄, T₃₁, T₃₂, T₃₃, n31, n32, n33, R₃₂, R₃₃, R₃₄, a32, a33, and a34 may respectively be understood by referring to the descriptions of M₃₁, CY₃₂, CY₃₃, CY₃₄, X₁₂, X₁₃, X₁₄, T₃₁, T₃₂, T₃₃, n31, n32, n33, R₃₂, R₃₃, R₃₄, a32, a33, and a34 provided herein, and
R₃₁₁ to R₃₁₇ may each be understood by referring to the description of R₃₁ provided herein.

In an embodiment, in Formulae 3-1 and 3-2,
R₃₁₁ to R₃₁₇ may each independently be:
hydrogen, deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, or a phosphoric acid group or a salt thereof;
a C₁-C₂₀ alkyl group or a C₁-C₂₀ alkoxy group, each unsubstituted or substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, or any combination thereof;
a C₃-C₁₀ cycloalkyl group, a C₁-C₁₀ heterocycloalkyl group, a C₆-C₆₀ aryl group, a C₇-C₆₀ arylalkyl group, a C₆-C₆₀ aryloxy group, a C₆-C₆₀ arylthio group, a C₁-C₆₀ heteroaryl group, a substituted or unsubstituted C₂-C₆₀ heteroaryl alkyl group, a monovalent non-aromatic condensed polycyclic group, a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a C₁-C₂₀ alkyl group, a C₁-C₂₀ alkoxy group, or any combination thereof; or
—N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

For example, in Formulae 3-1 and 3-2,
at least one of R₃₁₁ to R₃₁₇ may be a C₁-C₂₀ alkyl group, a C₆-C₆₀ aryl group, or a C₇-C₆₀ arylalkyl group, each unsubstituted or substituted with at least one of a methyl group, an ethyl group, an n-propyl group, an isopropyl group, an n-butyl group, an isobutyl group, a sec-butyl group, a tert-butyl group, a phenyl group, a cumyl group, or a combination thereof.

### Description of Formula 5

In the first and second embodiments, the organometallic compound may be an organometallic compound represented by Formula 5:

M₅₁(L₅₁)ₙ₅₁(L₅₂)ₙ₅₂ Formula 5
wherein, in Formula 5, M₅₁ may be a transition metal.

In some embodiments, M₅₁ may be a first-row transition metal, a second-row transition metal, or a third-row transition metal.

In some embodiments, M₅₁ may be iridium (Ir), platinum (Pt), osmium (Os), titanium (Ti), zirconium (Zr), hafnium (Hf), europium (Eu), terbium (Tb), thulium (Tm), or rhodium (Rh).

In some embodiments, M₅₁ may be Ir, Pt, Os, or Rh.

In one or more embodiments, M₅₁ may be Ir or Os.

In Formula 5, L₅₁ may be a ligand represented by Formula 5A, and L₅₂ may be a ligand represented by Formula 5B:

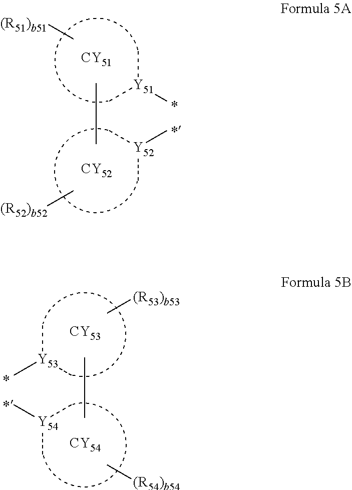

wherein Formulae 5A and 5B are each the same as described in the specification.

In Formula 5. n51 may be 1, 2, or 3, wherein, when n51 is 2 or more, two or more of L₅₁ may be identical to or different from each other.

In Formula 5, n52 may be 0, 1, or 2, wherein, when n52 is 2, two L₅₂ may be identical to or different from each other.

The sum of n51 and n52 in Formula 5 may be 2 or 3. For example, the sum of n51 and n52 may be 3.

In an embodiment, in Formula 5, i) M may be Ir, and n51+n52=3; or ii) M may be Pt, and n51+n52=2.

In one or more embodiments, in Formula 5, M may be Ir, and i) n51 may be 1, and n52 may be 2, or ii) n51 may be 2, and n52 may be 1.

L₅₁ and L₅₂ in Formula 5 may be different from each other.

Y₅₁ to Y₅₄ in Formulae 5A to 5B may each independently be C or N. For example, Y₅₁ and Y₅₃ may each be N, and Y₅₂ and Y₅₄ may each be C.

Ring CY₅₁ to ring CY₅₄ in Formulae 5A and 5B may each independently be a C₅-C₃₀ carbocyclic group or a C₁-C₃₀ heterocyclic group.

For example, ring CY₅₁ to ring CY₅₄ in Formulae 5A and 5B may each independently include i) a third ring, ii) a fourth ring, iii) a condensed ring in which two or more third rings are condensed with each other, iv) a condensed ring in which two or more fourth rings are condensed with each other, or v) a condensed ring in which at least one third ring is condensed with at least one fourth ring,
the third ring may be a cyclopentane group, a cyclopentene group, a furan group, a thiophene group, a pyrrole group, a silole group, a borole group, a phosphole group, a germole group, a selenophene group, an oxazole group, an oxadiazole group, an oxatriazole group, a thiazole group, a thiadiazole group, a thiatriazole group, a pyrazole group, an imidazole group, a triazole group, a tetrazole group, or an azasilole group, and
the fourth ring may be an adamantane group, a norbornane group, a norbornene group, a cyclohexane group, a cyclohexene group, a benzene group, a pyridine group, a pyrimidine group, a pyrazine group, a pyridazine group, or a triazine group.

In some embodiments, in Formulae 5A and 5B, ring CY₁ to ring CY₄ may each independently be a cyclopentane group, a cyclohexane group, a cyclohexene group, a benzene group, a naphthalene group, an anthracene group, a phenanthrene group, a triphenylene group, a pyrene group, a chrysene group, a 1,2,3,4-tetrahydronaphthalene group, a cyclopentadiene group, a pyrrole group, a furan group, a thiophene group, a silole group, a borole group, a phosphole group, a germole group, a selenophene group, an indene group, an indole group, a benzofuran group, a benzothiophene group, a benzosilole group, a benzoborole group, a benzophosphole group, a benzogermole group, a benzoselenophene group, a fluorene group, a carbazole group, a dibenzofuran group, a dibenzothiophene group, a dibenzosilole group, a dibenzoborole group, a dibenzophosphole group, a dibenzogermole group, a dibenzoselenophene group, a benzofluorene group, a benzocarbazole group, a naphthobenzofuran group, a naphthobenzothiophene group, a naphthobenzosilole group, a naphthobenzoborole group, a naphthobenzophosphole group, a naphthobenzogermole group, a naphthobenzoselenophene group, a dibenzofluorene group, a dibenzocarbazole group, a dinaphthofuran group, a dinaphthothiophene group, a dinaphthosilole group, a dinaphthoborole group, a dinaphthophosphole group, a dinaphthogermole group, a dinaphthoselenophene group, an indenophenanthrene group, an indolophenanthrene group, a phenanthrobenzofuran group, a phenanthrobenzothiophene group, a phenanthrobenzosilole group, a phenanthrobenzoborole group, a phenanthrobenzophosphole group, a phenanthrobenzogermole group, a phenanthrobenzoselenophene group, a dibenzothiophene 5-oxide group, a 9H-fluorene-9-one group, a dibenzothiophene 5,5-dioxide group, an azaindene group, an azaindole group, an azabenzofuran group, an azabenzothiophene group, an azabenzosilole group, an azabenzoborole group, an azabenzophosphole group, an azabenzogermole group, an azabenzoselenophene group, an azafluorene group, an azacarbazole group, an azadibenzofuran group, an azadibenzothiophene group, an azadibenzosilole group, an azadibenzoborole group, an azadibenzophosphole group, an azadibenzogermole group, an azadibenzoselenophene group, an azabenzofluorene group, an azabenzocarbazole group, an azanaphthobenzofuran group, an azanaphthobenzothiophene group, an azanaphthobenzosilole group, an azanaphthobenzoborole group, an azanaphthobenzophosphole group, an azanaphthobenzogermole group, an azanaphthobenzoselenophene group, an azadibenzofluorene group, an azadibenzocarbazole group, an azadinaphthofuran group, an azadinaphthothiophene group, an azadinaphthosilole group, an azadinaphthoborole group, an azadinaphthophosphole group, an azadinaphthogermole group, an azadinaphthoselenophene group, an azaindenophenanthrene group, an azaindolophenanthrene group, an azaphenanthrobenzofuran group, an azaphenanthrobenzothiophene group, an azaphenanthrobenzosilole group, an azaphenanthrobenzoborole group, an azaphenanthrobenzophosphole group, an azaphenanthrobenzogermole group, an azaphenanthrobenzoselenophene group, an azadibenzothiophene 5-oxide group, an aza9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a benzoquinoline group, a benzoisoquinoline group, a benzoquinoxaline group, a benzoquinazoline group, a phenanthroline group, a phenanthridine group, a pyrrole group, a pyrazole group, an imidazole group, a triazole group, an oxazole group, an isooxazole group, a thiazole group, an isothiazole group, an oxadiazole group, a thiadiazole group, an azasilole group, an azaborole group, an azaphosphole group, an azagermole group, an azaselenophene group, a benzopyrrole group, a benzopyrazole group, a benzimidazole group, a benzoxazole group, a benzisoxazole group, a benzothiazole group, a benzisothiazole group, a benzoxadiazole group, a benzothiadiazole group, a pyridinopyrrole group, a pyridinopyrazole group, a pyridinoimidazole group, a pyridinooxazole group, a pyridinoisoxazole group, a pyridinothiazole group, a pyridinoisothiazole group, a pyridinooxadiazole group, a pyridinothiadiazole group, a pyrimidinopyrrole group, a pyrimidinopyrazole group, a pyrimidinoimidazole group, a pyrimidinooxazole group, a pyrimidinoisoxazole group, a pyrimidinothiazole group, a pyrimidinoisothiazole group, a pyrimidinooxadiazole group, a pyrimidinothiadiazole group, a 5,6,7,8-tetrahydroisoquinoline group, a 5,6,7,8-tetrahydroquinoline group, an adamantane group, a norbornane group, a norbornene group, a benzene group condensed with a cyclohexane group, a benzene group condensed with a norbornane group, a pyridine group condensed with a cyclohexane group, or a pyridine group condensed with a norbornane group.

For example, ring CY₅₁ and ring CY₅₃ in Formulae 5A and 5B may be different from each other.

In one or more embodiments, ring CY₅₂ and ring CY₅₄ in Formulae 5A and 5B may be different from each other.

In one or more embodiments, ring CY₅₁ to ring CY₅₄ in Formulae 5A and 5B may be different from each other.

R₅₁ to R₅₄ in Formulae 5A and 5B may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, a substituted or unsubstituted monovalent non-aromatic condensed heteropolycyclic group, —N(Q₅₁)(Q₅₂), —Si(Q₅₃)(Q₅₄)(Q₅₅), —Ge(Q₅₃)(Q₅₄)(Q₅₅), —B(Q₅₆)(Q₅₇), —P(═O)(Q₅₈)(Q₅₉), or —P(Q₅₈)(Q₅₉). Q₅₁ to Q₅₉ are each the same as described in the specification.

In an embodiment, R₅₁ to R₅₄ in Formulae 5A and 5B may each independently be: hydrogen, deuterium, —F, —Cl, —Br, —I, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, a C₁-C₂₀ alkoxy group, or a C₁-C₂₀ alkylthio group; a C₁-C₂₀ alkyl group, a C₁-C₂₀ alkoxy group, or a C₁-C₂₀ alkylthio group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrimidinyl group, or any combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group or azadibenzothiophenyl group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a (phenyl)C₁-C₁₀ alkyl group, a C₁-C₂₀ alkoxy group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or any combination thereof; or
—N(Q₅₁)(Q₅₂), —Si(Q₅₃)(Q₅₄)(Q₅₅), —Ge(Q₅₃)(Q₅₄)(Q₅₅), —B(Q₅₆)(Q₅₇), —P(═O)(Q₅₈)(Q₅₉), or —P(Q₅₈)(Q₅₉), and
Q₅₁ to Q₅₉ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, a C₁-C₁₀ alkyl group, a phenyl group, or any combination thereof.

In one or more embodiments, R₅₁ to R₅₄ may each independently be:
hydrogen, deuterium, —F, or a cyano group;
a C₁-C₂₀ alkyl group unsubstituted or substituted with deuterium, a cyano group, a C₃-C₁₀ cycloalkyl group, a deuterated C₃-C₁₀ cycloalkyl group, a fluorinated C₃-C₁₀ cycloalkyl group, a (C₁-C₂₀ alkyl)C₃-C₁₀ cycloalkyl group, a C₁-C₁₀ heterocycloalkyl group, a deuterated C₁-C₁₀ heterocycloalkyl group, a fluorinated C₁-C₁₀ heterocycloalkyl group, a (C₁-C₂₀ alkyl)C₁-C₁₀ heterocycloalkyl group, a phenyl group, a deuterated a phenyl group, a fluorinated a phenyl group, a (C₁-C₂₀ alkyl)phenyl group, a biphenyl group, a deuterated a biphenyl group, a fluorinated a biphenyl group, a (C₁-C₂₀ alkyl)biphenyl group, a dibenzofuranyl group, a deuterated a dibenzofuranyl group, a fluorinated a dibenzofuranyl group, a (C₁-C₂₀ alkyl)dibenzofuranyl group, a dibenzothiophenyl group, a deuterated a dibenzothiophenyl group, a fluorinated a dibenzothiophenyl group, a (C₁-C₂₀ alkyl)dibenzothiophenyl group, or any combination thereof;
a C₃-C₁₀ cycloalkyl group, a C₁-C₁₀ heterocycloalkyl group, a phenyl group, or a biphenyl group, each unsubstituted or substituted with deuterium, a cyano group, a C₁-C₂₀ alkyl group, a deuterated C₁-C₂₀ alkyl group, fluorinated C₁-C₂₀ alkyl group, a C₁-C₂₀ alkoxy group, a deuterated C₁-C₂₀ alkoxy group, a fluorinated C₁-C₂₀ alkoxy group, a C₃-C₁ cycloalkyl group, a deuterated C₃-C₁₀ cycloalkyl group, a fluorinated C₃-C₁₀ cycloalkyl group, a (C₁-C₂₀ alkyl)C₃-C₁₀ cycloalkyl group, a C₁-C₁₀ heterocycloalkyl group, a deuterated C₁-C₁₀ heterocycloalkyl group, a fluorinated C₁-C₁₀ heterocycloalkyl group, a (C₁-C₂₀ alkyl)C₁-C₁₀ heterocycloalkyl group, a phenyl group, a deuterated phenyl group, a fluorinated a phenyl group, a (C₁-C₂₀ alkyl)phenyl group, a biphenyl group, a deuterated biphenyl group, a fluorinated a biphenyl group, a (C₁-C₂₀ alkyl)biphenyl group, a dibenzofuranyl group, a deuterated dibenzofuranyl group, a fluorinated a dibenzofuranyl group, a (C₁-C₂₀ alkyl)dibenzofuranyl group, a dibenzothiophenyl group, a deuterated dibenzothiophenyl group, a fluorinated a dibenzothiophenyl group, a (C₁-C₂₀ alkyl)dibenzothiophenyl group, or any combination thereof; or
—Si(Q₅₃)(Q₅₄)(Q₅₅) or —Ge(Q₅₃)(Q₅₄)(Q₅₅).

In Formulae 5A and 5B, b51 to b54 indicate the numbers of R₅₁ to R₅₄, respectively, and may each independently be an integer from 0 to 20. When b51 is 2 or more, two or more of R₅₁ may be identical to or different from each other, when b52 is 2 or more, two or more of R₅₂ may be identical to or different from each other, when b53 is 2 or more, two or more of R₅₃ may be identical to or different from each other, and when b54 is 2 or more, two or more of R₅₄ may be identical to or different from each other. For example, b51 to b54 may each independently be an integer from 0 to 8.

In an embodiment, in Formula 5A, Y₅₂ may be C, a bond between Y₅₂ and M₅₁ may be a covalent bond, and at least one of R₅₂ in the number of b52 may be a cyano group or —F.

In one or more embodiments, in Formula 5A, Y₅₁ may be N, a bond between Y₅₁ and M₅₁ may be a coordinate bond, CY₅₁ may be an imidazole group, a triazole group, a benzimidazole group, or a triazolopyridine group, and at least one of R₅₂ in the number of b52 may be a cyano group or —F.

In one or more embodiments, in Formula 5A, Y₅₁ may be C, and a bond between Y₅₁ and M₅₁ may be a coordinate bond.

In one or more embodiments, in Formula 5A, Y₅₁ may be C, a bond between Y₅₁ and M₅₁ may be a coordinate bond, and CY₅₁ may be a benzimidazole group or an imidazopyrazine group.

Specific examples of organometallic compound represented by Formula 3 or 5

For example, the organometallic compound represented by Formula 3 or 5 may be represented by one of Compounds P1 to P52:

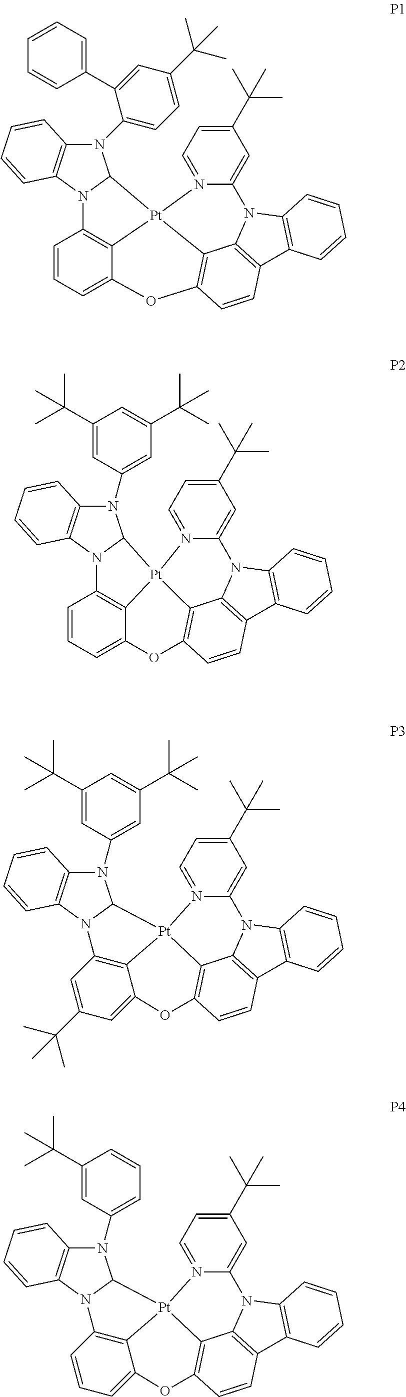

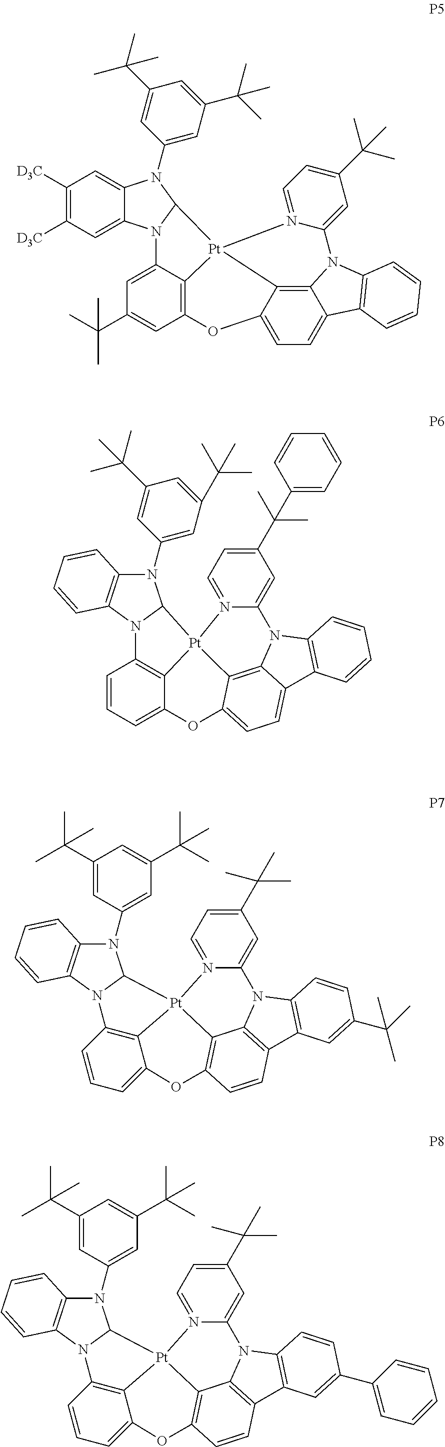

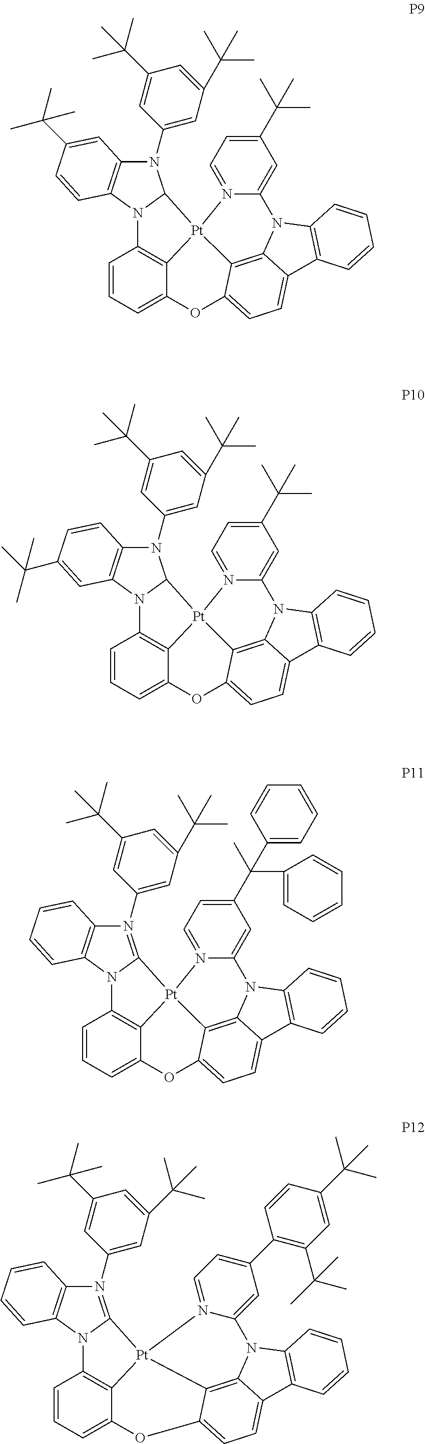

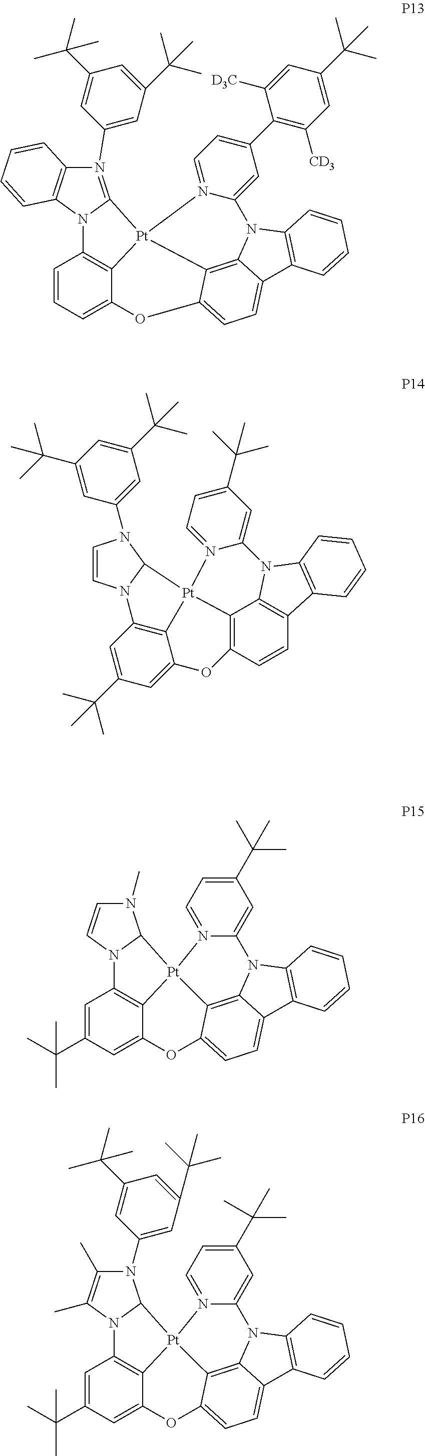

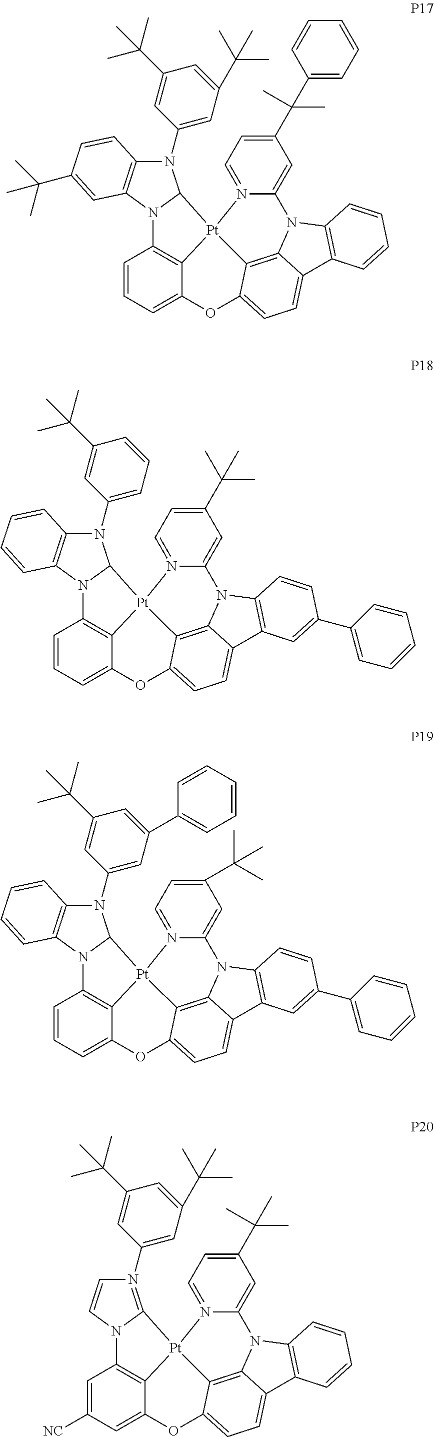

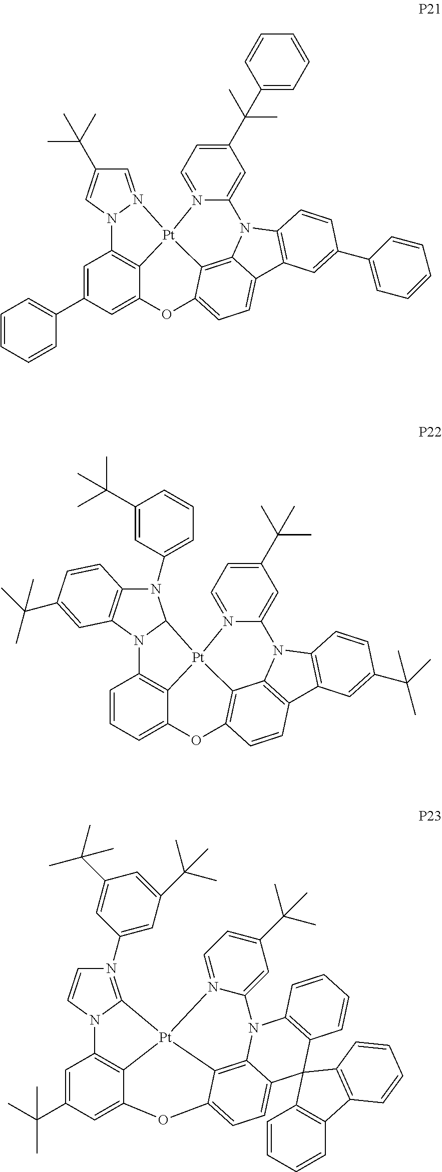

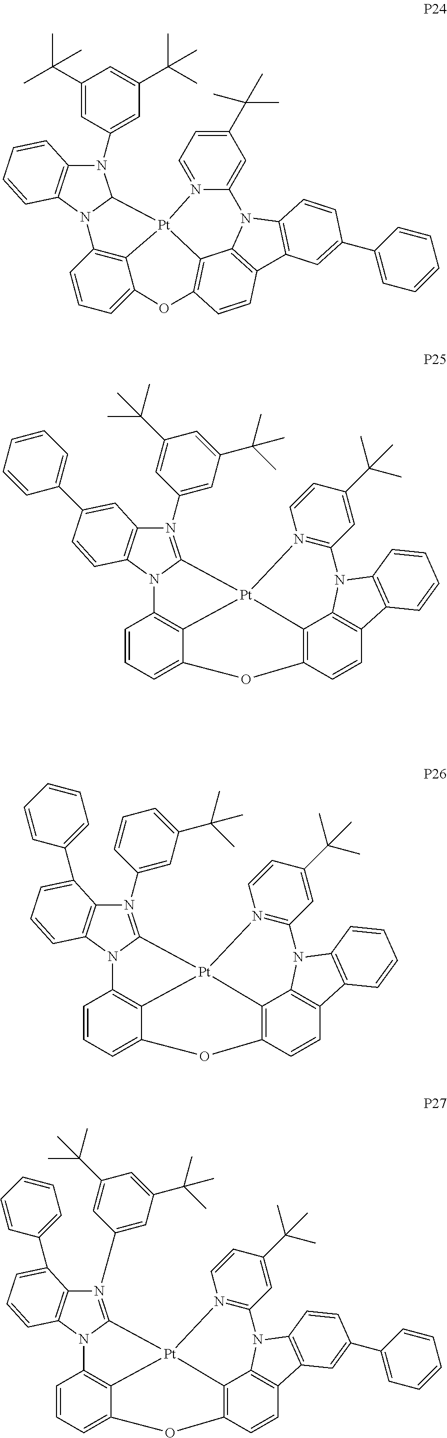

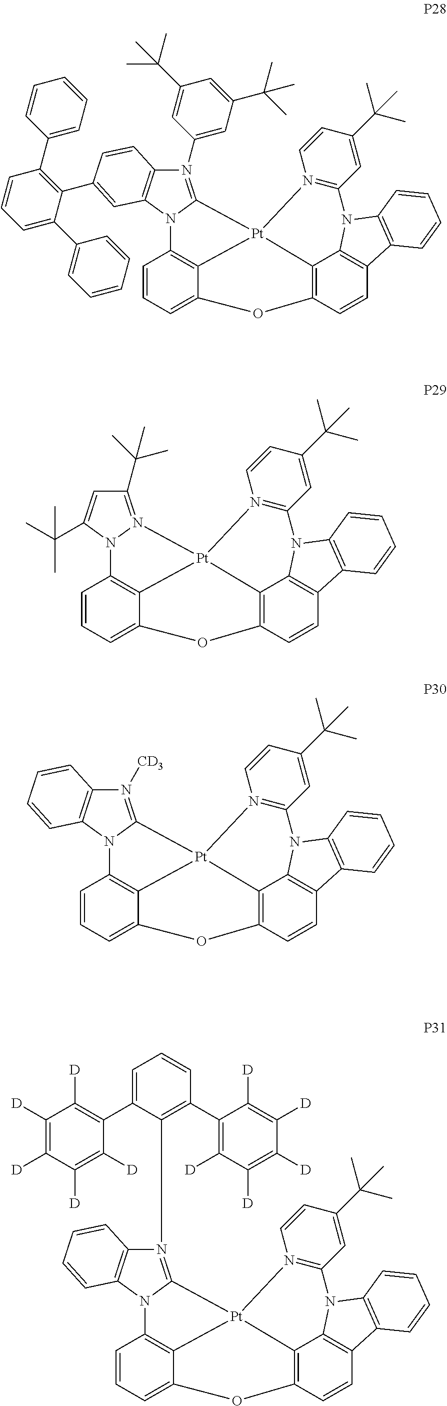

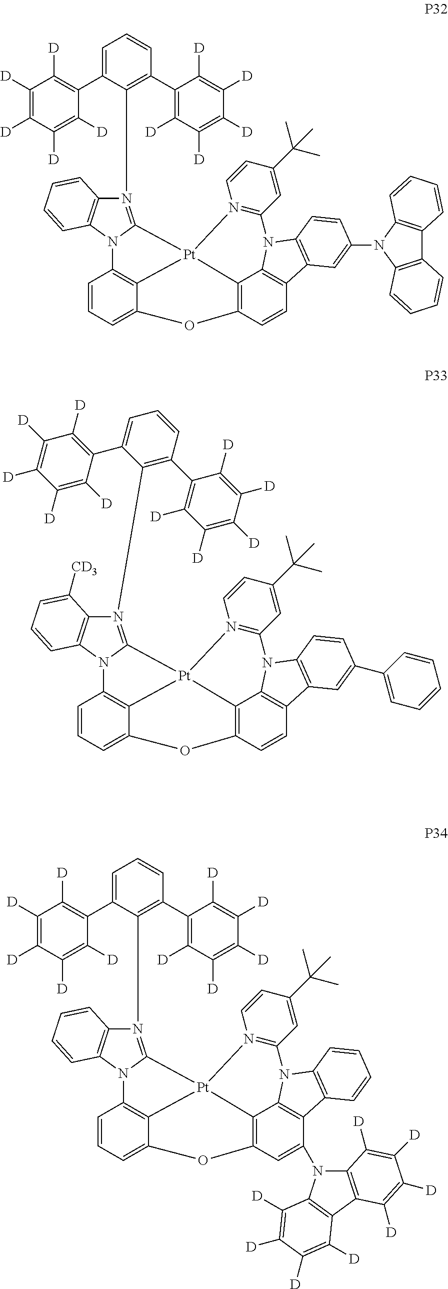

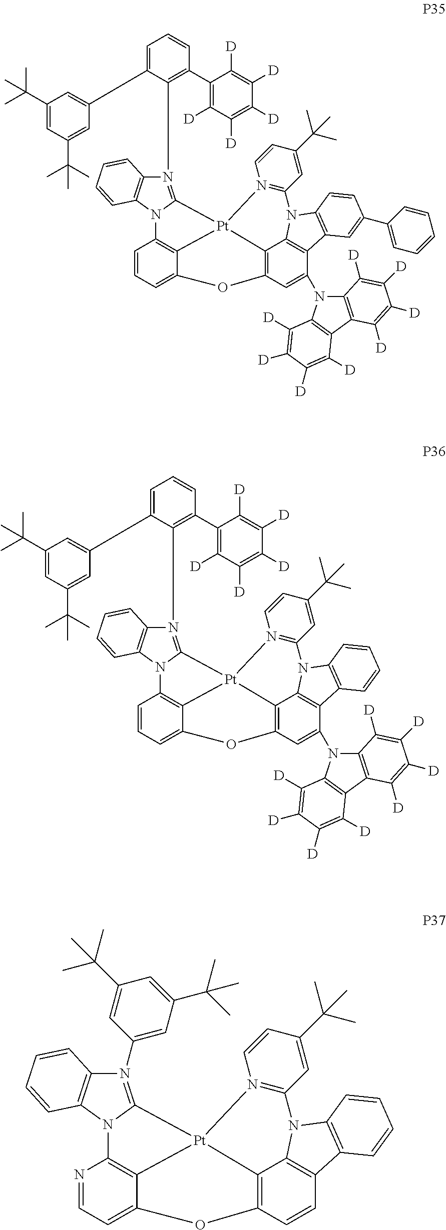

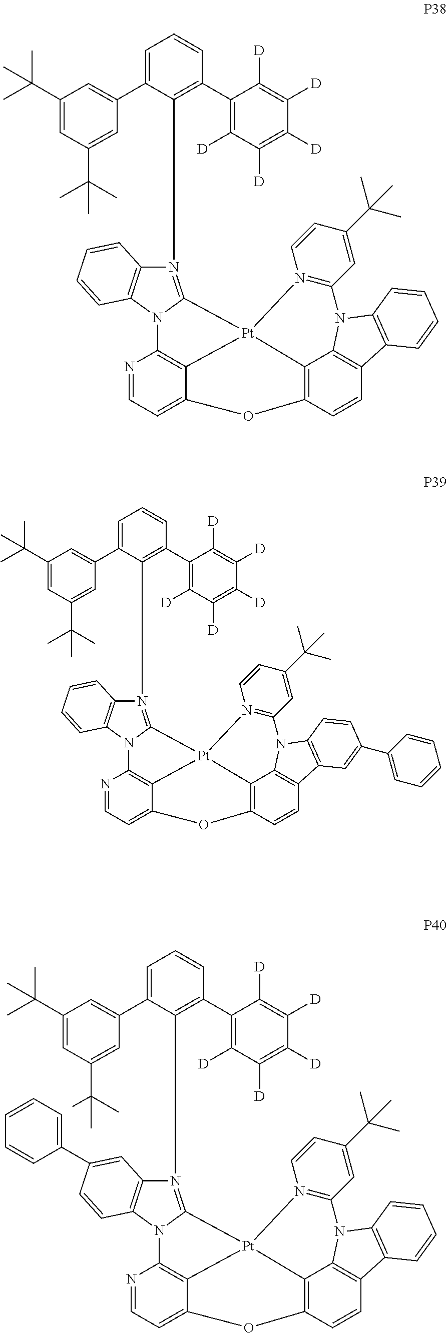

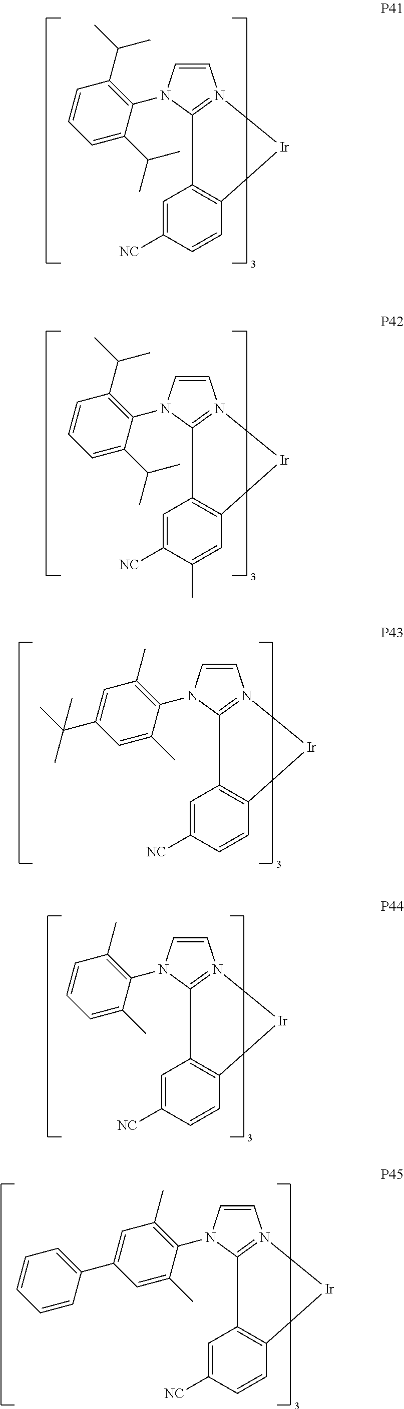

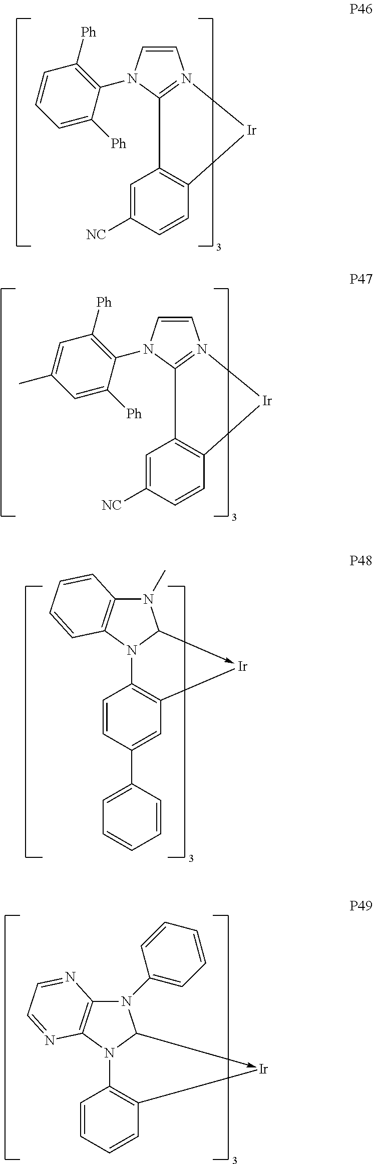

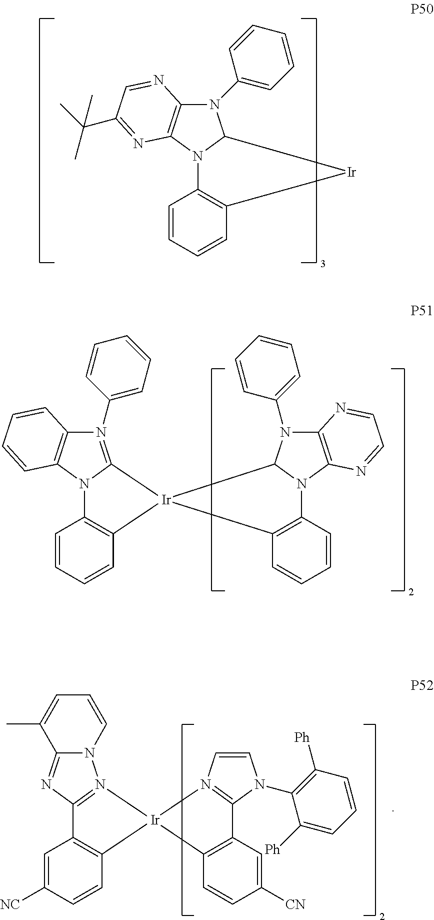

### Description of Formula 4

In the first and second embodiments, the delayed fluorescence material may be a polycyclic compound represented by Formula 4:

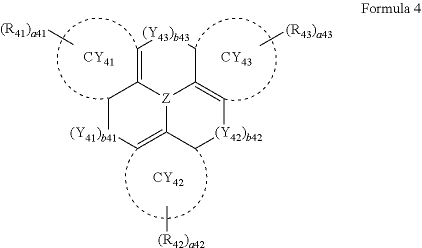

wherein, in Formula 4,
Z may be B or N,
ring CY₄₁ to ring CY₄₃ may each independently be a C₅-C₃₀ carbocyclic group or a C₁-C₃₀ heterocyclic group,
Y₄₁ may be a single bond, *—N(R₄₄)—*′, *—B(R₄₄)—*′, *—P(R₄₄)—*′, *—C(R₄₄)(R₄₅)—*′, *—Si(R₄₄)(R₄₅)—*′, *—Ge(R₄₄)(R₄₅)—*′, *—O—*′, *—S—*′, *—Se—*′, *—C(═O)—*′, or *—S(═O)₂—*′,
Y₄₂ may be a single bond, *—N(R₄₆)—*′, *—B(R₄₆)—*′, *—P(R₄₆)—*′, *—C(R₄₆)(R₄₇)—*′, *—Si(R₄₆)(R₄₇)—*′, *—Ge(R₄₆)(R₄₇)—*′, *—O—*′, *—S—*′, *—Se—*′, *—C(═O)—*′, or *—S(═O)₂—*′,
Y₄₃ may be a single bond, *—N(R₄₈)—*′, *—B(R₄₈)—*′, *—P(R₄₈)—*′, *—C(R₄₈)(R₄₉)—*′, *—Si(R₄₈)(R₄₉)—*′, *—Ge(R₄₈)(R₄₉)—*′, *—O—*′, *—S—*′, *—Se—*′, *—C(═O)—*′, or *—S(═O)₂—*′,
b41 to b43 may each independently be 0 or 1,
when b41 is 0, Y₄₁ may not be present, when b42 is 0, Y₄₂ may not be present, and when b43 is 0, Y₄₃ may not be present,
R₄₁ to R₄₉ may each independently be hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroaryl alkyl group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
a41 to a43 may each independently be an integer from 0 to 20,
two or more of R₄₁ in the number of a41 may optionally be bonded to each other to form a C₅-C₃₀ carbocyclic group unsubstituted or substituted with at least one R_{10b} or a C₁-C₃₀ heterocyclic group unsubstituted or substituted with at least one R_{10b},
two or more of R₄₂ in the number of a42 may optionally be bonded to each other to form a C₅-C₃₀ carbocyclic group unsubstituted or substituted with at least one R_{10b} or a C₁-C₃₀ heterocyclic group unsubstituted or substituted with at least one R_{10b},
two or more of R₄₃ in the number of a43 may optionally be bonded to each other to form a C₅-C₃₀ carbocyclic group unsubstituted or substituted with at least one R_{10b} or a C₁-C₃₀ heterocyclic group unsubstituted or substituted with at least one R_{10b},
two or more of R₄₁ to R₄₉ may optionally be bonded to each other to form a C₅-C₃₀ carbocyclic group unsubstituted or substituted with at least one R_{10b} or a C₁-C₃₀ heterocyclic group unsubstituted or substituted with at least one R_{10b},
R_{10b} is the same as described in connection with R₄₁,
* and *′ each indicate a binding site to a neighboring atom,
a substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ heteroaryl alkyl group, the substituted monovalent non-aromatic condensed polycyclic group, and the substituted monovalent non-aromatic condensed heteropolycyclic group may be:
deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid or a salt thereof, a sulfonic acid or a salt thereof, a phosphoric acid or a salt thereof, a C₁-C₆₀ alkyl group, a C₂-C₆₀ alkenyl group, a C₂-C₆₀ alkynyl group, or a C₁-C₆₀ alkoxy group;
a C₁-C₆₀ alkyl group, a C₂-C₆₀ alkenyl group, a C₂-C₆₀ alkynyl group, or a C₁-C₆₀ alkoxy group, each substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid or a salt thereof, a sulfonic acid or a salt thereof, a phosphoric acid or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁ cycloalkyl group, a C₁-C₁₀ heterocycloalkyl group, a C₃-C₁₀ cycloalkenyl group, a C₁-C₁ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
any combination thereof, and
Q₁ to Q₉, Q₁₁ to Q₁₉, Q₂₁ to Q₂₉, and Q₃₁ to Q₃₉ may each independently be: hydrogen; deuterium; —F; —Cl; —Br; —I; a hydroxyl group; a cyano group; a nitro group; an amidino group; a hydrazine group; a hydrazone group; a carboxylic acid group or a salt thereof; a sulfonic acid group or a salt thereof; a phosphoric acid group or a salt thereof; a C₁-C₆₀ alkyl group unsubstituted or substituted with deuterium, a C₁-C₆₀ alkyl group, a C₆-C₆₀ aryl group, or any combination thereof; a C₂-C₆₀ alkenyl group; a C₂-C₆₀ alkynyl group; a C₁-C₆₀ alkoxy group; a C₃-C₁₀ cycloalkyl group; a C₁-C₁₀ heterocycloalkyl group; a C₃-C₁₀ cycloalkenyl group; a C₁-C₁₀ heterocycloalkenyl group; a C₆-C₆₀ aryl group unsubstituted or substituted with deuterium, a C₁-C₆₀ alkyl group, a C₆-C₆₀ aryl group, or any combination thereof; a C₆-C₆₀ aryloxy group; a C₆-C₆₀ arylthio group; a C₁-C₆₀ heteroaryl group; a monovalent non-aromatic condensed polycyclic group; or a monovalent non-aromatic condensed heteropolycyclic group.

In an embodiment, ring CY₄₁ to ring CY₄₃ may each independently be i) a benzene group, or ii) a polycyclic group in which two or more C₃-C₆₀ cyclic groups are condensed with each other. In this regard, two C₃-C₆₀ cyclic groups of the polycyclic group may be condensed with each other while sharing boron (B) or nitrogen (N).

In one or more embodiments, at least one of b41 to b43 or at least two of b41 to b43 may each be 1. In one or more embodiments, two of b41 to b43 may be 1, and the other one may be 0.

In one or more embodiments, R₄₁ to R₄₉ may each independently be:
hydrogen, deuterium, —F, —Cl, —Br, —I, a hydroxyl group, a cyano group (CN), a nitro group, an amino group, a C₁-C₆₀ alkyl group, or a C₁-C₆₀ alkoxy group;
a C₁-C₆₀ alkyl group or a C₁-C₆₀ alkoxy group, each substituted with at least one of deuterium, —F, —Cl, —Br, —I, a hydroxyl group, a cyano group (CN), a nitro group, an amino group, a phenyl group, a biphenyl group, a naphthyl group, a fluorenyl group, a phenanthrenyl group, an anthracenyl group, a fluoranthenyl group, a triphenylenyl group, a pyrenyl group, and a chrysenyl group; or
a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group, each unsubstituted or substituted with at least one of deuterium, —F, —Cl, —Br, —I, a hydroxyl group, a cyano group (CN), a nitro group, an amino group, a C₁-C₆₀ alkyl group, a C₁-C₆₀ alkoxy group, a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, and a carbazolyl group.

In one or more embodiments, the polycyclic compound represented by Formula 4 may be a polycyclic compound represented by one of Formulae 4-1 to 4-9:

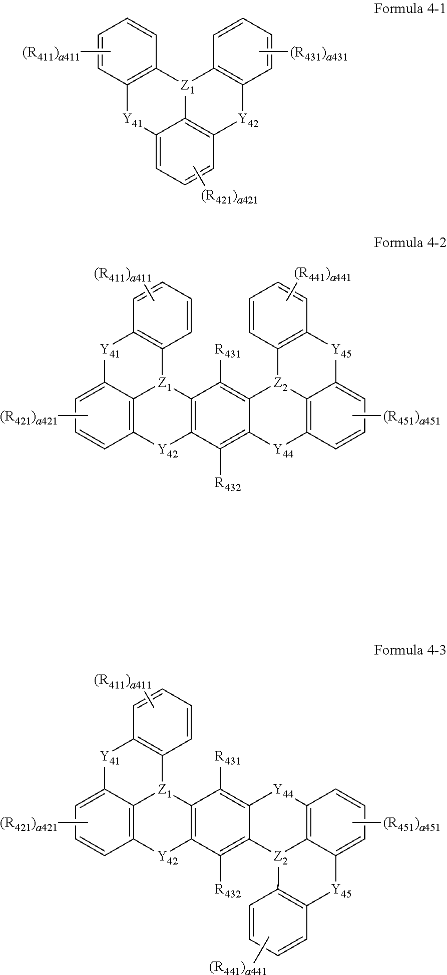

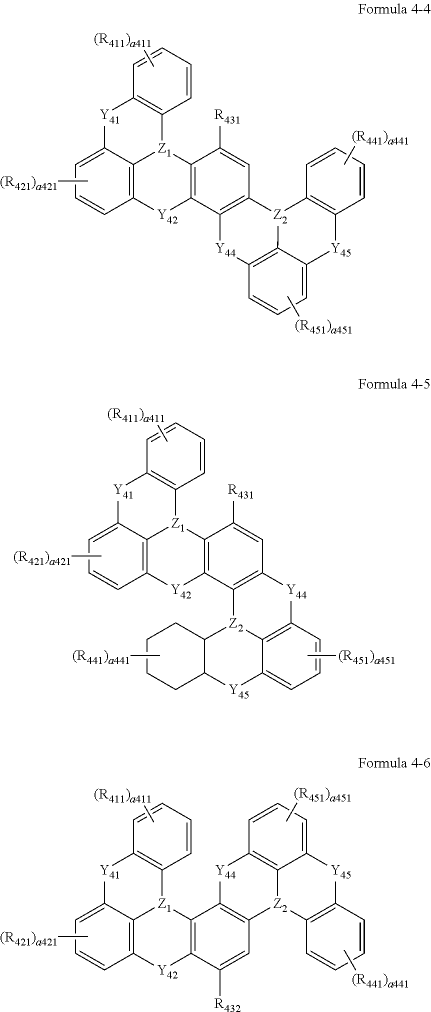

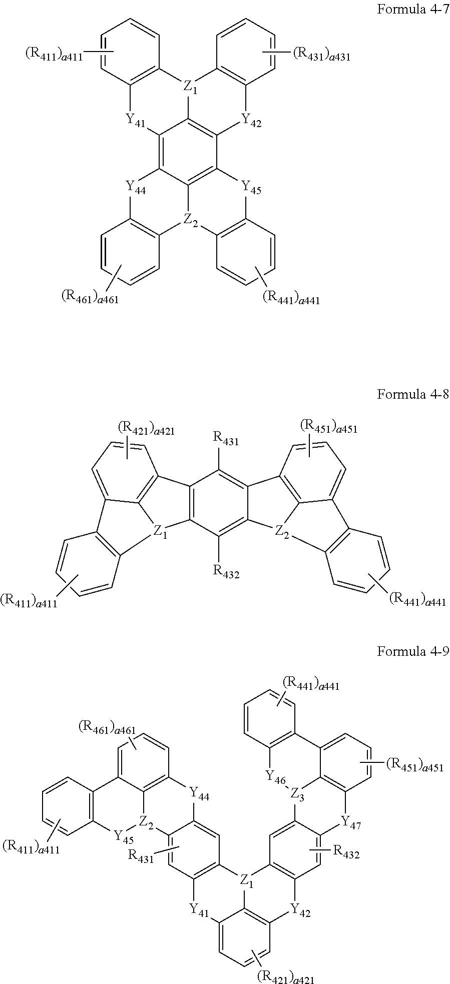

wherein, in Formulae 4-1 to 4-9,
Z₁ to Z₃ may each be understood by referring to the description of Z provided herein,
Y₄₁ and Y₄₂ are the same as described in the specification,
Y₄₄ to Y₄₇ may each be understood by referring to the description of Y₄₁ and Y₄₂,
R₄₁₁ is the same as described in connection with R₄₁, R₄₂₁ is the same as described in connection with R₄₂, R₄₃₁ and R₄₃₂ are each the same as described in connection with R₄₃, R₄₄₁ is the same as described in connection with R₄₁, R₄₅₁ is the same as described in connection with R₄₂, and R₄₆₁ is the same as described in connection with R₄₃,
a411 may be an integer from 0 to 4,
a421 may be an integer from 0 to 3,
a431 may be an integer from 0 to 4,
a441 may be an integer from 0 to 4,
a451 may be an integer from 0 to 3, and
a461 may be an integer from 0 to 3.

Specific examples of polycyclic compound represented by Formula 4

The polycyclic compound represented by Formula 4 may be represented by one of Compounds D1 to D30:

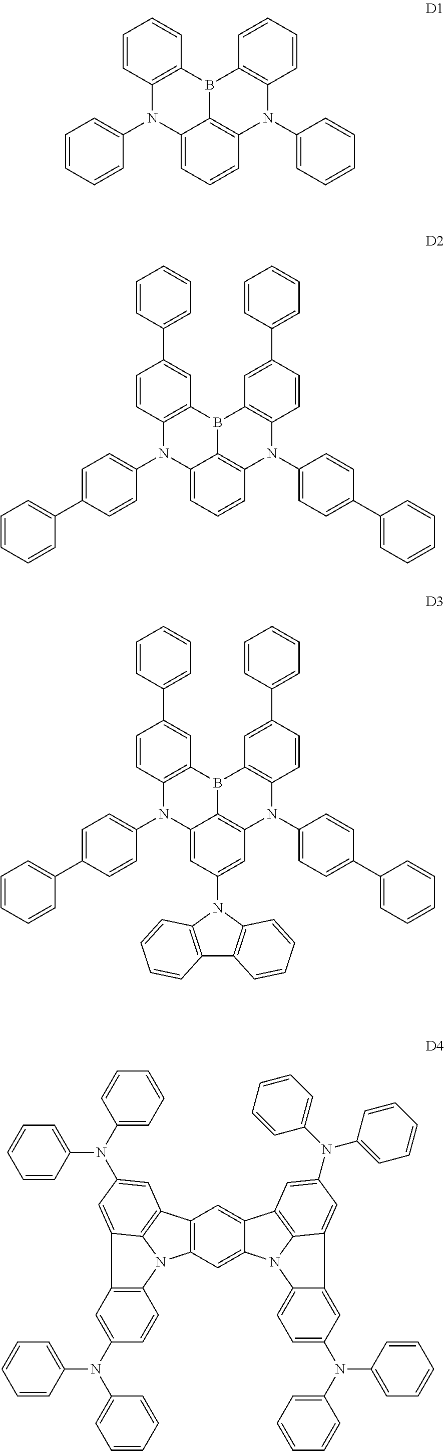 [SMILES: c1ccc(N2c3ccccc3B3c4ccccc4N(c4ccccc4)c4cccc2c43)cc1 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1 ; c1ccc(N(c2ccccc2)c2ccc3c(c2)c2cc(N(c4ccccc4)c4ccccc4)cc4c5cc6c7cc(N(c8ccccc8)c8ccccc8)cc8c9cc(N(c%10ccccc%10)c%10ccccc%10)ccc9n(c6cc5n3c24)c87)cc1]

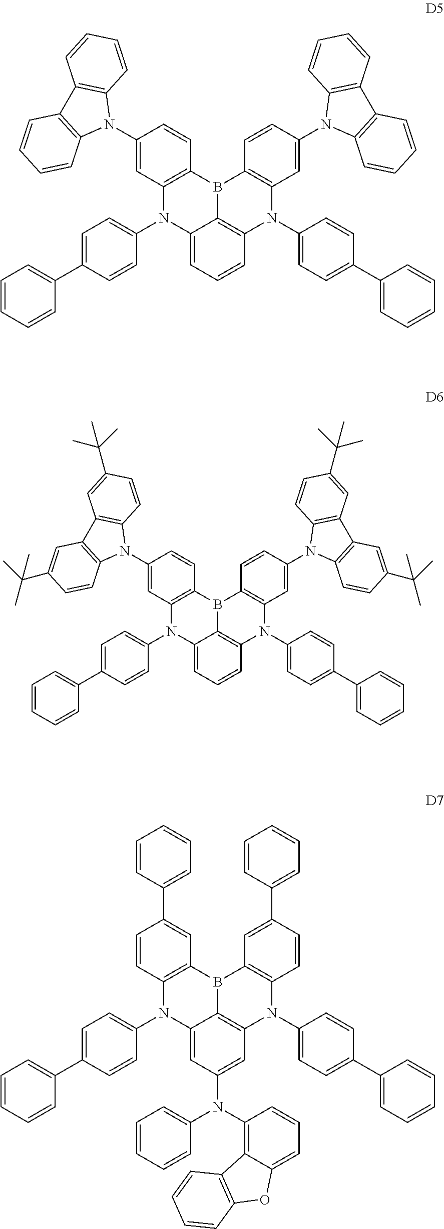 [SMILES: c1ccc(-c2ccc(N3c4cc(-n5c6ccccc6c6ccccc65)ccc4B4c5ccc(-n6c7ccccc7c7ccccc76)cc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ccc(-n2c4ccc(C(C)(C)C)cc4c4cc(C(C)(C)C)ccc42)cc1N3c1ccc(-c2ccccc2)cc1 ; c1ccc(-c2ccc(N3c4ccc(-c5ccccc5)cc4B4c5cc(-c6ccccc6)ccc5N(c5ccc(-c6ccccc6)cc5)c5cc(N(c6ccccc6)c6cccc7oc8ccccc8c67)cc3c54)cc2)cc1]

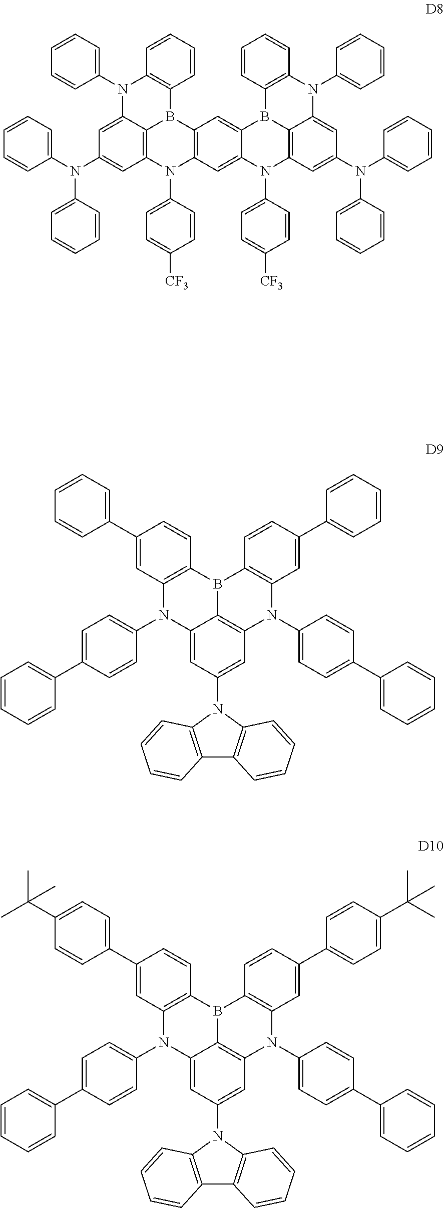 [SMILES: FC(F)(F)c1ccc(N2c3cc4c(cc3B3c5ccccc5N(c5ccccc5)c5cc(N(c6ccccc6)c6ccccc6)cc2c53)B2c3ccccc3N(c3ccccc3)c3cc(N(c5ccccc5)c5ccccc5)cc(c32)N4c2ccc(C(F)(F)F)cc2)cc1 ; c1ccc(-c2ccc(N3c4cc(-c5ccccc5)ccc4B4c5ccc(-c6ccccc6)cc5N(c5ccc(-c6ccccc6)cc5)c5cc(-n6c7ccccc7c7ccccc76)cc3c54)cc2)cc1 ; CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1]

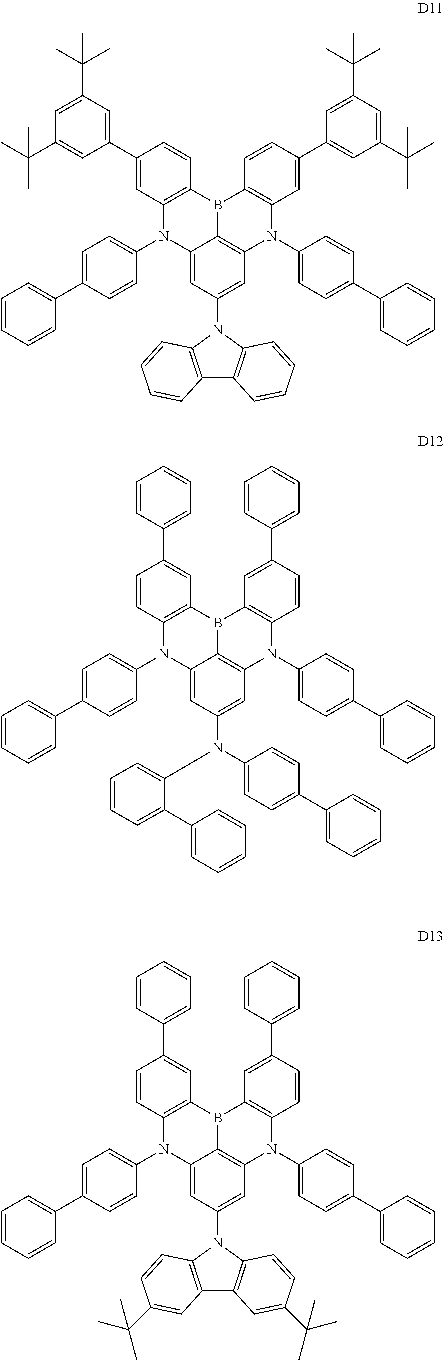 [SMILES: CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1 ; c1ccc(-c2ccc(N(c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)c3ccccc3-c3ccccc3)cc2)cc1 ; CC(C)(C)c1ccc2c(c1)c1cc(C(C)(C)C)ccc1n2-c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1]

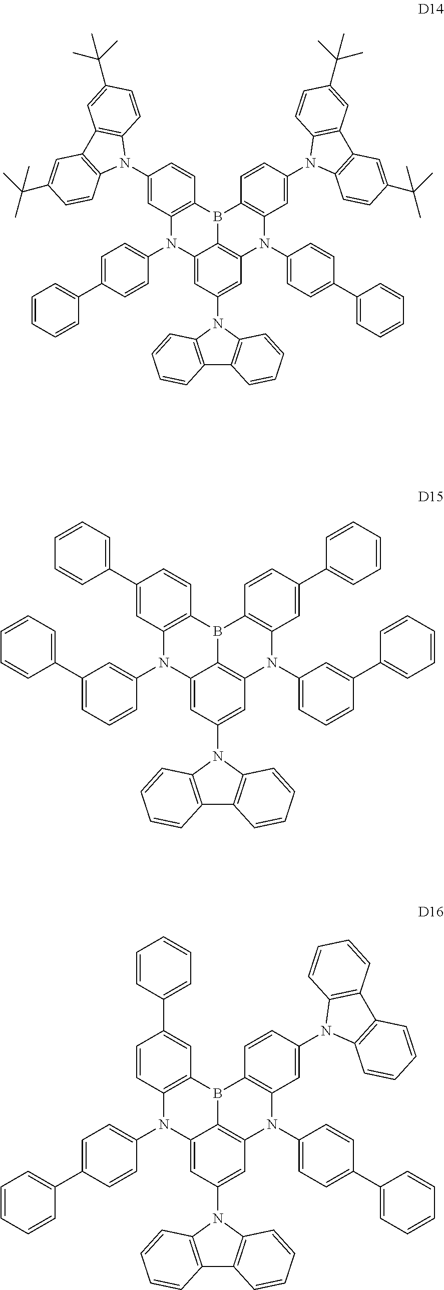 [SMILES: CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccccc2)cc1 ; c1ccc(-c2cccc(N3c4cc(-c5ccccc5)ccc4B4c5ccc(-c6ccccc6)cc5N(c5cccc(-c6ccccc6)c5)c5cc(-n6c7ccccc7c7ccccc76)cc3c54)c2)cc1 ; c1ccc(-c2ccc(N3c4ccc(-c5ccccc5)cc4B4c5ccc(-n6c7ccccc7c7ccccc76)cc5N(c5ccc(-c6ccccc6)cc5)c5cc(-n6c7ccccc7c7ccccc76)cc3c54)cc2)cc1]

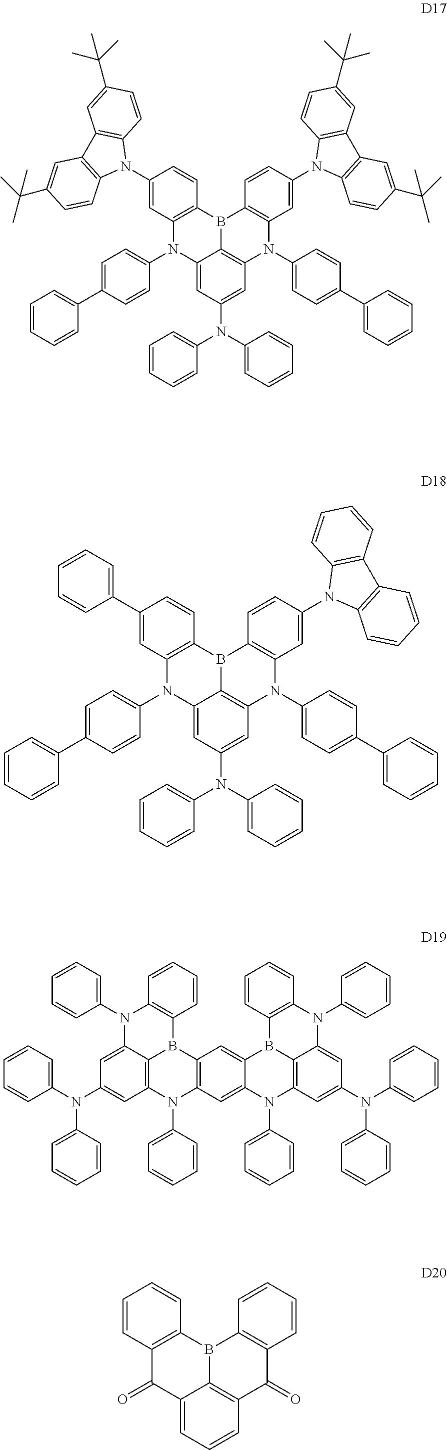 [SMILES: CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1ccc(-n2c4ccc(C(C)(C)C)cc4c4cc(C(C)(C)C)ccc42)cc1N3c1ccc(-c2ccccc2)cc1 ; c1ccc(-c2ccc(N3c4cc(-c5ccccc5)ccc4B4c5ccc(-n6c7ccccc7c7ccccc76)cc5N(c5ccc(-c6ccccc6)cc5)c5cc(N(c6ccccc6)c6ccccc6)cc3c54)cc2)cc1 ; c1ccc(N(c2ccccc2)c2cc3c4c(c2)N(c2ccccc2)c2cc5c(cc2B4c2ccccc2N3c2ccccc2)B2c3ccccc3N(c3ccccc3)c3cc(N(c4ccccc4)c4ccccc4)cc(c32)N5c2ccccc2)cc1 ; O=C1c2ccccc2B2c3ccccc3C(=O)c3cccc1c32]

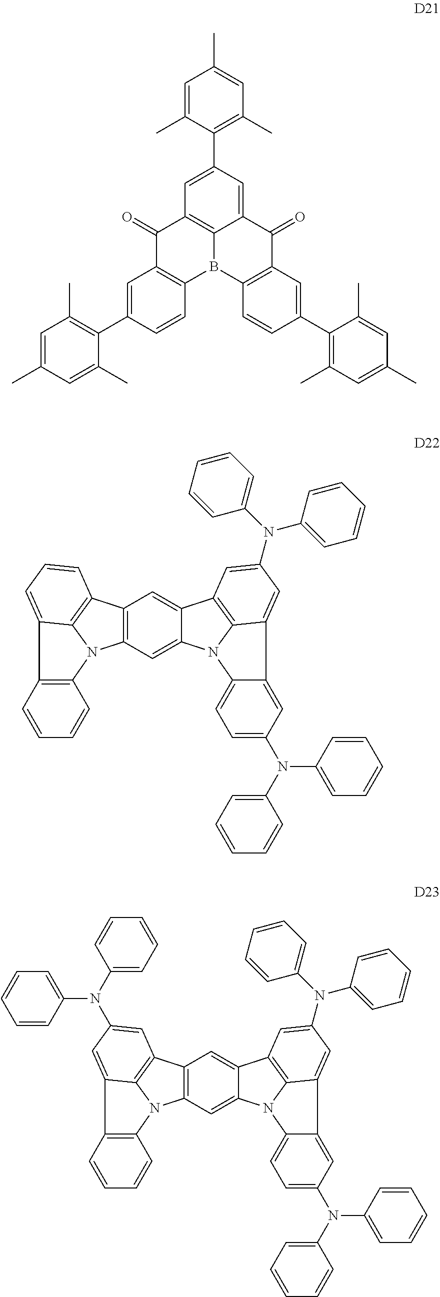

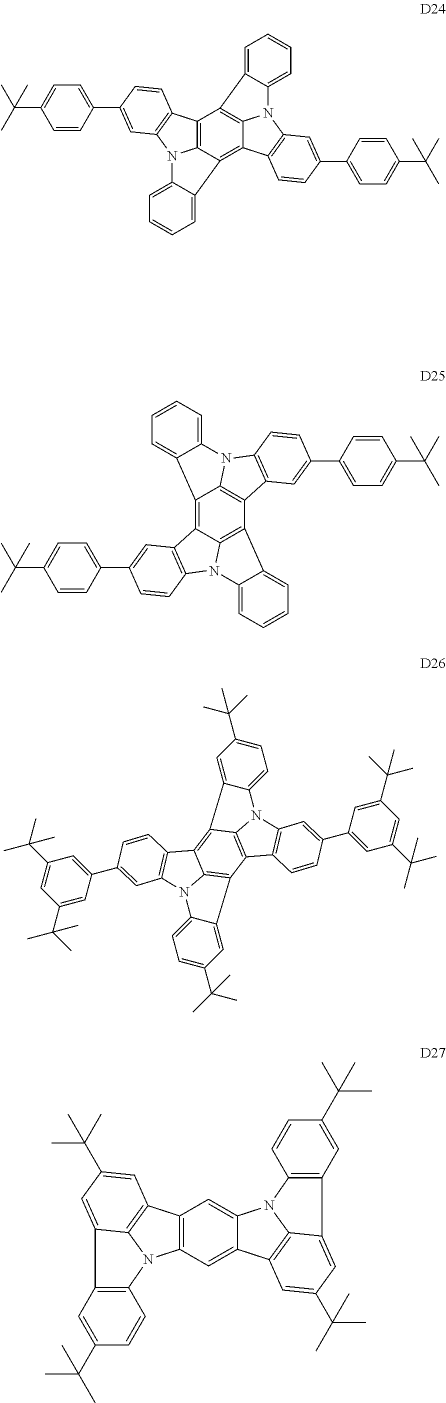

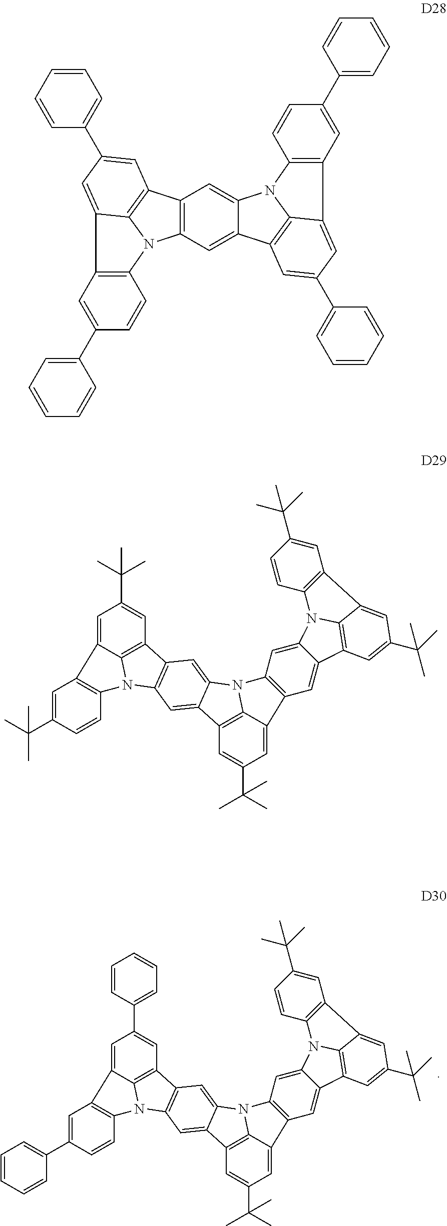 [SMILES: c1ccc(-c2ccc3c(c2)c2cc(-c4ccccc4)cc4c5cc6c(cc5n3c24)c2cc(-c3ccccc3)cc3c4cc(-c5ccccc5)ccc4n6c32)cc1 ; CC(C)(C)c1ccc2c(c1)c1cc(C(C)(C)C)cc3c4cc5c(cc4n2c13)c1cc(C(C)(C)C)cc2c3cc4c6cc(C(C)(C)C)cc7c8cc(C(C)(C)C)ccc8n(c4cc3n5c12)c76 ; CC(C)(C)c1ccc2c(c1)c1cc(C(C)(C)C)cc3c4cc5c6cc(C(C)(C)C)cc7c8cc9c(cc8n(c5cc4n2c13)c76)c1cc(-c2ccccc2)cc2c3cc(-c4ccccc4)ccc3n9c21]

### DESCRIPTION OF FIGURE

FIGURE is a schematic cross-sectional view of an embodiment of an organic light-emitting device 10. Hereinafter, the structure and manufacturing method of the organic light-emitting device 10 according to an embodiment of the disclosure will be described in connection with FIGURE.

In FIGURE, an organic light-emitting device 10 includes a first electrode 11, a second electrode 19 facing the first electrode 11, and an interlayer 10A between the first electrode 11 and the second electrode 19.

In FIGURE, the interlayer 10A includes an emission layer 15, a hole transport region 12 is between the first electrode 11 and an emission layer 15, and an electron transport region 17 is between the emission layer 15 and the second electrode 19.

A substrate may be additionally disposed under the first electrode 11 or on the second electrode 19. The substrate may be a conventional substrate used in organic light-emitting devices, e.g., a glass substrate or a transparent plastic substrate, each having excellent mechanical strength, thermal stability, transparency, surface smoothness, ease of handling, and water repellency.

### First Electrode 11

The first electrode 11 may be produced by depositing or sputtering, onto the substrate, a material for forming the first electrode 11. The first electrode 11 may be an anode. The material for forming the first electrode 11 may include materials with a high work function to facilitate hole injection.

The first electrode 11 may be a reflective electrode, a semi-transmissive electrode, or a transmissive electrode. When the first electrode 11 is a transmissive electrode, the material for forming the first electrode 11 may include indium tin oxide (ITO), indium zinc oxide (IZO), tin oxide (SnO₂), zinc oxide (ZnO), or any combinations thereof. In some embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 11 may have a single-layered structure or a multi-layered structure including a plurality of layers.

### Emission Layer 15

A thickness of the emission layer 15 may be in a range of about 100 angstroms (Å) to about 1,000 Å, for example, about 200 Å to about 600 Å. When the thickness of the emission layer is within these ranges, excellent light-emission characteristics may be obtained without a substantial increase in driving voltage.

In an embodiment, the condensed cyclic compound described above may be included in the emission layer 15. The emission layer 15 may have a feature described in the first and second embodiments.

The emission layer 15 may further include a host as described above in addition to the condensed cyclic compound, sensitizer, and emitter described above.

### Hole Transport Region 12

The hole transport region 12 may be disposed between the first electrode 11 and the emission layer 15 of the organic light-emitting device 10.

The hole transport region 12 may have a single-layered structure or a multi-layered structure.

For example, the hole transport region 12 may have a hole injection layer, a hole transport layer, a hole injection layer/hole transport layer structure, a hole injection layer/first hole transport layer/second hole transport layer structure, a hole injection layer/first hole transport layer/second hole transport layer/electron blocking layer structure, a hole transport layer/interlayer structure, a hole injection layer/hole transport layer/interlayer structure, a hole transport layer/electron blocking layer structure, or a hole injection layer/hole transport layer/electron blocking layer structure.

The hole transport region 12 may include any compound having hole-transporting properties.

For example, the hole transport region 12 may include an amine-based compound.

In an embodiment, the hole transport region 12 may include, for example, 4,4′,4″-tris(3-methylphenylphenylamino)triphenylamine (m-MTDATA), 4,4′,4″-tris(N,N-diphenylamino)triphenylamine (TDATA), 4,4′,4″-tris{N-(2-naphthyl)-N-phenylamino}-triphenylamine (2-TNATA), N,N′-di(1-naphthyl)-N,N′-diphenylbenzidine (NPB), N,N′-di(2-naphthyl)-N,N′-diphenylbenzidine β-NPB, N,N′-bis(3-methylphenyl)-N,N′-diphenyl-[1,1-biphenyl]-4,4′-diamine (TPD), N,N′-Bis(3-methylphenyl)-N,N′-bis(phenyl)-2,7-diamino-9,9-spirobifluorene (Spiro-TPD), N,N′-Bis(naphthalen-1-yl)-N,N′-bis(phenyl)-2,7-diamino-9,9-spirobifluorene (Spiro-NPB), N,N′-di(1-naphthyl)-N,N′-diphenyl-2,2′-dimethyl-(1,1′-biphenyl)-4,4′-diamine(methylated NPB), 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), one of a compound represented by Formula 201 to a compound represented by Formula 205, or any combination thereof:

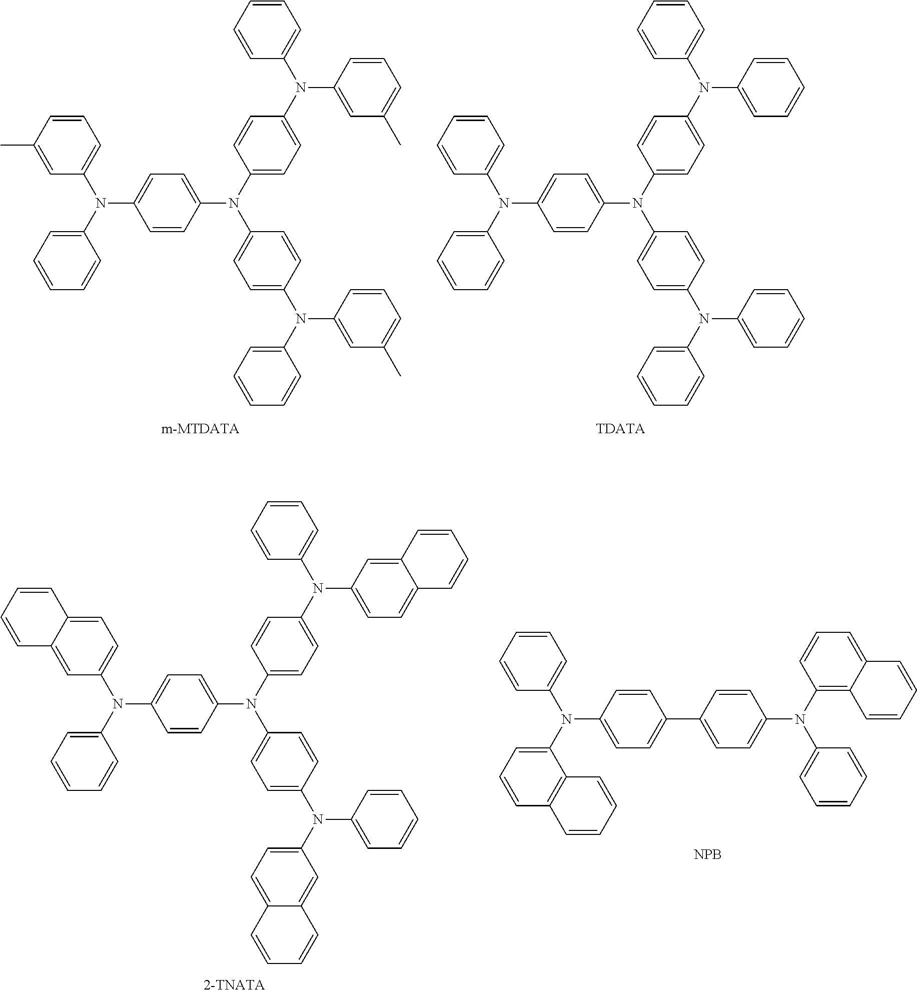

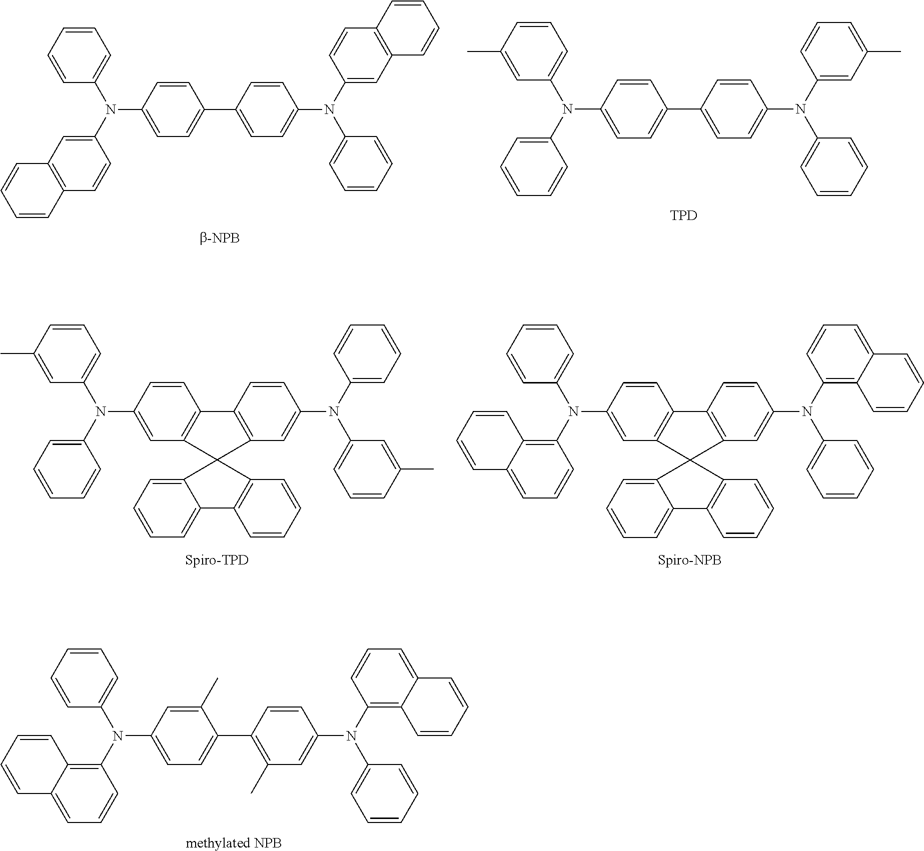

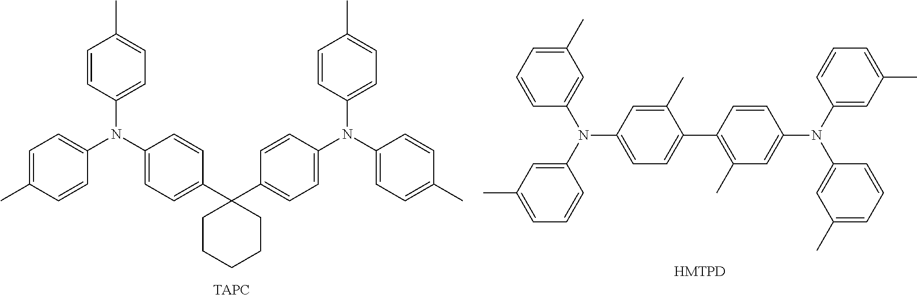

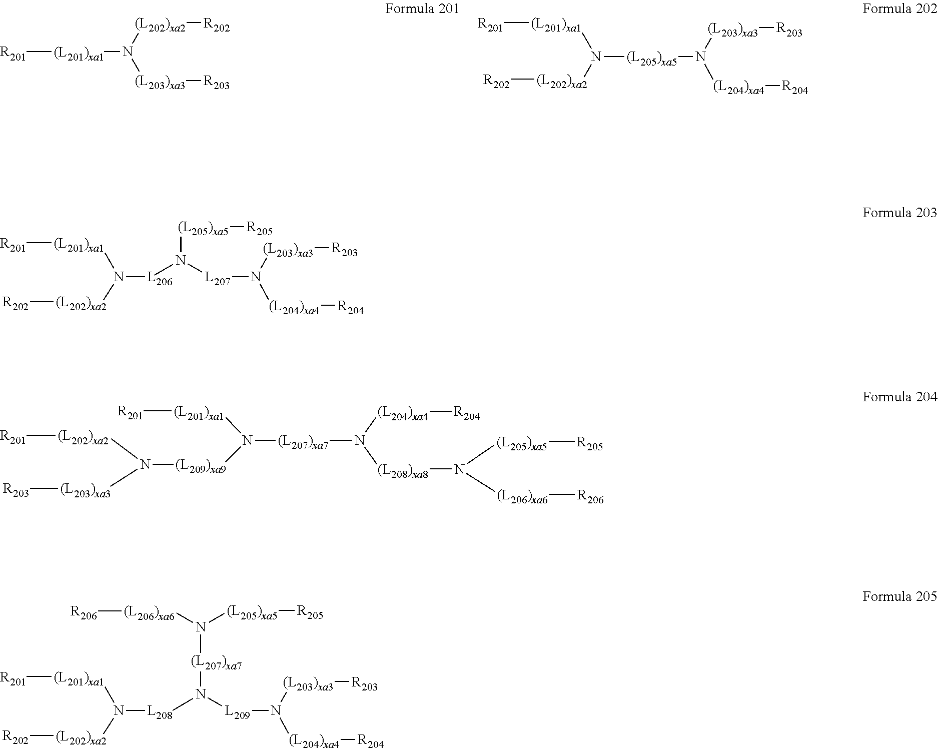

wherein, in Formulae 201 to 205,
L₂₀₁ to L₂₀₉ may each independently be *—O—*′, *—S—*′, a substituted or unsubstituted C₅-C₆₀ carbocyclic group, or a substituted or unsubstituted C₁-C₆₀ heterocyclic group,
xa1 to xa9 may each independently be an integer from 0 to 5,
R₂₀₁ to R₂₀₆ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group, wherein neighboring two groups of R₂₀₁ to R₂₀₆ may optionally be linked to each other via a single bond, a dimethyl-methylene group, or a diphenyl-methylene group.

For example, L₂₀₁ to L₂₀₉ may be a benzene group, a heptalene group, an indene group, a naphthalene group, an azulene group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a corogen group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, or a triindolobenzene group, each unsubstituted or substituted with deuterium, a C₁-C₁₀ alkyl group, a C₁-C₁₀ alkoxy group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, —Si(Q₁₁)(Q₁₂)(Q₁₃), or any combination thereof,
xa1 to xa9 may each independently be 0, 1, or 2, and
R₂₀₁ to R₂₀₆ may each independently be a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an indeno carbazolyl group, an indolocarbazolyl group, a benzofurocarbazolyl group, or a benzothienocarbazolyl group, each unsubstituted or substituted with deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), —N(Q₃₁)(Q₃₂), or any combination thereof.
Q₁₁ to Q₁₃ and Q₃₁ to Q₃₃ may each independently be a C₁-C₁₀ alkyl group, a C₁-C₁₀ alkoxy group, a phenyl group, a biphenyl group, a terphenyl group, or a naphthyl group.

In one or more embodiments, the hole transport region 12 may include a carbazole-containing amine-based compound.

In an embodiment, the hole transport region 12 may include a carbazole-containing amine-based compound and a carbazole-free amine-based compound.

The carbazole-containing amine-based compound may include, for example, compounds represented by Formula 201 including a carbazole group and further including at least one of a dibenzofuran group, a dibenzothiophene group, a fluorene group, a spiro-bifluorene group, an indenocarbazole group, an indolocarbazole group, a benzofurocarbazole group, and a benzothienocarbazole group.

The carbazole-free amine-based compound may include, for example, compounds represented by Formula 201 not including a carbazole group and including at least one of a dibenzofuran group, a dibenzothiophene group, a fluorene group, a spiro-bifluorene group, an indenocarbazole group, an indolocarbazole group, a benzofurocarbazole group, and a benzothienocarbazole group.

In one or more embodiments, the hole transport region 12 may include a compound represented by Formula 201, a compound represented by Formula 202, or any combination thereof.

In an embodiment, the hole transport region 12 may include a compound represented by Formula 201-1, 202-1, or 201-2, or any combination thereof:

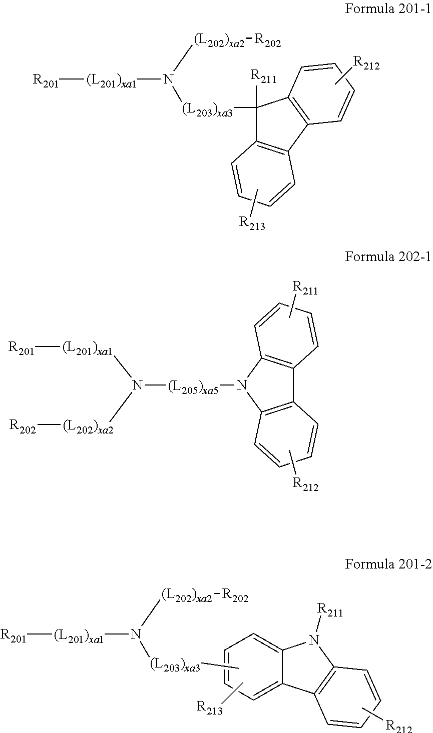

wherein, in Formulae 201-1, 202-1, and 201-2, L₂₀₁ to L₂₀₃, L₂₀₅, xa1 to xa3, xa5, R₂₀₁ and R₂₀₂ are each the same as described herein, and R₂₁₁ to R₂₁₃ may each independently be hydrogen, deuterium, —F, —Cl, —Br, —I, a hydroxyl group, a cyano group, a nitro group, an amidino group, a hydrazine group, a hydrazone group, a C₁-C₂₀ alkyl group, a C₁-C₂₀ alkoxy group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a dimethylfluorenyl group, a diphenylfluorenyl group, a triphenylenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, or a pyridinyl group.

In an embodiment, the hole transport region 12 may include one of Compounds HT1 to HT39 or any combination thereof:

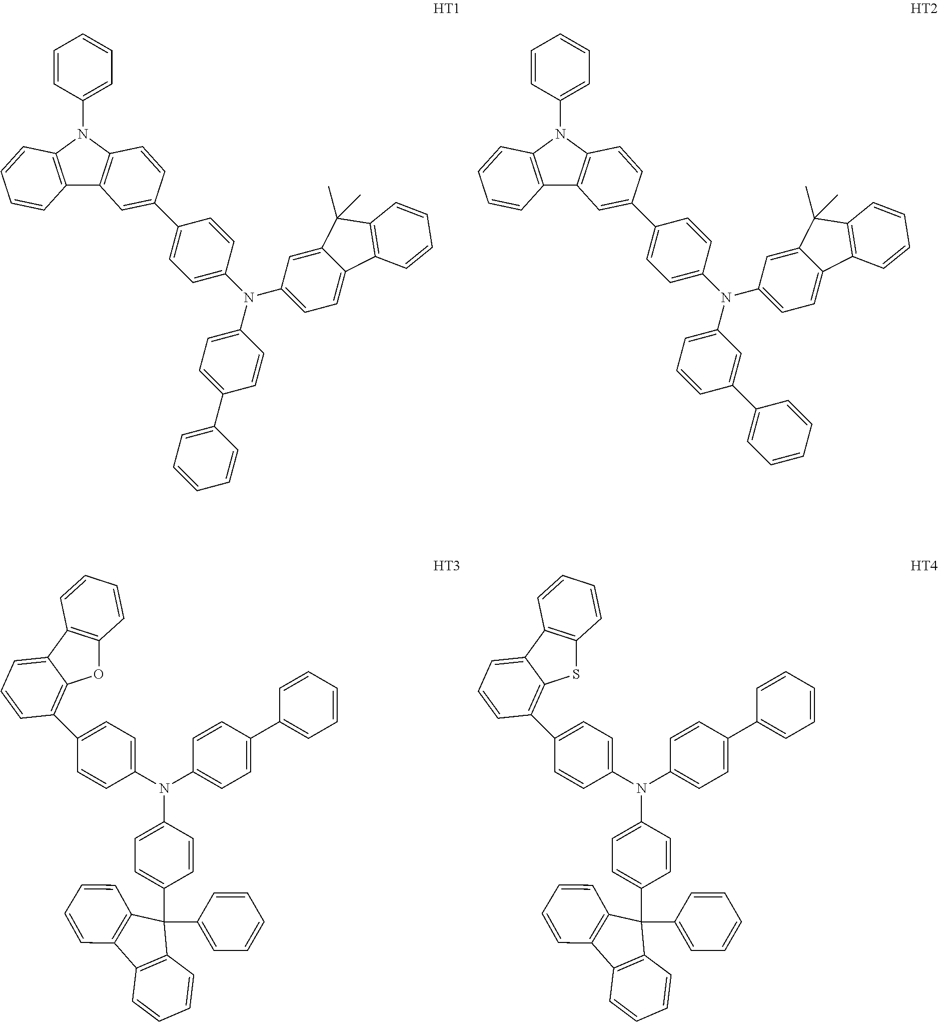 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; c1ccc(-c2ccc(N(c3ccc(-c4cccc5c4sc4ccccc45)cc3)c3ccc(C4(c5ccccc5)c5ccccc5-c5ccccc54)cc3)cc2)cc1]

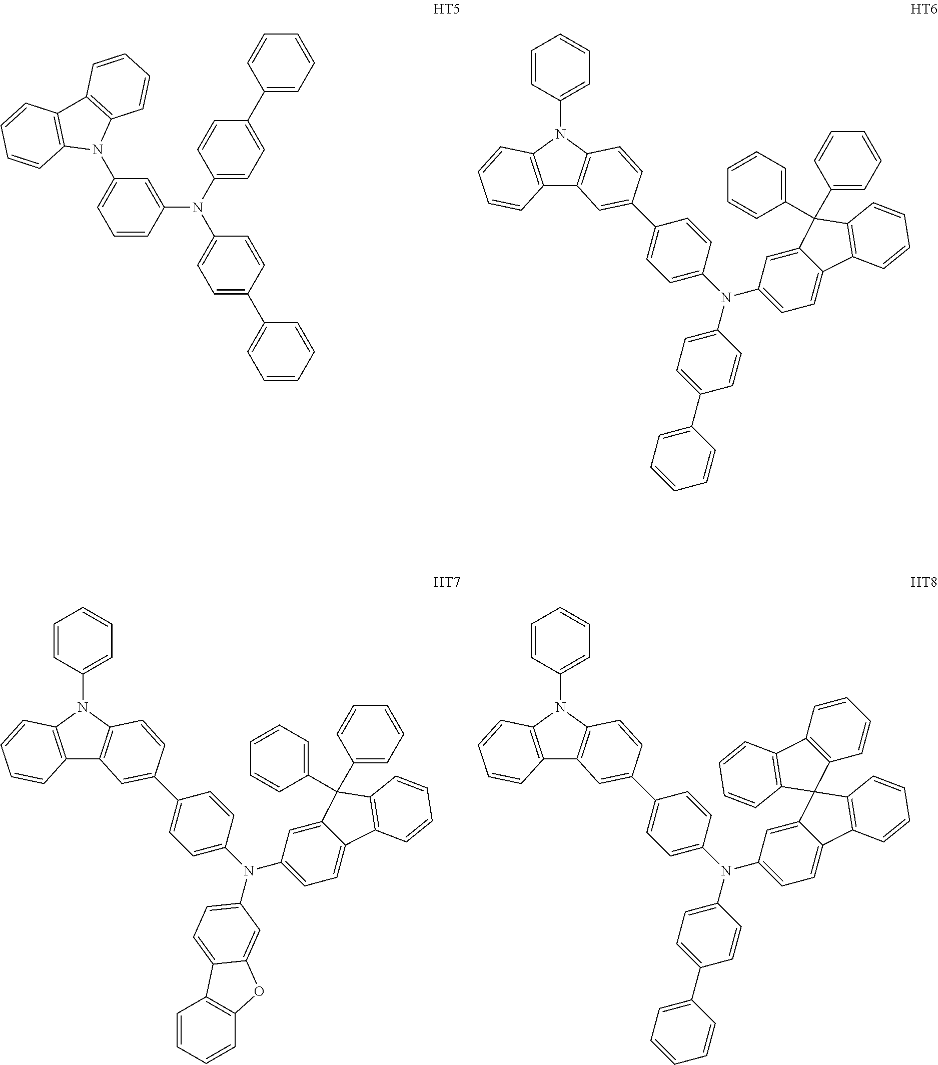 [SMILES: c1ccc(-c2ccc(N(c3ccc(-c4ccccc4)cc3)c3cccc(-n4c5ccccc5c5ccccc54)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

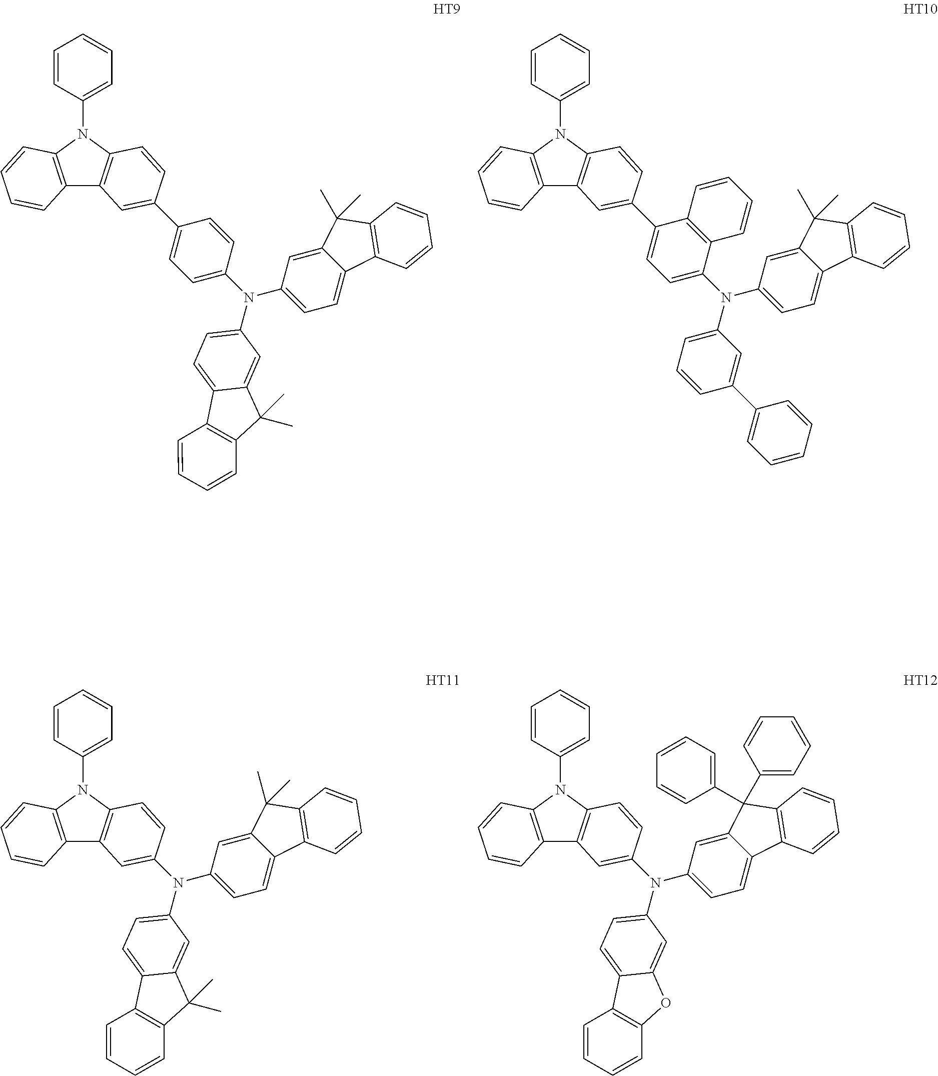 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

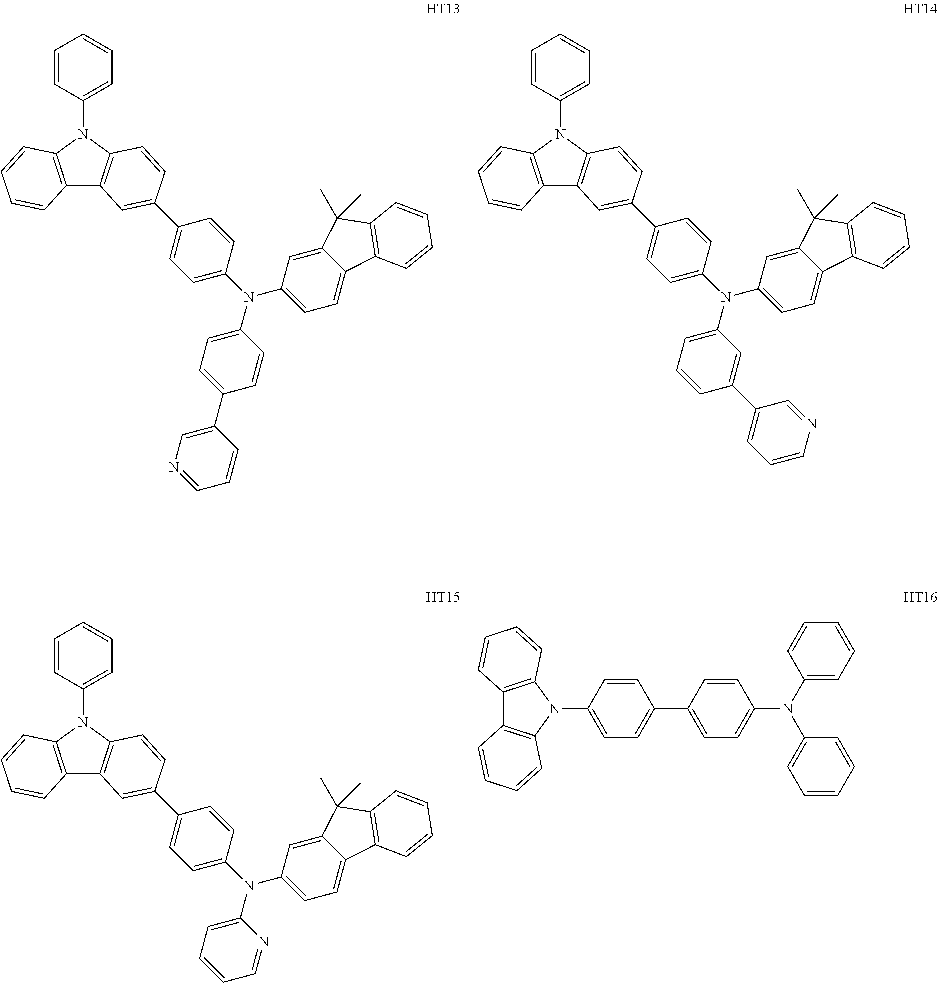 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

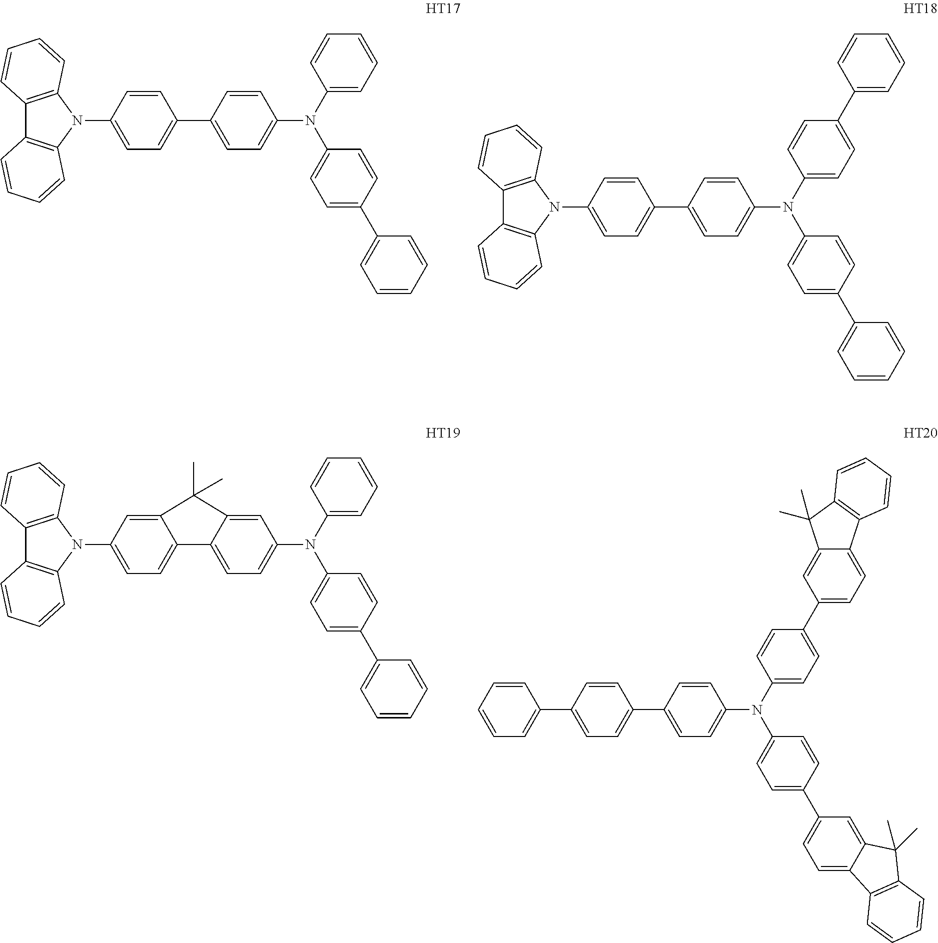 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

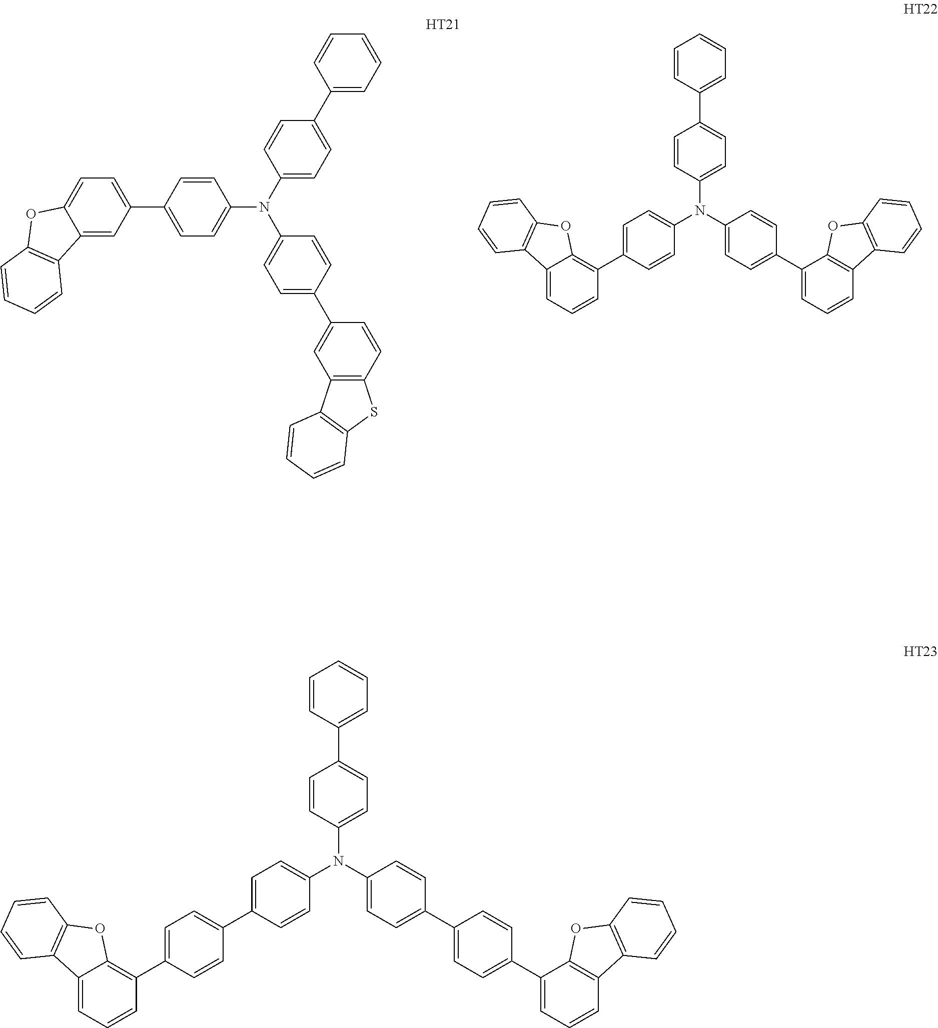 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

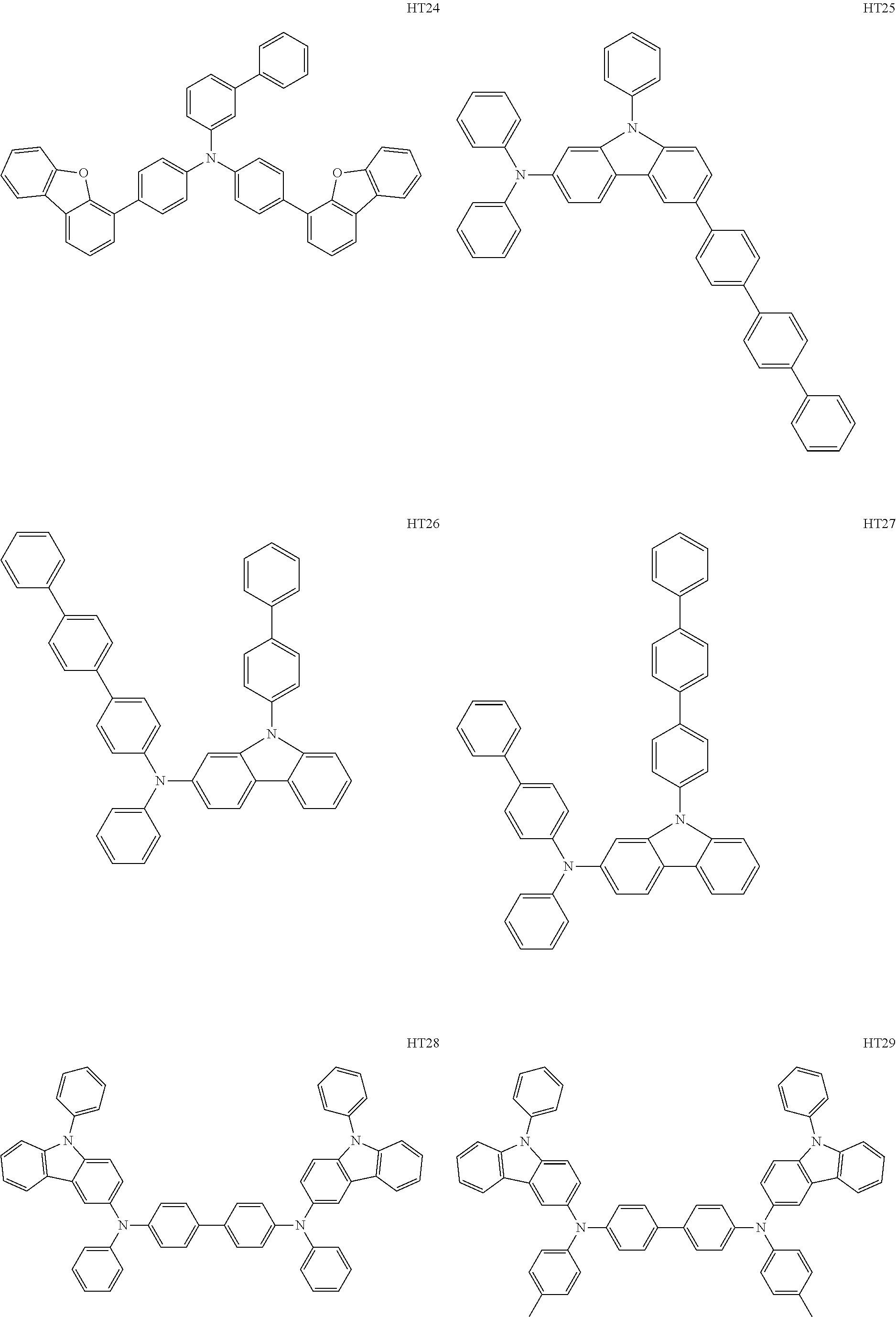

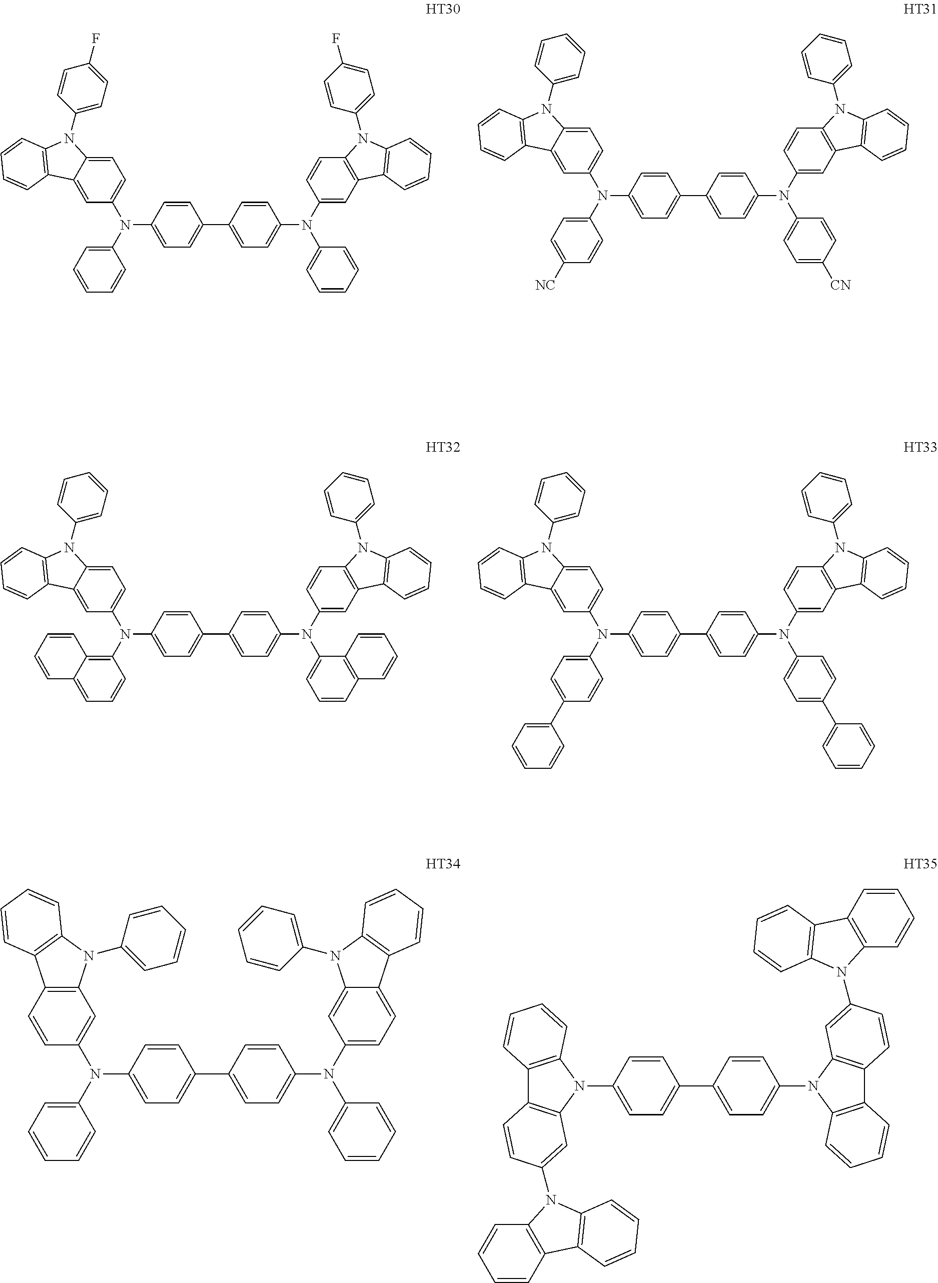

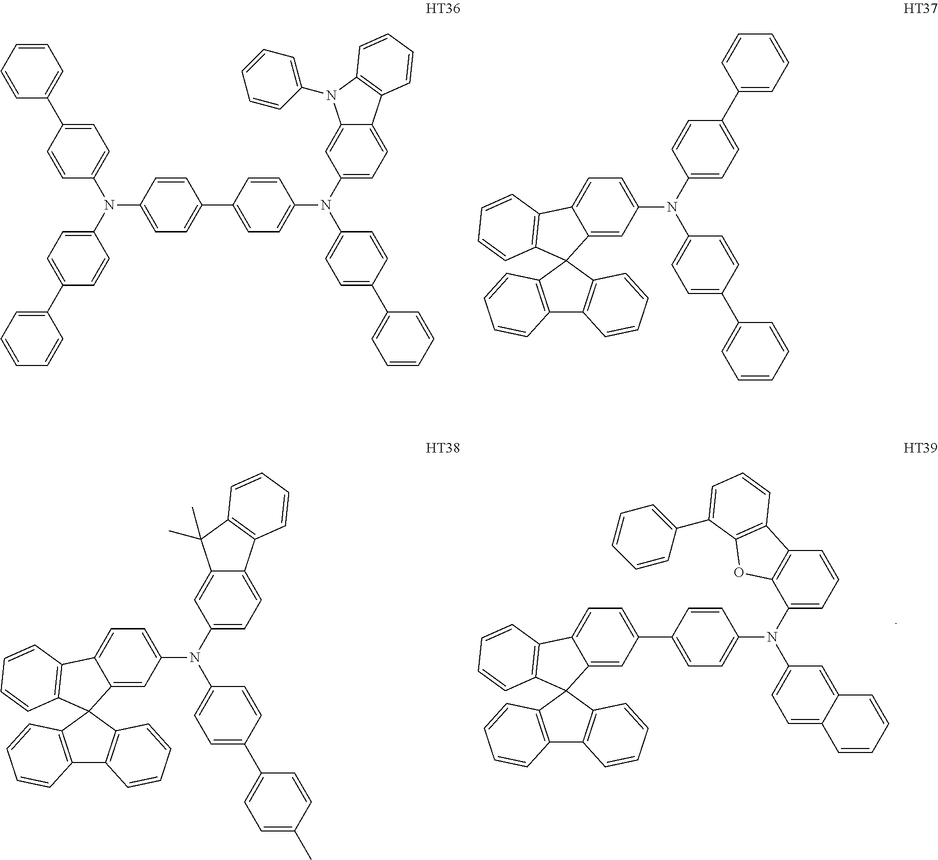 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

In one or more embodiments, hole transport region 12 of the organic light-emitting device 10 may further include a p-dopant. When the hole transport region 12 further includes a p-dopant, the hole transport region 12 may have a matrix (for example, at least one of compounds represented by Formulae 201 to 205) and a p-dopant included in the matrix. The p-dopant may be uniformly or non-uniformly doped in the hole transport region 12.

In an embodiment, the lowest unoccupied molecular orbital (LUMO) energy level of the p-dopant may be −3.5 eV or less.

The p-dopant may include a quinone derivative, a metal oxide, a cyano group-containing compound, or any combination thereof.

In an embodiment, the p-dopant may include:
a quinone derivative, such as tetracyanoquinodimethane (TCNQ),2,3,5,6-tetrafluoro-7,7,8,8-tetracyanoquinodimethane (F4-TCNQ), and 1,3,4,5,7,8-hexafluorotetracyanonaphthoquinodimethane (F6-TCNNQ);
a metal oxide, such as tungsten oxide or molybdenum oxide;
1,4,5,8,9,12-hexaazatriphenylene-hexacarbonitrile (HAT-CN);
a compound represented by Formula 221; or
any combination thereof:

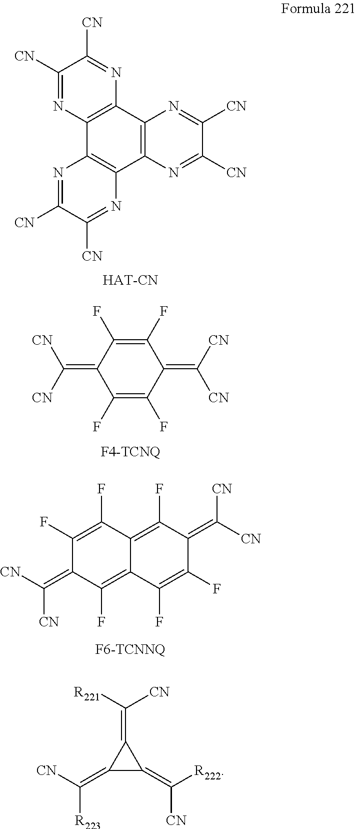

In Formula 221,
R₂₂₁ to R₂₂₃ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group, wherein at least one substituent of R₂₂₁ to R₂₂₃ may be: a cyano group; —F; —Cl; —Br; —I; a C₁-C₂₀ alkyl group substituted with —F; a C₁-C₂₀ alkyl group substituted with —C₁; a C₁-C₂₀ alkyl group substituted with —Br; a C₁-C₂₀ alkyl group substituted with —I; or any combination thereof.

The compound represented by Formula 221 may include, for example, Compound HT-D2:

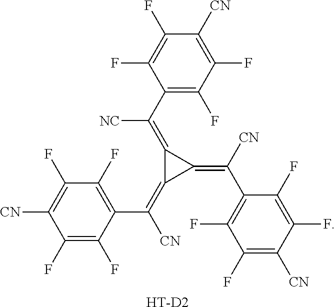

The hole transport region 12 may have a thickness of about 100 Å to about 10,000 Å, for example, about 400 Å to about 2,000 Å, and the emission layer 15 may have a thickness of about 100 Å to about 3,000 Å, for example, about 300 Å to about 1,000 Å.

When the thickness of each of the hole transport region 12 and the emission layer 15 is within these ranges described above, satisfactory hole transportation characteristics and/or luminescent characteristics may be obtained without a substantial increase in driving voltage.

The hole transport region 12 may further include a buffer layer.

Also, the buffer layer may compensate for an optical resonance distance according to a wavelength of light emitted from the emission layer, and thus, efficiency of a formed organic light-emitting device may be improved.

The hole transport region 12 may further include an electron blocking layer. The electron blocking layer may include a known material, for example, mCP or DBFPO:

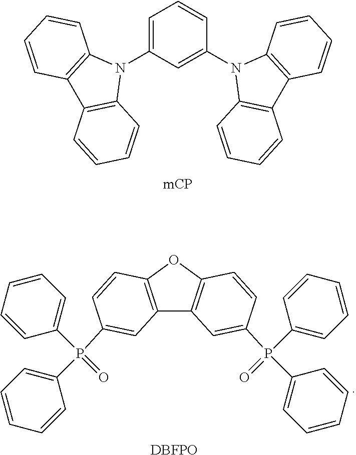

### Electron Transport Region 17

The electron transport region 17 may be disposed between the emission layer 15 and the second electrode 19 of the organic light-emitting device 10.

The electron transport region 17 may have a single-layered structure or a multi-layered structure.

For example, the electron transport region 17 may have an electron transport layer, an electron transport layer/electron injection layer structure, a buffer layer/electron transport layer structure, hole blocking layer/electron transport layer structure, a buffer layer/electron transport layer/electron injection layer structure, or a hole blocking layer/electron transport layer/electron injection layer structure. The electron transport region 17 may further include an electron control layer.

The electron transport region 17 may include known electron-transporting materials.

The electron transport region 17 (for example, a buffer layer, a hole blocking layer, an electron control layer, or an electron transport layer in the electron transport region) may include a metal-free compound containing at least one π electron-deficient nitrogen-containing C₁-C₆₀ cyclic group. The π electron-deficient nitrogen-containing C₁-C₆₀ cyclic group is the same as described above.

For example, the electron transport region 17 may include a compound represented by Formula 601:

[Ar₆₀₁]ₓₑ₁₁-[(L₆₀₁)ₓₑ₁-R₆₀₁]ₓₑ₂₁ Formula 601
wherein, in Formula 601,
Ar₆₀₁ and L₆₀₁ may each independently be a substituted or unsubstituted C₅-C₆₀ carbocyclic group unsubstituted or substituted with at least one R₆₀₁ₐ or a C₁-C₆₀ heterocyclic group unsubstituted or substituted with at least one R₆₀₁ₐ,
xe11 may be 1, 2, or 3,
xe1 may be an integer from 0 to 5,
R₆₀₁ₐ and R₆₀₁ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ may each independently be a C₁-C₁₀ alkyl group, a C₁-C₁₀ alkoxy group, a phenyl group, a biphenyl group, a terphenyl group, or a naphthyl group, and
xe21 may be an integer from 1 to 5.

In an embodiment, at least one of Ar₆₀₁ in the number of xe11 and R₆₀₁ in the number of xe21 may include the π electron-deficient nitrogen-containing C₁-C₆₀ cyclic group.

In an embodiment, Ar₆₀₁ and L₆₀₁ in Formula 601 may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —S(═O)₂(Q₃₁), —P(═O)(Q₃₁)(Q₃₂), or any combination thereof,
Q₃₁ to Q₃₃ may each independently be a C₁-C₁₀ alkyl group, a C₁-C₁₀ alkoxy group, a phenyl group, a biphenyl group, a terphenyl group, or a naphthyl group.

When xe11 in Formula 601 is 2 or more, two or more of Ar₆₀₁ may be linked to each other via a single bond.

In one or more embodiments, Ar₆₀₁ in Formula 601 may be an anthracene group.

In one or more embodiments, the compound represented by Formula 601 may be represented by Formula 601-1:

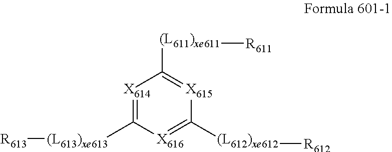

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ may each independently be the same as described in connection with L₆₀₁,
xe611 to xe613 may each independently be the same as described in connection with xe1,
R₆₁₁ to R₆₁₃ may each independently be the same as described in connection with R₆₀₁, and
R₆₁₄ to R₆₁₆ may each independently be hydrogen, deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, or a naphthyl group.

In one or more embodiments, xe1 and xe611 to xe613 in Formulae 601 and 601-1 may each independently be 0, 1, or 2.

In one or more embodiments, R₆₀₁ and R₆₁₁ to R₆₁₃ in Formulae 601 and 601-1 may each independently be a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group, each unsubstituted or substituted with deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, or any combination thereof; or
—S(═O)₂(Q₆₀₁), or —P(═O)(Q₆₀₁)(Q₆₀₂), and
Q₆₀₁ and Q₆₀₂ are the same as described above.

The electron transport region 17 may include one of Compounds ET1 to ET36 or any combination thereof:

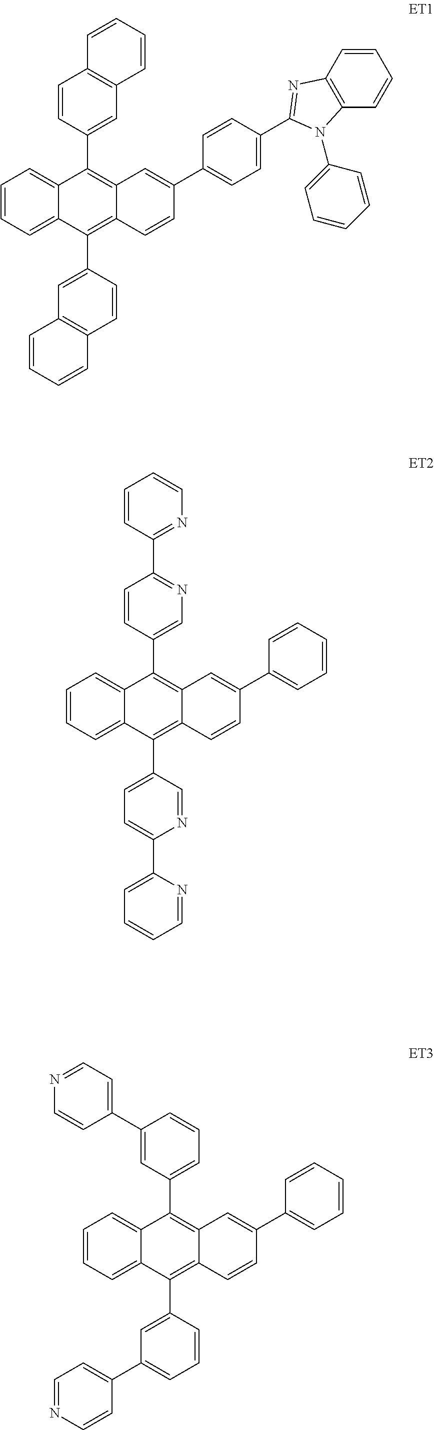 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

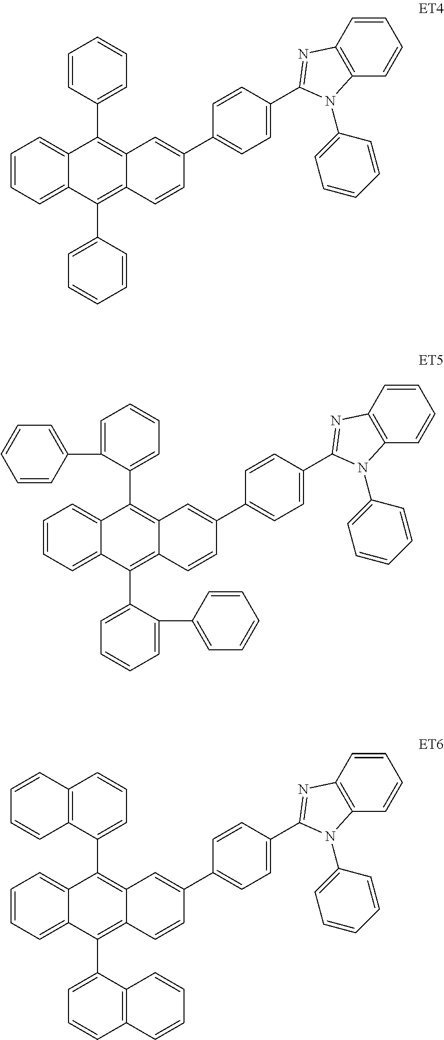 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

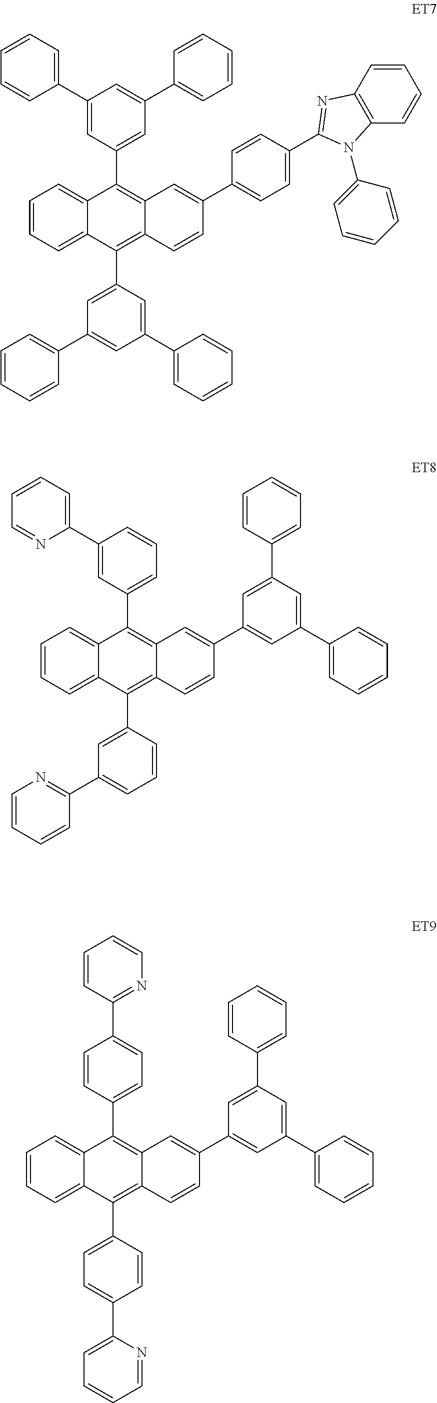 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

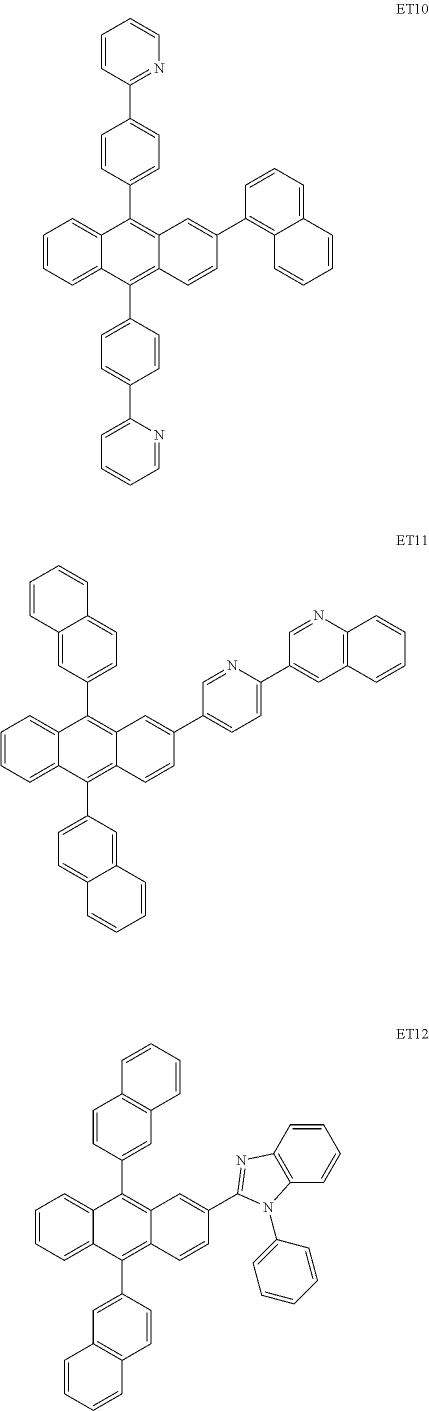 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

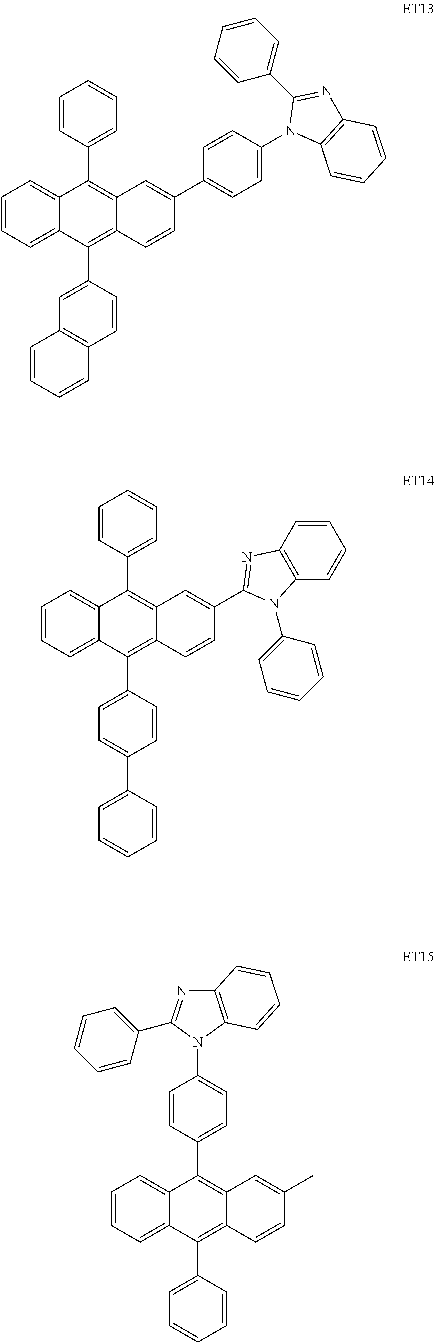 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

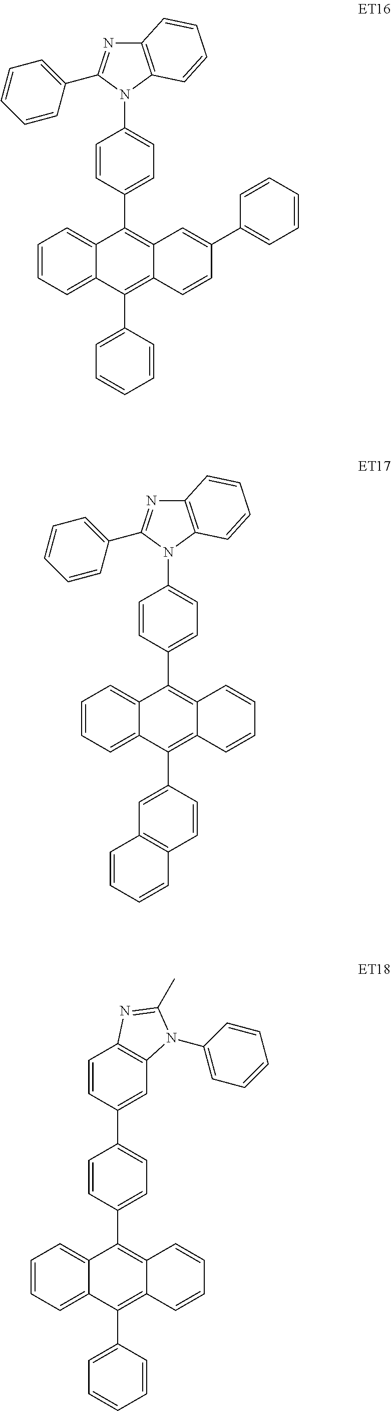 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

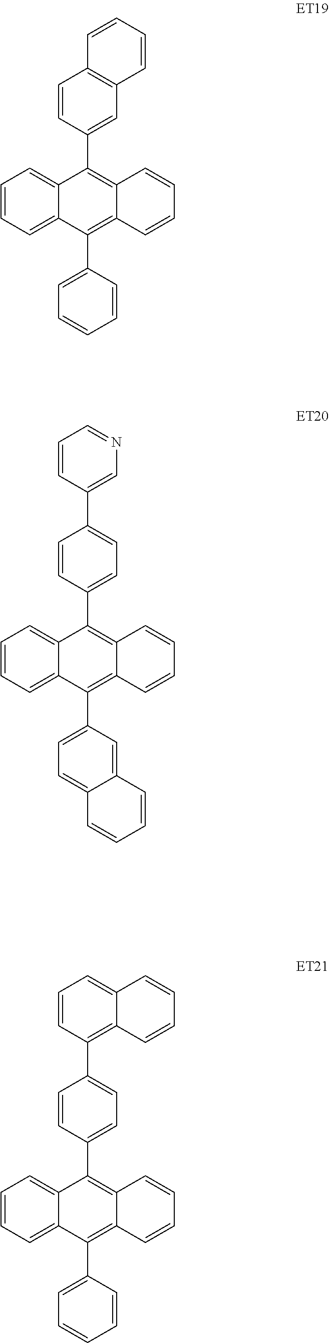

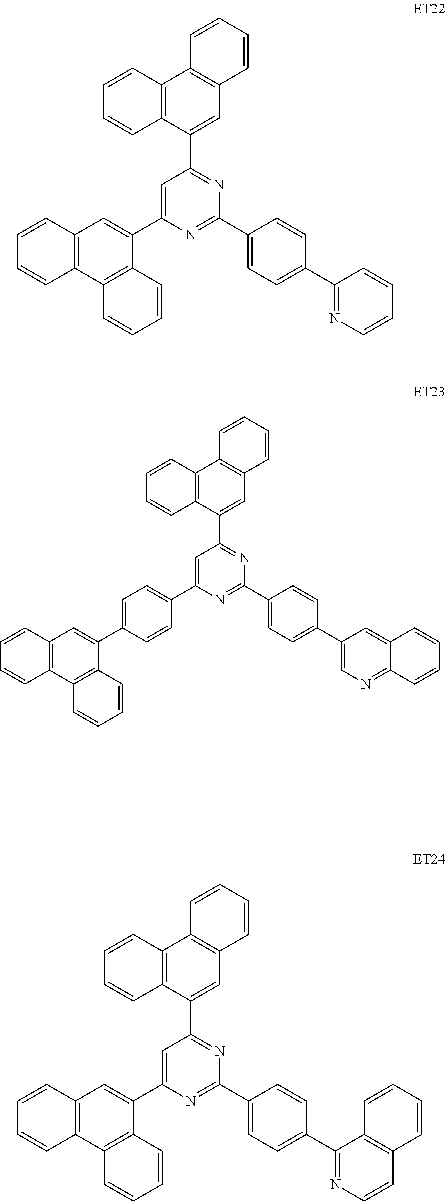 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

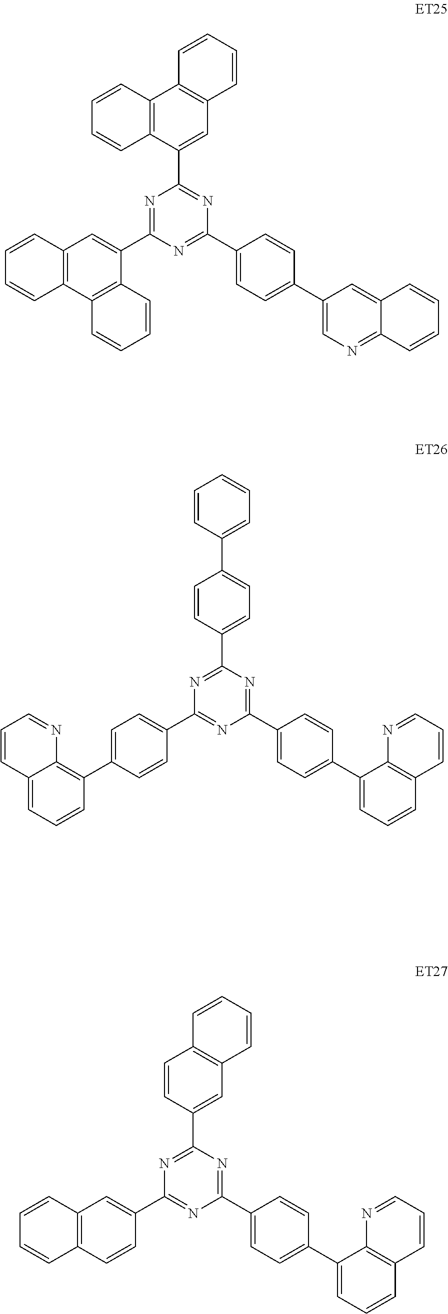 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

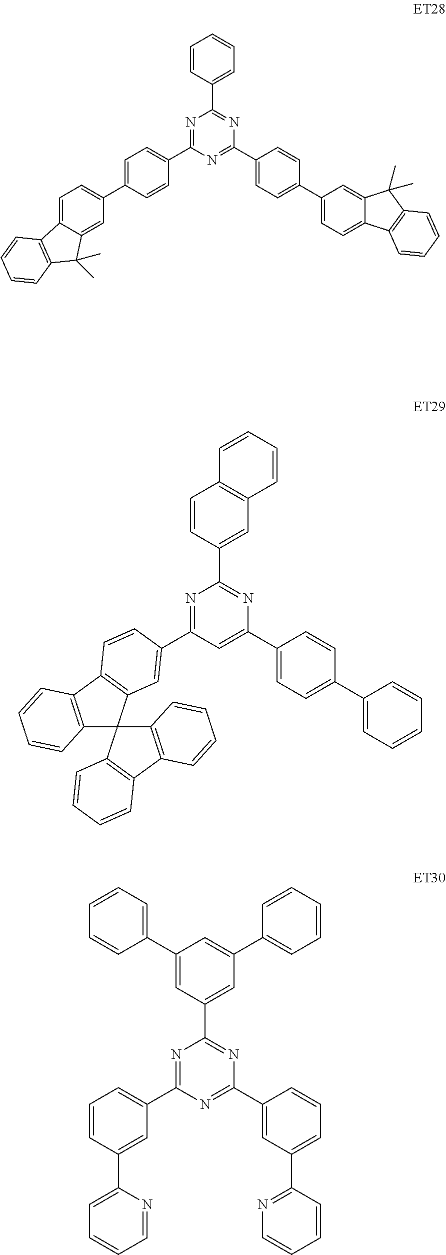 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

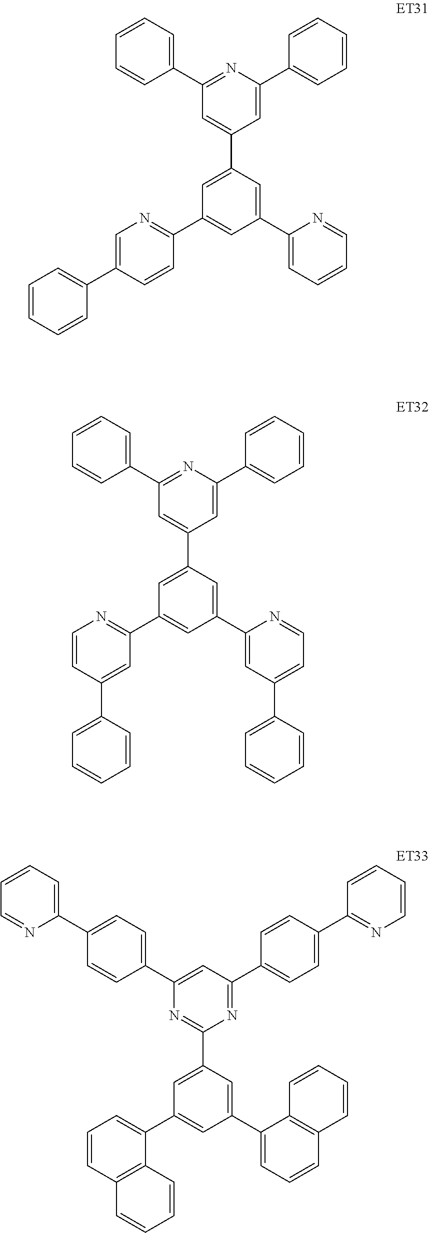 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

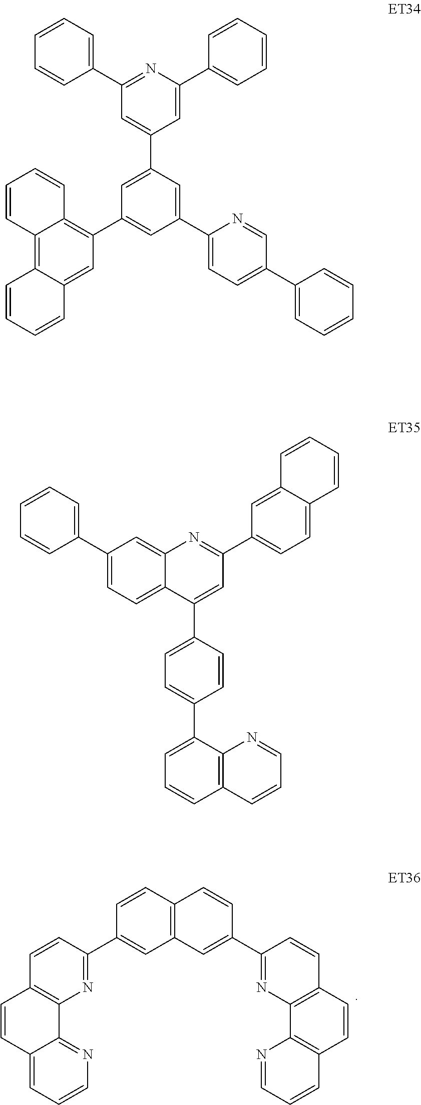 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In one or more embodiments, the electron transport region 17 may include 2,9-dimethyl-4,7-diphenyl-1,10-phenanthroline (BCP), 4,7-dphenyl-1,10-phenanthroline (Bphen), tris(8-hydroxy-quinolinato)aluminum (Alq₃), bis(2-methyl-8-quinolinolato-N1,O8)-(1,1′-biphenyl-4-olato)aluminum (BAlq), 3-(biphenyl-4-yl)-5-(4-tert-butylphenyl)-4-phenyl-4H-1,2,4-triazole (TAZ), 4-(naphthalen-1-yl)-3,5-diphenyl-4H-1,2,4-triazole (NTAZ), 2,8-bis(diphenyl-phosphoryl)-dibenzo[b,d]furan (DBFPO), or any combination thereof. For example, when the electron transport region 17 includes a hole blocking layer, the hole blocking layer may include 2,9-dimethyl-4,7-diphenyl-1,10-phenanthroline (BCP) or 4,7-diphenyl-1,10-phenanthroline (Bphen):

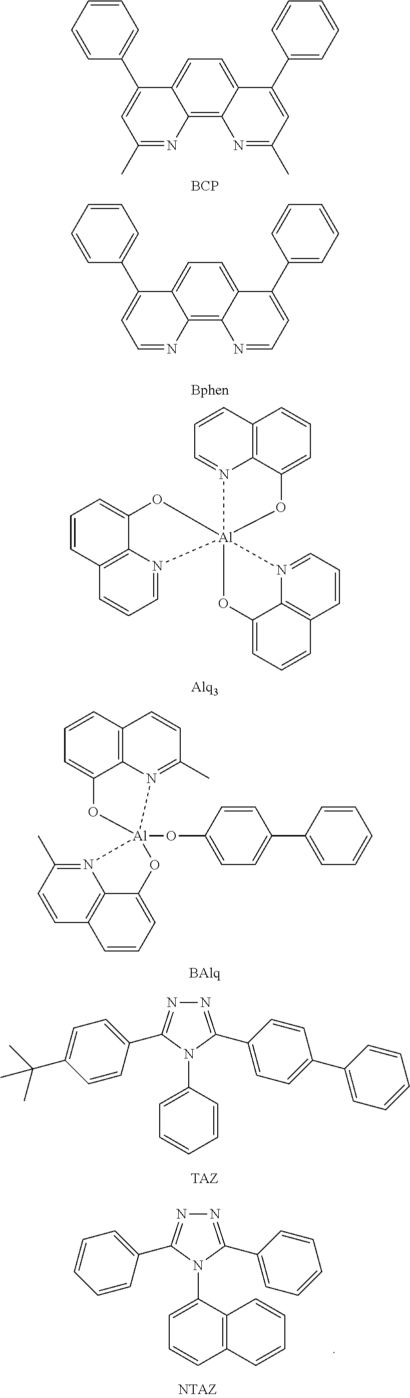

Thicknesses of the buffer layer, the hole blocking layer, and the electron control layer may each independently be in the range of about 20 Å to about 1,000 Å, for example, about 30 Å to about 300 Å. When the thicknesses of the buffer layer, the hole blocking layer, and the electron control layer are within these ranges, excellent hole blocking characteristics or excellent electron control characteristics may be obtained without a substantial increase in driving voltage.

A thickness of the electron transport layer may be in the range of about 100 Å to about 1,000 Å, for example, about 150 Å to about 500 Å. When the thickness of the electron transport layer is within the range described above, the electron transport layer may have satisfactory electron transporting characteristics without a substantial increase in driving voltage.

The electron transport region 17 (for example, the electron transport layer in the electron transport region 17) may further include, in addition to the materials described above, a metal-containing material.

The metal-containing material may include an alkali metal complex, an alkaline earth metal complex, or any combination thereof. A metal ion of the alkali metal complex may include a Li ion, a Na ion, a K ion, a Rb ion, a Cs ion, or any combination thereof, and a metal ion of the alkaline earth metal complex may include a Be ion, a Mg ion, a Ca ion, a Sr ion, a Ba ion, or any combination thereof. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxydiphenyloxadiazole, a hydroxydiphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In an embodiment, the metal-containing material may include a Li complex. The Li complex may include, for example, Compound ET-D1 (LiQ) or ET-D2:

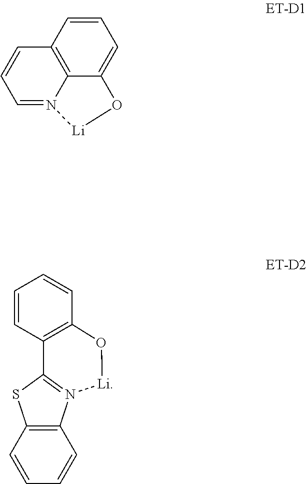

The electron transport region 17 may include an electron injection layer that facilitates the injection of electrons from the second electrode 19. The electron injection layer may directly contact the second electrode 19.

The electron injection layer may have i) a single-layered structure including a single layer including a single material, ii) a single-layered structure including a single layer including a plurality of different materials, or iii) a multi-layered structure having a plurality of layers including a plurality of different materials.

The electron injection layer may include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combinations thereof.

The alkali metal may include Li, Na, K, Rb, Cs, or any combination thereof. In an embodiment, the alkali metal may be Li, Na, or Cs. In one or more embodiments, the alkali metal may be Li or Cs.

The alkaline earth metal may include Mg, Ca, Sr, Ba, or any combination thereof.

The rare earth metal may include Sc, Y, Ce, Tb, Yb, Gd, or any combination thereof.

The alkali metal compound, the alkaline earth metal compound, and the rare earth metal compound may include oxides and halides (for example, fluorides, chlorides, bromides, or iodides) of the alkali metal, the alkaline earth metal, and the rare earth metal, or any combination thereof.

The alkali metal compound may include: one of alkali metal oxides such as Li₂O, Cs₂O, or K₂O; one of alkali metal halides such as LiF, NaF, CsF, KF, Lil, Nal, Csl, or KI; or any combination thereof. In an embodiment, the alkali metal compound may include LiF, Li₂O, NaF, Lil, Nal, Csl, KI, or any combination thereof.

The alkaline earth-metal compound may include one of alkaline earth-metal compounds, such as BaO, SrO, CaO, BaxSri-xO (wherein 0<x<1), or BaxCai-xO (wherein 0<x<1), or any combination thereof. In an embodiment, the alkaline earth metal compound may include BaO, SrO, CaO, or any combination thereof.

The rare earth metal compound may include YbF₃, ScF₃, ScO₃, Y₂O₃, Ce₂O₃, GdF₃, TbF₃, or any combination thereof. In an embodiment, the rare earth metal compound may include YbF₃, ScF₃, TbF₃, YbI₃, ScI₃, TbI₃, or any combination thereof.

The alkali metal complex, the alkaline earth metal complex, and the rare earth metal complex may include an ion of alkali metal, alkaline earth metal, and rare earth metal as described above, and a ligand coordinated with a metal ion of the alkali metal complex, the alkaline earth metal complex, or the rare earth metal complex may include hydroxy quinoline, hydroxy isoquinoline, hydroxy benzoquinoline, hydroxy acridine, hydroxy phenanthridine, hydroxy phenyloxazole, hydroxy phenylthiazole, hydroxy diphenyloxadiazole, hydroxy diphenylthiadiazole, hydroxy phenylpyridine, hydroxy phenylbenzimidazole, hydroxy phenylbenzothiazole, bipyridine, phenanthroline, cyclopentadiene, or any combination thereof.

The electron injection layer may consist of an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combinations thereof, as described above. In one or more embodiments, the electron injection layer may further include an organic material. When the electron injection layer further includes an organic material, an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or any combination thereof may be homogeneously or non-homogeneously dispersed in a matrix including the organic material.

A thickness of the electron injection layer may be in a range of about 1 Å to about 100 Å, and, for example, about 3 Å to about 90 Å. When the thickness of the electron injection layer is within the ranges described above, satisfactory electron injection characteristics may be obtained without a substantial increase in driving voltage.

### Second Electrode 19

The second electrode 19 is arranged on the aforementioned interlayer 10A. The second electrode 19 may be a cathode which is an electron injection electrode, and in this regard, a material for forming the second electrode 19 may be selected from a metal, an alloy, an electrically conductive compound, and a combination thereof, which have a relatively low work function.

The second electrode 19 may include lithium (Li), silver (Ag), magnesium (Mg), aluminum (AI), aluminum-lithium (Al—Li), calcium (Ca), magnesium-indium (Mg—In), magnesium-silver (Mg—Ag), ITO, IZO, or any combination thereof. The second electrode 19 may be a transmissive electrode, a semi-transmissive electrode, or a reflective electrode.

The second electrode 19 may have a single-layered structure having a single layer or a multi-layered structure including two or more layers.

### Explanation of Terms

The term “C₁-C₆₀ alkyl group” as used herein refers to a linear or branched saturated aliphatic hydrocarbons monovalent group having 1 to 60 carbon atoms, and the term “C₁-C₆₀ alkylene group, as used here refers to a divalent group having the same structure as the C₁-C₆₀ alkyl group.

Examples of the C₁-C₆₀ alkyl group, the C₁-C₂₀ alkyl group, and/or the C₁-C₁₀ alkyl group are a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group, each unsubstituted or substituted with a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, or any combination thereof.

The term “C₁-C₆₀ alkoxy group” as used herein refers to a monovalent group represented by —OA₁₀₁ (wherein A₁₀₁ is the C₁-C₆₀ alkyl group), and examples thereof are a methoxy group, an ethoxy group, a propoxy group, a butoxy group, a pentoxy group, or the like.

The term “C₂-C₆₀ alkenyl group” as used herein refers to a hydrocarbon group formed by substituting at least one carbon-carbon double bond in the middle or at the terminus of the C₂-C₆₀ alkyl group, and examples thereof include an ethenyl group, a propenyl group, and a butenyl group. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and examples thereof include an ethynyl group, and a propynyl group. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon cyclic group having 3 to 10 carbon atoms, and the term “C₃-C₁₀ cycloalkylene group” as used herein refers to a divalent group having the same structure as the C₃-C₁₀ cycloalkyl group.

Examples of the C₃-C₁₀ cycloalkyl group may include a cyclopropyl group, a cyclobutyl group, a cyclopentyl, cyclohexyl group, a cycloheptyl group, a cyclooctyl group, an adamantanyl group, a norbornanyl group (a bicyclo[2.2.1]heptyl group), a bicyclo[1.1.1]pentyl group, a bicyclo[2.1.1]hexyl group, a bicyclo[2.2.2]octyl group, or the like.

The term “C₁-C₁₀ heterocycloalkyl group” as used herein refers to a monovalent monocyclic group that includes at least one heteroatom selected from N, O, P, Si, S, Se, Ge, and B as a ring-forming atom and 1 to 10 carbon atoms, and the term “the C₁-C₁₀ heterocycloalkylene group” as used herein refers to a divalent group having the same structure as the C₁-C₁₀ heterocycloalkyl group.

Examples of the C₁-C₁₀ heterocycloalkyl group are a silolanyl group, a silinanyl group, tetrahydrofuranyl group, a tetrahydro-2H-pyranyl group, a tetrahydrothiophenyl group, or the like.

The term “C₃-C₁₀ cycloalkenyl group” as used herein refers to a monovalent cyclic group that includes 3 to 10 carbon atoms and at least one carbon-carbon double bond in the ring thereof and has no aromaticity, and examples thereof include a cyclopentenyl group, a cyclohexenyl group, a cycloheptenyl group, or the like. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one hetero atom selected from N, O, P, Si, S, Se, Ge, and B as a ring-forming atom, 1 to 10 carbon atoms, and at least one carbon-carbon double bond in the ring thereof. Examples of the C₁-C₁₀ heterocycloalkenyl group are a 2,3-dihydrofuranyl group, a 2,3-dihydrothiophenyl group, or the like. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic system having 6 to 60 carbon atoms, and the term “C₆-C₆₀ arylene group” as used herein refers to a divalent group having a carbocyclic aromatic system having 6 to 60 carbon atoms. Examples of the C₆-C₆₀ aryl group include a phenyl group, a naphthyl group, an anthracenyl group, a phenanthrenyl group, a pyrenyl group, a chrysenyl group, or the like. When the C₆-C₆₀ aryl group and the C₆-C₆₀ arylene group each include two or more rings, the rings may be fused to each other.

The term “C₇-C₆₀ alkyl aryl group” as used herein refers to a C₆-C₆₀ aryl group substituted with at least one C₁-C₆₀ alkyl group.

The term “C₇-C₆₀ aryl alkyl group” as used herein refers to a C₁-C₆₀ alkyl group substituted with at least one C₆-C₆₀ aryl group.

The term “C₁-C₆₀ heteroaryl group” as used herein refers to a monovalent group that includes at least one heteroatom selected from N, O, P, Si, S, Se, Ge, and B as a ring-forming atom and a heterocyclic aromatic system having 1 to 60 carbon atoms, and the term “C₁-C₆₀ heteroarylene group” as used herein refers to a divalent group that includes at least one heteroatom selected from N, O, P, Si, S, Se, Ge, and B as a ring-forming atom and a heterocyclic aromatic system having 1 to 60 carbon atoms. Examples of the C₁-C₆₀ heteroaryl group include a pyridinyl group, a pyrimidinyl group, a pyrazinyl group, a pyridazinyl group, a triazinyl group, a quinolinyl group, an isoquinolinyl group, or the like. When the C₁-C₆₀ heteroaryl group and the C₁-C₆₀ heteroarylene group each include two or more rings, the rings may be fused to each other.

The term “C₂-C₆₀ alkyl heteroaryl group” as used herein refers to a C₁-C₆₀ heteroaryl group substituted with at least one C₁-C₆₀ alkyl group.

The term “C₂-C₆₀ heteroaryl alkyl group” as used herein refers to a C₁-C₆₀ alkyl group substituted with at least one C₆-C₆₀ heteroaryl group.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “C₁-C₆₀ heteroaryloxy group” as used herein indicates —OA₁₀₄ (wherein A₁₀₄ is the C₁-C₆₀ heteroaryl group), and the term “C₁-C₆₀ heteroarylthio group” as used herein indicates —SA₁₀₅ (wherein A₁₀₅ is the C₁-C₆₀ heteroaryl group).

The term “monovalent non-aromatic condensed polycyclic group” as used herein refers to a monovalent group in which two or more rings are condensed with each other, only carbon is used as a ring-forming atom (for example, the number of carbon atoms may be 8 to 60) and the whole molecule is a non-aromaticity group. Examples of the monovalent non-aromatic condensed polycyclic group include a fluorenyl group. The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group having two or more rings condensed with each other, a heteroatom selected from N, O, P, Si, S, Se, Ge, and B, other than carbon atoms (for example, having 1 to 60 carbon atoms), as a ring-forming atom, and no aromaticity in its entire molecular structure. Examples of the monovalent non-aromatic condensed heteropolycyclic group include a carbazolyl group. The term “divalent non-aromatic condensed heteropolycyclic group” as used herein refers to a divalent group having the same structure as the monovalent non-aromatic condensed heteropolycyclic group described above.

The term “π electron-depleted nitrogen-containing C₁-C₆₀ cyclic group” as used herein refers to a cyclic group having 1 to 60 carbon atoms and including at least one *—N═*′ (wherein * and *′ each indicate a binding site to an adjacent atom) as a ring-forming moiety. For example, the π electron-depleted nitrogen-containing C₁-C₆₀ cyclic group may be a) a first ring, b) a condensed ring in which at least two first rings are condensed, or c) a condensed ring in which at least one first ring and at least one second ring are condensed.

The term “π electron-rich C₃-C₆₀ cyclic group” as used herein refers to a cyclic group having 3 to 60 carbon atoms and not including at least one *—N═*′ (wherein * and *′ each indicate a binding site to an adjacent atom) as a ring-forming moiety. For example, the π electron-rich C₃-C₆₀ cyclic group may be a) a second ring or b) a condensed ring in which at least two second rings are condensed.

The term “C₅-C₆₀ cyclic group” as used herein refers to a monocyclic or polycyclic group having 5 to 60 carbon atoms, and may be, for example, a) a third ring or b) a condensed ring in which two or more third rings are condensed with each other.

The term “C₁-C₆ heterocyclic group” as used herein refers to a monocyclic or polycyclic group that has 1 to 60 carbon atoms and includes at least one heteroatom, and may be, for example, a) a fourth ring, b) a condensed ring in which two or more fourth rings are condensed with each other, or c) a condensed ring in which at least one third ring is condensed with at least one fourth ring.

The “first ring” as used herein may be an imidazole group, a pyrazole group, a thiazole group, an isothiazole group, an oxazole group, an isoxazole group, a pyridine group, a pyrazine group, a pyridazine group, a pyrimidine group, a triazole group, a tetrazole group, an oxadiazole group, a triazine group, or a thiadiazole group.

The “second ring” as used herein may be a benzene group, a cyclopentadiene group, a pyrrole group, a furan group, a thiophene group, or a silole group.

The “third ring” as used herein may be a cyclopentane group, a cyclopentadiene group, an indene group, an adamantane group, a norbornene group, a bicyclo[1.1.1]pentane group, a bicyclo[2.1.1]hexane group, a bicyclo[2.2.1]heptane group (a norbornane group), a bicyclo[2.2.2]octane group, a cyclohexane group, a cyclohexene group, or a benzene group.

The “fourth ring” as used herein may be a furan group, a thiophene group, a pyrrole group, a silole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, a triazasilole group, a pyridine group, a pyrimidine group, a pyrazine group, a pyridazine group, or a triazine group.

In some embodiments, the π electron-depleted nitrogen-containing C₁-C₆₀ cyclic group may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a benzoisoquinoline group, a phthalazine group, a naphthyridine group, a quinoxaline group, a benzoquinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, an azadibenzofuran group, an azadibenzothiophene group, an azadibenzosilole group, an acridine group, or a pyridopyrazine group.

In some embodiments, the π electron-rich C₃-C₆₀ cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, a furan group, a thiophene group, an isoindole group, an indole group, an indene group, a benzofuran group, a benzothiophene group, a benzosilole group, a naphthopyrrole group, a naphthofuran group, a naphthothiophene group, a naphthosilole group, a benzocarbazole group, a dibenzocarbazole group, a dibenzofuran group, a dibenzothiophene group, a carbazole group, a dibenzosilole group, an indenocarbazole group, an indolocarbazole group, a benzofurocarbazole group, a benzothienocarbazole group, a benzosilolocarbazole group, a triindolobenzene group, a pyrrolophenanthrene group, a furanophenanthrene group, a thienophenanthrene group, a benzonaphthofuran group, a benzonapthothiophene group, an (indolo)phenanthrene group, a (benzofurano)phenanthrene group, or a (benzothieno)phenanthrene group.

For example, the C₅-C₆₀ carbocyclic group may be a cyclopentane group, a cyclohexane group, a cyclohexene group, a benzene group, a naphthalene group, an anthracene group, a phenanthrene group, a triphenylene group, a pyrene group, a chrysene group, a 1,2,3,4-tetrahydronaphthalene group, a cyclopentadiene group, an indene group, a fluorene group, a 5,6,7,8-tetrahydroisoquinoline group, a 5,6,7,8-tetrahydroquinoline group, an adamantane group, a norbornane group, or a norbornene group.

For example, the C₁-C₆₀ heterocyclic group may be a thiophene group, a furan group, a pyrrole group, a cyclopentadiene group, a silole group, a borole group, a phosphole group, a selenophene group, a germole group, a benzothiophene group, a benzofuran group, an indole group, an indene group, a benzosilole group, a benzoborole group, a benzophosphole group, a benzoselenophene group, a benzogermole group, a dibenzothiophene group, a dibenzofuran group, a carbazole group, a dibenzosilole group, a dibenzoborole group, a dibenzophosphole group, a dibenzoselenophene group, a dibenzogermole group, a dibenzothiophene 5-oxide group, a 9H-fluorene-9-one group, a dibenzothiophene 5,5-dioxide group, an azabenzothiophene group, an azabenzofuran group, an azaindole group, an azaindene group, an azabenzosilole group, an azabenzoborole group, an azabenzophosphole group, an azabenzoselenophene group, an azabenzogermole group, an azadibenzothiophene group, an azadibenzofuran group, an azacarbazole group, an azafluorene group, an azadibenzosilole group, an azadibenzoborole group, an azadibenzophosphole group, an azadibenzoselenophene group, an azadibenzogermole group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group.

The term “a π electron-deficient nitrogen-containing C₁-C₆₀ cyclic group, a rr electron-rich C₃-C₆₀ cyclic group, a C₅-C₆₀ cyclic group, and a C₁-C₆₀ heterocyclic group” may be part of a condensed cycle or may be a monovalent, a divalent, a trivalent, a tetravalent, a pentavalent, or a hexavalent group, depending on the formula structure.

Substituents of the substituted π electron-deficient nitrogen-containing C₁-C₆₀ cyclic group, the substituted π electron-rich C₃-C₆₀ cyclic group, the substituted C₅-C₆₀ cyclic group, the substituted C₁-C₆₀ heterocyclic group, the substituted C₁-C₆₀ alkylene group, the substituted C₂-C₆₀ alkenylene group, the substituted C₂-C₆₀ alkynylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂—Co alkynyl group, the substituted C₁-C₆₀ alkoxy group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may each independently be:
deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁a cycloalkenyl group, a C₁-C₁a heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
any combination thereof.

Q₁ to Q₉, Q₁₁ to Q₁₉, Q₂₁ to Q₂₉, and Q₃₁ to Q₃₉ may each independently be: hydrogen; deuterium; —F; or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁a cycloalkyl group, a C₁-C₁a heterocycloalkyl group, a C₃-C₁a cycloalkenyl group, a C₁-C₁a heterocycloalkenyl group, a C₆-C₆₀ aryl group, a C₆-C₆₀ aryloxy group, a C₆-C₆₀ arylthio group, a C₁-C₆a heteroaryl group, a monovalent non-aromatic condensed polycyclic group, or a monovalent non-aromatic condensed heteropolycyclic group, each unsubstituted or substituted with deuterium, —F, a cyano group, a C₁-C₆₀ alkyl group, a C₆-C₆₀ aryl group, or any combination thereof.

Unless otherwise defined, Q₁ to Q₉, Q₁₁ to Q₁₉, Q₂₁ to Q₂₉, and Q₃₁ to Q₃₉ described herein may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with deuterium, a C₁-C₁₀ alkyl group, a phenyl group, or any combination thereof.

The term “room temperature” used herein refers to a temperature of about 25° C.

The terms “a biphenyl group, a terphenyl group, and a tetraphenyl group” used herein respectively refer to monovalent groups in which two, three, or four phenyl groups which are linked together via a single bond.

Hereinafter, a compound and an organic light-emitting device according to embodiments are described in detail with reference to Synthesis Examples and Examples.

However, the organic light-emitting device is not limited thereto. The wording “‘B’ was used instead of ‘A’” used in describing Synthesis Examples means that an amount of ‘A’ used was identical to an amount of ‘B’ used, in terms of a molar equivalent.

### EXAMPLES

### Synthesis Example 1 (Compound 1)

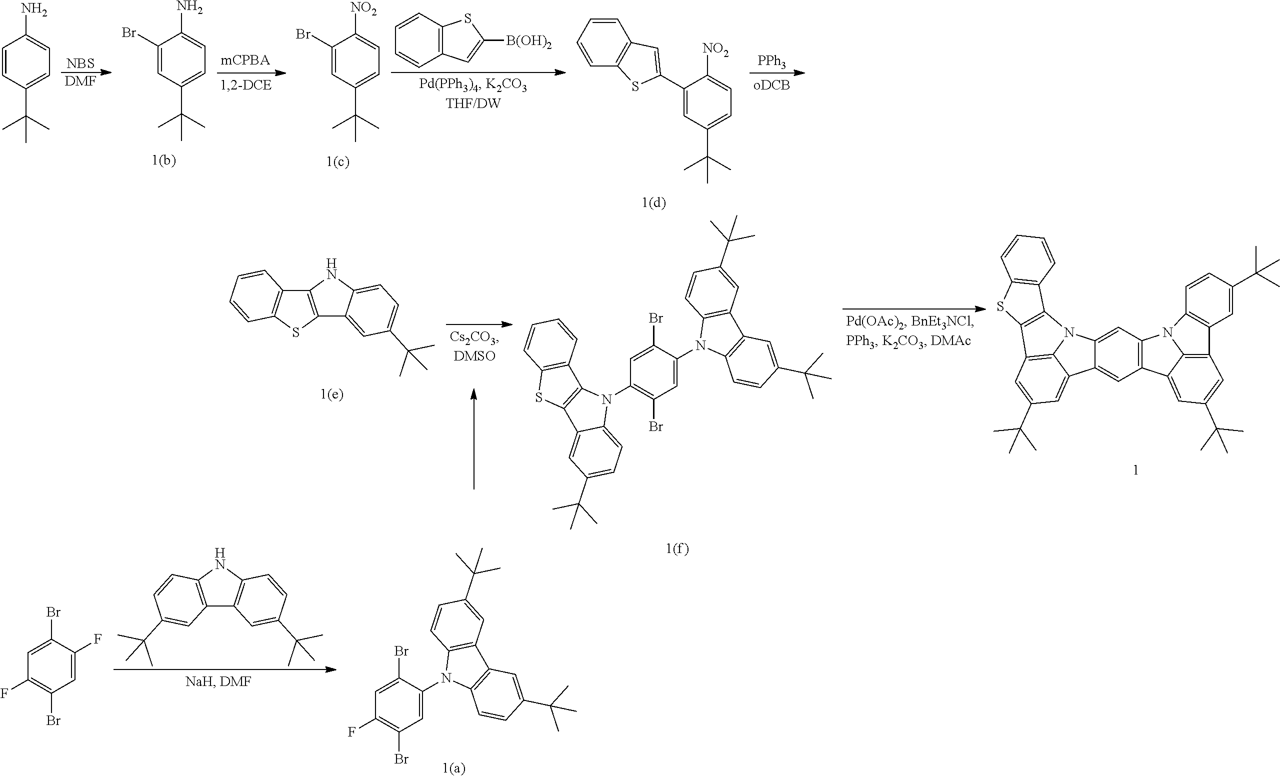

### Synthesis of Intermediate 1(a)

3,6-di-tert-butyl-9H-carbazole (10.00 grams (g), 35.79 millimoles (mmol)) and sodium hydride (NaH) (0.85 g, 35.42 mmol) were combined in a round flask, and 100 milliliters (mL) of N,N-dimethylformamide (DMF) was added thereto under a nitrogen atmosphere. After stirring the mixture at room temperature for 30 minutes, 1,4-dibromo-2,5-difluorobenzene (5.00 g, 18.39 mmol) dissolved in 50 mL of DMF was slowly added dropwise into the round flask. After completion of the reaction, dichloromethane and deionized water (DW) were added thereto to extract an organic layer. The organic layer was dried over anhydrous magnesium sulfate. The dried organic layer was filtered under reduced pressure and the filtrate was concentrated under reduced pressure to provide a residue. The residue was then purified by silica gel column chromatography to provide Intermediate 1(a) as a white solid (9.50 gr, 60% yield).

LC-Mass (calculated: 530.05, found: 530.06 (M+1)).

### Synthesis of Intermediate 1(b)

4-(tert-butyl)aniline (20.00 g, 134.02 mmol) was dissolved in 200 mL of DMF in a round flask, and the mixture was cooled to 0° C. and stirred. The cooled state of the mixture was maintained as N-bromosuccinimide (25.00 g, 140.47 mmol) dissolved in 60 mL of DMF was slowly added thereto dropwise in the dark. After completion of the dropwise addition, the mixture was stirred at room temperature. After the reaction was completed, 260 mL of an aqueous 2 moles per liter (M) solution of sodium thiosulfate (82.22 g, 520.00 mmol) was added thereto dropwise. Dichloromethane and DW were added thereto to extract an organic layer. The organic layer was dried over anhydrous magnesium sulfate. The dried organic layer was filtered under reduced pressure and the filtrate was concentrated under reduced pressure to provide a residue. The concentrated residue was then purified by silica gel column chromatography to provide Intermediate 1(b) as a red liquid (27.52 g, 90% yield).

LC-Mass (calculated: 228.04, found: 228.07 (M+1)).

### Synthesis of Intermediate 1(c)

3-chloroperoxybenzoic acid (mCPBA) (77%) (15.13 g, 87.67 mmol) and 150 mL of 1,2-dichloroethane (1,2-DCE) were combined in a round flask and then heated and stirred until the mCPBA was completely dissolved. Then, Intermediate 1(b) (5.00 g, 21.92 mmol) dissolved in 1,2-DCE was slowly added thereto dropwise, and the mixture was stirred under reflux. After completion of the reaction, 60 mL of an aqueous 2 M solution of sodium thiosulfate (18.97 g, 120.00 mmol) was added dropwise thereto. Dichloromethane and DW were added thereto to extract an organic layer. The organic layer was dried over anhydrous magnesium sulfate. The dried organic layer was filtered under reduced pressure and the filtrate was concentrated under reduced pressure to provide a residue. The residue was then purified by silica gel column chromatography to provide Intermediate 1(c) as a white solid (5.50 g, 97% yield).

LC-Mass (calculated: 258.01, found: 258.01 (M+1)).

### Synthesis of Intermediate 1(d)

Intermediate 1(b) (2.50 g, 9.69 mmol), benzo[b]thiophen-2-ylboronic acid (2.58 g, 14.49 mmol), tetrakis(triphenylphosphine)palladium(0) (Pd(PPh₃)₄) (0.34 g, 0.29 mmol), and 40 mL of tetrahydrofuran (THF) were combined in a round flask. Then, 20 mL of an aqueous 2 M solution of potassium carbonate (K₂CO₃) (5.53 g, 40.00 mmol) was added to the mixture. The reaction mixture was stirred under reflux under a nitrogen atmosphere. After completion of the reaction, the mixture was cooled to room temperature and extracted with dichloromethane and DW. The organic layer was dried over anhydrous magnesium sulfate. The dried organic layer was concentrated under reduced pressure to provide a residue. The residue was purified by silica gel column chromatography to provide Intermediate 1(d) as a yellow solid (3.00 g, 99% yield).

LC-Mass (calculated: 312.11, found: 312.12 (M+1)).

### Synthesis of Intermediate 1(e)

Intermediate 1(d) (3.10 g, 9.95 mmol), triphenylphosphine (PPh₃) (6.53 g, 24.90 mmol), and 15 mL of ortho-dichlorobenzene (oDCB) were combined in a round flask and stirred under reflux under a nitrogen atmosphere. After 12 hours, triphenylphosphine (6.53 g, 24.90 mmol) was added thereto, and the reaction was allowed to proceed for another 12 hours. The cooled reaction mixture was extracted as an organic layer with dichloromethane and DW. The organic layer was dried over anhydrous magnesium sulfate and filtered. The filtrate was concentrated under reduced pressure to provide a residue. The residue was purified by silica gel column chromatography to provide Intermediate 1(e) as a yellow solid (2.21 g, 79% yield).

LC-Mass (calculated: 280.12, found: 280.10 (M+1)).

### Synthesis of Intermediate 1(f)

Intermediate 1(a) (0.40 g, 0.75 mmol), Intermediate 1(e) (0.25 g, 0.89 mmol), cesium carbonate (Cs₂CO₃) (0.61 g, 1.87 mmol), and 7 mL of dimethyl sulfoxide (DMSO) were combined in a round flaks and stirred under reflux under a nitrogen atmosphere. After completion of the reaction, the reaction mixture was cooled to room temperature. The organic layer was extracted with dichloromethane and DW. The organic layer was dried over anhydrous magnesium sulfate. The dried organic layer was filtered and the filtrate r was concentrated under reduced pressure to provide a residue. The residue was purified by silica gel column chromatography to provide Intermediate 1(f) as a white solid was obtained therefrom as (0.35 g, 59% yield).

LC-Mass (calculated: 789.15, found: 789.15 (M+1)).

### Synthesis of Compound 1

Intermediate 1(f) (0.70 g, 0.89 mmol), palladium (II) acetate (Pd(OAc)₂) (0.06 g, 0.26 mmol), benzyltriethylammonium chloride (BnEt₃NCl) (0.46 g, 2.02 mmol), triphenylphosphine (0.23 g, 0.88 mmol), potassium carbonate (1.22 g, 8.83 mmol), and 9 mL of N,N-dimethylacetamide (DMAc) were combined in a round flask and stirred under reflux under a nitrogen atmosphere. After completion of the reaction, the reaction mixture was diluted with dichloromethane and then filtered under reduced pressure with a short filter plug of silica gel and celite. The filtrate was concentrated under reduced pressure to provide a residue. The residue was purified by silica gel column chromatography. The material isolated from the column chromatography purification was concentrated and dissolved in dichloromethane. Methanol was added to the solution to precipitate a solid.

The solid was filtered under reduced pressure to provide Compound 1 as a pale yellow solid (0.35 g, 63% yield).

LC-Mass (calculated: 628.29, found: 628.29 (M+1)).

### Synthesis Example 2 (Compound 2)

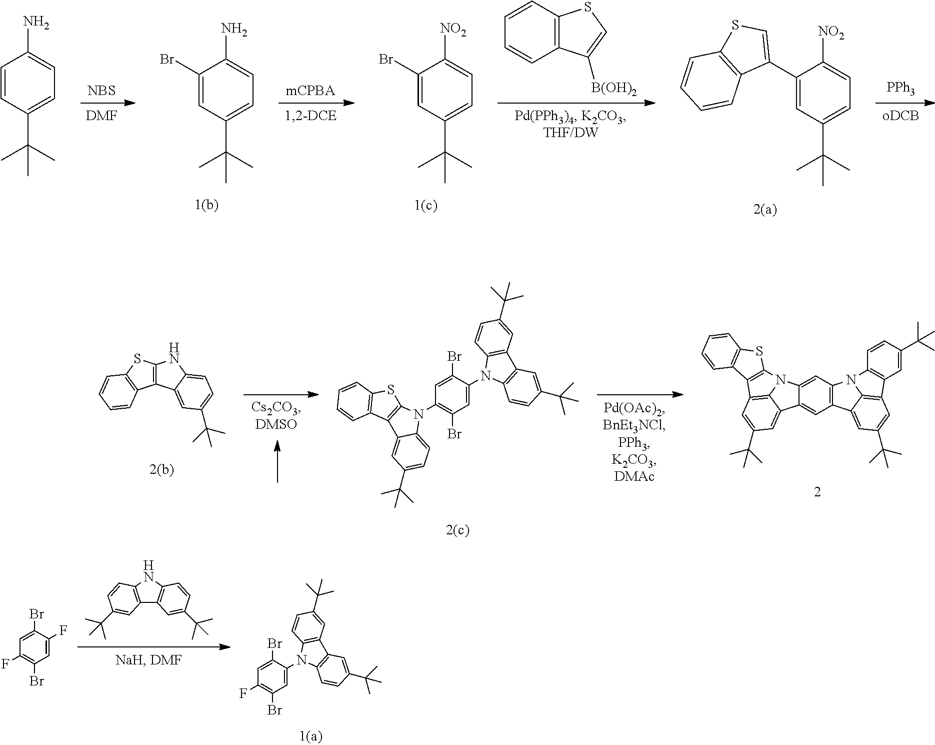

### Synthesis of Intermediate 2(a)

Intermediate 1(c) (2.50 g, 9.69 mmol), benzo[b]thiophen-3-ylboronic acid (2.58 g, 14.49 mmol), tetrakis(triphenylphosphine)palladium (0.34 g, 0.29 mmol), 40 mL of tetrahydrofuran, and 20 mL of an aqueous 2 M solution of potassium carbonate (5.53 g, 40.00 mmol) were combined in a round flask and stirred under reflux. After completion of the reaction, the reaction mixture was purified by the process described in the synthesis of Intermediate 1(d). Intermediate 2(a) was obtained as a yellow solid (3.00 g, 99% yield).

LC-Mass (calculated: 312.11, found: 312.12 (M+1)).

### Synthesis of Intermediate 2(b)

Intermediate 2(a) (3.10 g, 9.95 mmol), triphenylphosphine (6.53 g, 24.90 mmol), and 15 mL of ortho-dichlorobenzene were combined in a round flask and stirred under reflux under a nitrogen atmosphere. After 12 hours, triphenylphosphine (6.53 g, 24.90 mmol) was added thereto, and the reaction was allowed to proceed for an additional 12 hours. The reaction product was purified by the process described in the synthesis of Intermediate 1(e). Intermediate 2(b) was obtained as a yellow solid (2.36 g, 85% yield).

LC-Mass (calculated: 280.12, found: 280.12 (M+1)).

### Synthesis of Intermediate 2(c)

Intermediate 1(a) (0.40 g, 0.75 mmol), Intermediate 2(b) (0.25 g, 0.89 mmol), cesium carbonate (0.61 g, 1.87 mmol), and 7 mL of dimethylsulfoxide were combined in a round flask and stirred under reflux under a nitrogen atmosphere. After completion of the reaction, the reaction mixture was purified by the process described in the synthesis of Intermediate 1(f). Intermediate 2(c) was obtained as a white solid (0.39 g, 66% yield).

LC-Mass (calculated: 789.15, found: 789.15 (M+1)).

### Synthesis of Compound 2

Intermediate 2(c) (0.80 g, 1.01 mmol), palladium (II) acetate (Pd(OAc)₂) (0.07 g, 0.31 mmol), benzyltriethylammonium chloride (BnEt₃NCl) (0.46 g, 2.02 mmol), triphenylphosphine (0.27 g, 1.03 mmol), potassium carbonate (1.40 g, 10.13 mmol), and 10 mL of DMAc were combined in a round flask and stirred under reflux under a nitrogen atmosphere. After completion of the reaction, the reaction mixture was purified by the process described in the synthesis of Compound 1. Compound 2 was obtained as a white solid (0.27 g, 42% yield).

LC-Mass (calculated: 628.29, found: 628.29 (M+1)).

### Synthesis Example 3 (Compound 3)

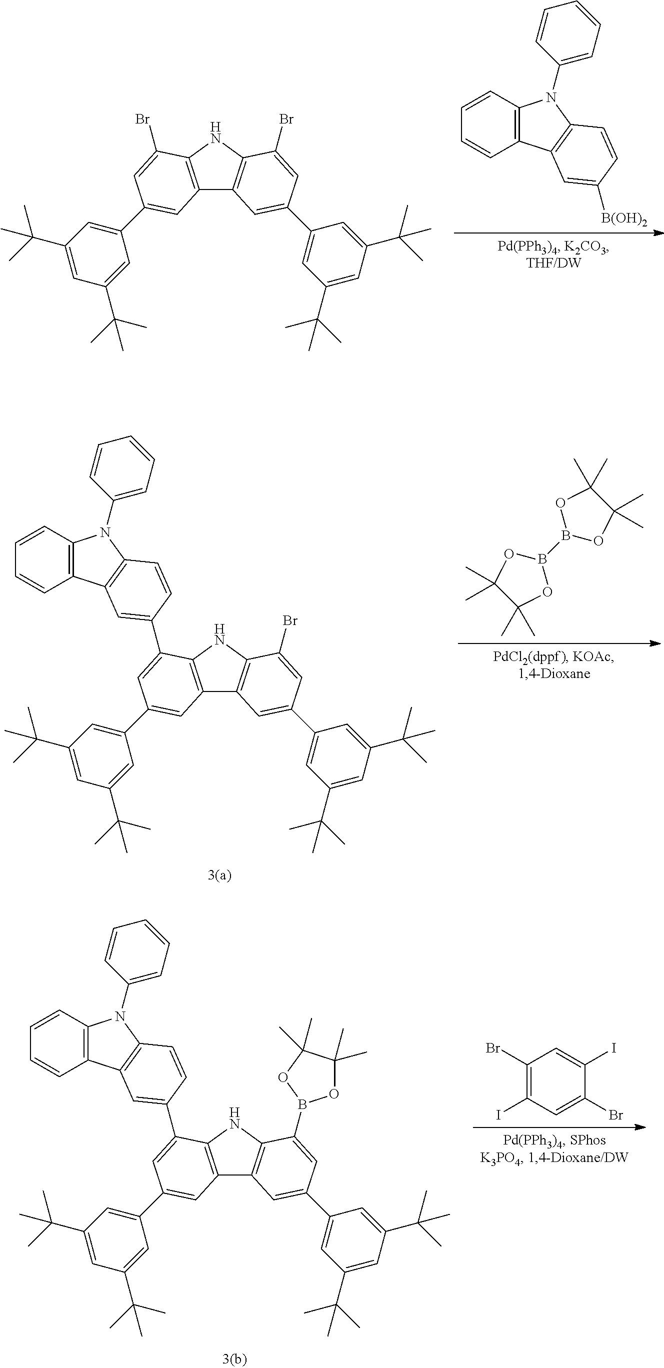

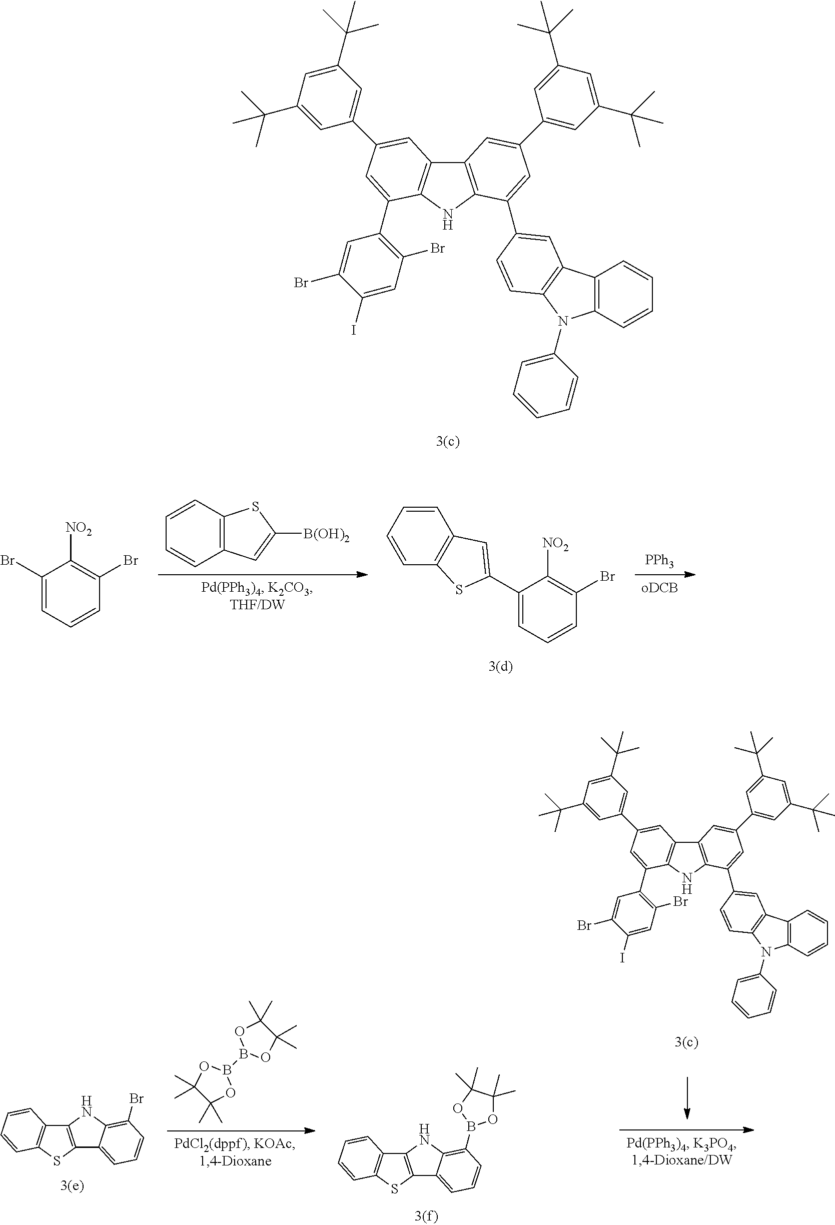

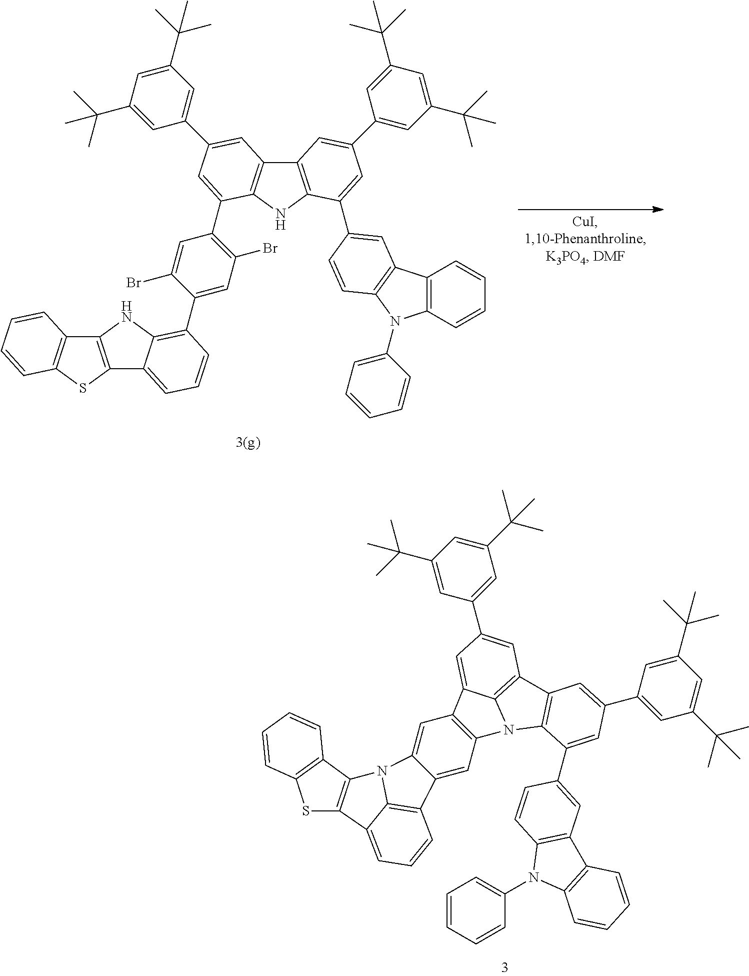 [SMILES: CC(C)(C)c1cc(-c2cc(-c3ccc4c(c3)c3ccccc3n4-c3ccccc3)c3[nH]c4c(-c5cc(Br)c(-c6cccc7c6[nH]c6c8ccccc8sc76)cc5Br)cc(-c5cc(C(C)(C)C)cc(C(C)(C)C)c5)cc4c3c2)cc(C(C)(C)C)c1 ; "CuI,1,10-Phenanthroline,K3PO4, DMF" ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4sc5ccccc5c4n6c23)cc(C(C)(C)C)c1]

### Synthesis of Intermediate 3(a)

1,8-dibromo-3,6-bis(3,5-di-tert-butylphenyl)-9H-carbazole (10.00 g, 14.25 mmol), (9-phenyl-9H-carbazol-3-yl)boronic acid (2.05 g, 7.13 mmol), tetrakis(triphenylphosphine)palladium (0.25 g, 0.21 mmol), and 60 mL of tetrahydrofuran were combined in a round flask. Then, 30 mL of an aqueous 2 M solution of potassium carbonate (8.29 g, 60.00 mmol) solution was added to the mixture. The reaction mixture was stirred under reflux under a nitrogen atmosphere. After completion of the reaction, the reaction mixture was purified by the process described in the synthesis of Intermediate 1(d). Intermediate 3(a) was obtained as a white solid (2.96 g, 48% yield).

LC-Mass (calculated: 863.39, found: 863.38 (M+1)).

### Synthesis of Intermediate 3(b)

Intermediate 3(a) (7.00 g, 8.10 mmol), bis(pinacolato)diboron (3.09 g, 12.15 mmol), [1,1′-bis(diphenylphosphino)ferrocene]dichloropalladium(II) (PdCl₂(dppf)) (0.18 g, 0.24 mmol), potassium acetate (KOAc) (1.59 g, 16.20 mmol), and 30 mL of 1,4-dioxane were combined in a round flask and stirred under reflux under a nitrogen atmosphere. After completion of the reaction, the reaction product was diluted with dichloromethane and then subjected to filtration under reduced pressure with a short filter plug of silica gel and celite. The filtrate was concentrated under reduced pressure and purified by silica gel column chromatography. Intermediate 3(b) was obtained as a white solid (4.80 g, 65% yield).

LC-Mass (calculated: 911.57, found: 911.57 (M+1)).

### Synthesis of Intermediate 3(c)

Intermediate 3(b) (3.00 g, 3.29 mmol), 1,4-dibromo-2,5-diiodobenzene (3.21 g, 6.59 mmol), tetrakis(triphenylphosphine)palladium (0.19 g, 0.16 mmol), dicyclohexyl(2′,6′-dimethoxy[1,1′-biphenyl]-2-yl)phosphane (SPhos) (0.13 g, 0.33 mmol), and 30 mL of 1,4-dioxane were combined in a round flask. Then, 15 mL of an aqueous 2 M solution of potassium phosphate tribasic (K₃PO₄) (6.37 g, 30.00 mmol) was added to the mixture. The reaction mixture was stirred under reflux under a nitrogen atmosphere. After completion of the reaction, the reaction mixture was purified by the process described in the synthesis of Intermediate 1(d). Intermediate 3(c) was obtained as a white solid (1.51 g, 40% yield).

LC-Mass (calculated: 1143.23, found: 1143.23 (M+1)).

### Synthesis of Intermediate 3(d)

1,3-dibromo-2-nitrobenzene (10.00 g, 35.60 mmol), benzothiophene-2-boronic acid (3.17 g, 17.80 mmol), tetrakis(triphenylphosphine)palladium (0.62 g, 0.53 mmol), and 100 mL of tetrahydrofuran were combined in a round flask. Then, 50 mL of an aqueous 2 M solution of potassium carbonate (13.82 g, 100.00 mmol) was added to the mixture. The reaction mixture was stirred under reflux under a nitrogen atmosphere. After completion of the reaction, the reaction mixutre was purified by the process described in the synthesis of Intermediate 1(d). Intermediate 3(d) was obtained as a white solid (3.07 g, 52% yield).

LC-Mass (calculated: 333.95, found: 333.95 (M+1)).

### Synthesis of Intermediate 3(e)

Intermediate 3(d) (2.70 g, 8.08 mmol), triphenylphosphine (5.30 g, 20.20 mmol), and 12 mL of ortho-dichlorobenzene were combined in a round flask and stirred under reflux under a nitrogen atmosphere. After 12 hours, triphenylphosphine (5.30 g, 20.20 mmol) was added thereto, and the reaction was allowed to proceed for an additional 12 hours. The reaction product was purified by the process described in the synthesis of Intermediate 1(e). Intermediate 3(e) was obtained as a yellow solid (1.03 g, 42% yield).

LC-Mass (calculated: 301.96, found: 301.96 (M+1)).

### Synthesis of Intermediate 3(f)

Intermediate 3(e) (0.50 g, 1.65 mmol), bis(pinacolato)diboron (0.63 g, 2.48 mmol), [1,1′-bis(diphenylphosphino)ferrocene]dichloropalladium(II) (0.04 g, 0.05 mmol), potassium acetate (0.32 g, 3.26 mmol), and 6.6 mL of 1,4-dioxane were combined in a round flask and stirred under reflux under a nitrogen atmosphere. After completion of the reaction, the reaction mixture was purified by the process described in the synthesis of Intermediate 3(c). Intermediate 3(f) was obtained as a yellow solid (0.46 g, 80% yield).

LC-Mass (calculated: 350.14, found: 350.15 (M+1)).

### Synthesis of Intermediate 3(g)

Intermediate 3(f) (0.40 g, mmol), Intermediate 3(c) (1.48 g, 1.29 mmol), tetrakis(triphenylphosphine)palladium (0.06 g, 0.05 mmol), and 12 mL of 1,4-dioxane were combined in a pressure tube. Then, 6 mL of an aqueous 2 M solution of potassium phosphate tribasic (2.55 g, 12.00 mmol) was added to the mixture. The reaction mixture was heated and stirred at 100° C. After completion of the reaction, the reaction mixture was purified by the process described in the synthesis of Intermediate 1(d). Intermediate 3(g) was obtained as a white solid (0.65 g, 49% yield).

LC-Mass (calculated: 1238.37, found: 1238.37 (M+1)).

### Synthesis of Compound 3

Intermediate 3(g) (0.6 g, 0.48 mmol), copper (1) iodide (CuI) (0.09 g, 0.48 mmol), 1,10-phenanthroline (0.09 g, 0.48 mmol), potassium phosphate tribasic (0.41 g, 1.92 mmol), and 10 mL of DMF were combined in a pressure tube. The reaction mixture was heated and stirred at 120° C. After completion of the reaction, the reaction mixture was purified by the process described in the synthesis of Compound 1. Compound 3 was obtained as a yellow solid (0.49 g, 94% yield).

LC-Mass (calculated: 1078.51, found: 1078.51 (M+1)).

### Synthesis Example 4 (Compound 4)

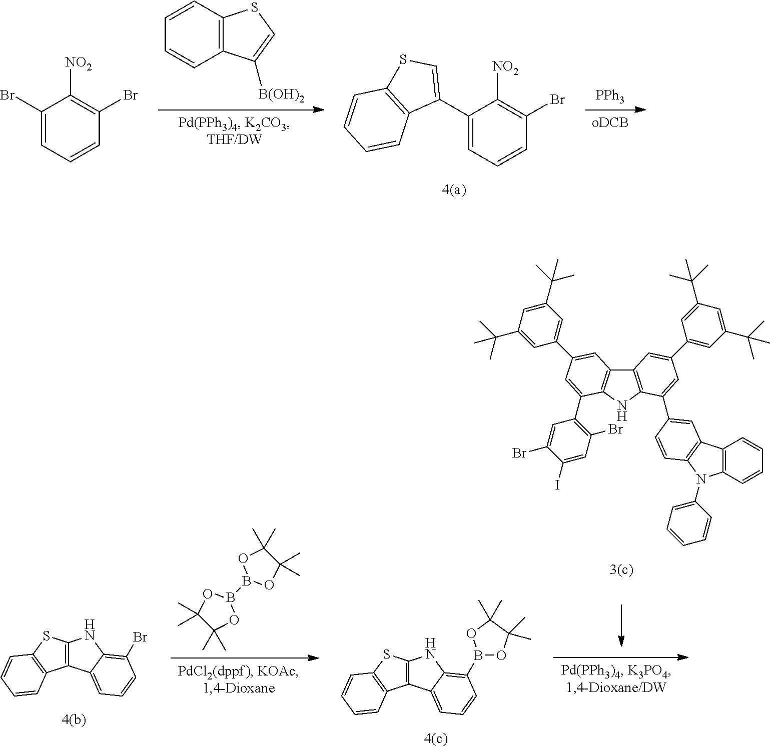

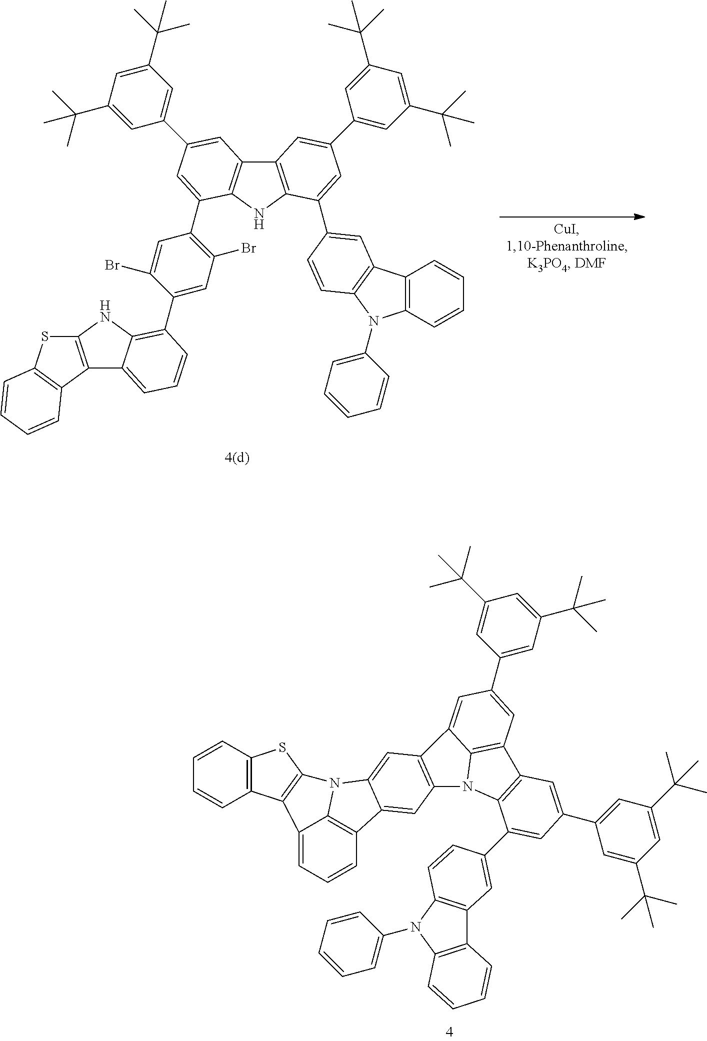 [SMILES: CC(C)(C)c1cc(-c2cc(-c3ccc4c(c3)c3ccccc3n4-c3ccccc3)c3[nH]c4c(-c5cc(Br)c(-c6cccc7c6[nH]c6sc8ccccc8c67)cc5Br)cc(-c5cc(C(C)(C)C)cc(C(C)(C)C)c5)cc4c3c2)cc(C(C)(C)C)c1 ; "CuI,1,10-Phenanthroline,K3PO4, DMF" ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4c5ccccc5sc4n6c23)cc(C(C)(C)C)c1]

### Intermediate 4(a)

1,3-dibromo-2-nitrobenzene (10.00 g, 35.60 mmol), benzothiophene-3-boronic acid (3.17 g, 17.80 mmol), tetrakis(triphenylphosphine)palladium (0.62 g, 0.53 mmol), and 100 mL of tetrahydrofuran were combined in a round flask. Then, 50 mL of an aqueous 2 M solution of potassium carbonate (13.82 g, 100.00 mmol) was added to the mixture. The reaction mixture was stirred under reflux under a nitrogen atmosphere. After completion of the reaction, the reaction mixture was purified by the process described in the synthesis of Intermediate 1(d). Intermediate 4(a) was obtained as a white solid (2.52 g, 42% yield).

LC-Mass (calculated: 333.95, found: 333.95 (M+1)).

### Intermediate 4(b)

Intermediate 4(a) (2.10 g, 6.28 mmol), triphenylphosphine (4.12 g, 15.71 mmol), and 10 mL of ortho-dichlorobenzene were combined in a round flask and stirred under reflux under a nitrogen atmosphere. After 12 hours, triphenylphosphine (4.12 g, 15.71 mmol) was added thereto, and the reaction was allowed to proceed for an additional 12 hours. After completion of the reaction, the reaction mixture was purified by the process described in the synthesis of Intermediate 1(e). Intermediate 4(b) was obtained as a yellow solid (0.85 g, 45% yield).

LC-Mass (calculated: 301.96, found: 301.96 (M+1)).

### Intermediate 4(c)

Intermediate 4(b) (0.50 g, 1.65 mmol), bis(pinacolato)diboron (0.63 g, 2.48 mmol), [1,1′-bis(diphenylphosphino)ferrocene]dichloropalladium(II) (0.04 g, 0.05 mmol), potassium acetate (0.32 g, 3.26 mmol), and 6.6 mL of 1,4-dioxane were combined in a round flask and stirred under reflux under a nitrogen atmosphere. After completion of the reaction, the reaction mixture was purified by the process described in the synthesis of Intermediate 3(c). Intermediate 4(c) was obtained as a yellow solid (0.35 g, 60% yield).

LC-Mass (calculated: 350.14, found: 350.15 (M+1)).

### Intermediate 4(d)

Intermediate 4(c) (0.30 g, 0.86 mmol), Intermediate 3(c) (1.18 g, 1.03 mmol), tetrakis(triphenylphosphine)palladium (0.05 g, 0.04 mmol), and 10 mL of 1,4-dioxane were combined in a pressure tube. Then, 5 mL of an aqueous 2 M solution of potassium phosphate tribasic (2.12 g, 10.00 mmol) was added to the mixture. The reaction mixture was heated and stirred at 100° C. After completion of the reaction, the reaction mixture was purified by the process described in the synthesis of Intermediate 1(d). Intermediate 4(d) was obtained as a white solid (0.33 g, 31% yield).

LC-Mass (calculated: 1238.37, found: 1238.37 (M+1)).

### Synthesis of Compound 4

Intermediate 4(d) (0.3 g, 0.24 mmol), copper (1) iodide (CuI) (0.05 g, 0.24 mmol), 1,10-phenanthroline (0.04 g, 0.24 mmol), potassium phosphate tribasic (0.21 g, 0.97 mmol), and 6 mL of DMF were combined in a pressure tube. The reaction mixture was heated and stirred at 120° C. After completion of the reaction, the reaction mixture was purified by the process described in the synthesis of Compound 1. Compound 4 was obtained as a yellow solid (0.21 g, 82% yield).

LC-Mass (calculated: 1078.51, found: 1078.51 (M+1)).

### Evaluation Example 1

With respect to Compounds 1 to 2 and Compound R1, the absorption spectrum, luminescence spectrum, and current-potential curve according to an oxidation reaction were measured according to the methods described in Table 1. The peak wavelength (λₘₐₓ) having a maximum emission intensity in the luminescence spectrum of each compound and a full width at half maximum (FWHM) of the peak having a maximum emission intensity were determined. The max of the fluorescence and phosphorescence spectrum, the S₁ energy level, T₁ energy level, and ΔE_{ST} (absolute value of difference between S₁ energy and T₁ energy) were also evaluated. In addition, based on the initial voltage of the current-potential curve, the HOMO energy level of each compound was obtained, and the LUMO energy level was evaluated based on the optical band gap obtained from the initial wavelength of the absorption spectrum. The results are shown in Table 2.

TABLE 1

| | |
|---|---|
| Absorption | Each sample was diluted in tetrahydrofuran (THF) at a |
| spectrum | concentration of 1 × 10⁻⁵M. Measurements were obtained with |
| evaluation | JASCO's V-730 spectrophotometer with a halogen lamp, the |
| method | absorption spectrum for each compound was measured at |
| | room temperature. |
| Emission | Each sample was diluted in THF at a concentration of 1 × 10⁻⁵M. |
| spectrum | Measurements were obtained with PerkinElmer's LS-55 |
| evaluation | fluorescence photometer with a xenon lamp, the |
| method | photoluminescence (PL) spectrum for each compound was |
| | measured at 77 kelvins (K). |
| Current-potential | Each sample was diluted in tetrabutylammonium perchlorate |
| curve evaluation | and dichloromethane (DCM) at concentrations of 1 × 10⁻³M and |
| method | 0.1M, respectively. Measurements were taken with Ivium |
| | Technologies' Iviumstat, the current-potential curve for each |
| | compound was measured. |

TABLE 2

| | HOMO | LUMO | S₁ | T₁ | | |
|---|---|---|---|---|---|---|
| | energy | energy | energy | energy | | |
| Compound | level | level | level | level | ΔEst | |
| No. | (eV) | (eV) | (eV) | (eV) | (eV) | λₘₐₓ |
| 1 | −5.77 | −2.73 | 3.02 | 2.32 | 0.70 | 410 |
| 2 | −5.66 | −2.58 | 3.15 | 2.40 | 0.75 | 393 |
| R1 | −5.82 | −2.69 | 3.15 | 2.71 | 0.44 | 393 |

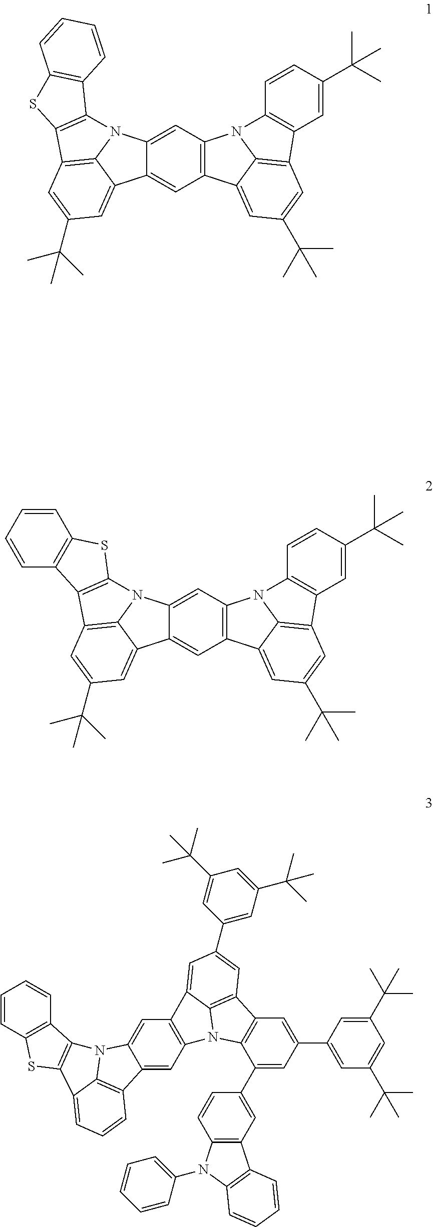 [SMILES: CC(C)(C)c1ccc2c(c1)c1cc(C(C)(C)C)cc3c4cc5c6cc(C(C)(C)C)cc7c8sc9ccccc9c8n(c5cc4n2c13)c67 ; CC(C)(C)c1ccc2c(c1)c1cc(C(C)(C)C)cc3c4cc5c6cc(C(C)(C)C)cc7c8c9ccccc9sc8n(c5cc4n2c13)c67 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4sc5ccccc5c4n6c23)cc(C(C)(C)C)c1]

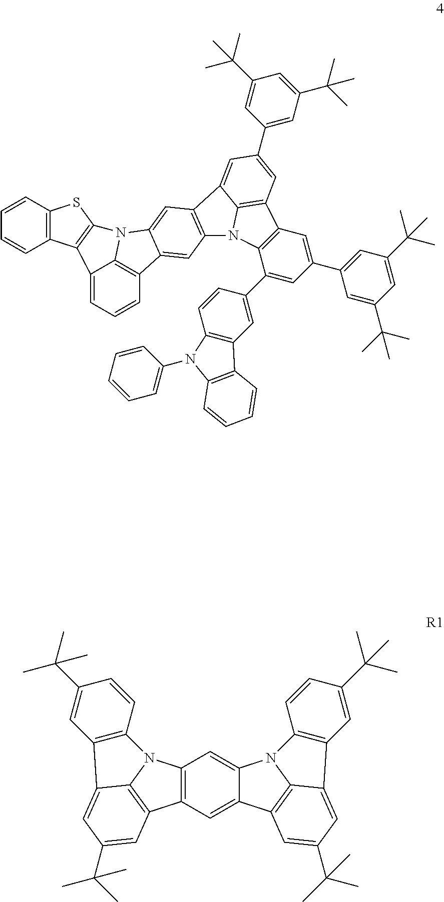

As shown in Table 2, Compounds 1 and 2 have a lower T₁ energy level and higher ΔE_{ST}, compared to Compound R1. Due to a low T₁ energy level and high ΔE_{ST}, when Compounds 1 and 2 are employed in a light-emitting device, the fluorescence emission mechanism thereof may be improved.

### Example 1

A glass substrate having a thickness of 500 Å with an ITO electrode formed thereon was cut to a size of 50 millimeters (mm)×50 mm×0.7 mm, sonicated in acetone isopropyl alcohol and pure water, each for 10 minutes, and then washed by exposure to UV ozone for 20 minutes.

Then, PEDOT:PSS was deposited on the ITO electrode (anode) on the glass substrate to form a hole injection layer having a thickness of 400 Å, 4,4′-(cyclohexane-1,1-diyl)bis(N,N-di-p-tolylaniline) (TAPC) and tris(4-(9H-carbazol-9-yl)phenyl)amine (TCTA) were sequentially deposited on the hole injection layer to form hole transport layers having thicknesses of 100 Å and 50 Å, respectively, and 1,3-di(9H-carbazol-9-yl)benzene (mCP) was deposited on the hole transport layers to form an electron blocking layer having a thickness of 50 Å.

Compound 3 (emitter) and mCP:TSPO1 (diphenyl(4-(triphenylsilyl)phenyl)phosphine oxide) (hole-type host:electron-type host) were co-deposited on the hole transport layer at a weight ratio of 1:48.5:48.5 to form an emission layer having a thickness of 250 Å.

TSPO1 was deposited on the emission layer to form an electron transport layer having a thickness of 250 Å, LiF was deposited on the electron transport layer to form an electron injection layer having a thickness of 15 Å, and Al was vacuum-deposited on the electron injection layer to form a second electrode (cathode) having a thickness of 2,000 Å, thereby completing the manufacture of a light-emitting device having a structure of ITO /PEDOT:PSS (400 Å)/TAPC (100 Å)/TCTA (50 Å)/mCP (50 Å)/mCP:TSPO1+Compound 1 (1 wt %) (250 Å)/TSPO1 (250 Å)/LiF (15 Å)/Al (2,000 Å).

### Example 2

A light-emitting device was manufactured in the same manner as in Example 1, except that Compound 4 was used instead of Compound 3 as an emitter when forming an emission layer.

### Example 3 (Hyper OLED Example)

A light-emitting device was manufactured in the same manner as in Example 1, except that Compound 3, 2,6-bis(3-(9H-carbazol-9-yl)phenoxy)benzonitrile (3CzPB) (host), and PtON7-dtb compound were co-deposited at a weight ratio of 1:89:10 when forming an emission layer.

### Example 4 (Hyper OLED Example)

A light-emitting device was manufactured in the same manner as in Example 1, except that Compound 4, 3CzPB (host), and PtON7-dtb compound (sensitizer) were co-deposited at a weight ratio of 1:89:10 when forming an emission layer.

### Example 5 (Fluorescent OLED Example)

A glass substrate having a thickness of 500 Å with an ITO electrode formed thereon was cut to a size of 50 mm×50 mm×0.7 mm, sonicated in acetone isopropyl alcohol and pure water, each for 10 minutes, and then washed by exposure to UV ozone for 20 minutes.

Then, N-([1,1′-biphenyl]-4-yl)-9,9-dimethyl-N-(4-(9-phenyl-9H-carbazol-3-yl)phenyl)-9H-fluoren-2-amine (BCFN):dipyrazino[2,3-f:2′,3′-h]quinoxaline-2,3,6,7,10,11-hexacarbonitrile (HATCN) (30 wt %) were co-deposited on the ITO electrode (anode) on the glass substrate to form a hole injection layer having a thickness of 400 Å, and BCFN and 9,9′-diphenyl-9H,9′H-3,3′-bicarbazole (DCzPh) were sequentially deposited on the hole injection layer to form hole transport layers having thicknesses of 100 Å and 100 Å, respectively.

9-(3-(phenanthren-9-yl)phenyl)-10-phenylanthracene (TTF-Phen):Compound 3 (3 wt %) were co-deposited on the hole transport layer to form an emission layer having a thickness of 300 Å.

2,4-diphenyl-6-(3-(triphenylsilyl)phenyl)-1,3,5-triazine (SiTrz) and 2-(4-(9,10-di(naphthalen-2-yl)anthracen-2-yl)phenyl)-1-phenyl-1H-benzo[d]imidazole (ZADN) were deposited on the emission layer to form electron transport layers having thicknesses of 50 Å and 200 Å, respectively, LiF was deposited on the electron transport layers to form an electron injection layer having a thickness of 15 Å, and Al was vacuum-deposited on the electron injection layer to form a second electrode (cathode) having a thickness of 2,000 Å, thereby completing the manufacture of a light-emitting device having a structure of ITO/BCFN:HATCN (30 wt %) (400 Å)/BCFN (100 Å)/DCzPh (100 Å)/TTF-Phen+Compound 3 (3 wt %) (300 Å)/SiTrz (50 Å)/ZADN (200 Å)/LiF (15 Å)/Al (2,000 Å).

### Example 6 (Fluorescent OLED Example)

A light-emitting device was manufactured in the same manner as in Example 5, except that Compound 4 was used instead of Compound 3 in Example 5.

### Comparative Example 1

A light-emitting device was manufactured in the same manner as in Example 1, except that Compound R1 was used instead of Compound 3 as an emitter when forming an emission layer.

### Evaluation Example 2

For each of the light-emitting devices manufactured in Examples 1 to 6 and Comparative Example 1, the emission peak wavelength (maximum emission peak wavelength) in the EL spectrum, FWHM, driving voltage, and maximum value of external quantum efficiency (Max EQE) were evaluated, and the results thereof are shown in Tables 3, 4, and 5. For each of the light-emitting devices, the emission peak wavelength of the EL spectrum and FWHM were evaluated from the EL spectrum (at 1,000 candela per square meter (cd/m²)) measured with a luminance meter (Minolta Cs-2000A). The driving voltage and the EQE were evaluated with a current-voltmeter (Keithley 2400) and a luminance meter (Minolta Cs-2000A).

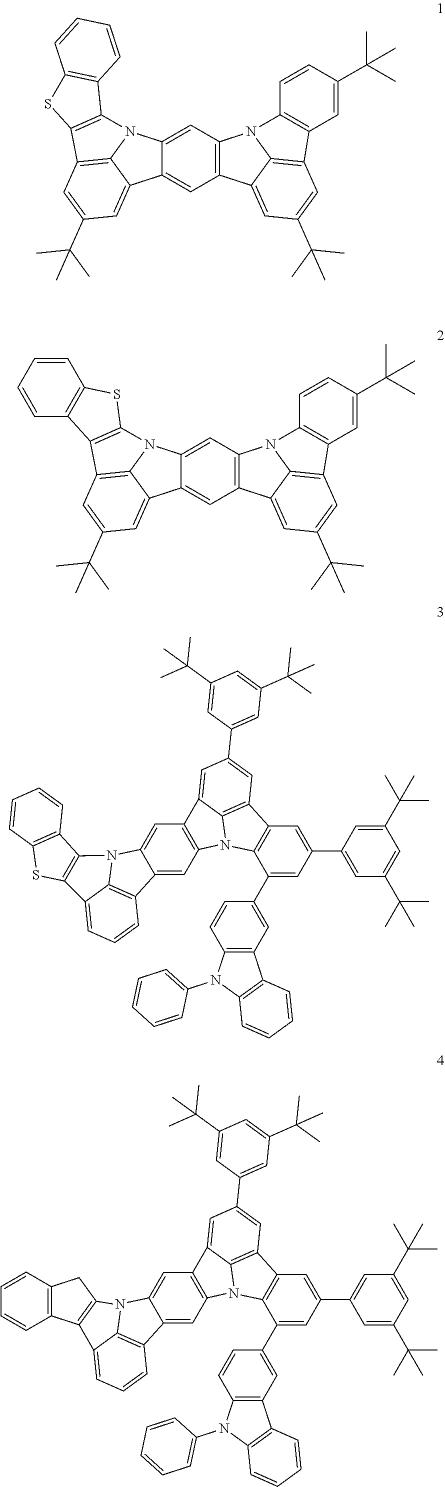 [SMILES: CC(C)(C)c1ccc2c(c1)c1cc(C(C)(C)C)cc3c4cc5c6cc(C(C)(C)C)cc7c8sc9ccccc9c8n(c5cc4n2c13)c67 ; CC(C)(C)c1ccc2c(c1)c1cc(C(C)(C)C)cc3c4cc5c6cc(C(C)(C)C)cc7c8c9ccccc9sc8n(c5cc4n2c13)c67 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4sc5ccccc5c4n6c23)cc(C(C)(C)C)c1 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4c(n6c32)Cc2ccccc2-4)cc(C(C)(C)C)c1]

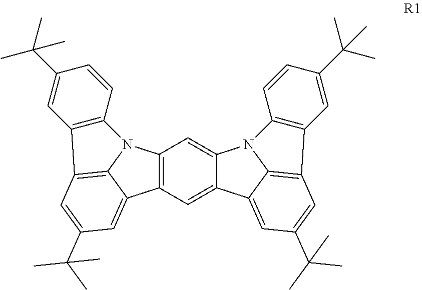 [SMILES: CC(C)(C)c1ccc2c(c1)c1cc(C(C)(C)C)cc3c4cc5c6cc(C(C)(C)C)cc7c8cc(C(C)(C)C)ccc8n(c5cc4n2c13)c76]

TABLE 3

| | | Emission | | | |
|---|---|---|---|---|---|
| | Emitter | peak | | Driving | Max |
| | Compound | wavelength | FWHM | voltage | EQE |
| | No. | (nm) | (nm) | (volts, V) | (%) |
| Example 1 | 3 | 460 | 26 | 5.7 | 3.7 |
| Example 2 | 4 | 450 | 30 | 5.3 | 10.1 |
| Comparative | R1 | 401 | — | — | 2.7 |
| Example R1 | | | | | |

As shown in Table 3, the light-emitting devices of Examples 1 to 2, as delayed fluorescence OLEDs, emit blue light and have a lower driving voltage and improved external quantum efficiency, compared to the light-emitting device of Comparative Example 1.

In particular, in the light-emitting devices of Examples 1 and 2, due to different core structures of Compounds 3 and 4, lower triplet energy and higher ΔE_{ST} may be achieved in comparison with Compound R1, and accordingly, it is confirmed that the light-emitting devices of Examples 1 and 2 emit blue light in a target range and have a lower driving voltage and improved external quantum efficiency, compared to the light-emitting device of Comparative Example 1.

TABLE 4

| | Emitter | Emission peak | | Driving | Max |
|---|---|---|---|---|---|
| | Compound | wavelength | FWHM | voltage | EQE |
| | No. | (nm) | (nm) | (V) | (%) |
| Example 3 | 3 | 454 | 26 | 6.0 | 9.4 |
| Example 4 | 4 | 452 | 23 | 5.8 | 17.5 |

As shown in Table 4, the hyper light-emitting devices employing Compounds 3 and 4 emit blue light and have a lower driving voltage and excellent external quantum efficiency. Due to the short wavelength characteristics of Compound R1, there may be a difficulty in implementing the hyper light-emitting device employing Compound R1.

TABLE 5

| | Emitter | Emission peak | | Driving | Max |
|---|---|---|---|---|---|
| | Compound | wavelength | FWHM | voltage | EQE |
| | No. | (nm) | (nm) | (V) | (%) |
| Example 5 | 3 | 462 | 27 | 6.4 | 4.2 |
| Example 6 | 4 | 452 | 38 | 6.8 | 5.8 |

As shown in Table 5, the fluorescence light-emitting devices employing Compounds 3 and 4 emit blue light and have a lower driving voltage and excellent external quantum efficiency. Due to the short wavelength characteristics of Compound R1, there may be a difficulty in implementing the hyper light-emitting device employing Compound R1.

For the condensed cyclic compound, by reducing triplet energy, fluorescence emission mechanism may be improved, and an electronic device employing the condensed cyclic compound, for example, a light-emitting device may have improved driving voltage and external quantum efficiency.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the FIGURES, it will be understood by those of ordinary skill in the art that various changes in form and details may be made therein without departing from the spirit and scope as defined by the following claims.

What is claimed is:

1. A condensed cyclic compound represented by Formula 1:
Formula 1 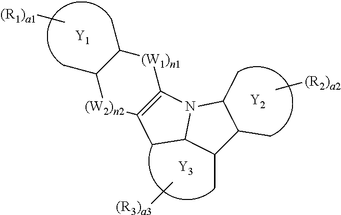
wherein, in Formula 1,
ring Y₁ to ring Y₃ are each independently a C₅-C₆₀ carbocyclic group or a C₃-C₆₀ heterocyclic group,
W₁ is O, S, N(T₁₁), C(T₁₂)(T₁₃), or Si(T₁₂)(T₁₃),
W₂ is O, S, N(T₂₁), C(T₂₂)(T₂₃), or Si(T₂₂)(T₂₃),
n1 and n2 are each independently 0 or 1,
when n1 is 0, *—(W₁)ₙ₁—*′ is a single bond,
when n2 is 0, *—(W₂)ₙ₂—*′ is a single bond,
the sum of n1 and n2 is 1,
R₁ to R₃, T₁₁ to T₁₃, and T₂₁ to T₂₃ are each independently hydrogen, deuterium, —F, —Cl, —Br, —I, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), wherein R₁ in the number of a1, R₂ in the number of a2, and R₃ in the number of a3 are each identical to or different from each other,
a1 to a3 are each independently an integer from 0 to 30,
two or more of R₁ to R₃, T₁₁ to T₁₃, and T₂₁ to T₂₃ are optionally linked to each other to form a substituted or unsubstituted C₅-C₃₀ carbocyclic group or a substituted or unsubstituted C₁-C₃₀ heterocyclic group,
at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or any combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, or a monovalent non-aromatic condensed heteropolycyclic group, each unsubstituted or substituted with deuterium, —F, —Cl, —Br, —I, —CD₃, —CD₂H, —CDH₂, —CF₃, —CF₂H, —CFH₂, —SF₅, a hydroxyl group, a cyano group, a nitro group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁—C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or any combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
any combination thereof, and
Q₁ to Q₉, Q₁₁ to Q₁₉, Q₂₁ to Q₂₉, and Q₃₁ to Q₃₉ are each independently: hydrogen;
deuterium; —F; or a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with deuterium, —F, a cyano group, a C₁-C₆₀ alkyl group, a C₆-C₆₀ aryl group, or any combination thereof.

2. The condensed cyclic compound of claim 1, wherein ring Y₁ to ring Y₃ are each independently a benzene group, a naphthalene group, a phenanthrene group, a pyridine group, a pyrimidine group, a pyridazine group, a pyrazine group, a quinoline group, an isoquinoline group, a dibenzofuran group, a dibenzothiophene group, a carbazole group, a fluorene group, a dibenzosilole group, an azadibenzofuran group, an azadibenzothiophene group, an azacarbazole group, an azafluorene group, an azadibenzosilole group, or an indolocarbazole group.

3. The condensed cyclic compound of claim 1, wherein ring Y₂ is a group represented by one of Formulae 2-1 to 2-6: 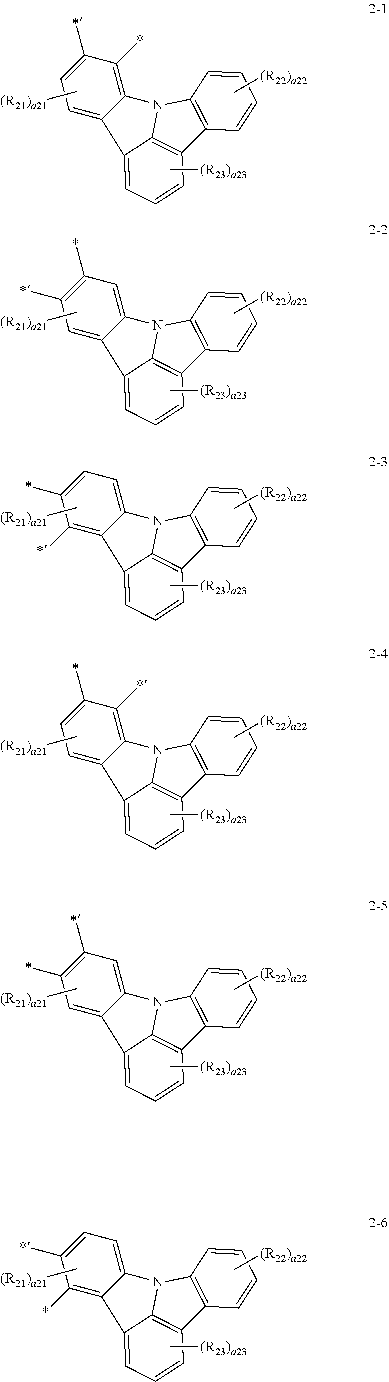
wherein, in Formulae 2-1 to 2-6,
R₂₁ to R₂₃ are each as described in connection with R₂ in claim 1,
a21 is an integer from 0 to 2,
a22 is an integer from 0 to 4,
a23 is an integer from 0 to 3,
* indicates a binding site to N in Formula 1, and
*′ indicates a binding site to a neighboring atom in ring Y₃ in Formula 1.

4. The condensed cyclic compound of claim 1, wherein the condensed cyclic compound satisfies Condition 1 Å or Condition 1B:
Condition 1 Å
n1 is 1, n2 is 0, and W₁ is O or S; or
Condition 1B
n1 is 0, n2 is 1, and W₂ is O or S.

5. The condensed cyclic compound of claim 1, wherein the condensed cyclic compound satisfies at least one of Conditions 1C to 1E:
Condition 1C
at least one of O and S is included;
Condition 1 D
at least one of O and S is included, and simultaneously, a tert-butyl group is included; and
Condition 1E
at least one of O and S is included, and simultaneously, a carbazolyl group is included.

6. The condensed cyclic compound of claim 1, wherein R₁ to R₃, T₁₁ to T₁₃, and T₂₁ to T₂₃ are each independently:
hydrogen, deuterium, —F, or a cyano group;
a C₁-C₂₀ alkyl group, a phenyl group, a biphenyl group, a dibenzofuranyl group, a dibenzothiophenyl group, a carbazolyl group, a fluorenyl group, or a dibenzosilolyl group, each unsubstituted or substituted with deuterium, —F, a cyano group, a C₁-C₂₀ alkyl group, a phenyl group, a biphenyl group, a dibenzofuranyl group, a dibenzothiophenyl group, a carbazolyl group, a fluorenyl group, a dibenzosilolyl group, —N(Q₁)(Q₂), or any combination thereof; or
—N(Q₁)(Q₂), and
Q₁ and Q₂ are each independently a C₁-C₂₀ alkyl group, a phenyl group, a biphenyl group, a dibenzofuranyl group, a dibenzothiophenyl group, a carbazolyl group, a fluorenyl group, or a dibenzosilolyl group, each unsubstituted or substituted with deuterium, —F, a cyano group, a C₁-C₂₀ alkyl group, a phenyl group, a dibenzofuranyl group, a dibenzothiophenyl group, a carbazolyl group, a fluorenyl group, a dibenzosilolyl group, or any combination thereof.

7. The condensed cyclic compound of claim 1, wherein R₁ to R₃, T₁₁ to T₁₃, and T₂₁ to T₂₃ are each independently:
hydrogen, deuterium, —F, or a cyano group;
a C₁-C₂₀ alkyl group that is unsubstituted or substituted with deuterium, —F, a cyano group, or any combination thereof;
a phenyl group unsubstituted or substituted with deuterium, —F, a cyano group, a C₁-C₂₀ alkyl group, a phenyl group, —N(Q₁)(Q₂), or any combination thereof; or
a carbazolyl group unsubstituted or substituted with deuterium, —F, a cyano group, a C₁-C₂₀ alkyl group, a phenyl group, a biphenyl group, or any combination thereof, and
Q₁ and Q₂ are each independently a C₁-C₂₀ alkyl group, a phenyl group, a biphenyl group, a dibenzofuranyl group, a dibenzothiophenyl group, a carbazolyl group, a fluorenyl group, or a dibenzosilolyl group, each unsubstituted or substituted with deuterium, —F, a cyano group, a C₁-C₂₀ alkyl group, a phenyl group, a dibenzofuranyl group, a dibenzothiophenyl group, a carbazolyl group, a fluorenyl group, a dibenzosilolyl group, or any combination thereof.

8. The condensed cyclic compound of claim 1, wherein R₁ to R₃, T₁₁ to T₁₃, and T₂₁ to T₂₃ are each independently:
hydrogen, deuterium, —F, or a cyano group;
a methyl group, an ethyl group, an n-butyl group, a sec-butyl group, or a tert-butyl group, each unsubstituted or substituted with deuterium, —F, a cyano group, or any combination thereof;
a phenyl group unsubstituted or substituted with deuterium, —F, a cyano group, a methyl group, an ethyl group, an n-butyl group, a sec-butyl group, a tert-butyl group, a phenyl group, —N(Q₁)(Q₂), or any combination thereof; or
a carbazolyl group unsubstituted or substituted with deuterium, —F, a cyano group, a methyl group, an ethyl group, an n-butyl group, a sec-butyl group, a tert-butyl group, a phenyl group, a biphenyl group, or any combination thereof, and
Q₁ and Q₂ are each independently a C₁-C₂₀ alkyl group, a phenyl group, a biphenyl group, a dibenzofuranyl group, a dibenzothiophenyl group, a carbazolyl group, a fluorenyl group, or a dibenzosilolyl group, each unsubstituted or substituted with deuterium, —F, a cyano group, a C₁-C₂₀ alkyl group, a phenyl group, a dibenzofuranyl group, a dibenzothiophenyl group, a carbazolyl group, a fluorenyl group, a dibenzosilolyl group, or any combination thereof.

9. The condensed cyclic compound of claim 1, wherein the condensed cyclic compound is represented by any one of Formulae 3A and 3B: 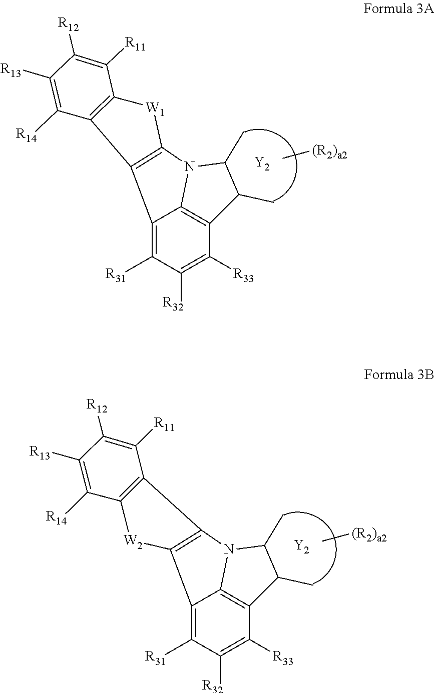
wherein, in Formulae 3A and 3B,
W₁, W₂, Y₂, R₂, and a2 are each as described in claim 1,
R₁₁ to R₁₄ are each as described in connection with R₁ in claim 1, and
R₃₁ to R₃₃ are each as described in connection with R₃ in claim 1.

10. The condensed cyclic compound of claim 1, wherein the condensed cyclic compound is represented by any one of Formulae 3-1 to 3-12: 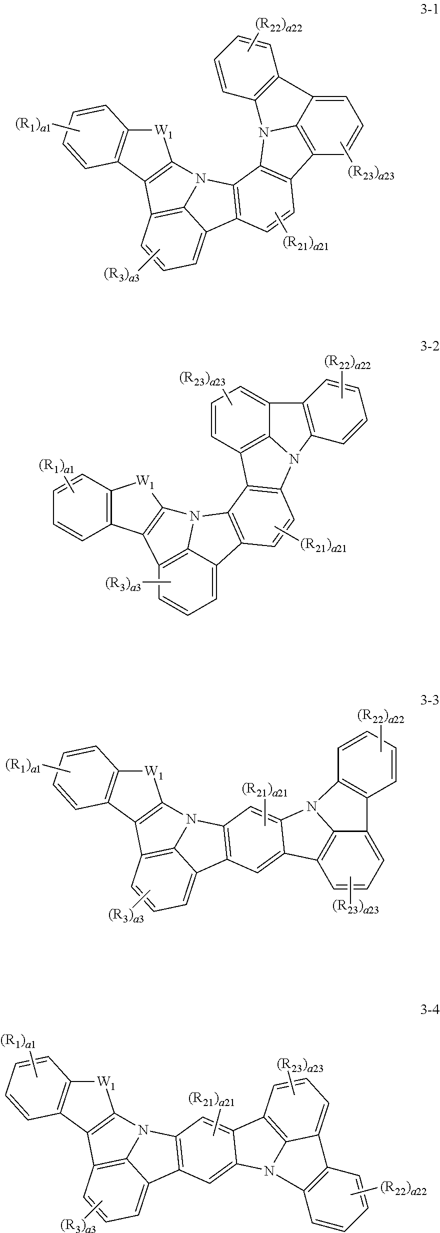 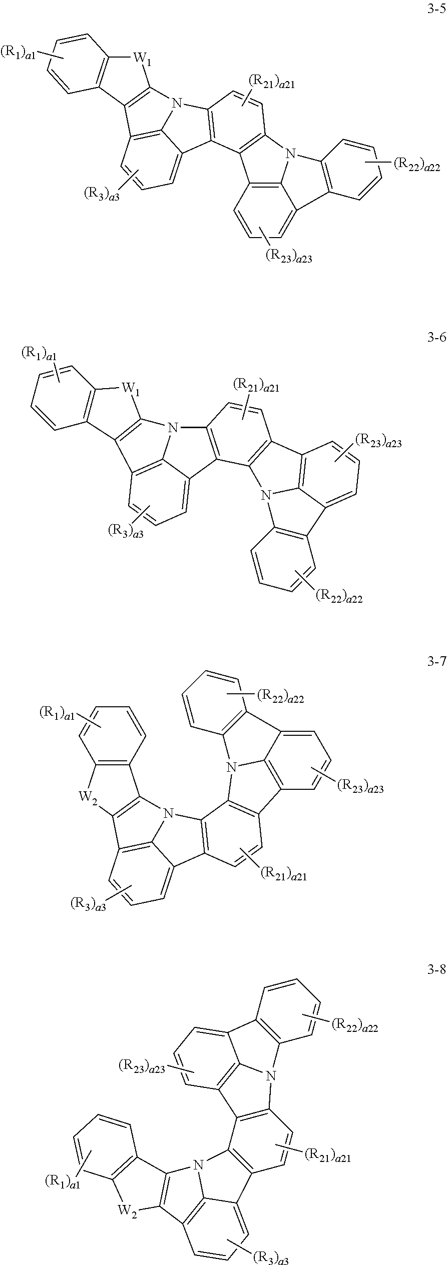 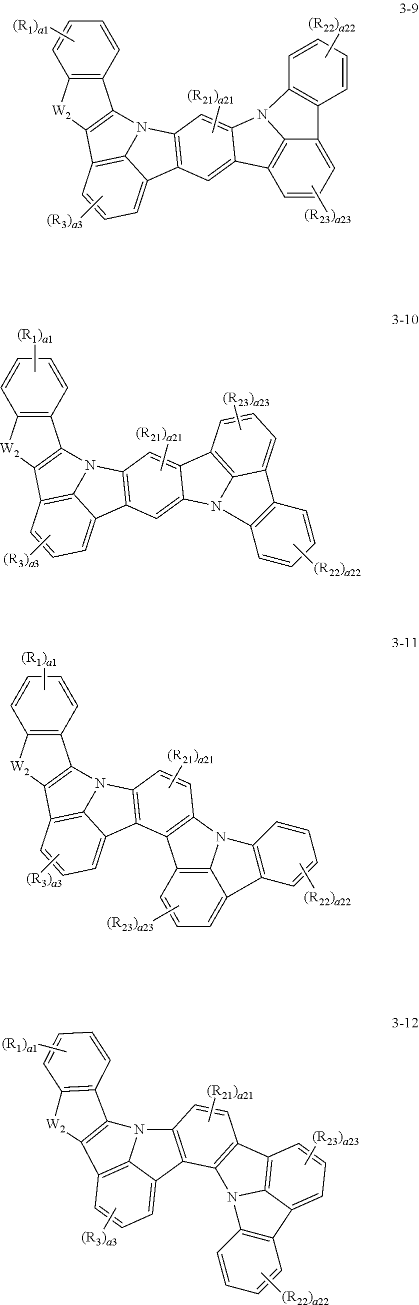
wherein, in Formulae 3-1 to 3-12,
W₁, W₂, R₁, R₃, a1, and a3 are each as described in claim 1,
R₂₁ to R₂₃ are each as described in connection with R₂ in claim 1,
a21 is an integer from 0 to 2,
a22 is an integer from 0 to 4, and
a23 is an integer from 0 to 3.

11. The condensed cyclic compound of claim 1, wherein the condensed cyclic compound is represented by one of Compounds 1 to 172:

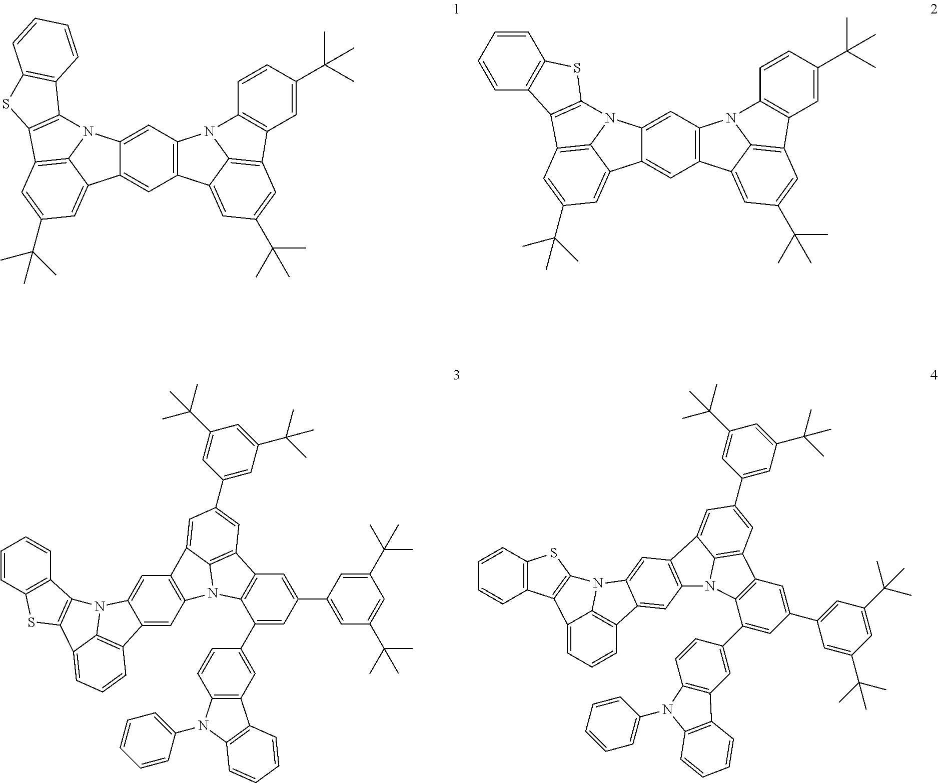 [SMILES: CC(C)(C)c1ccc2c(c1)c1cc(C(C)(C)C)cc3c4cc5c6cc(C(C)(C)C)cc7c8sc9ccccc9c8n(c5cc4n2c13)c67 ; CC(C)(C)c1ccc2c(c1)c1cc(C(C)(C)C)cc3c4cc5c6cc(C(C)(C)C)cc7c8c9ccccc9sc8n(c5cc4n2c13)c67 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4sc5ccccc5c4n6c23)cc(C(C)(C)C)c1 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5cc6c(cc5n3c42)c2cccc3c4c5ccccc5sc4n6c23)cc(C(C)(C)C)c1]

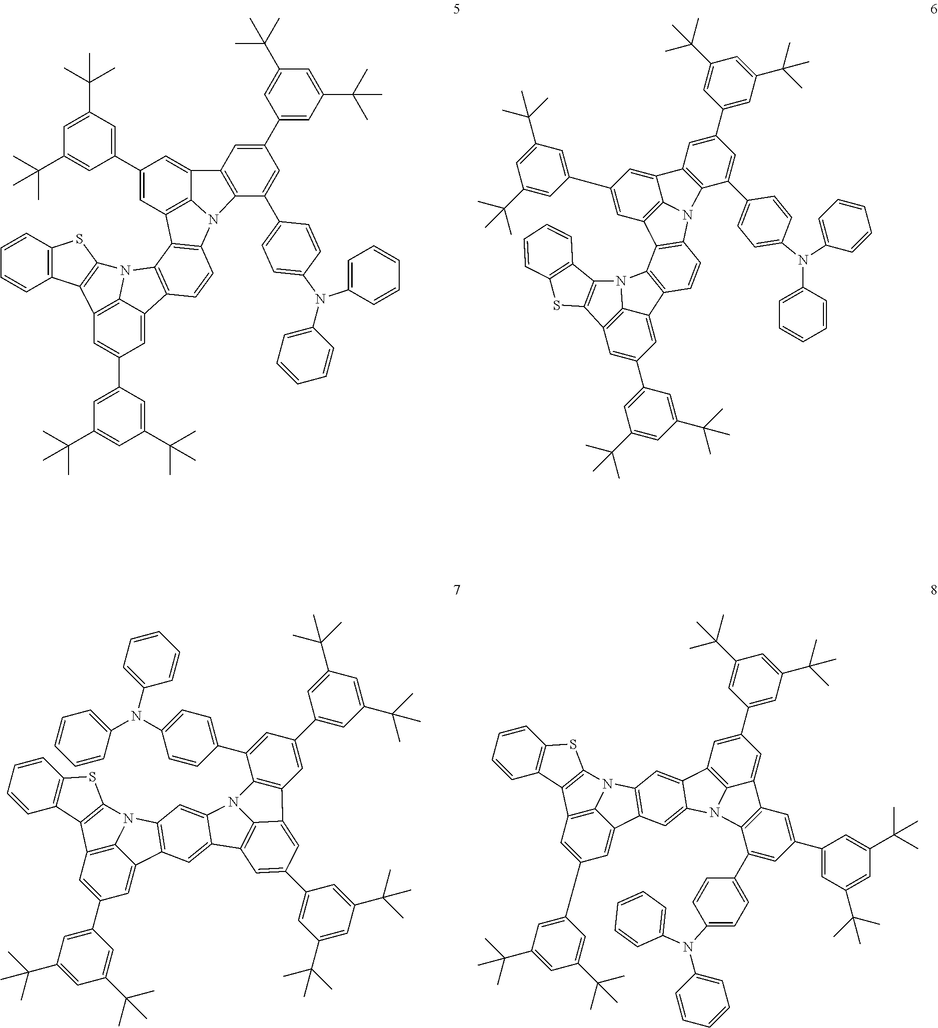

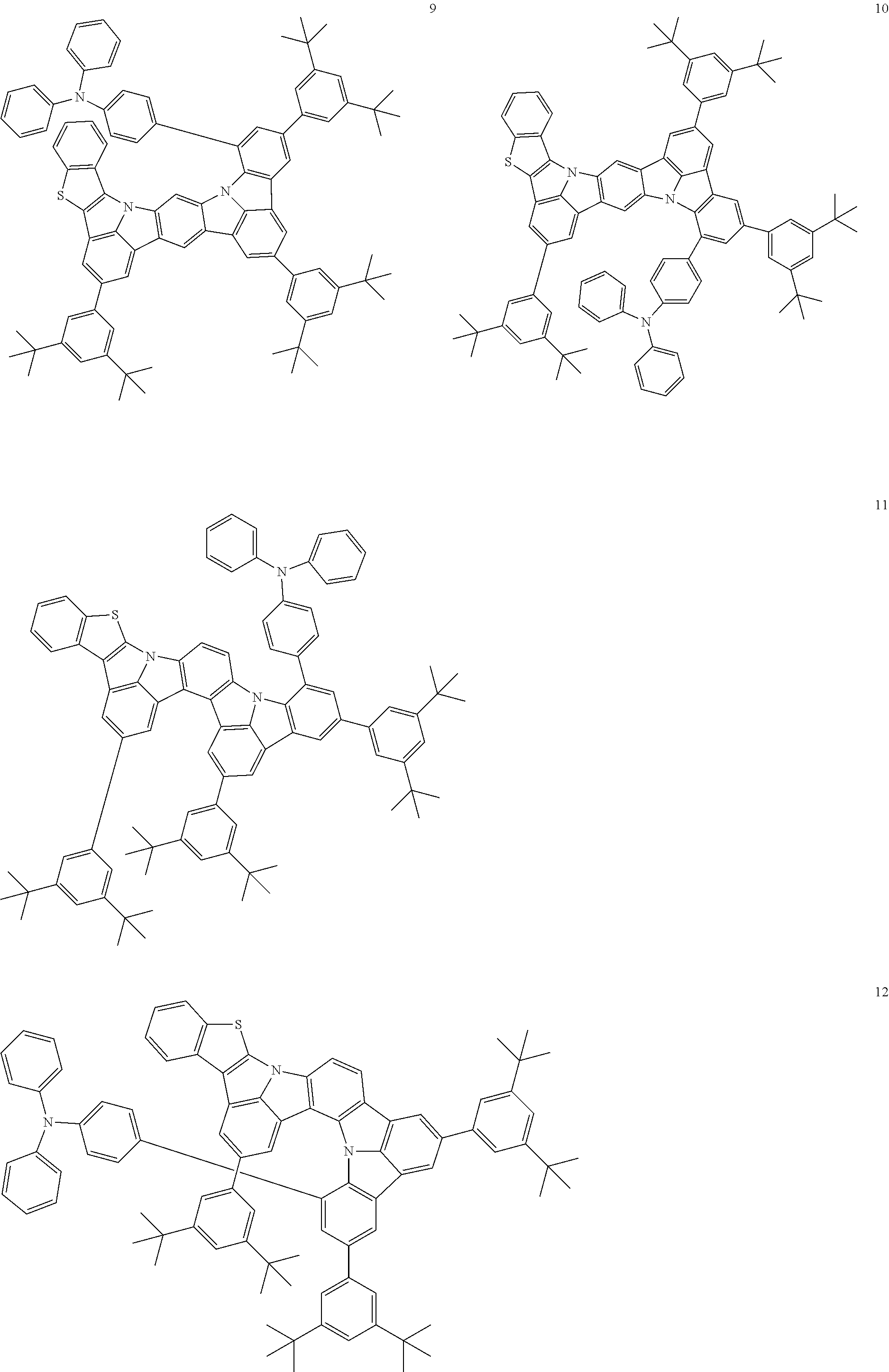

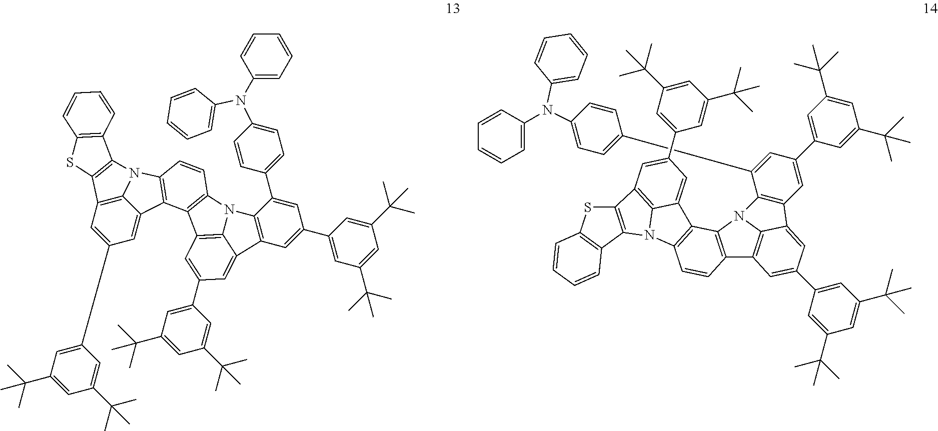 [SMILES: CC(C)(C)c1cc(-c2cc(-c3ccc(N(c4ccccc4)c4ccccc4)cc3)c3c(c2)c2cc(-c4cc(C(C)(C)C)cc(C(C)(C)C)c4)cc4c5c6c7cc(-c8cc(C(C)(C)C)cc(C(C)(C)C)c8)cc8c9sc%10ccccc%10c9n(c6ccc5n3c24)c87)cc(C(C)(C)C)c1 ; CC(C)(C)c1cc(-c2cc(-c3ccc(N(c4ccccc4)c4ccccc4)cc3)c3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9sc%10ccccc%10c9n6c87)c5n3c24)cc(C(C)(C)C)c1]

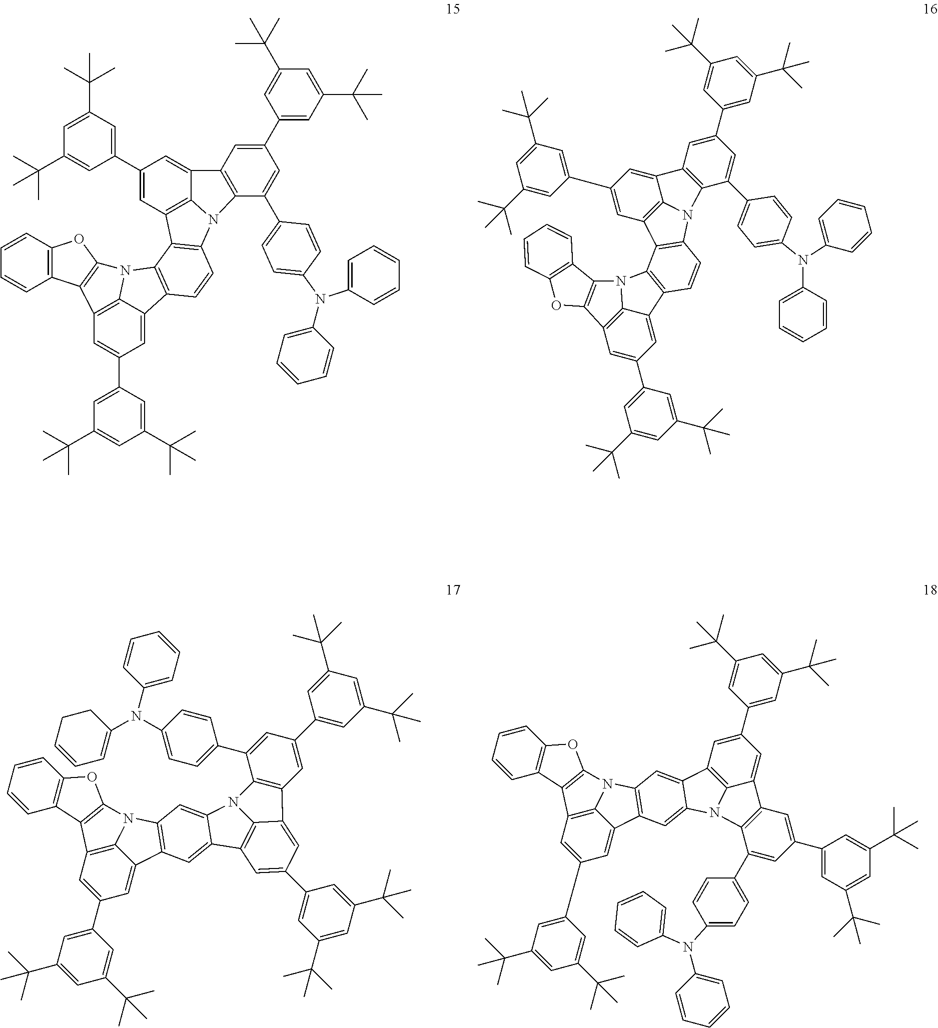

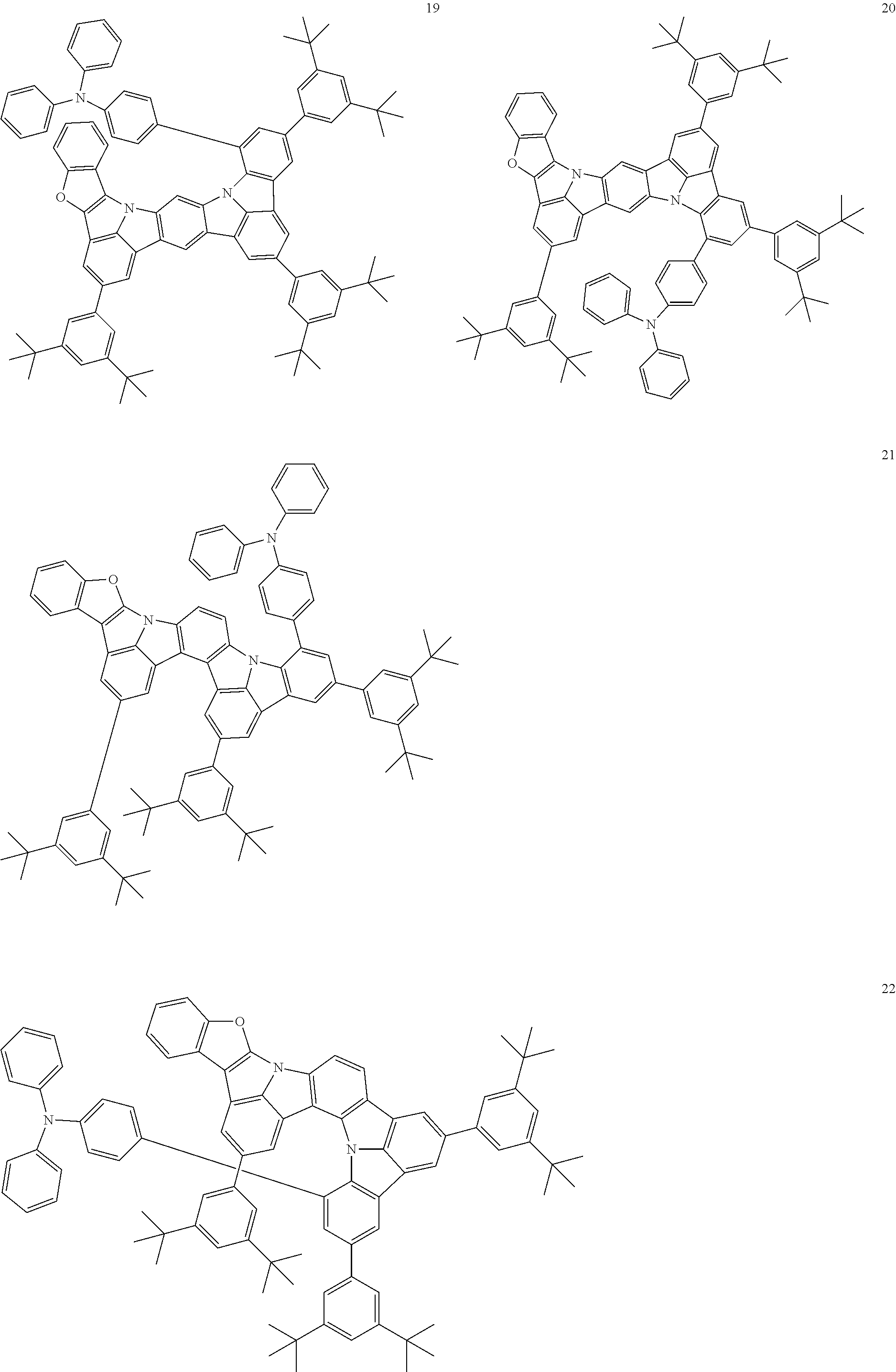

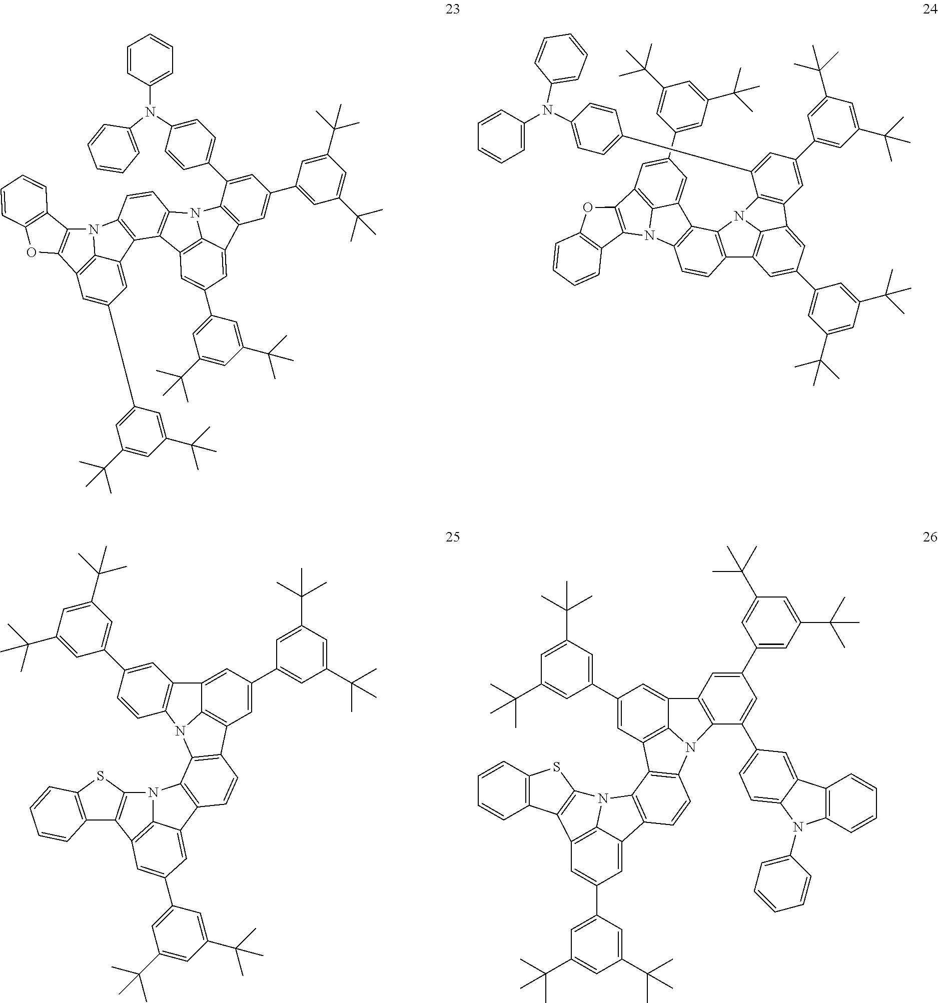

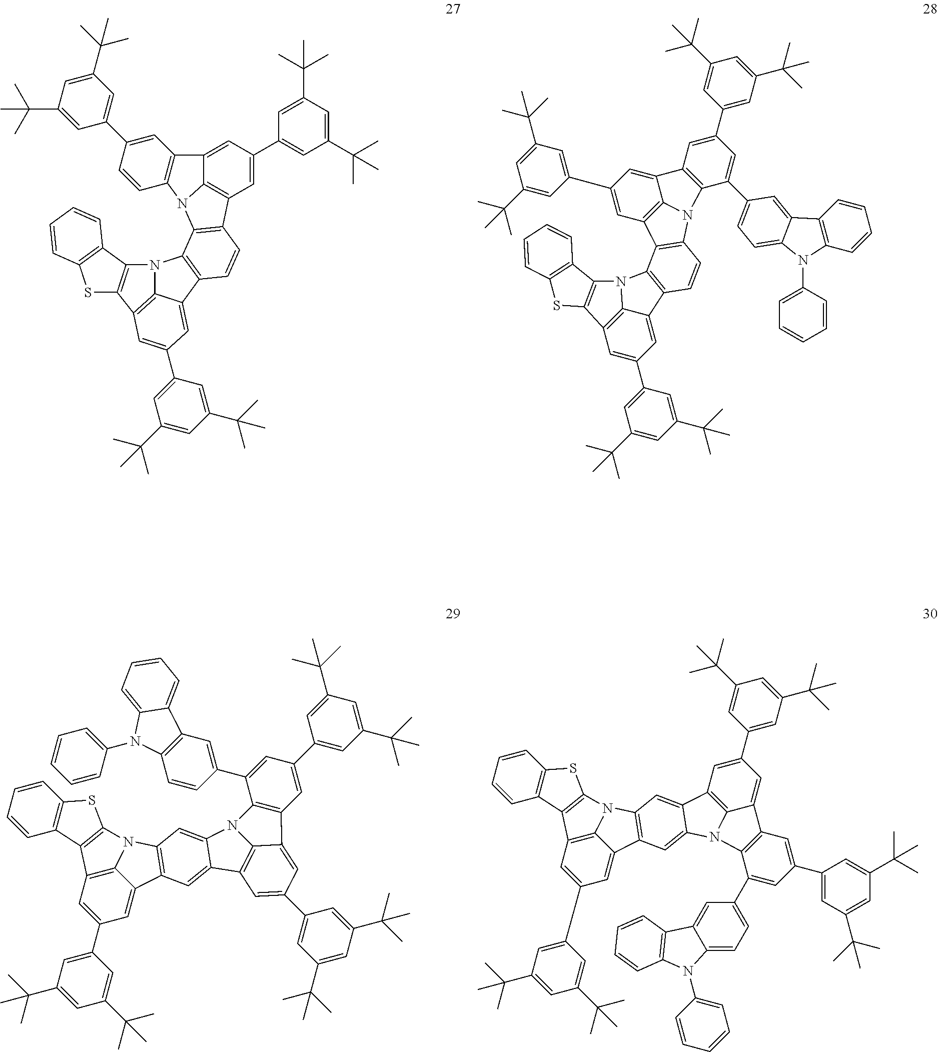

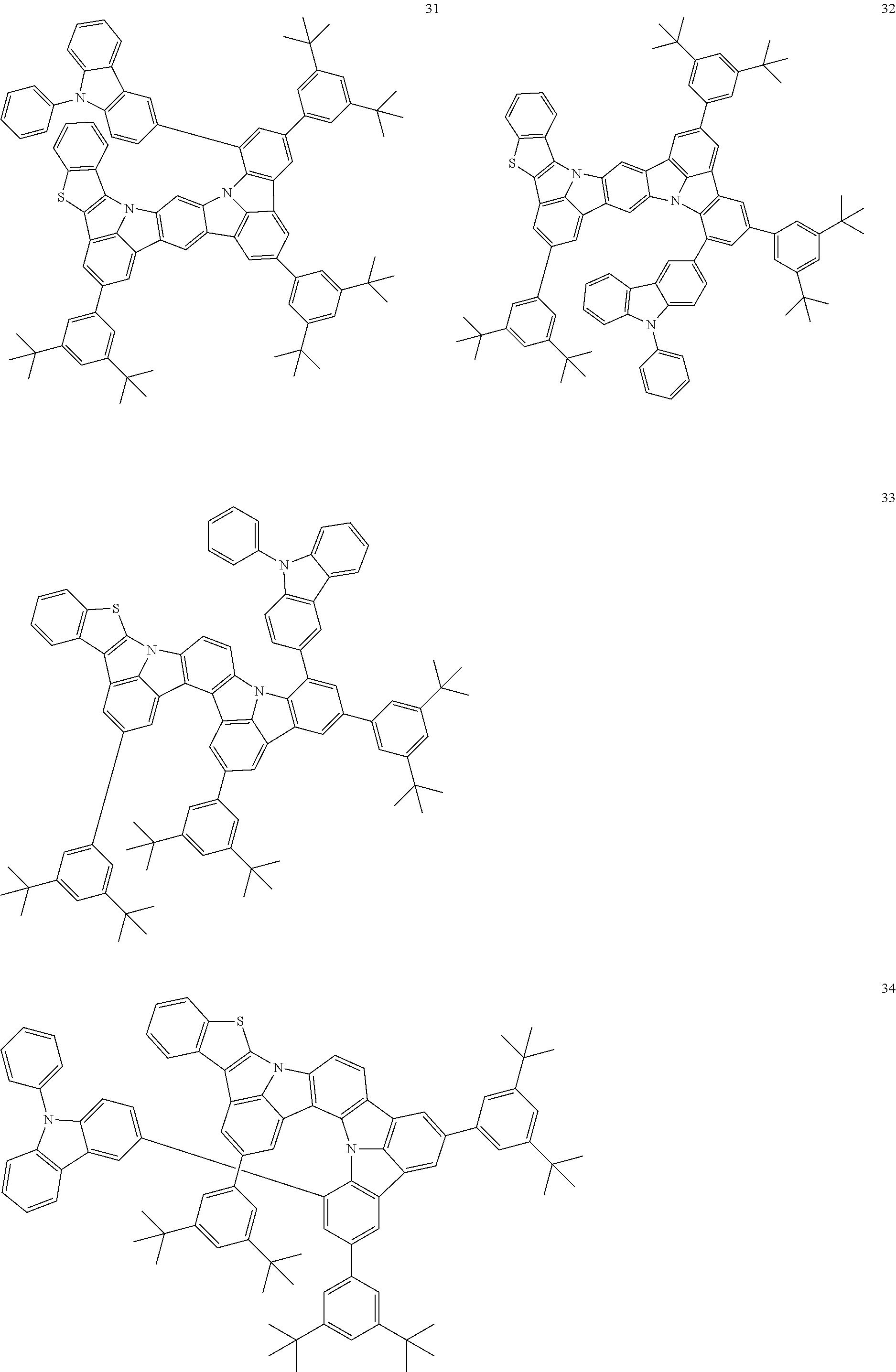

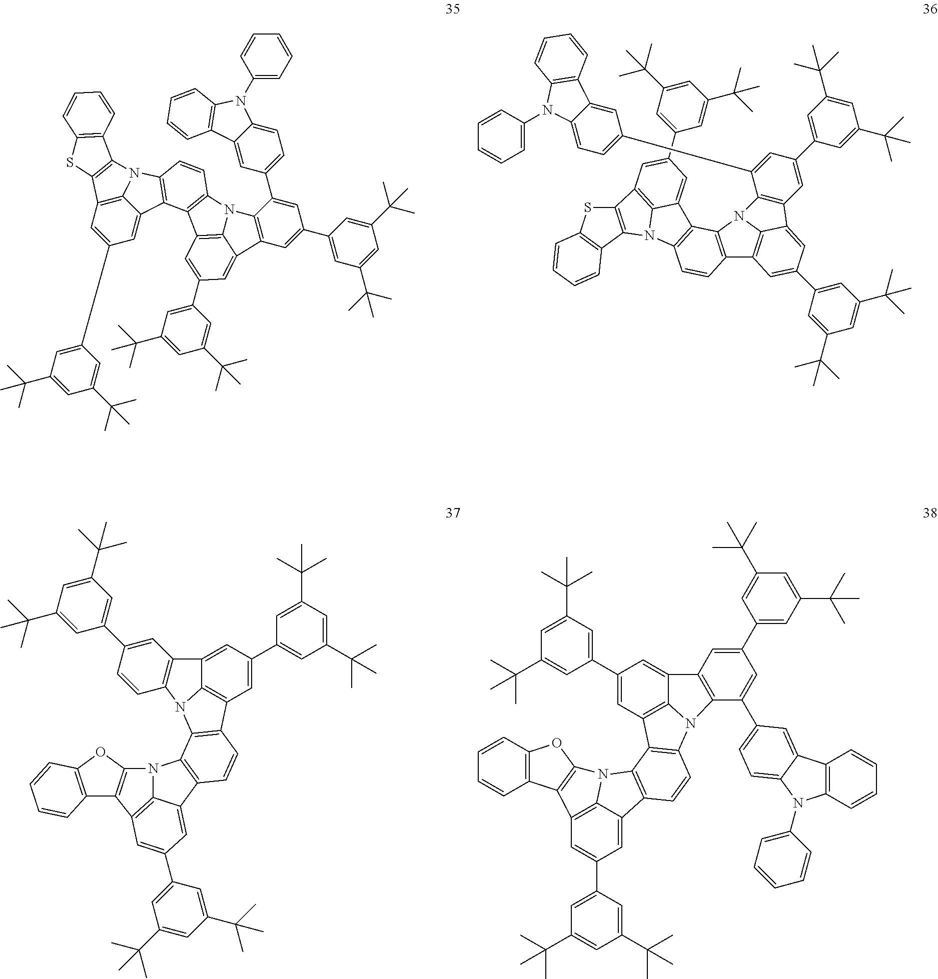

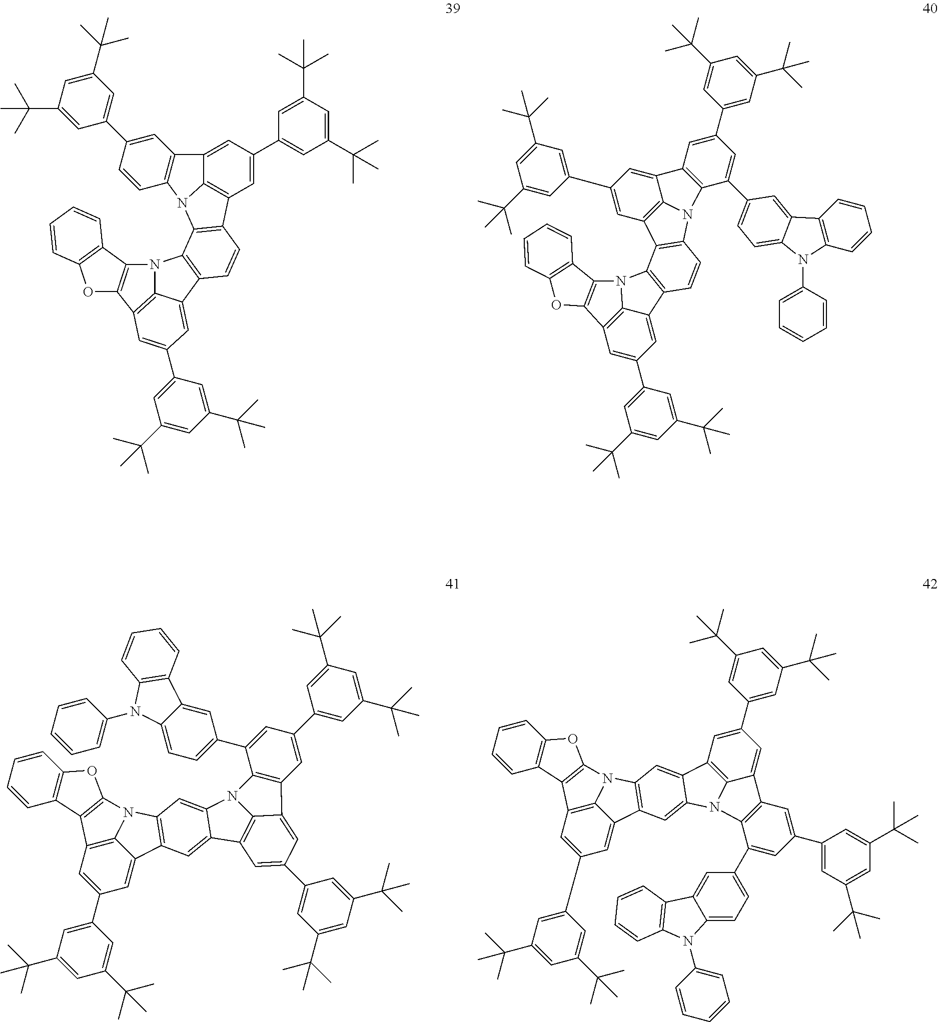

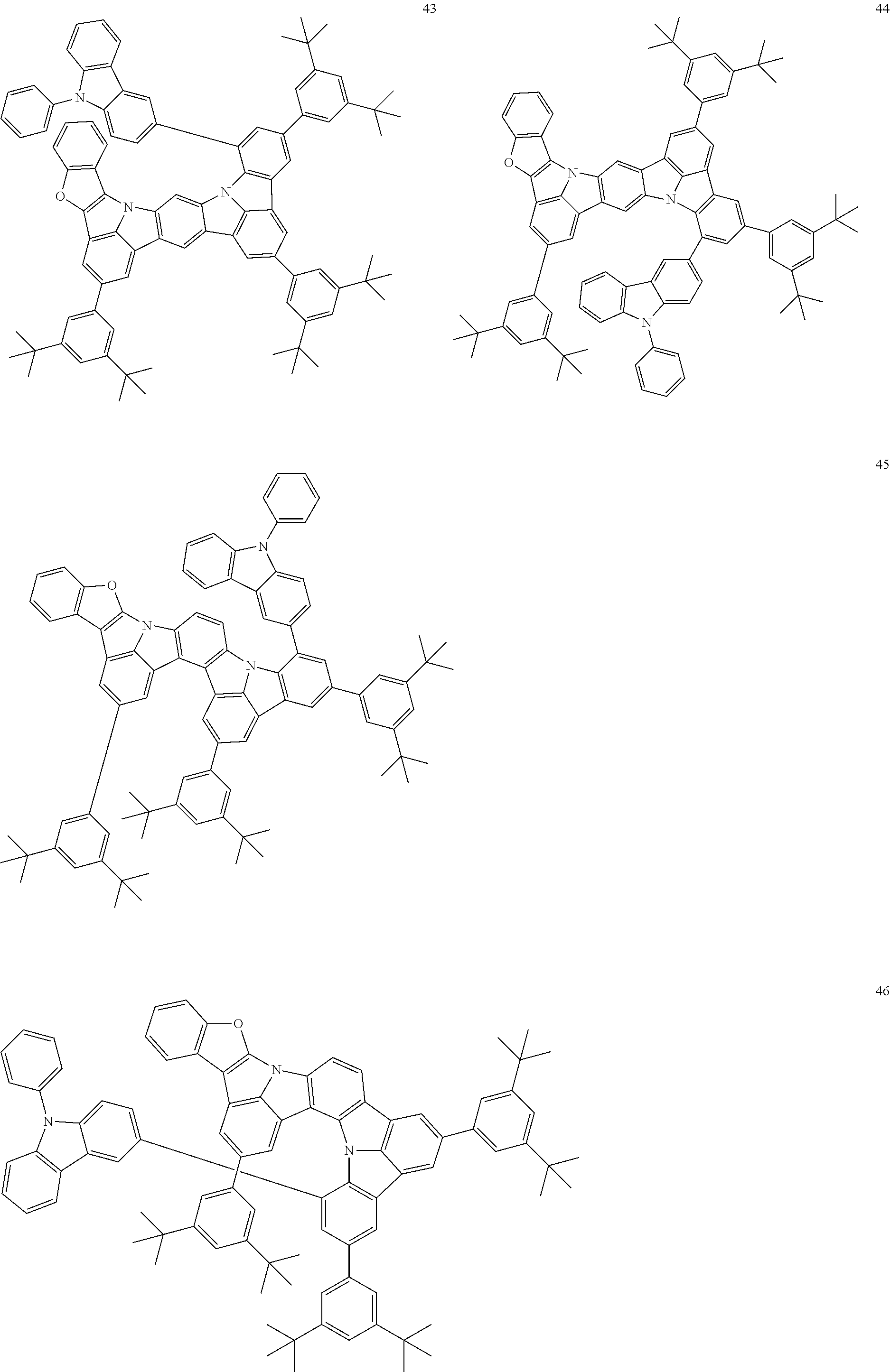

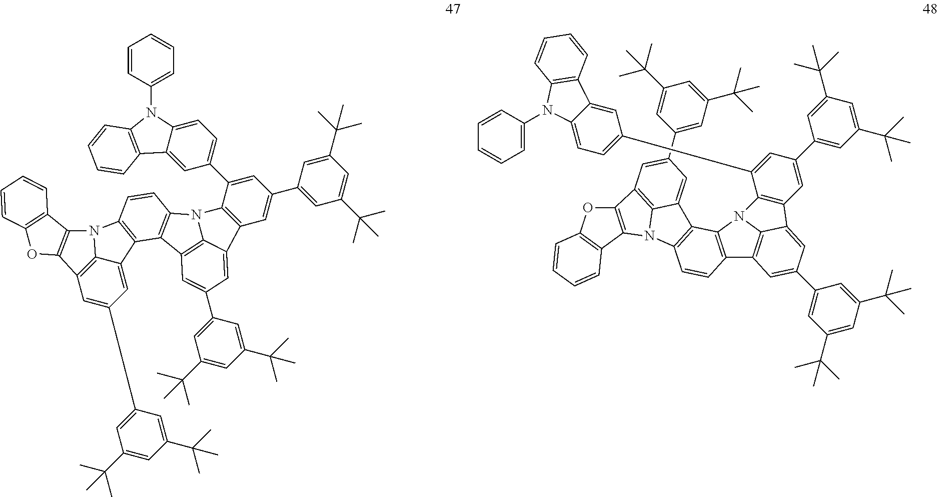 [SMILES: CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5c6c7cc(-c8cc(C(C)(C)C)cc(C(C)(C)C)c8)cc8c9oc%10ccccc%10c9n(c6ccc5n3c24)c87)cc(C(C)(C)C)c1 ; CC(C)(C)c1cc(-c2cc(-c3ccc4c(c3)c3ccccc3n4-c3ccccc3)c3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9oc%10ccccc%10c9n6c87)c5n3c24)cc(C(C)(C)C)c1]

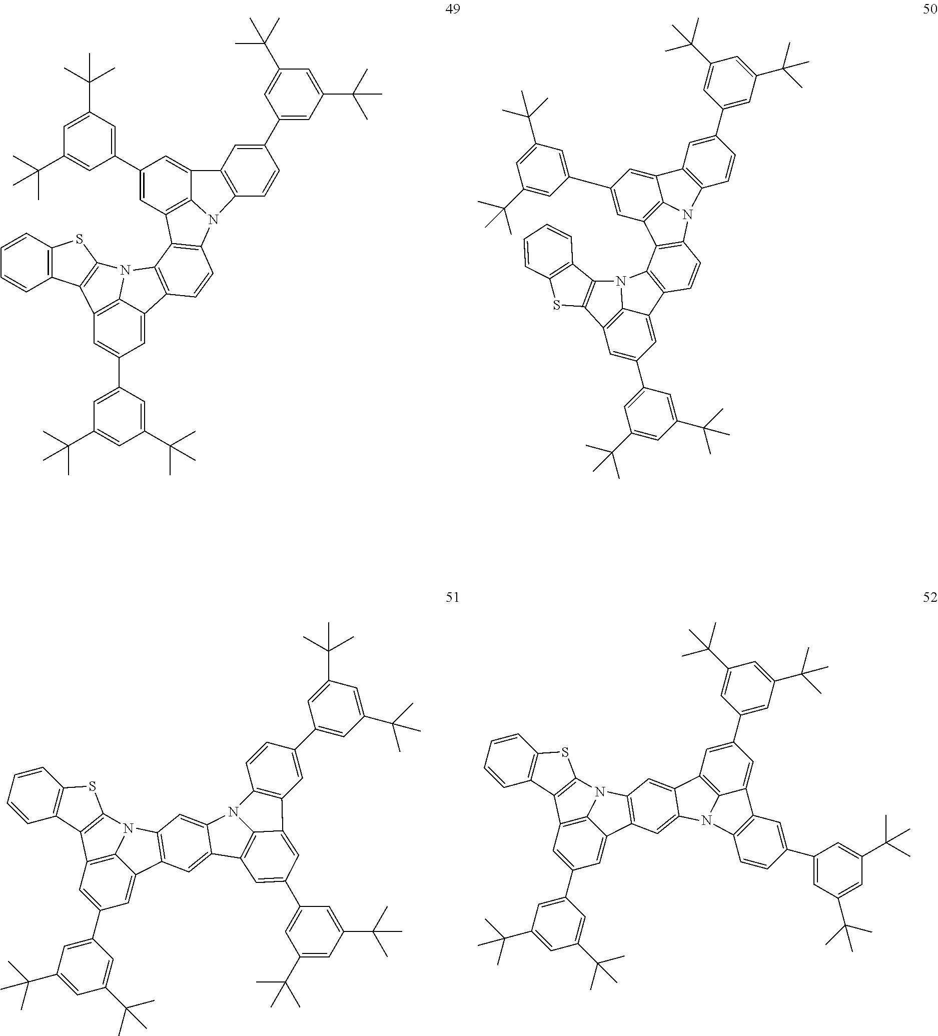

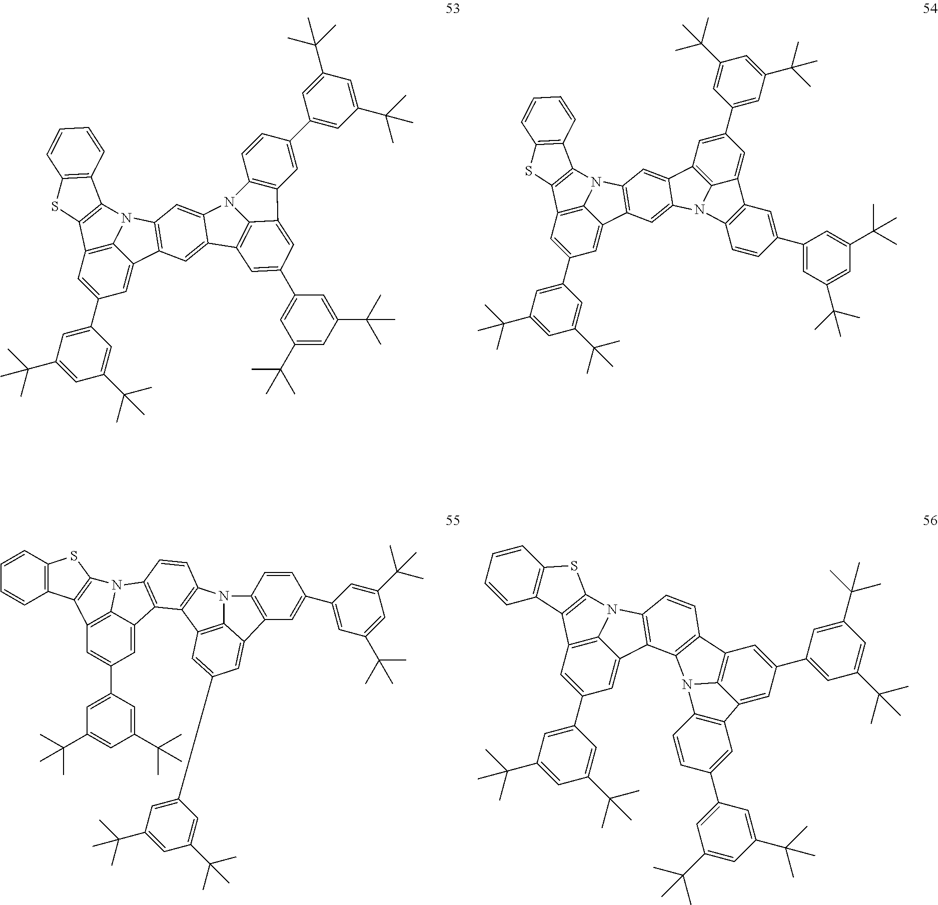

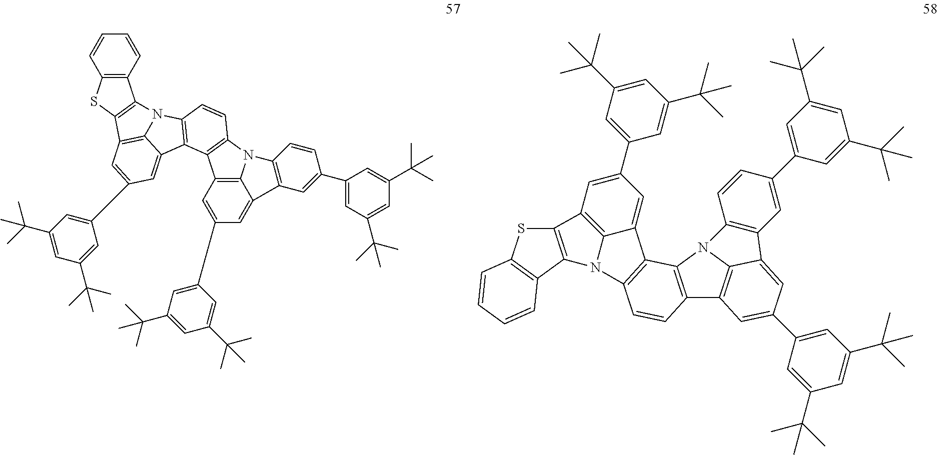 [SMILES: CC(C)(C)c1cc(-c2ccc3c(c2)c2cc(-c4cc(C(C)(C)C)cc(C(C)(C)C)c4)cc4c5c6c7cc(-c8cc(C(C)(C)C)cc(C(C)(C)C)c8)cc8c9sc%10ccccc%10c9n(c6ccc5n3c24)c87)cc(C(C)(C)C)c1 ; CC(C)(C)c1cc(-c2ccc3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9sc%10ccccc%10c9n6c87)c5n3c24)cc(C(C)(C)C)c1]

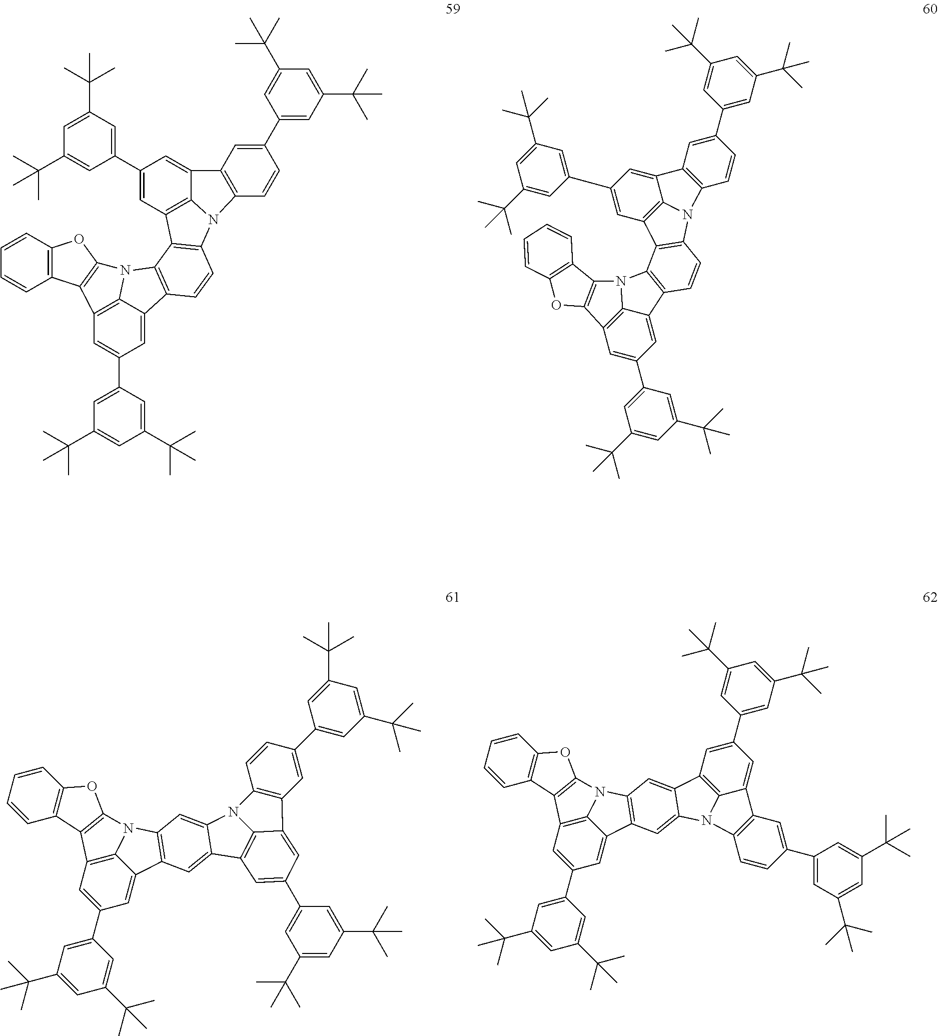

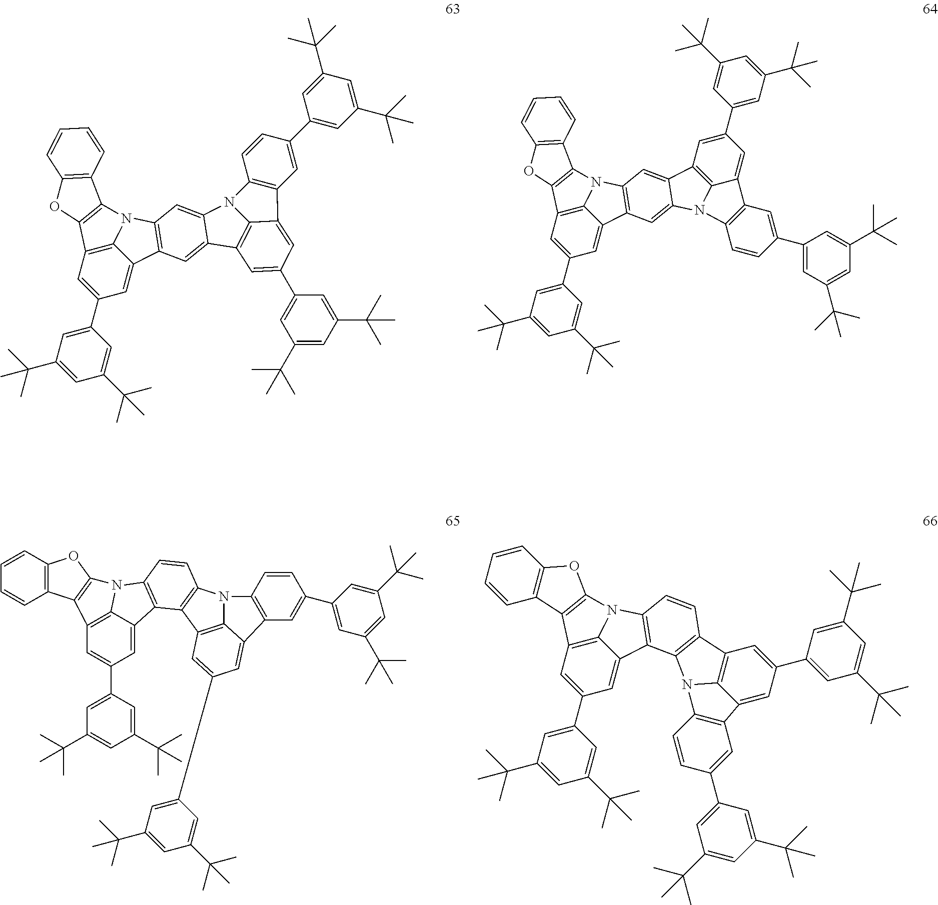

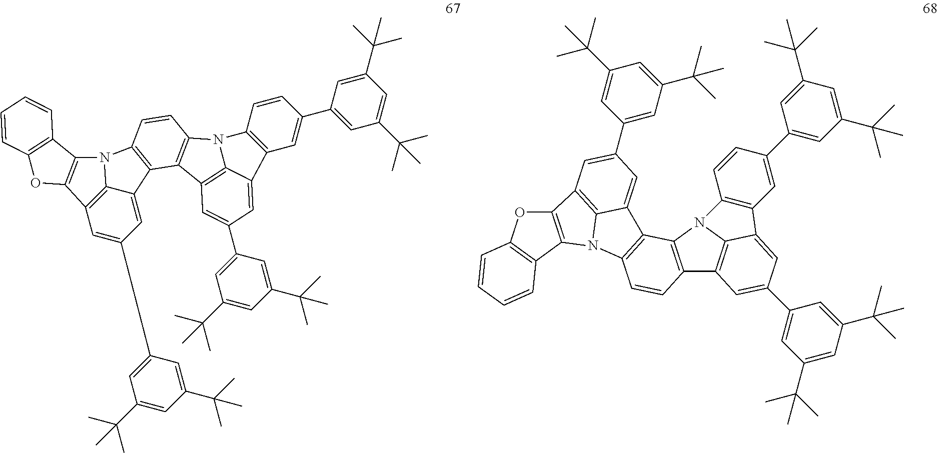 [SMILES: CC(C)(C)c1cc(-c2ccc3c(c2)c2cc(-c4cc(C(C)(C)C)cc(C(C)(C)C)c4)cc4c5c6c7cc(-c8cc(C(C)(C)C)cc(C(C)(C)C)c8)cc8c9oc%10ccccc%10c9n(c6ccc5n3c24)c87)cc(C(C)(C)C)c1 ; CC(C)(C)c1cc(-c2ccc3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9oc%10ccccc%10c9n6c87)c5n3c24)cc(C(C)(C)C)c1]

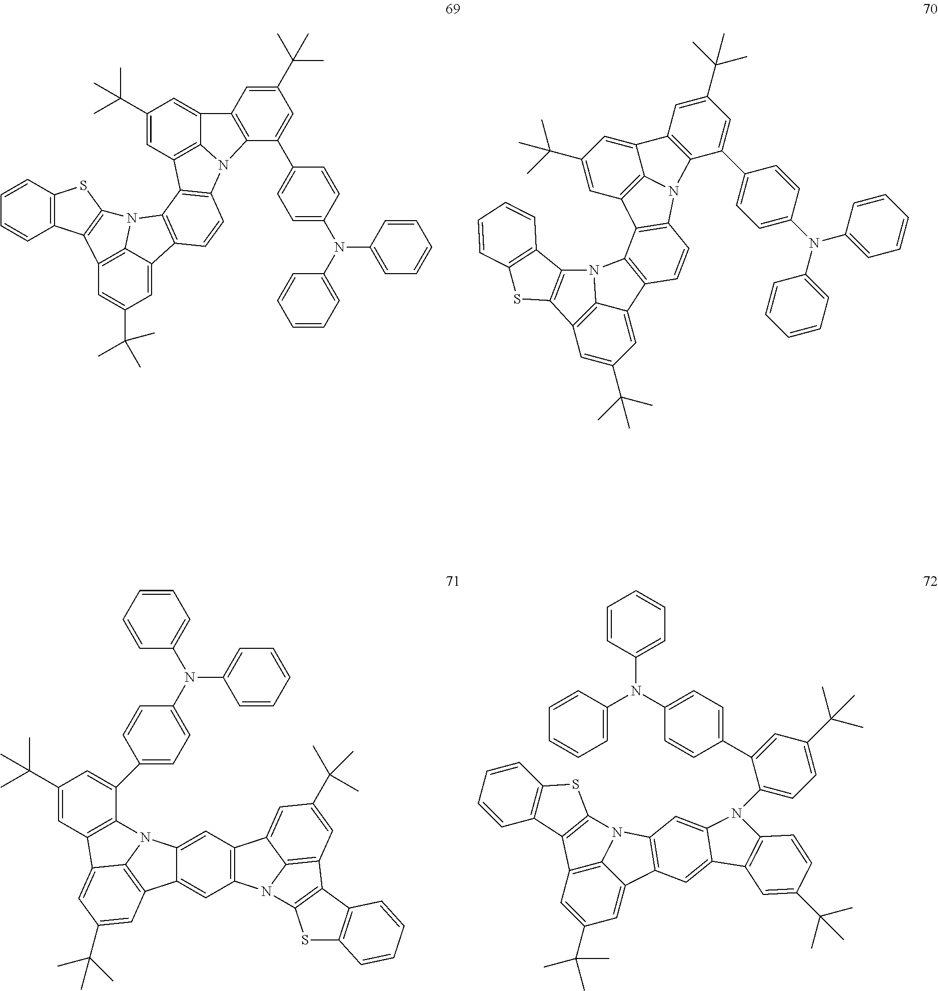 [SMILES: CC(C)(C)c1cc(-c2ccc(N(c3ccccc3)c3ccccc3)cc2)c2c(c1)c1cc(C(C)(C)C)cc3c4c(ccc5c6cc(C(C)(C)C)cc7c8c9ccccc9sc8n(c67)c54)n2c13 ; CC(C)(C)c1cc(-c2ccc(N(c3ccccc3)c3ccccc3)cc2)c2c(c1)c1cc(C(C)(C)C)cc3c4c(ccc5c6cc(C(C)(C)C)cc7c8sc9ccccc9c8n(c67)c54)n2c13 ; CC(C)(C)c1cc(-c2ccc(N(c3ccccc3)c3ccccc3)cc2)c2c(c1)c1cc(C(C)(C)C)cc3c4cc5c(cc4n2c31)c1cc(C(C)(C)C)cc2c3c4ccccc4sc3n5c12 ; CC(C)(C)c1ccc(-n2c3ccc(C(C)(C)C)cc3c3cc4c5cc(C(C)(C)C)cc6c7c8ccccc8sc7n(c4cc32)c56)c(-c2ccc(N(c3ccccc3)c3ccccc3)cc2)c1]

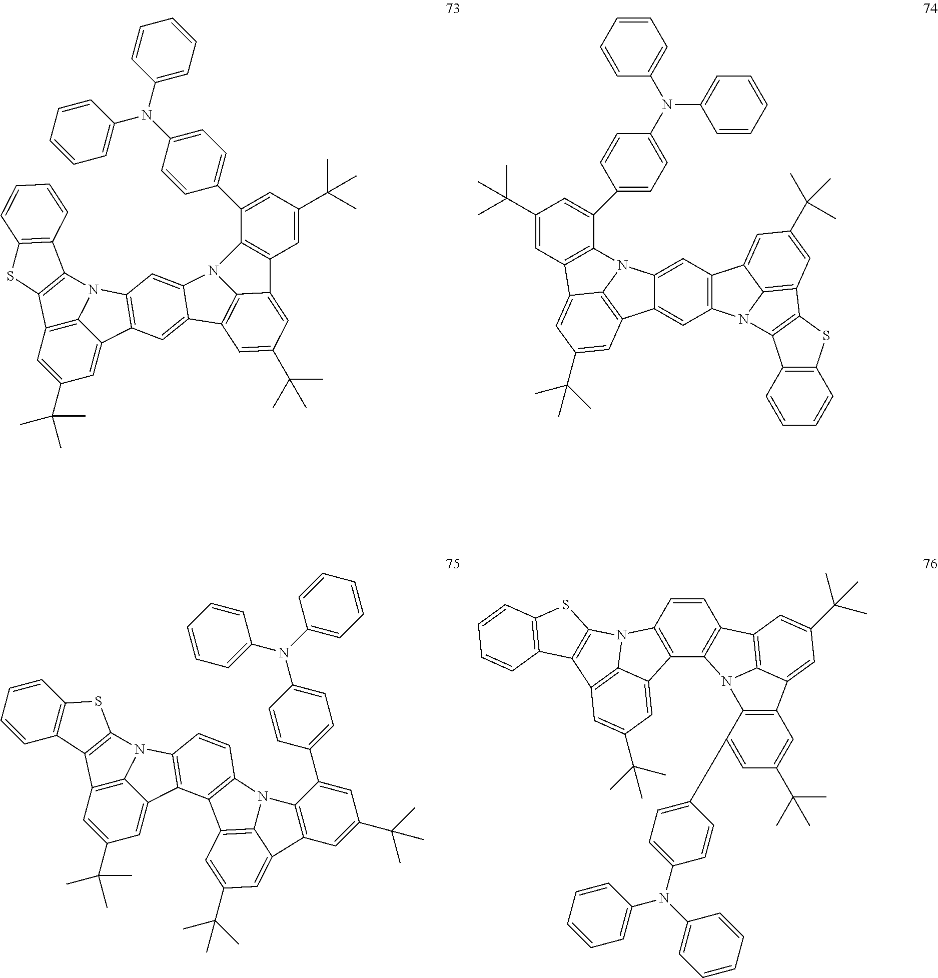 [SMILES: CC(C)(C)c1cc(-c2ccc(N(c3ccccc3)c3ccccc3)cc2)c2c(c1)c1cc(C(C)(C)C)cc3c4cc5c6cc(C(C)(C)C)cc7c8sc9ccccc9c8n(c5cc4n2c31)c67 ; CC(C)(C)c1cc(-c2ccc(N(c3ccccc3)c3ccccc3)cc2)c2c(c1)c1cc(C(C)(C)C)cc3c4cc5c(cc4n2c31)c1cc(C(C)(C)C)cc2c3sc4ccccc4c3n5c12 ; CC(C)(C)c1cc(-c2ccc(N(c3ccccc3)c3ccccc3)cc2)c2c(c1)c1cc(C(C)(C)C)cc3c4c5c6cc(C(C)(C)C)cc7c8c9ccccc9sc8n(c5ccc4n2c13)c67 ; CC(C)(C)c1cc(-c2ccc(N(c3ccccc3)c3ccccc3)cc2)c2c(c1)c1cc(C(C)(C)C)cc3c4ccc5c(c6cc(C(C)(C)C)cc7c8c9ccccc9sc8n5c76)c4n2c13]

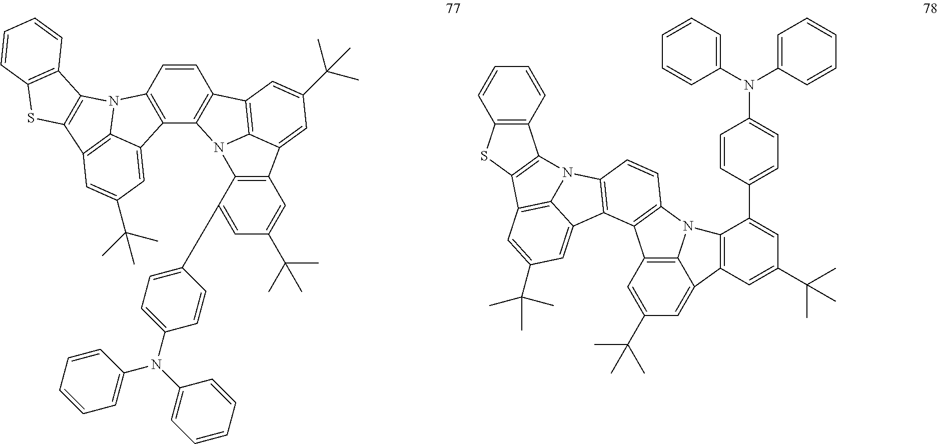 [SMILES: CC(C)(C)c1cc(-c2ccc(N(c3ccccc3)c3ccccc3)cc2)c2c(c1)c1cc(C(C)(C)C)cc3c4ccc5c(c6cc(C(C)(C)C)cc7c8sc9ccccc9c8n5c76)c4n2c13 ; CC(C)(C)c1cc(-c2ccc(N(c3ccccc3)c3ccccc3)cc2)c2c(c1)c1cc(C(C)(C)C)cc3c4c5c6cc(C(C)(C)C)cc7c8sc9ccccc9c8n(c5ccc4n2c13)c76]

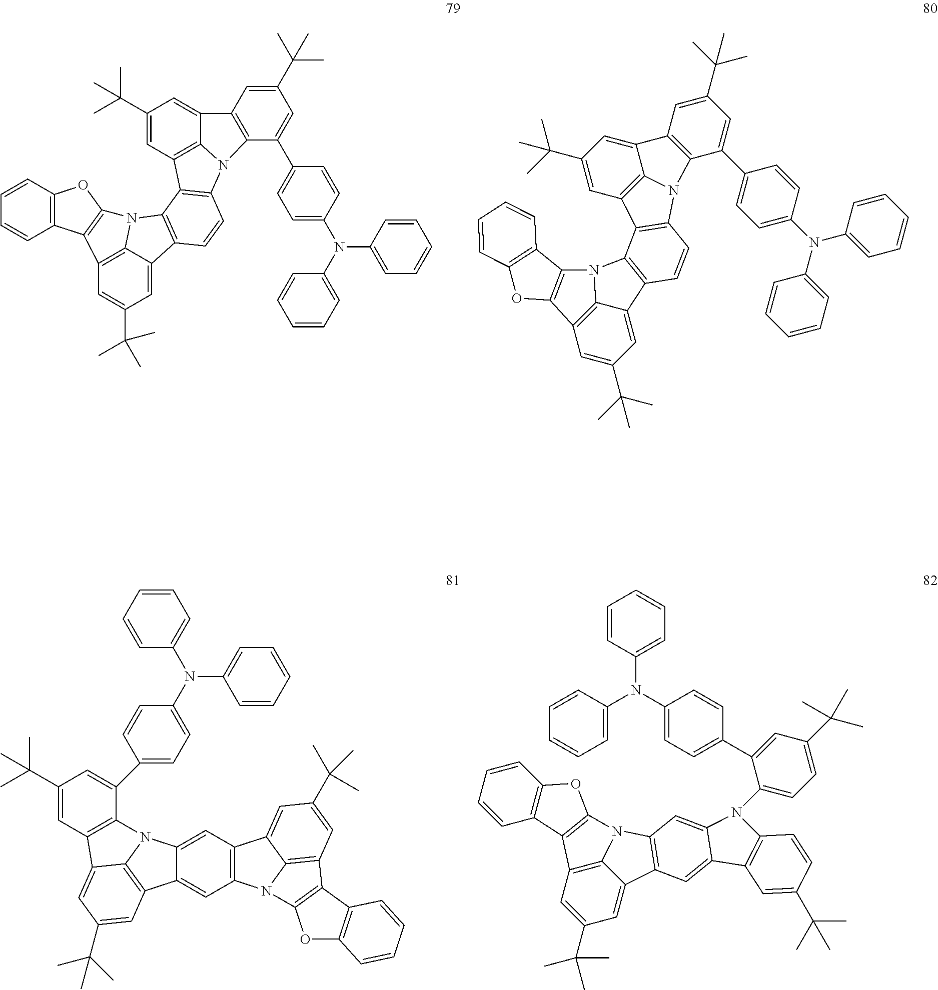 [SMILES: CC(C)(C)c1cc(-c2ccc(N(c3ccccc3)c3ccccc3)cc2)c2c(c1)c1cc(C(C)(C)C)cc3c4c(ccc5c6cc(C(C)(C)C)cc7c8c9ccccc9oc8n(c67)c54)n2c13 ; CC(C)(C)c1cc(-c2ccc(N(c3ccccc3)c3ccccc3)cc2)c2c(c1)c1cc(C(C)(C)C)cc3c4c(ccc5c6cc(C(C)(C)C)cc7c8oc9ccccc9c8n(c67)c54)n2c13 ; CC(C)(C)c1cc(-c2ccc(N(c3ccccc3)c3ccccc3)cc2)c2c(c1)c1cc(C(C)(C)C)cc3c4cc5c(cc4n2c31)c1cc(C(C)(C)C)cc2c3c4ccccc4oc3n5c12 ; CC(C)(C)c1ccc(-n2c3ccc(C(C)(C)C)cc3c3cc4c5cc(C(C)(C)C)cc6c7c8ccccc8oc7n(c4cc32)c56)c(-c2ccc(N(c3ccccc3)c3ccccc3)cc2)c1]

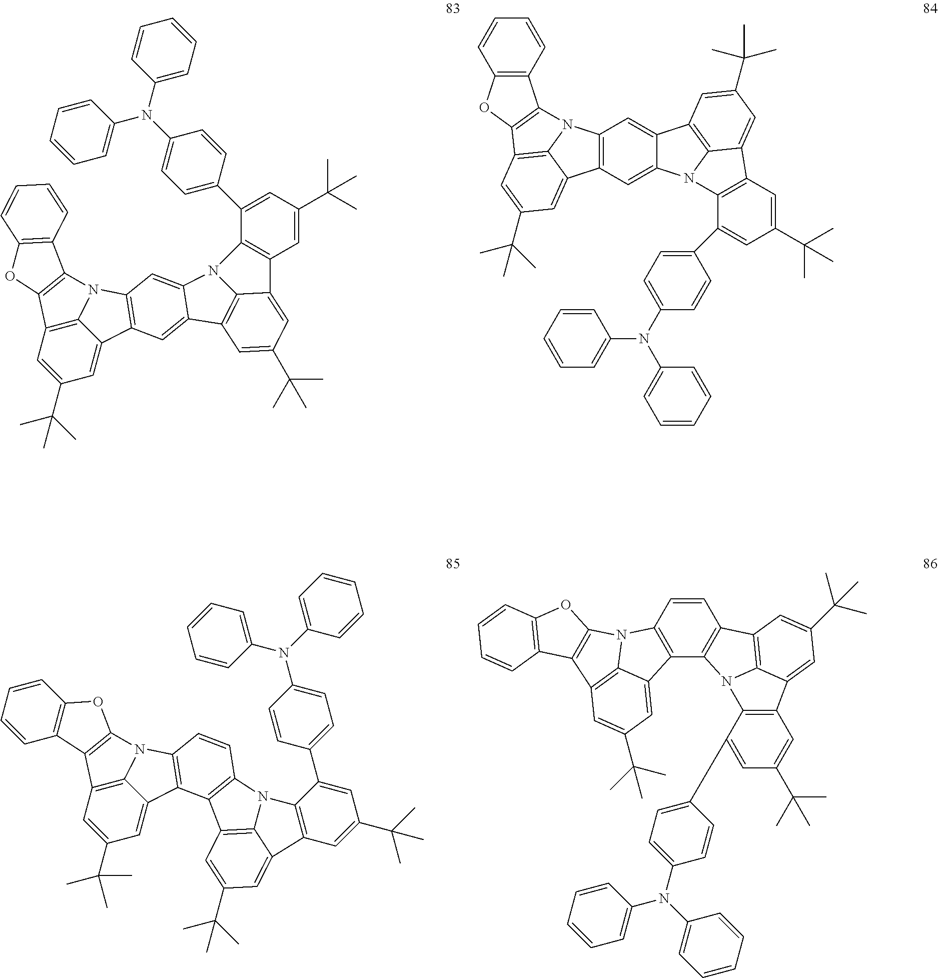 [SMILES: CC(C)(C)c1cc(-c2ccc(N(c3ccccc3)c3ccccc3)cc2)c2c(c1)c1cc(C(C)(C)C)cc3c4cc5c6cc(C(C)(C)C)cc7c8oc9ccccc9c8n(c5cc4n2c31)c67 ; CC(C)(C)c1cc(-c2ccc(N(c3ccccc3)c3ccccc3)cc2)c2c(c1)c1cc(C(C)(C)C)cc3c4cc5c(cc4n2c31)c1cc(C(C)(C)C)cc2c3oc4ccccc4c3n5c12 ; CC(C)(C)c1cc(-c2ccc(N(c3ccccc3)c3ccccc3)cc2)c2c(c1)c1cc(C(C)(C)C)cc3c4c5c6cc(C(C)(C)C)cc7c8c9ccccc9oc8n(c5ccc4n2c13)c67 ; CC(C)(C)c1cc(-c2ccc(N(c3ccccc3)c3ccccc3)cc2)c2c(c1)c1cc(C(C)(C)C)cc3c4ccc5c(c6cc(C(C)(C)C)cc7c8c9ccccc9oc8n5c76)c4n2c13]

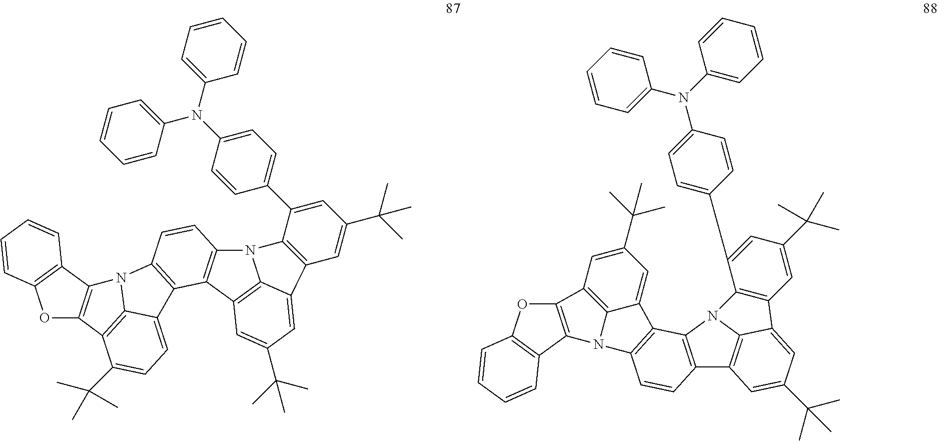 [SMILES: CC(C)(C)c1cc(-c2ccc(N(c3ccccc3)c3ccccc3)cc2)c2c(c1)c1cc(C(C)(C)C)cc3c4c5c6ccc(C(C)(C)C)c7c8oc9ccccc9c8n(c5ccc4n2c13)c67 ; CC(C)(C)c1cc(-c2ccc(N(c3ccccc3)c3ccccc3)cc2)c2c(c1)c1cc(C(C)(C)C)cc3c4ccc5c(c6cc(C(C)(C)C)cc7c8oc9ccccc9c8n5c76)c4n2c13]

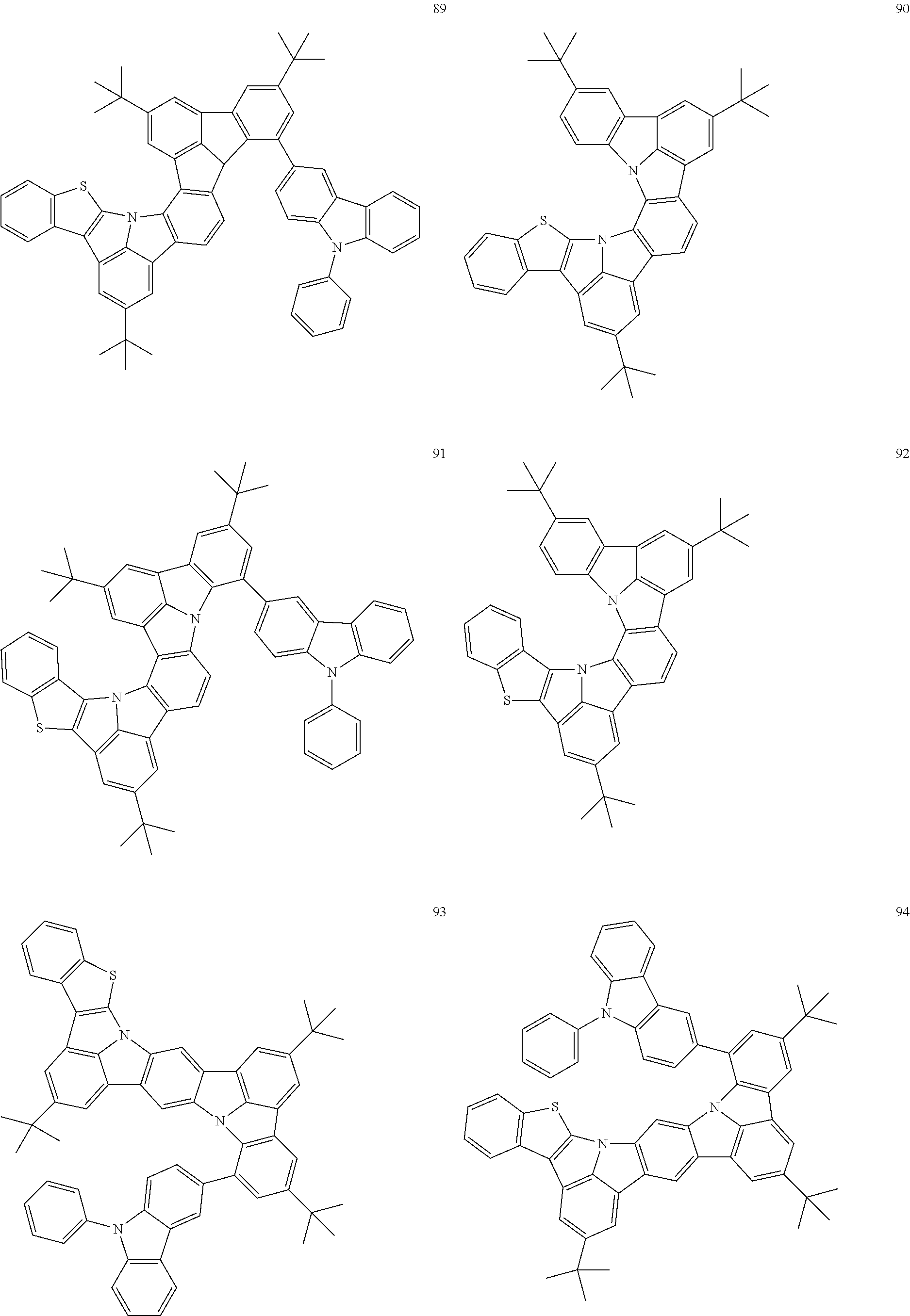

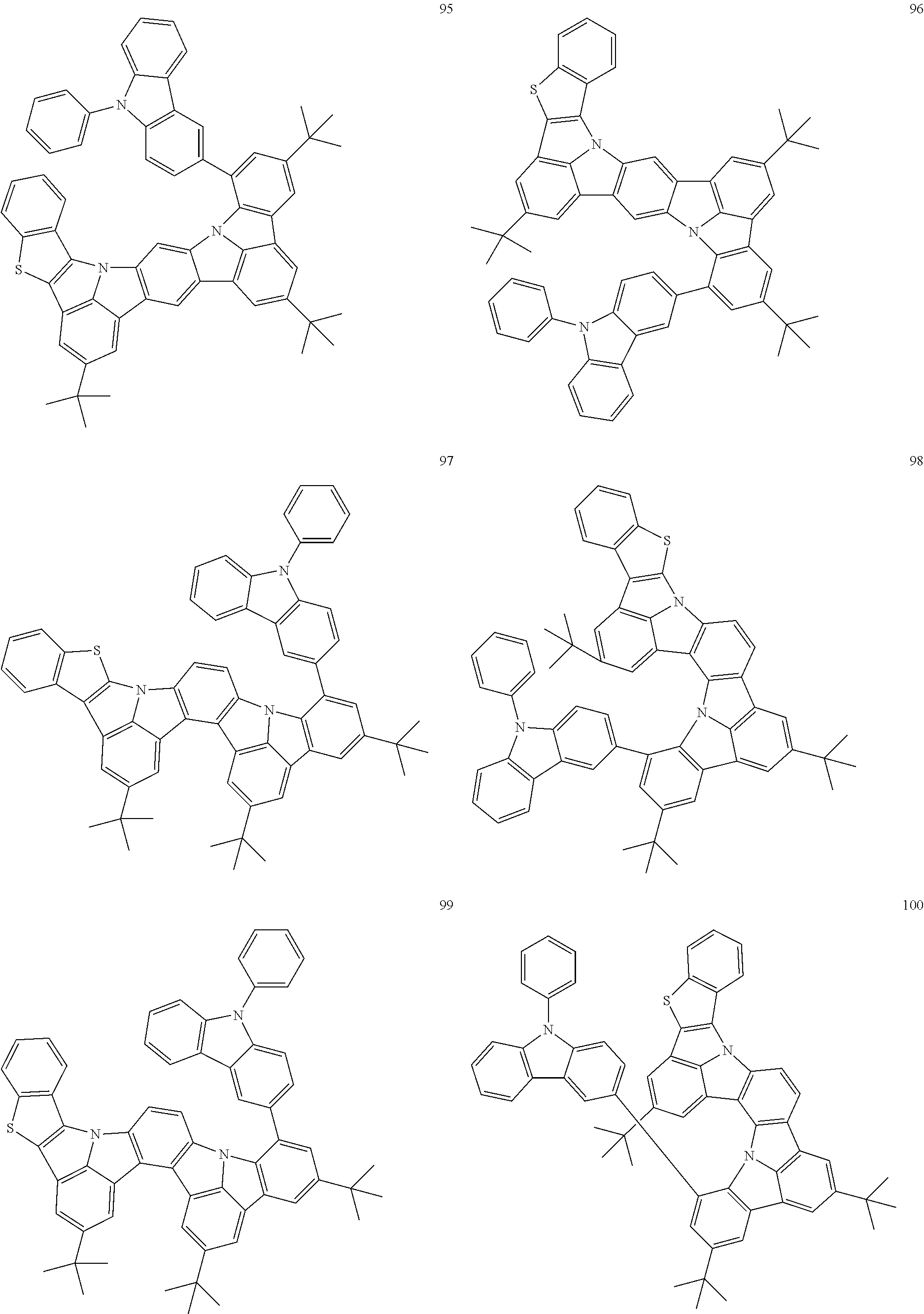

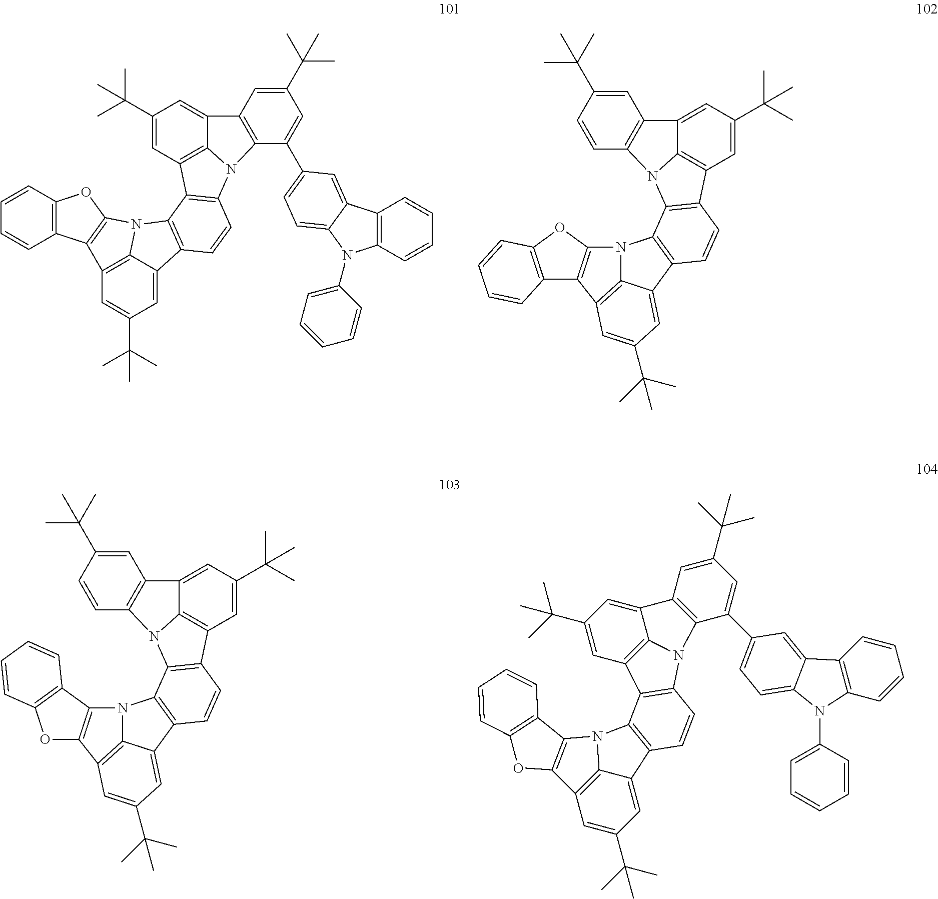 [SMILES: CC(C)(C)c1cc(-c2ccc3c(c2)c2ccccc2n3-c2ccccc2)c2c(c1)c1cc(C(C)(C)C)cc3c4c(ccc5c6cc(C(C)(C)C)cc7c8c9ccccc9oc8n(c67)c54)n2c13 ; CC(C)(C)c1ccc2c(c1)c1cc(C(C)(C)C)cc3c4ccc5c6cc(C(C)(C)C)cc7c8c9ccccc9oc8n(c67)c5c4n2c13 ; CC(C)(C)c1ccc2c(c1)c1cc(C(C)(C)C)cc3c4ccc5c6cc(C(C)(C)C)cc7c8oc9ccccc9c8n(c67)c5c4n2c13 ; CC(C)(C)c1cc(-c2ccc3c(c2)c2ccccc2n3-c2ccccc2)c2c(c1)c1cc(C(C)(C)C)cc3c4c(ccc5c6cc(C(C)(C)C)cc7c8oc9ccccc9c8n(c67)c54)n2c13]

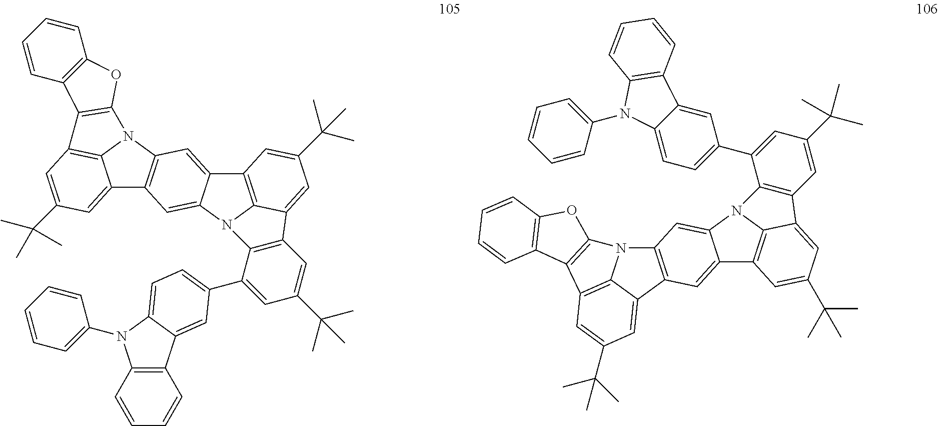 [SMILES: CC(C)(C)c1cc(-c2ccc3c(c2)c2ccccc2n3-c2ccccc2)c2c(c1)c1cc(C(C)(C)C)cc3c4cc5c(cc4n2c31)c1cc(C(C)(C)C)cc2c3c4ccccc4oc3n5c12 ; CC(C)(C)c1cc(-c2ccc3c(c2)c2ccccc2n3-c2ccccc2)c2c(c1)c1cc(C(C)(C)C)cc3c4cc5c6cc(C(C)(C)C)cc7c8c9ccccc9oc8n(c5cc4n2c31)c67]

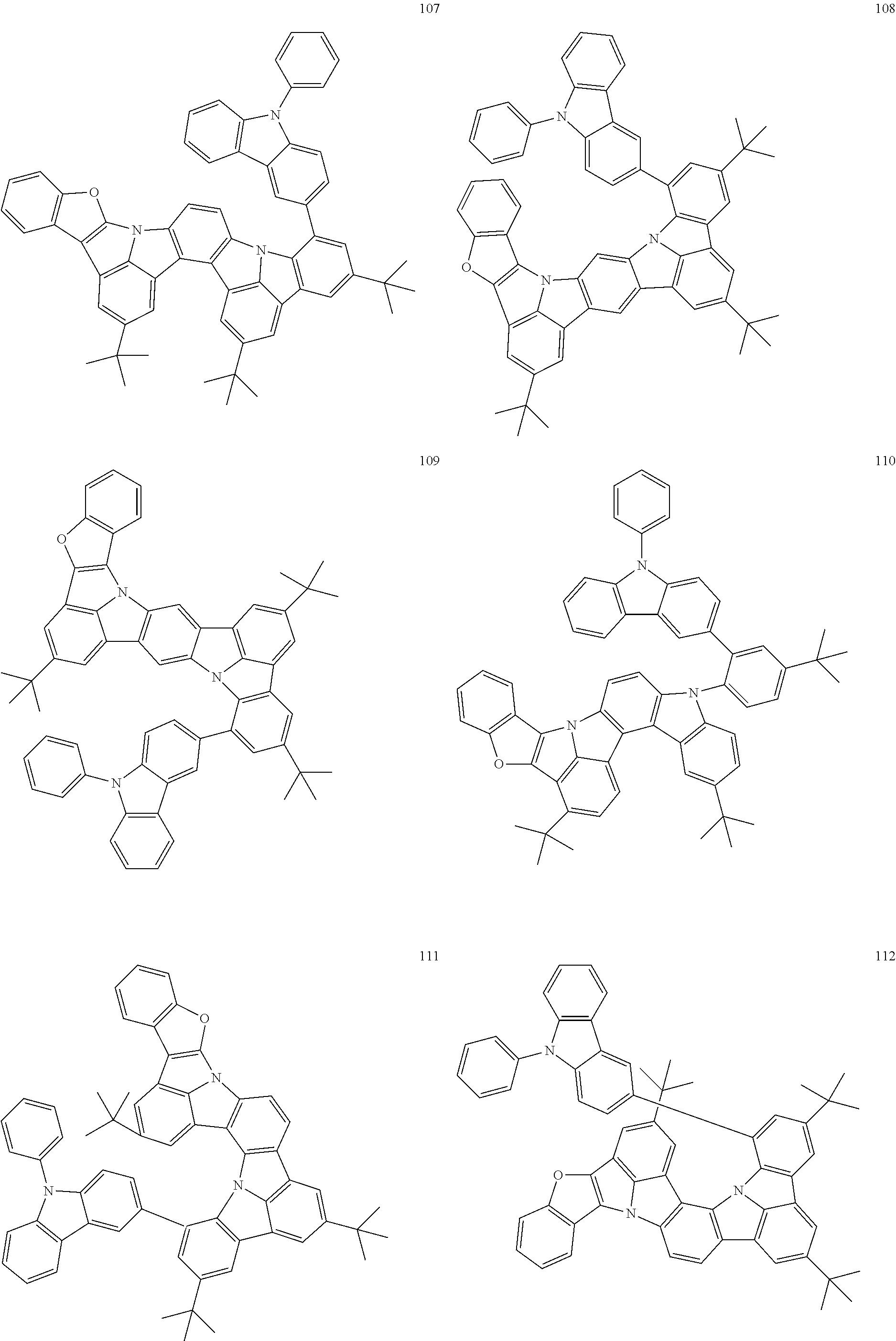

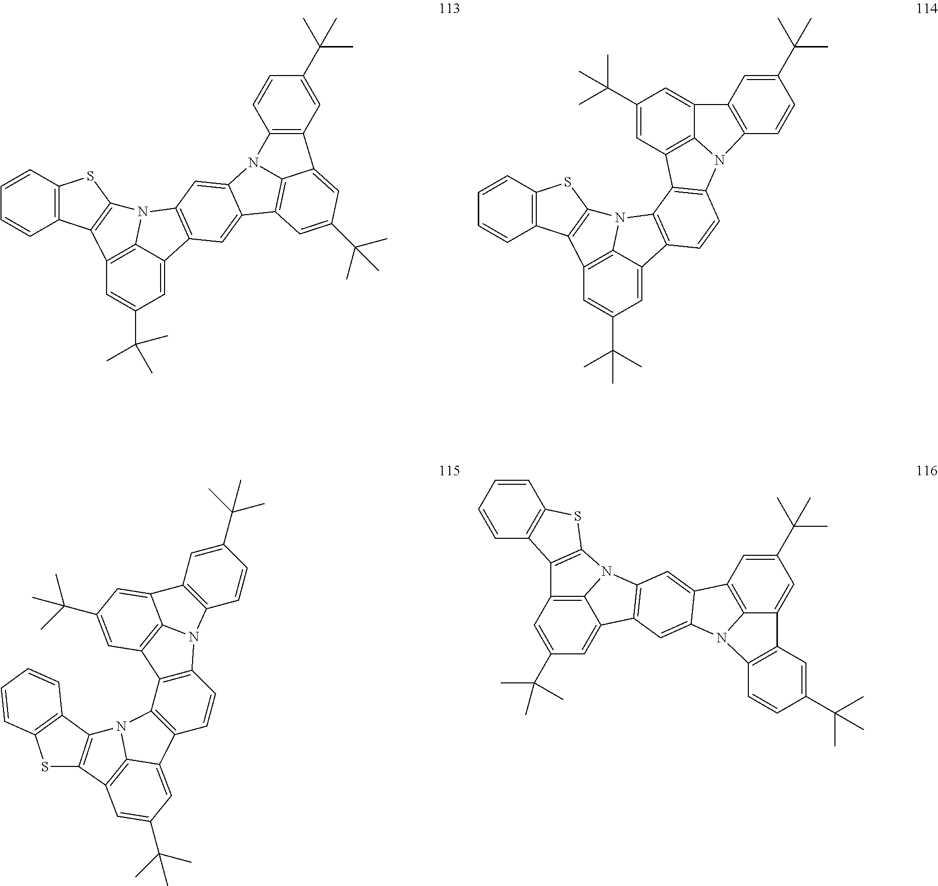 [SMILES: CC(C)(C)c1ccc2c(c1)c1cc(C(C)(C)C)cc3c4cc5c6cc(C(C)(C)C)cc7c8c9ccccc9sc8n(c5cc4n2c13)c67 ; CC(C)(C)c1ccc2c(c1)c1cc(C(C)(C)C)cc3c4c(ccc5c6cc(C(C)(C)C)cc7c8c9ccccc9sc8n(c67)c54)n2c13 ; CC(C)(C)c1ccc2c(c1)c1cc(C(C)(C)C)cc3c4c(ccc5c6cc(C(C)(C)C)cc7c8sc9ccccc9c8n(c67)c54)n2c13 ; CC(C)(C)c1ccc2c(c1)c1cc(C(C)(C)C)cc3c4cc5c(cc4n2c13)c1cc(C(C)(C)C)cc2c3c4ccccc4sc3n5c12]

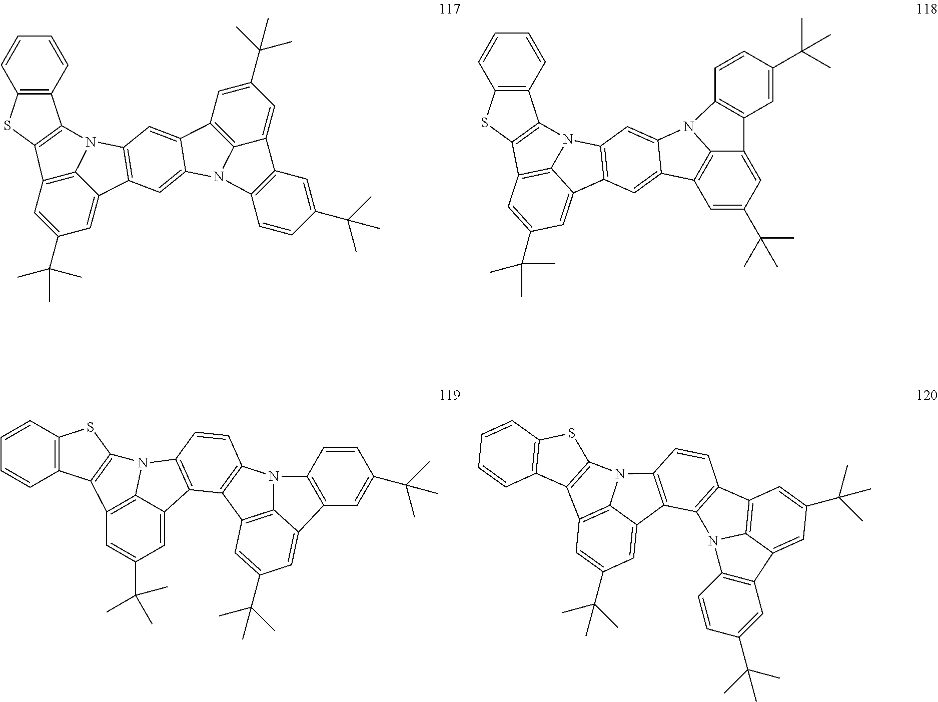 [SMILES: CC(C)(C)c1ccc2c(c1)c1cc(C(C)(C)C)cc3c4cc5c(cc4n2c13)c1cc(C(C)(C)C)cc2c3sc4ccccc4c3n5c12 ; CC(C)(C)c1ccc2c(c1)c1cc(C(C)(C)C)cc3c4cc5c6cc(C(C)(C)C)cc7c8sc9ccccc9c8n(c5cc4n2c13)c67 ; CC(C)(C)c1ccc2c(c1)c1cc(C(C)(C)C)cc3c4c5c6cc(C(C)(C)C)cc7c8c9ccccc9sc8n(c5ccc4n2c13)c67 ; CC(C)(C)c1ccc2c(c1)c1cc(C(C)(C)C)cc3c4ccc5c(c6cc(C(C)(C)C)cc7c8c9ccccc9sc8n5c76)c4n2c13]

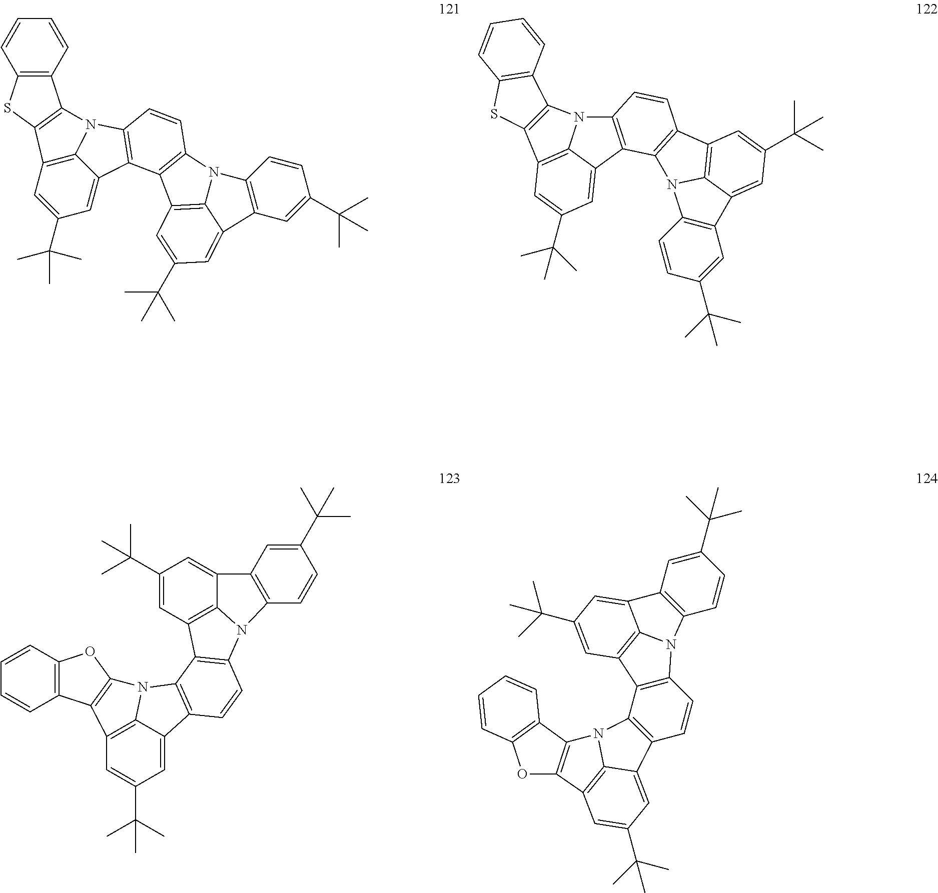 [SMILES: CC(C)(C)c1ccc2c(c1)c1cc(C(C)(C)C)cc3c4c5c6cc(C(C)(C)C)cc7c8sc9ccccc9c8n(c5ccc4n2c13)c76 ; CC(C)(C)c1ccc2c(c1)c1cc(C(C)(C)C)cc3c4ccc5c(c6cc(C(C)(C)C)cc7c8sc9ccccc9c8n5c76)c4n2c13 ; CC(C)(C)c1ccc2c(c1)c1cc(C(C)(C)C)cc3c4c(ccc5c6cc(C(C)(C)C)cc7c8c9ccccc9oc8n(c67)c54)n2c13 ; CC(C)(C)c1ccc2c(c1)c1cc(C(C)(C)C)cc3c4c(ccc5c6cc(C(C)(C)C)cc7c8oc9ccccc9c8n(c67)c54)n2c13]

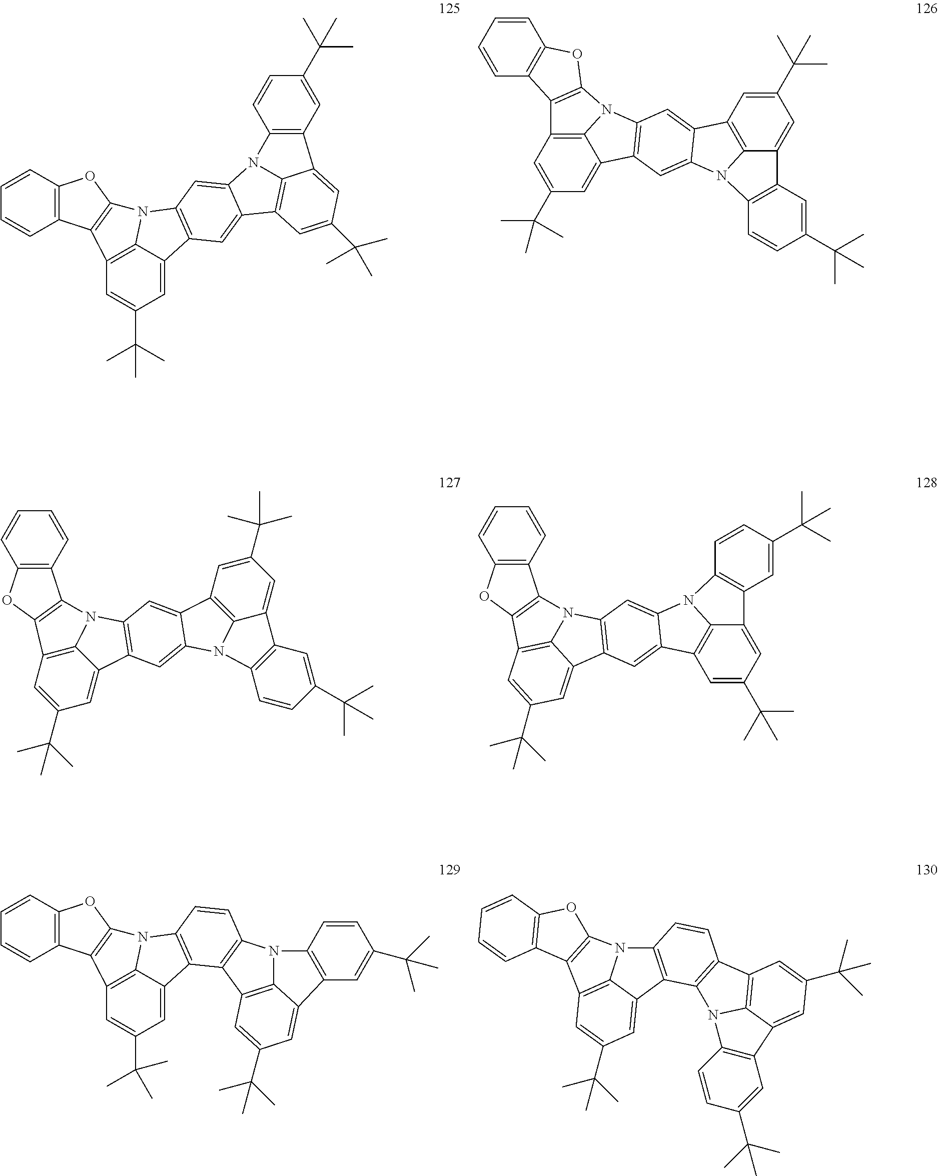

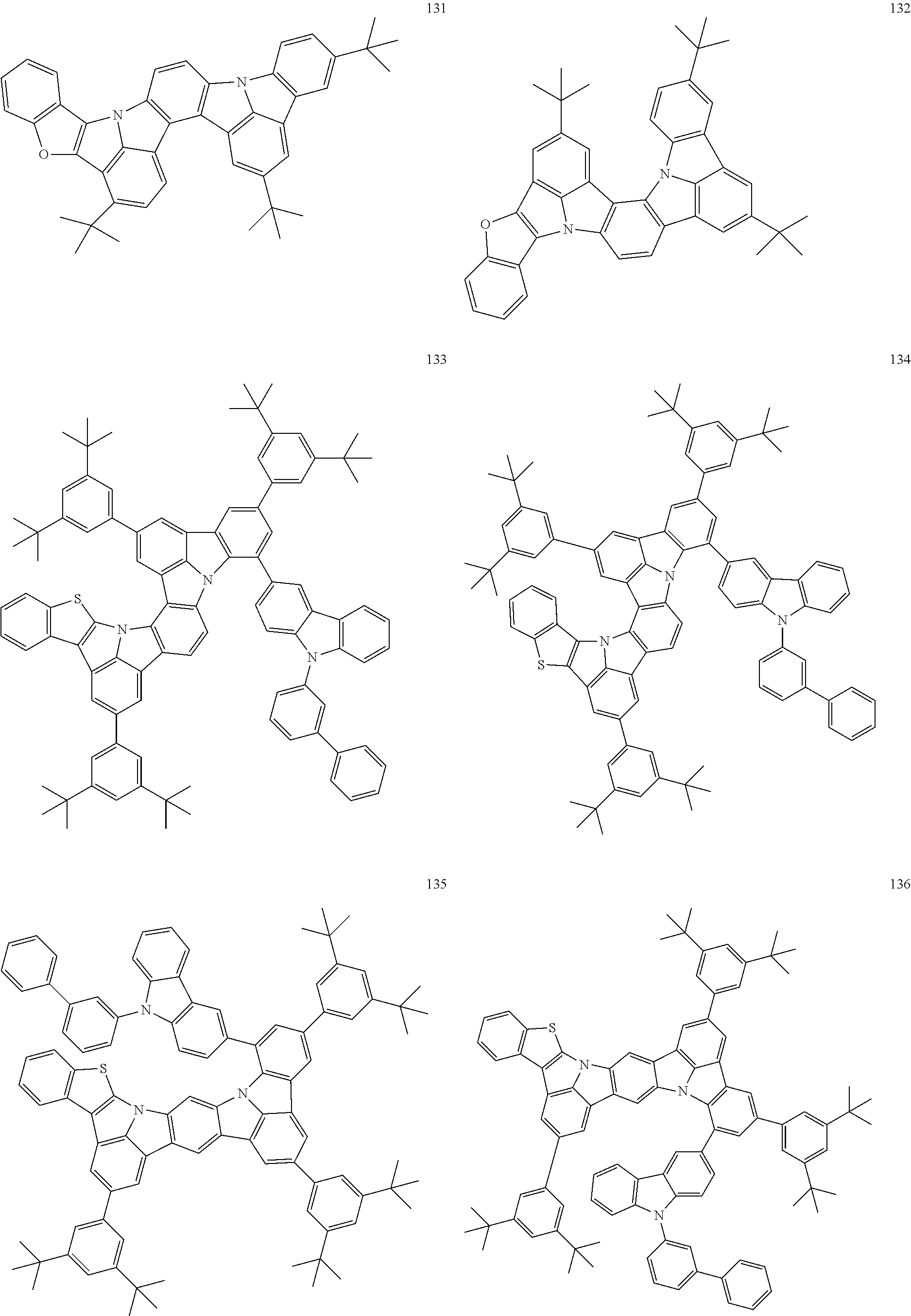

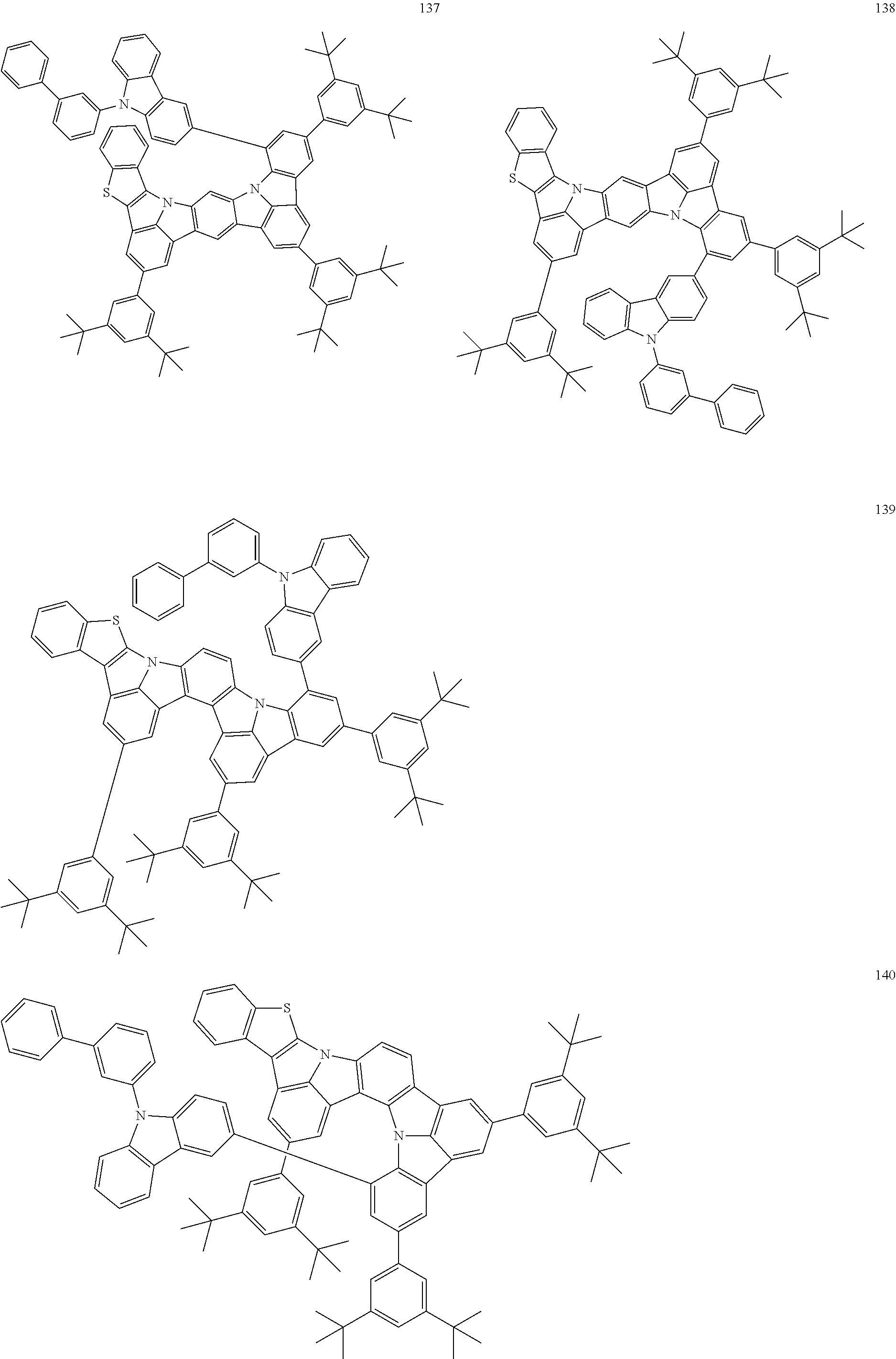

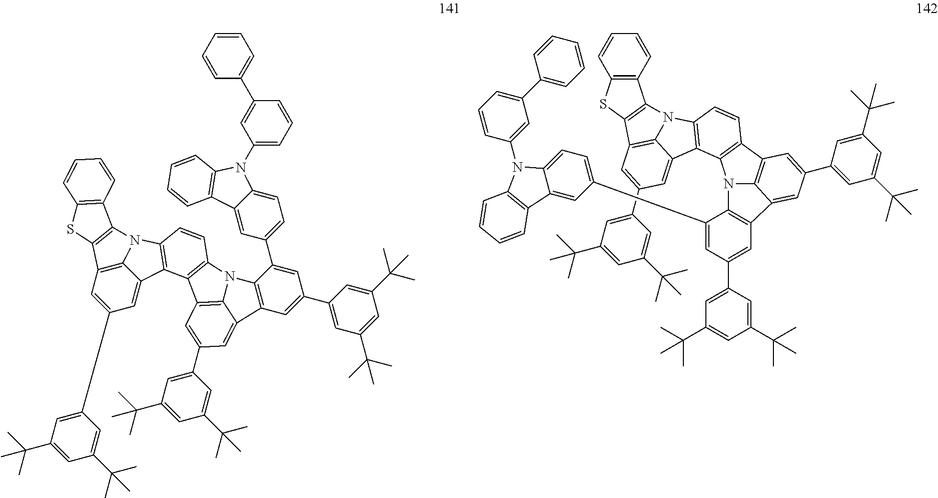 [SMILES: CC(C)(C)c1cc(-c2cc(-c3ccc4c(c3)c3ccccc3n4-c3cccc(-c4ccccc4)c3)c3c(c2)c2cc(-c4cc(C(C)(C)C)cc(C(C)(C)C)c4)cc4c5c6c7cc(-c8cc(C(C)(C)C)cc(C(C)(C)C)c8)cc8c9sc%10ccccc%10c9n(c6ccc5n3c24)c87)cc(C(C)(C)C)c1 ; CC(C)(C)c1cc(-c2cc(-c3ccc4c(c3)c3ccccc3n4-c3cccc(-c4ccccc4)c3)c3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9sc%10ccccc%10c9n6c87)c5n3c24)cc(C(C)(C)C)c1]

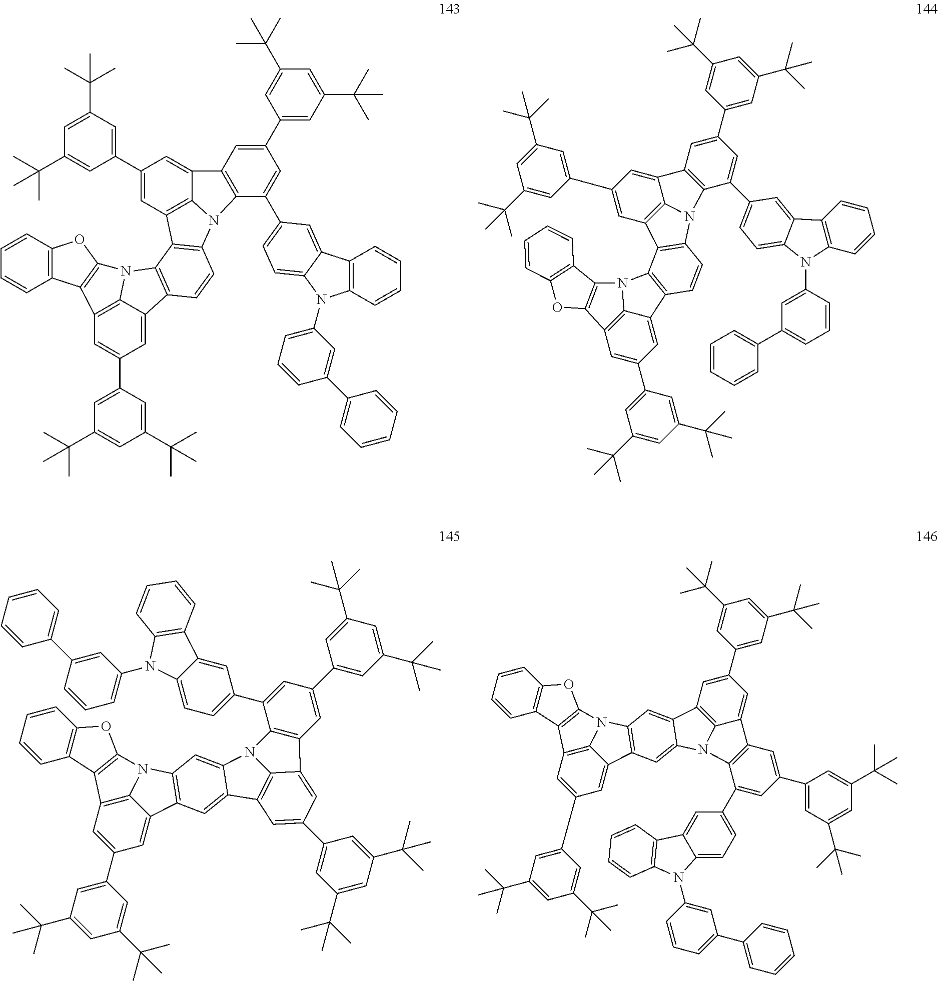

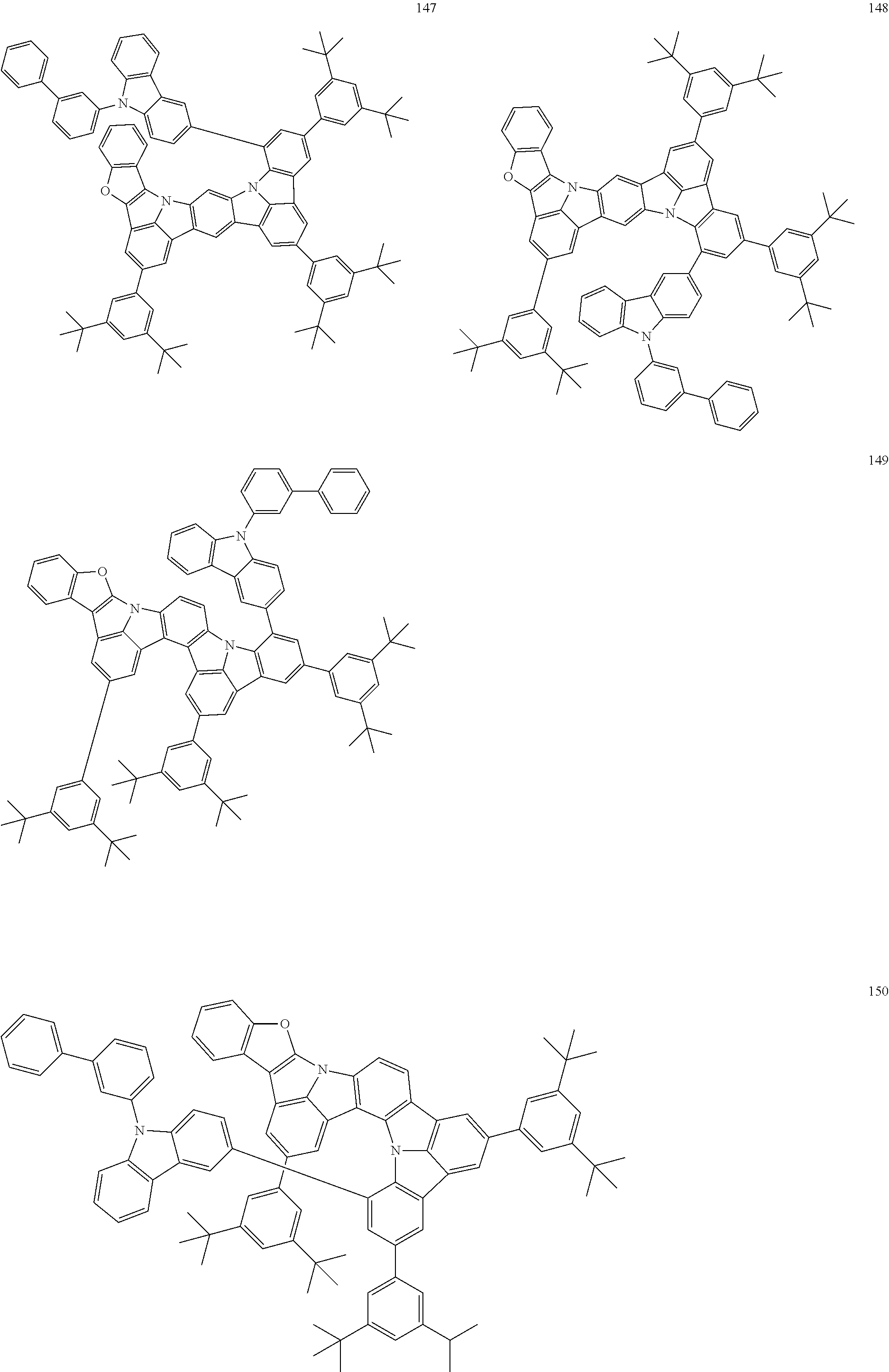

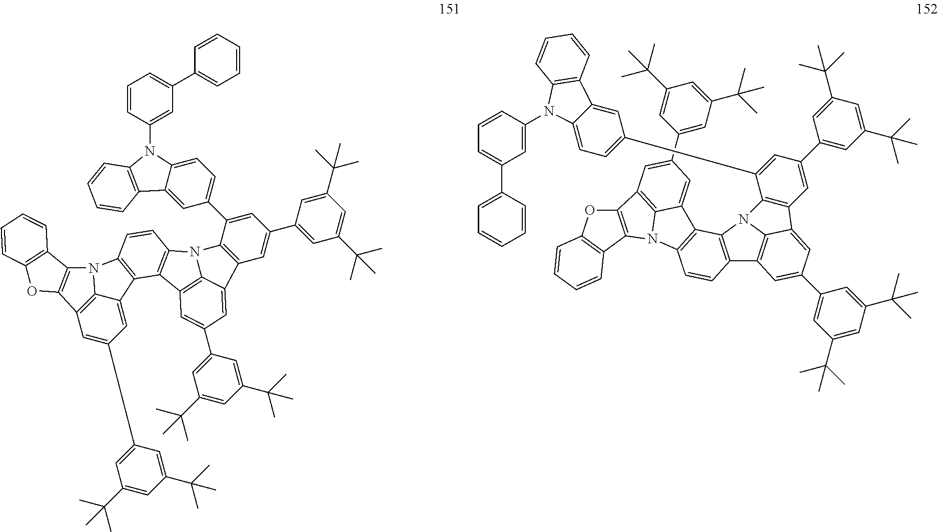 [SMILES: CC(C)(C)c1cc(-c2cc(-c3ccc4c(c3)c3ccccc3n4-c3cccc(-c4ccccc4)c3)c3c(c2)c2cc(-c4cc(C(C)(C)C)cc(C(C)(C)C)c4)cc4c5c6c7cc(-c8cc(C(C)(C)C)cc(C(C)(C)C)c8)cc8c9oc%10ccccc%10c9n(c6ccc5n3c24)c87)cc(C(C)(C)C)c1 ; CC(C)(C)c1cc(-c2cc(-c3ccc4c(c3)c3ccccc3n4-c3cccc(-c4ccccc4)c3)c3c(c2)c2cc(-c4cc(C(C)(C)C)cc(C(C)(C)C)c4)cc4c5ccc6c(c7cc(-c8cc(C(C)(C)C)cc(C(C)(C)C)c8)cc8c9oc%10ccccc%10c9n6c87)c5n3c24)cc(C(C)(C)C)c1]

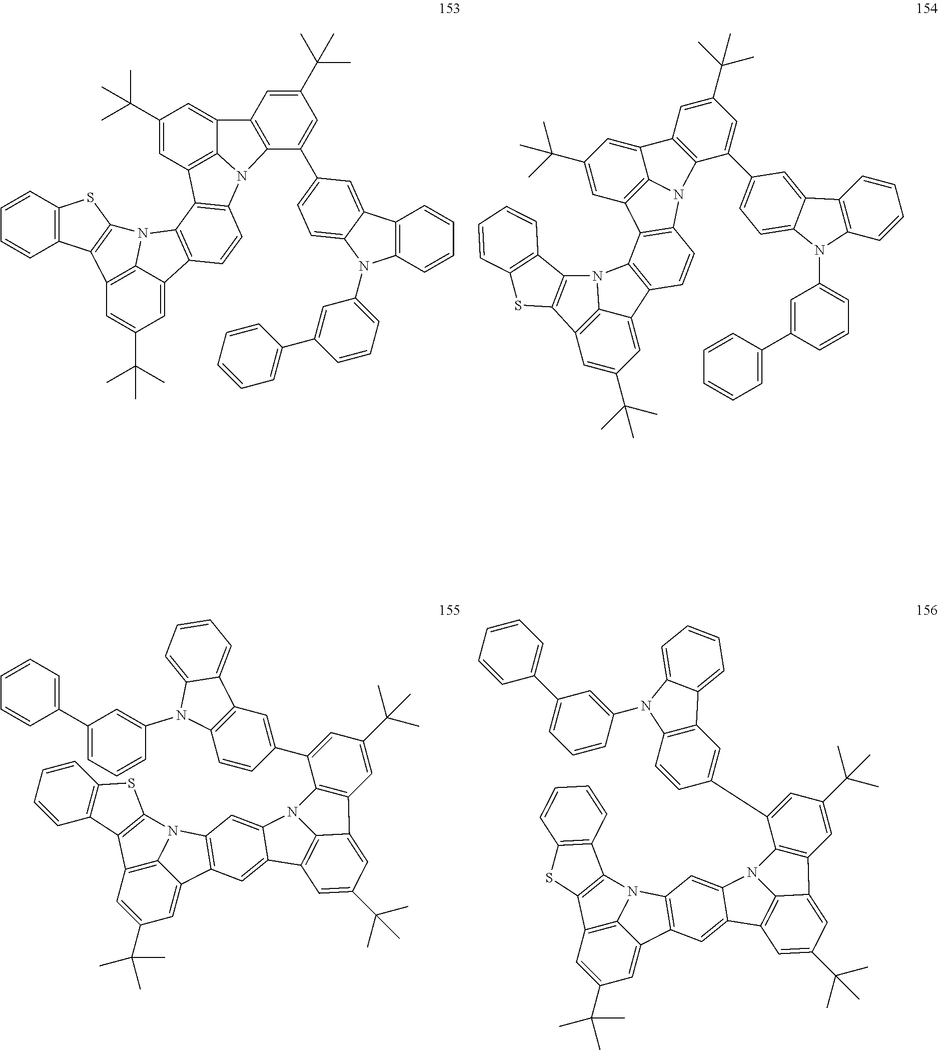

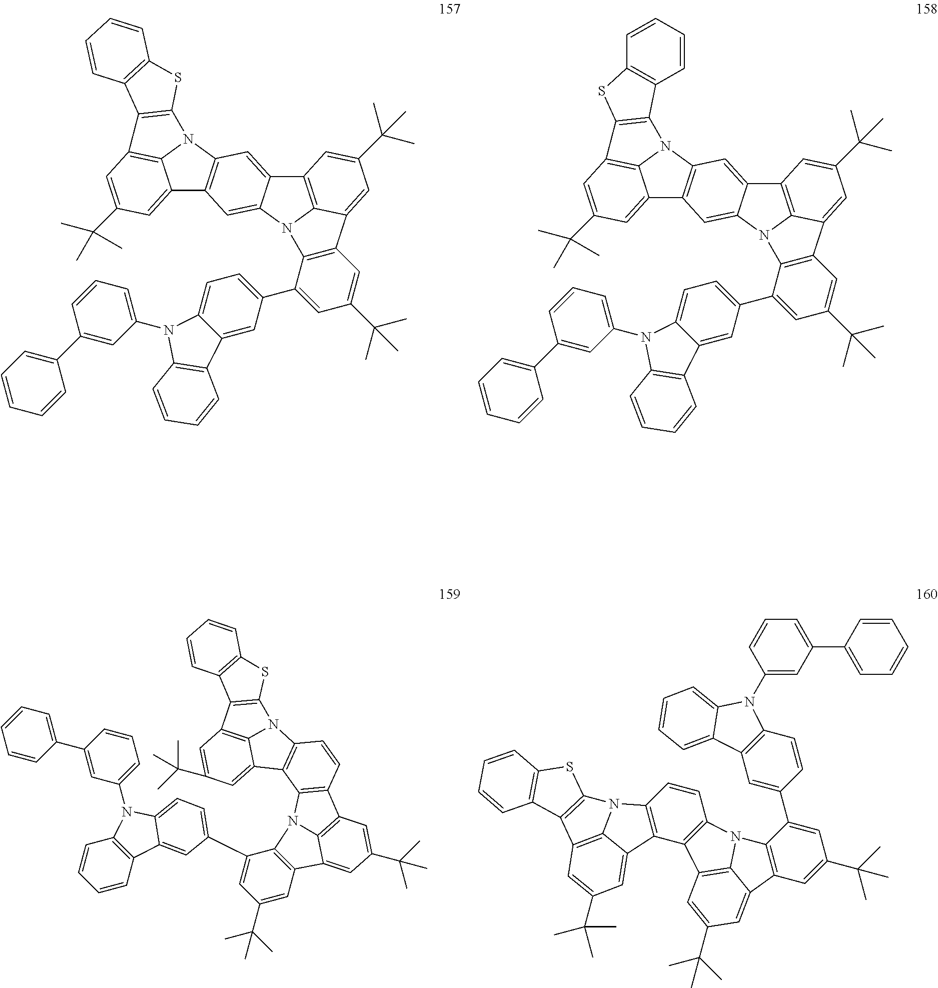

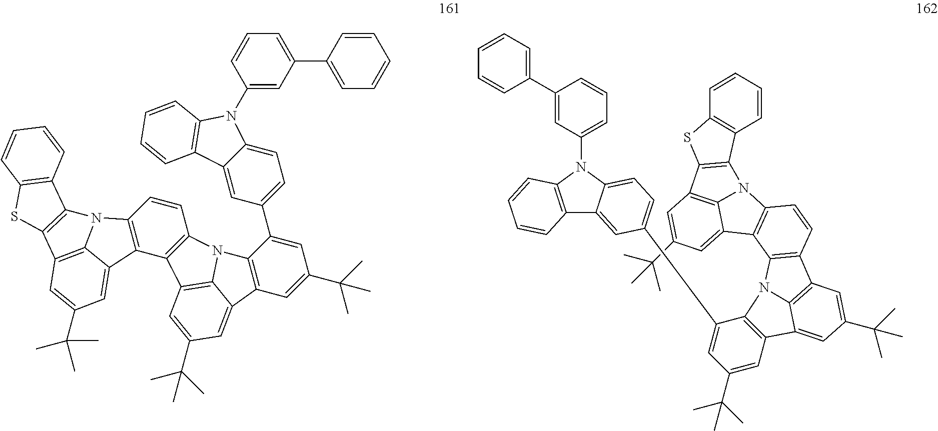 [SMILES: CC(C)(C)c1cc(-c2ccc3c(c2)c2ccccc2n3-c2cccc(-c3ccccc3)c2)c2c(c1)c1cc(C(C)(C)C)cc3c4c5c6cc(C(C)(C)C)cc7c8sc9ccccc9c8n(c5ccc4n2c13)c76 ; CC(C)(C)c1cc(-c2ccc3c(c2)c2ccccc2n3-c2cccc(-c3ccccc3)c2)c2c(c1)c1cc(C(C)(C)C)cc3c4ccc5c(c6cc(C(C)(C)C)cc7c8sc9ccccc9c8n5c76)c4n2c13]

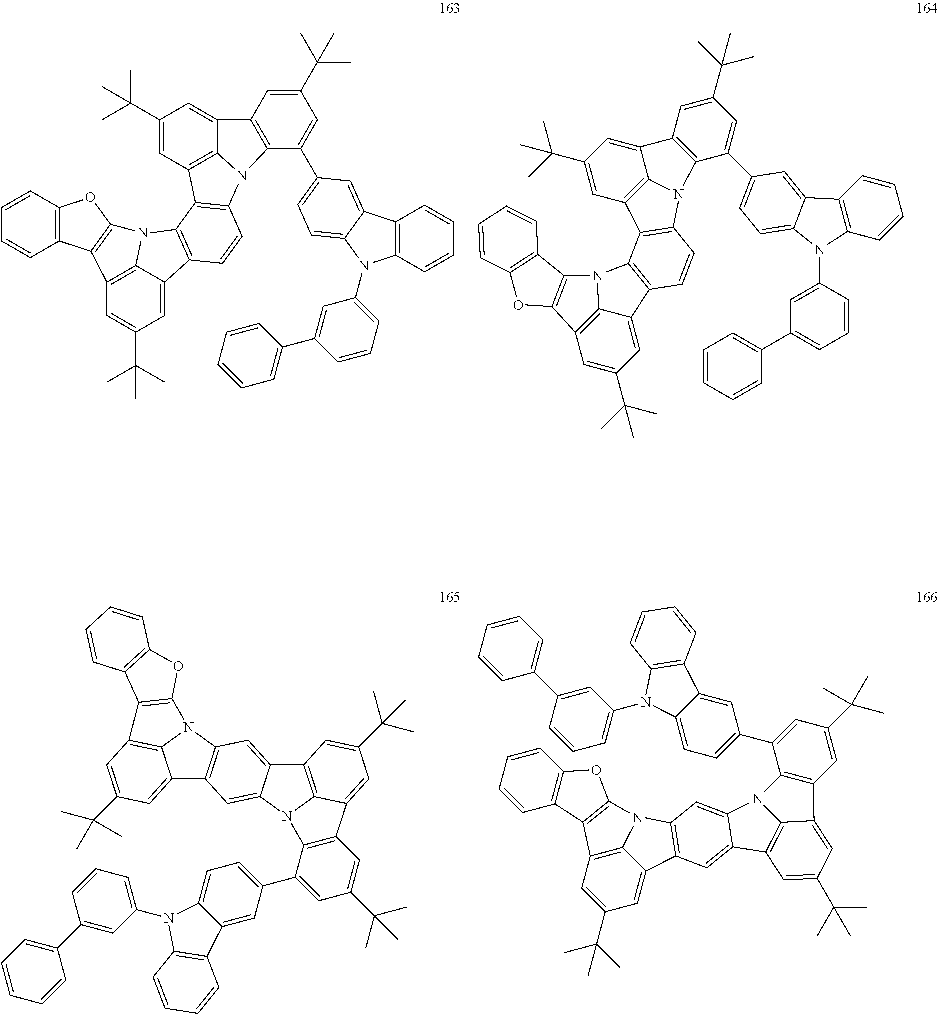

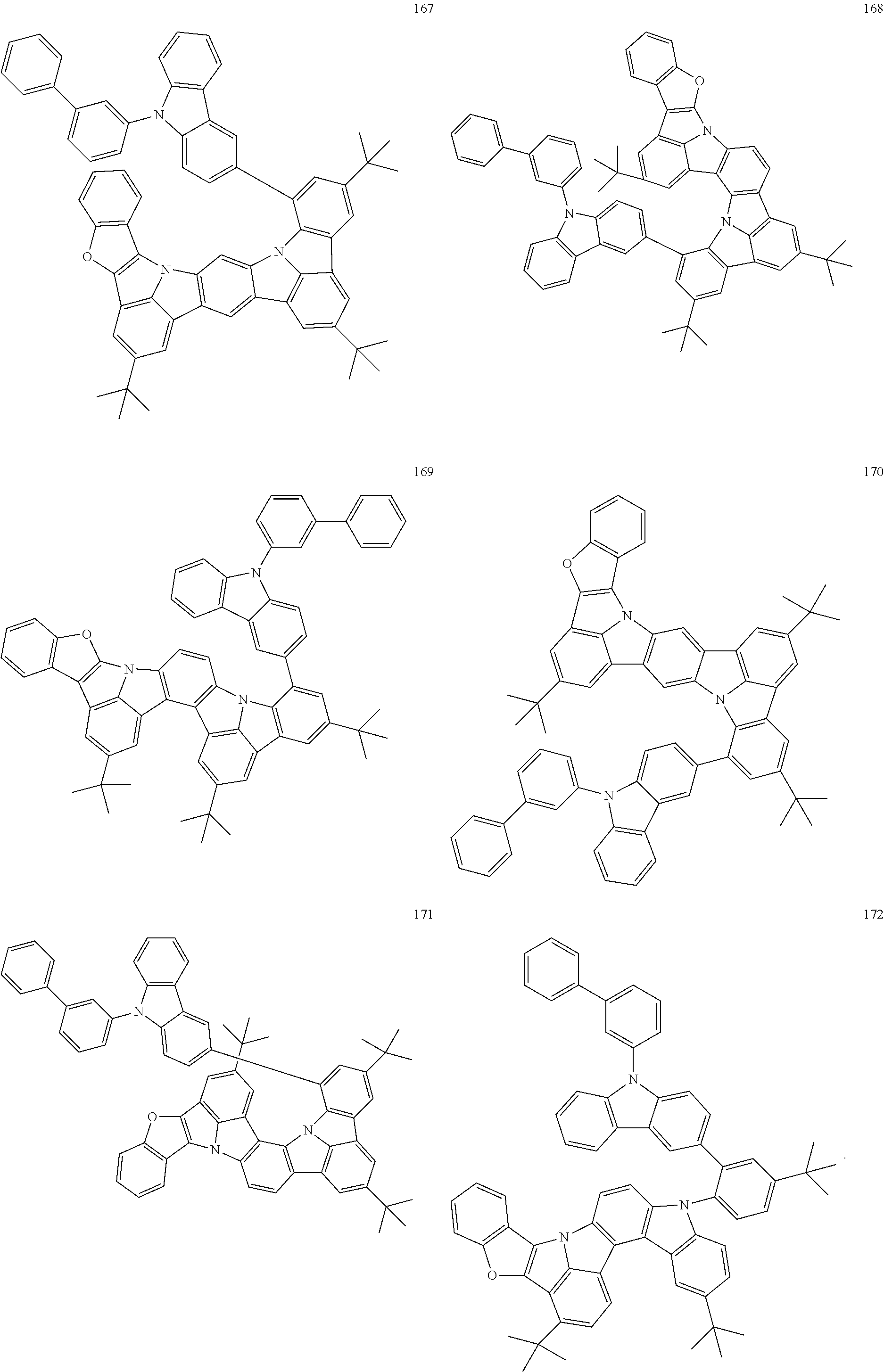

12. A light-emitting device comprising:
a first electrode;
a second electrode; and
an interlayer arranged between the first electrode and the second electrode,
wherein the interlayer comprises an emission layer,
wherein the interlayer comprises at least one of the condensed cyclic compound of claim 1.

13. The light-emitting device of claim 12, wherein the emission layer comprises the at least one of the condensed cyclic compound.

14. The light-emitting device of claim 12, wherein an emission peak wavelength of light emitted from the emission layer is about 440 nanometers to about 470 nanometers.

15. The light-emitting device of claim 12, wherein the at least one of the condensed cyclic compound included in the emission layer is an emitter.

16. The light-emitting device of claim 12, wherein the emission layer further comprises a sensitizer, and the sensitizer is different from the condensed cyclic compound.

17. The light-emitting device of claim 16, wherein the sensitizer is an organometallic compound, a delayed fluorescence material, a prompt fluorescence material, or any combination thereof.

18. The light-emitting device of claim 17, wherein the organometallic compound comprises a transition metal and a tetradentate ligand bonded to the transition metal,
wherein the transition metal is platinum or palladium, and
wherein the tetradentate ligand comprises a carbene moiety bonded to the transition metal.

19. The light-emitting device of claim 12, wherein the at least one of the condensed cyclic compound included in the emission layer is a sensitizer.

20. An electronic apparatus, comprising the light-emitting device of claim 12.
